(19) United States
(12) Patent Application Publication
(10) Pub. No.: US 2025/0194416 A1
(43) Pub. Date: Jun. 12, 2025

(54) ORGANIC COMPOUND, AND ELECTRONIC COMPONENT AND ELECTRONIC APPARATUS USING SAME

(71) Applicant: SHAANXI LIGHTE OPTOELECTRONICS MATERIAL CO., LTD., Xi'an, Shaanxi (CN)

(72) Inventors: Meiling ZHANG, Xi'an, Shaanxi (CN); Youngkook KIM, Xi'an, Shaanxi (CN); Yun LIU, Xi'an, Shaanxi (CN); Yingwen LI, Xi'an, Shaanxi (CN)

(21) Appl. No.: 18/866,796

(22) PCT Filed: Sep. 8, 2023
(86) PCT No.: PCT/CN2023/117883

§ 371 Date: Nov. 18, 2024

(30) Foreign Application Priority Data
Feb. 28, 2023 (CN) 202310179451.5

(51) Int. Cl. H10K 85/60; C07C 211/54; C07C 211/58; C07C 211/61; C07D 209/86; C07D 209/88; C07D 307/91; C07D 333/76; C07F 7/08; H10K 50/15

(52) U.S. Cl. CPC H10K 85/633; C07C 211/54; C07C 211/58; C07C 211/61; C07D 209/86; C07D 209/88; C07D 307/91; C07D 333/76; C07F 7/081; H10K 85/636; C07B 2200/05; C07C 2603/18; C07C 2603/26; C07C 2603/74; C07C 2603/97; H10K 50/15; H10K 85/615; H10K 85/626; H10K 85/6572; H10K 85/6574; H10K 85/6576

(57) ABSTRACT

The present invention relates to an organic compound, and an electronic element and an electronic apparatus using same. The organic compound has a structure represented by a formula (I). The organic compound is applied to an organic electroluminescent device, and can significantly improve the performance of the device.

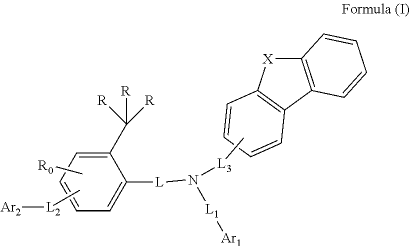

### CROSS-REFERENCE TO RELATED APPLICATIONS

The present disclosure claims the priority of Chinese patent application No. 2023101794515 filed on Feb. 28, 2023, which is incorporated herein by reference in its entirety as a part of the present disclosure.

### FIELD OF THE INVENTION

The present disclosure relates to the technical field of organic electroluminescence, and specifically to an organic compound and an electronic element and an electronic apparatus using the same.

### BACKGROUND OF THE INVENTION

With the development of electronic technology and the progress of material science, the application range of electronic elements used to realize electroluminescence or photoelectric conversion is more and more extensive. This type of electronic elements usually comprise a cathode and an anode disposed opposite to each other, and a functional layer disposed between the cathode and the anode. The functional layer is composed of multiple organic or inorganic film layers, and generally comprises an energy conversion layer, a hole transport layer located between the energy conversion layer and the anode, and an electron transport layer located between the energy conversion layer and the cathode.

Taking organic electroluminescent devices as an example, they generally comprise an anode, a hole transport layer, an organic light-emitting layer, an electron transport layer, and a cathode sequentially stacked. When a voltage is applied to the anode and cathode, an electric field is generated between the two electrodes. Under the influence of the electric field, electrons on the cathode side move towards the electroluminescent layer, and holes on the anode side also move towards the light-emitting layer. Electrons and holes combine in the electroluminescent layer to form excitons, which are in an excited state and release energy outward, thereby causing the electroluminescent layer to emit light externally.

In the prior art, hole transport materials that can be used in an organic electroluminescent device are disclosed in WO2016087017A1, KR1020110110508A, CN111094234A, etc. However, it is still necessary to continue developing new hole transport materials to further improve the performance of electronic elements.

### SUMMARY OF THE INVENTION

The objective of the present disclosure is to provide an organic compound and an electronic element and an electronic apparatus using the organic compound, and the application of the organic compound in an organic electroluminescent device can improve the performance of the device.

A first aspect of the present disclosure provides an organic compound having a structure shown in a Formula I:

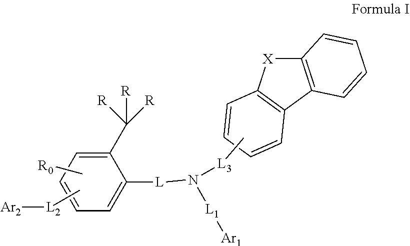

wherein, X is selected from O, S, C(R₁R₂), or N(R₃);
R₁, R₂, and R₃ are identical or different, and are each independently selected from an alkyl having 1 to 5 carbon atoms, a deuteroalkyl having 1 to 5 carbon atoms, a deuteroaryl having 6 to 20 carbon atoms, an aryl having 6 to 20 carbon atoms, or a heteroaryl having 3 to 20 carbon atoms; or R₁ and R₂ are interconnected together with the atoms to which they are commonly connected to form a 5- to 13-membered saturated or unsaturated ring;
each R is independently selected from a deuterium, an alkyl having 1 to 5 carbon atoms, or a deuteroalkyl having 1 to 5 carbon atoms;
R₀ is selected from a hydrogen, an alkyl having 1 to 5 carbon atoms, a deuteroalkyl having 1 to 5 carbon atoms, a haloalkyl having 1 to 5 carbon atoms, a deuteroaryl having 6 to 18 carbon atoms, an aryl having 6 to 18 carbon atoms, or a heteroaryl having 3 to 18 carbon atoms;
L is selected from a single bond, a substituted or unsubstituted arylene having 6 to 18 carbon atoms, or a substituted or unsubstituted heteroarylene having 3 to 18 carbon atoms;
the substituent(s) of L are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 10 carbon atoms, an aryl having 6 to 12 carbon atoms, or a heteroaryl having 3 to 12 carbon atoms;
Ar₂ is selected from a substituted or unsubstituted aryl having 6 to 18 carbon atoms, a substituted or unsubstituted dibenzofuranyl, or a substituted or unsubstituted dibenzothienyl;
the substituent(s) of Ar₂ are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 10 carbon atoms, an aryl having 6 to 12 carbon atoms, a dibenzofuranyl, or a dibenzothienyl;
L₂ is selected from a single bond, a substituted or unsubstituted arylene having 6 to 12 carbon atoms, or a substituted or unsubstituted heteroarylene having 3 to 18 carbon atoms;
the substituent(s) of L₂ are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 6 carbon atoms, or a phenyl;
Ar₁ is selected from a substituted or unsubstituted aryl having 6 to 30 carbon atoms, or a substituted or unsubstituted heteroaryl having 3 to 30 carbon atoms;
the substituent(s) of Ar₁ are each independently selected from a deuterium, a halogen group, a cyano, an alkyl having 1 to 10 carbon atoms, a deuteroalkyl having 1 to 10 carbon atoms, a trialkylsilyl having 3 to 12 carbon atoms, a haloalkyl having 1 to 10 carbon atoms, a cycloalkyl having 3 to 12 carbon atoms, a deuteroaryl having 6 to 20 carbon atoms, an aryl having 6 to 20 carbon atoms, or a heteroaryl having 3 to 20 carbon atoms; optionally, any two adjacent substituents of Ar₁ form a saturated or unsaturated 3- to 15-membered ring;
L₁ and L₃ are identical or different, and are each independently selected from a single bond, a substituted or unsubstituted arylene having 6 to 18 carbon atoms, or a substituted or unsubstituted heteroarylene having 3 to 18 carbon atoms;
the substituent(s) of L₁ and L₃ are each independently selected from a deuterium, a halogen group, a cyano, a trialkylsilyl having 3 to 12 carbon atoms, a cycloalkyl having 3 to 10 carbon atoms, an aryl having 6 to 12 carbon atoms, or a heteroaryl having 3 to 12 carbon atoms.

A second aspect of the present disclosure provides an electronic element, comprising an anode and a cathode disposed opposite to each other, and a functional layer disposed between the anode and the cathode; the functional layer contains the organic compound of the first aspect of the present disclosure.

A third aspect of the present disclosure provides an electronic apparatus, comprising the electronic element of the second aspect of the present disclosure.

In the organic compound of the present disclosure, an alkyl-substituted benzene ring is centered, and an aromatic amine is connected to the benzene ring at an ortho-position of the alkyl and at least one of substituents on the aromatic amine is a dibenzo-penta membered ring. Such a configuration enhances the spatial configuration of the molecule and increases the glass transition temperature of the material. At the same time, the introduction of an aromatic group onto a benzene ring can effectively avoid intermolecular stacking, making the material have an excellent film-forming property. Applying the organic compound as the material of a hole transport layer in organic electroluminescent devices can improve the efficiency and service life of the devices.

The other features and advantages of the present disclosure will be described in detail in the following detailed description of the embodiments.

### BRIEF DESCRIPTION OF THE DRAWINGS

The drawings are provided to provide a further understanding of the present disclosure and constitute a part of the specification, and together with the following detailed description, serve to explain the present disclosure, but do not constitute a limitation of the present disclosure.

FIG. 1 is a schematic structural diagram of an organic electroluminescent device according to an embodiment of the present disclosure.

FIG. 2 is a schematic diagram of a first electronic apparatus according to an embodiment of the present disclosure.

FIG. 3 is a schematic structural diagram of a photoelectric conversion device according to an embodiment of the present disclosure.

FIG. 4 is a schematic diagram of a second electronic apparatus according to an embodiment of the present disclosure.

### NOTE OF REFERENCE SIGNS

100: Anode; 200: Cathode; 300: Functional layer; 310: Hole injection layer; 320: Hole transport layer; 321: First hole transport layer; 322: Second hole transport layer; 330: Organic light-emitting layer; 340: Electron transport layer; 350: Electron injection layer; 360: Photoelectric conversion layer; 400: First electronic apparatus; 500: Second electronic apparatus

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present disclosure more comprehensive and complete, and to convey the concepts of these exemplary embodiments fully to those of ordinary skill in the art. Features, structures, or characteristics described herein can be combined in one or more embodiments in any suitable manner. In the following description, var specific details are provided to give a full understanding of the embodiments of the present disclosure.

In a first aspect, the present disclosure provides an organic compound having a structure shown in a Formula I:

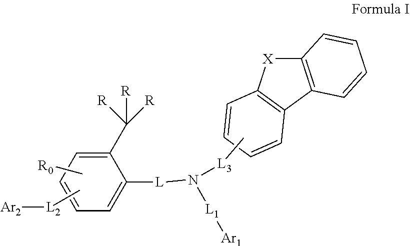

wherein, X is selected from O, S, C(R₁R₂), or N(R₃);
R₁, R₂, and R₃ are identical or different, and are each independently selected from an alkyl having 1 to 5 carbon atoms, a deuteroalkyl having 1 to 5 carbon atoms, a deuteroaryl having 6 to 20 carbon atoms, an aryl having 6 to 20 carbon atoms, or a heteroaryl having 3 to 20 carbon atoms; or R₁ and R₂ are interconnected together with the atoms to which they are commonly connected to form a 5- to 13-membered saturated or unsaturated ring;
each R is independently selected from a deuterium, an alkyl having 1 to 5 carbon atoms, or a deuteroalkyl having 1 to 5 carbon atoms;
R₀ is selected from a hydrogen, an alkyl having 1 to 5 carbon atoms, a deuteroalkyl having 1 to 5 carbon atoms, a haloalkyl having 1 to 5 carbon atoms, a deuteroaryl having 6 to 18 carbon atoms, an aryl having 6 to 18 carbon atoms, or a heteroaryl having 3 to 18 carbon atoms;
L is selected from a single bond, a substituted or unsubstituted arylene having 6 to 18 carbon atoms, or a substituted or unsubstituted heteroarylene having 3 to 18 carbon atoms;
the substituent(s) of L are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 10 carbon atoms, an aryl having 6 to 12 carbon atoms, or a heteroaryl having 3 to 12 carbon atoms;
Ar₂ is selected from a substituted or unsubstituted aryl having 6 to 18 carbon atoms, a substituted or unsubstituted dibenzofuranyl, or a substituted or unsubstituted dibenzothienyl;
the substituent(s) of Ar₂ are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 10 carbon atoms, an aryl having 6 to 12 carbon atoms, a dibenzofuranyl, or a di benzothienyl;
L₂ is selected from a single bond, a substituted or unsubstituted arylene having 6 to 12 carbon atoms, or a substituted or unsubstituted heteroarylene having 3 to 18 carbon atoms;
the substituent(s) of L₂ are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 6 carbon atoms, or a phenyl;
Ar₁ is selected from a substituted or unsubstituted aryl having 6 to 30 carbon atoms, or a substituted or unsubstituted heteroaryl having 3 to 30 carbon atoms;
the substituent(s) of Ar₁ are each independently selected from a deuterium, a halogen group, a cyano, an alkyl having 1 to 10 carbon atoms, a deuteroalkyl having 1 to 10 carbon atoms, a trialkylsilyl having 3 to 12 carbon atoms, a haloalkyl having 1 to 10 carbon atoms, a cycloalkyl having 3 to 12 carbon atoms, a deuteroaryl having 6 to 20 carbon atoms, an aryl having 6 to 20 carbon atoms, or a heteroaryl having 3 to 20 carbon atoms; optionally, any two adjacent substituents of Ar₁ form a saturated or unsaturated 3- to 15-membered ring;
L₁ and L₃ are identical or different, and are each independently selected from a single bond, a substituted or unsubstituted arylene having 6 to 18 carbon atoms, or a substituted or unsubstituted heteroarylene having 3 to 18 carbon atoms;
the substituent(s) of L₁ and L₃ are each independently selected from a deuterium, a halogen group, a cyano, a trialkylsilyl having 3 to 12 carbon atoms, a cycloalkyl having 3 to 10 carbon atoms, an aryl having 6 to 12 carbon atoms, or a heteroaryl having 3 to 12 carbon atoms.

In the present disclosure, the terms “optional” and “optionally” mean that the event or circumstance described later can but does not necessarily occur, and the description includes the situations where the event or circumstance occurs or does not occur. For example, “optionally, any two adjacent substituents form a ring” means that these two substituents may form a ring but not necessarily, including scenarios both where two adjacent substituents form a ring and where two adjacent substituents do not form a ring. For example, “optionally, any two adjacent substituents of Ar₁ form a saturated or unsaturated 3- to 15-membered ring” means that any two adjacent substituents of Ar₁ can be connected to form a saturated or unsaturated 3- to 15-membered ring, or any two adjacent substituents of Ar₁ can exist independently.

In the present disclosure, the descriptive expression “be . . . each independently selected from” may be used interchangeably with the descriptive expressions “be . . . respectively independently selected from”, and all these expressions should be interpreted in a broad sense. They can not only mean that, for the same symbol in a different group, specific options expressed by the same symbols are mutul non-influential, but also mean that for the same symbol in the same group, specific options expressed by the same symbols are mutul non-influential, and the groups can be identical or different. For example,

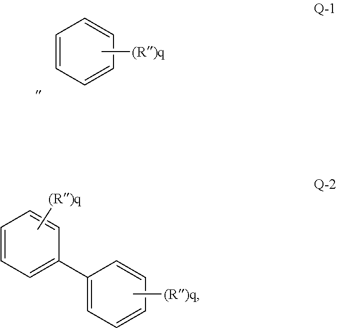

in which each q is independently 0, 1, 2, or 3, and each R″ is independently selected from a hydrogen, a deuterium, a fluorine, and a chlorine “, means that the Formula Q-1 represents that there are q substituents R” on a benzene ring, and each R″ can be identical or different, with mutual non-influence between the options for each R″; Formula Q-2 represents that there are q substituents R″ on each benzene ring of biphenyl, and the number of R″ substituents on the two benzene rings can be identical or different, with mutual non-influence between the options for each R″.

In the present disclosure, such a term “substituted or unsubstituted” means that the functional group defined by the term may or may not have a substituent (hereinafter referred to as Rc for ease of description). For example, “a substituted or unsubstituted aryl” means an aryl having a substituent Rc or an unsubstituted aryl. Among them, the above substituent, i.e., Rc, may be, for example, a deuterium, a halogen group, a cyano, a heteroaryl, an aryl, an alkyl, a trialkylsilyl, a haloalkyl, a deuteroalkyl, a cycloalkyl, etc.

In the present disclosure, the number of carbon atoms in a substituted or unsubstituted functional group refers to the number of all carbon atoms. For example, if L₁ is a substituted arylene having 12 carbon atoms, the number of all carbon atoms of the arylene and the substituents thereon is 12.

In the present disclosure, aryl refers to optional functional group or substituent derived from an aromatic carbon ring. An aryl may be a monocyclic aryl (e.g., phenyl) or a polycyclic aryl. In other words, an aryl may be a monocyclic aryl, a fused cycloaryl, two or more monocyclic aryls linked by carbon-carbon bond, a monocyclic aryl and a fused cycloaryl linked by carbon-carbon bond, or two or more fused cycloaryls linked by carbon-carbon bond. That is, unless otherwise specified, two or more aromatic groups linked by carbon-carbon bond may also be regarded as an aryl in the present disclosure. Among them, a fused cycloaryl may include, for example, a bicyclic fused aryl (e.g., naphthyl), a tricyclic fused aryl (e.g., phenanthryl, fluorenyl, anthryl), etc. For example, in the present disclosure, biphenyl, terphenyl and the like belong to an aryl. Examples of an aryl include, but are not limited to, a phenyl, a naphthyl, a fluorenyl, an anthryl, a phenanthryl, a biphenyl, a terphenyl, a benzo[9,10] phenanthryl, a pyrenyl, a benzofluoranthryl, a chrysenyl, a spirobifluorenyl, etc. In the present disclosure, “an arylene” involved refers to a divalent group formed by further removing one hydrogen atom from an aryl.

In the present disclosure, a substituted aryl may mean that one or more than two hydrogen atoms in the aryl group are replaced by a group such as a deuterium atom, a halogen group, a cyano, an aryl, a heteroaryl, a trialkylsilyl, an alkyl, a cycloalkyl, a haloalkyl, a deuteroalkyl, etc. Specific examples of an aryl substituted with a heteroaryl include, but are not limited to a phenyl substituted with a dibenzofuranyl, a phenyl substituted with a dibenzothienyl, a phenyl substituted with a pyridyl, etc. It should be understood that the number of carbon atoms in a substituted aryl refers to the number of all carbon atoms of an aryl and the substituents on the aryl. For example, a substituted aryl having 18 carbon atoms, refers to the number of all carbon atoms of the aryl and the substituents thereon is 18.

In the present disclosure, a heteroaryl refers to a monovalent aromatic ring containing at least one heteroatom or a derivative thereof. The heteroatom(s) may be one or more selected from B, O, N, P, Si, Se, and S. A heteroaryl may be a monocyclic heteroaryl or a polycyclic heteroaryl. In other words, a heteroaryl may be a single aromatic ring system, or multiple aromatic ring systems linked by carbon-carbon bond, with any of the aromatic ring systems being an aromatic monocyclic ring or a fused aromatic ring. For example, a heteroaryl may include, thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, N-phenylcarbazolyl, N-pyridylcarbazolyl, N-methylcarbazolyl, etc, but not limited to thereto. In the present disclosure, “a heteroarylene” involved refers to a divalent group formed by further removing one hydrogen atom from a heteroaryl.

In the present disclosure, a substituted heteroaryl may mean that one or more than two hydrogen atoms in the heteroaryl are replaced by a group such as a deuterium atom, a halogen group, a cyano, an aryl, a heteroaryl, a trialkylsilyl, an alkyl, a cycloalkyl, a haloalkyl, a deuteroalkyl, etc. Specific examples of a heteroaryl substituted with an aryl include, but are not limited to a dibenzofuranyl substituted with a phenyl, a dibenzothienyl substituted with a phenyl, a pyridyl substituted with a phenyl, etc. It should be understood that the number of carbon atoms in a substituted heteroaryl is the number of all carbon atoms of the heteroaryl group and substituents on the heteroaryl group.

In the present disclosure, the number of the carbon atoms of an aryl as a substituent may be 6 to 20. For example, the number of carbon atoms may be 6, 7, 8, 9, 10, 11, 12, 13, 14, 15, 16, 17, 18, 19, or 20. The specific examples of an aryl as a substituent include, but are not limited to a phenyl, a biphenyl, a naphthyl, an anthryl, and a chrysenyl.

In the present disclosure, the number of the carbon atoms of a heteroaryl as a substituent may be 3 to 20. For example, the number of carbon atoms may be 3, 4, 5, 6, 7, 8, 9, 10, 11, 12, 13, 14, 15, 16, 17, 18, 19, or 20. The specific examples of a heteroaryl as a substituent include, but are not limited to a pyridyl, a pyrimidinyl, a carbazolyl, a dibenzofuranyl, a dibenzothienyl, a quinolyl, a quinazolinyl, a quinoxalinyl, and an isoquinolyl.

In the present disclosure, the number of carbon atoms of an alkyl having 1 to 10 carbon atoms may be, for example, 1, 2, 3, 4, 5, 6, 7, 8, 9, or 10. Specific examples of an alkyl include, but are not limited to a methyl, an ethyl, a n-propyl, an isopropyl, a n-butyl, an isobutyl, a tert-butyl, a n-pentyl, an isopentyl, a neopentyl, a n-hexyl, a n-heptyl, a n-octyl, a 2-ethylhexyl, a nonyl, a decyl, a 3,7-dimethyloctyl, etc.

In the present disclosure, a halogen group may be a fluorine, a chlorine, a bromine, and an iodine.

In the present disclosure, specific examples of a trialkylsilyl include, but are not limited to, a trimethylsilyl, a triethylsilyl, etc.

In the present disclosure, specific examples of a haloalkyl group include, but are not limited to, trifluoromethyl.

In the present disclosure, specific examples of a deuteroalkyl include, but are not limited to, a trideuteromethyl.

In the present disclosure, specific examples of a deuteroalkyl include, but are not limited to, a pentadeuterophenyl.

In the present disclosure, the number of carbon atoms of a cycloalkyl having 3 to 12 carbon atoms may be, for example, 3, 4, 5, 6, 7, 8, 9, 10, 11, or 12. Specific examples of a cycloalkyl include, but are not limited to, a cyclopentane, a cyclohexane, and an adamantane.

In the present disclosure, a non-positioned bond refers to a single bond

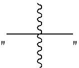

extending from the ring system, which represents that one end of the bond can connect to any position in the ring system through which the bond passes, and the other end connects to the rest of the compound molecule. For example, as shown in Formula (f) below, the naphthyl represented by Formula (f) is connected to other positions of the molecule through two non-positioned bonds passing through the two rings, which indicates any one of possible connection forms shown in Formulae (f-1) to (f-10):

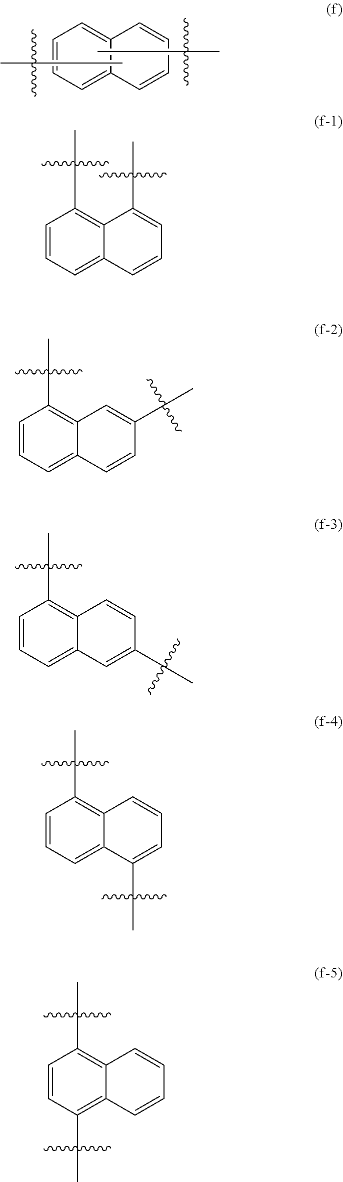

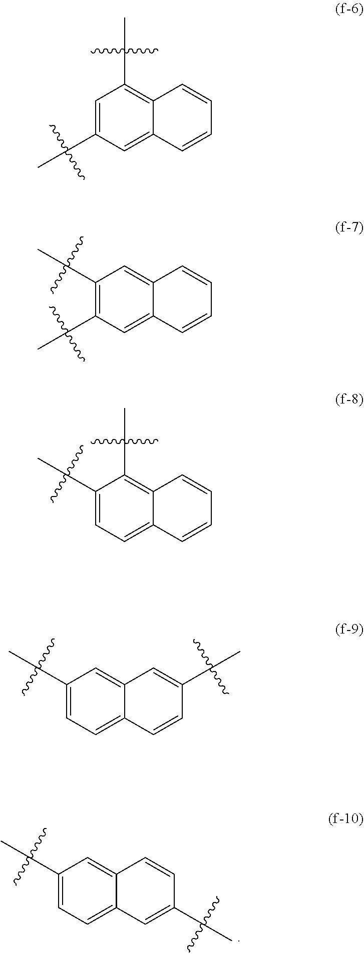

As another example, as shown in Formula (X′) below, the dibenzofuranyl represented by Formula (X′) is connected to other positions of the molecule via a non-positioned connection bond extending from the middle of a side benzene ring, which indicates any of possible connection forms shown in Formulae (X′-1) to (X′-4):

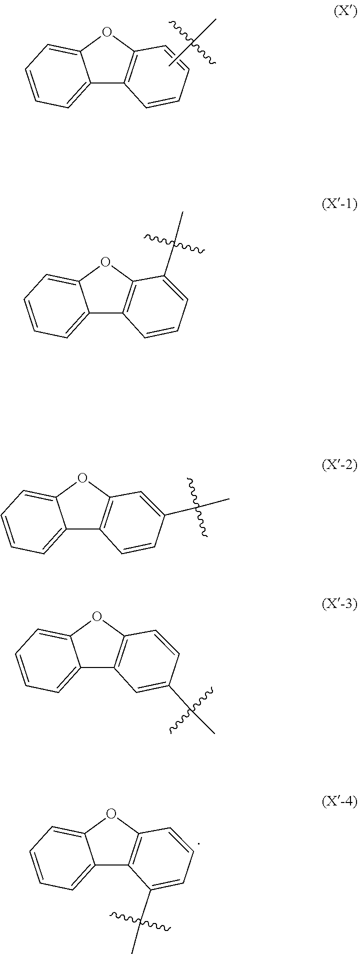

In some embodiments, the organic compound has the structures shown in a Formula I-a:

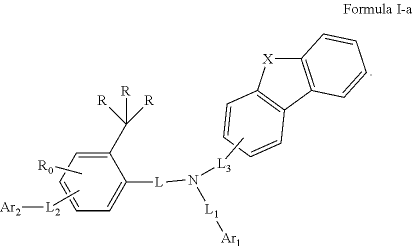

In some embodiments, the organic compound has a structure selected from those shown in a Formula I-1, a Formula I-2, a Formula I-3, a Formula I-4, a Formula I-5, a Formula I-6, a Formula I-7, a Formula I-8, a Formula I-9, a Formula I-10, or a Formula I-11:

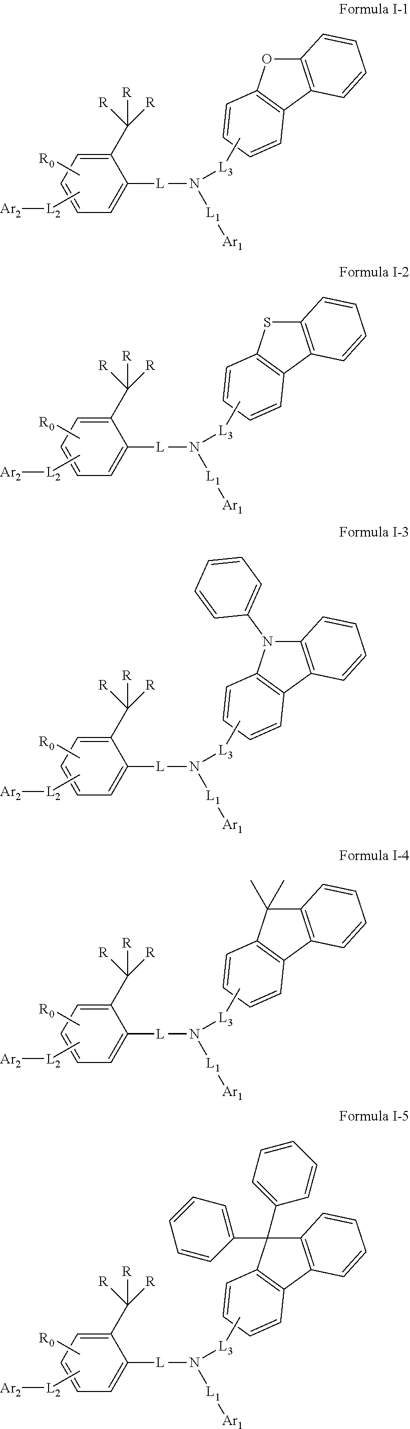

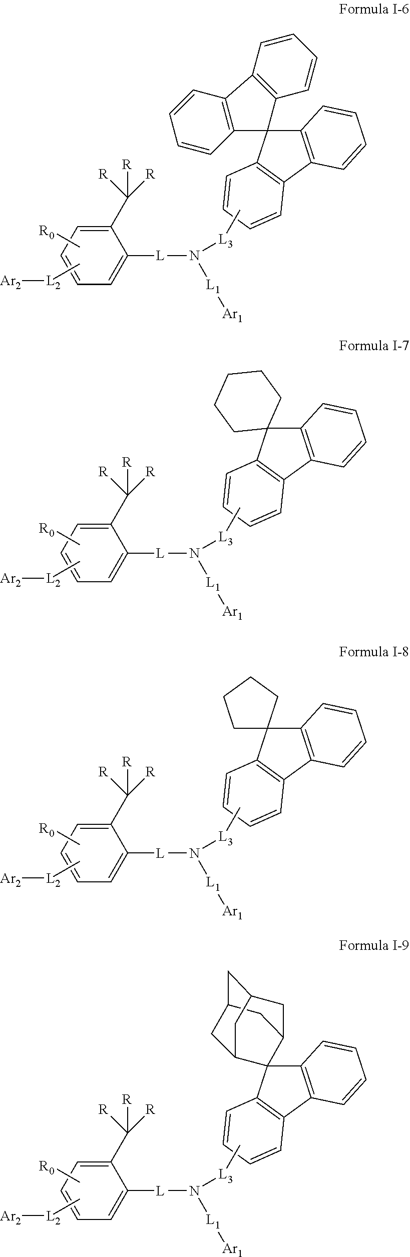

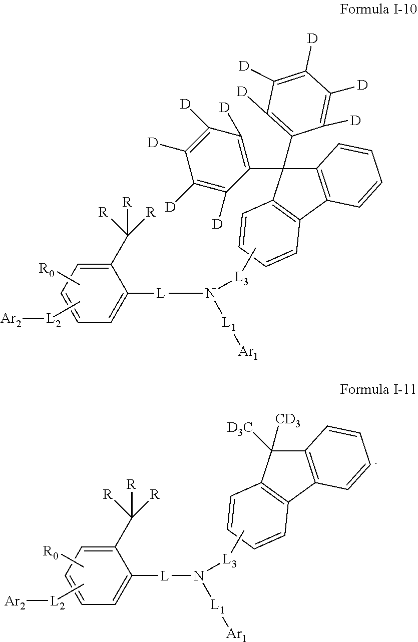

In some embodiments of the present disclosure, L is selected from a single bond, a substituted or unsubstituted arylene having 6, 7, 8, 9, 10, 11, 12, 13, 14, 15, 16, 17, or 18 carbon atoms, or a substituted or unsubstituted heteroarylene having 3, 4, 5, 6, 7, 8, 9, 10, 11, 12, 13, 14, 15, 16, 17, or 18 carbon atoms.

Optionally, the substituent(s) of L are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 10 carbon atoms, an aryl having 6 to 12 carbon atoms, or a heteroaryl having 3 to 12 carbon atoms.

In some embodiments of the present disclosure, L is selected from a single bond, a substituted or unsubstituted phenylene, a substituted or unsubstituted naphthylene, a substituted or unsubstituted biphenylene, a substituted or unsubstituted fluorenylene, a substituted or unsubstituted dibenzofuranylene, a substituted or unsubstituted dibenzothienyl, or a substituted or unsubstituted carbazolylene.

Optionally, the substituent(s) of L are each independently selected from a deuterium, a fluorine, a cyano, a methyl, an ethyl, an isopropyl, a tert-butyl, a trideuteromethyl, a trimethylsilyl, a trifluoromethyl, a cyclopentyl, a cyclohexyl, a phenyl, a naphthyl, a biphenyl, a dibenzofuranyl, a dibenzothienyl, or a carbazolyl.

Optionally, L is selected from a single bond or the group consisting of the following groups:

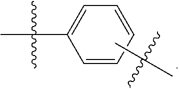

Further optionally, L is selected from a single bond or the group consisting of the following groups:

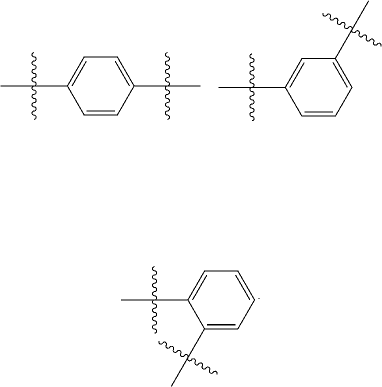 [SMILES: *c1ccc(*)cc1 ; *c1cccc(*)c1 ; *c1ccccc1*]

In some embodiments of the present disclosure, L is selected from a single bond.

In some embodiments of the present disclosure, L₂ is selected from a single bond, a substituted or unsubstituted arylene having 6, 7, 8, 9, 10, 11, or 12 carbon atoms, or a substituted or unsubstituted heteroarylene having 3, 4, 5, 6, 7, 8, 9, 10, 11, 12, 13, 14, 15, 16, 17, or 18 carbon atoms.

Optionally, the substituent(s) of L₂ are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 6 carbon atoms, or a phenyl.

Optionally, L₂ is selected from a single bond, a substituted or unsubstituted phenylene, a substituted or unsubstituted naphthylene, a biphenylene, a substituted or unsubstituted dibenzofuranylene, a substituted or unsubstituted dibenzothienylene, or a substituted or unsubstituted carbazolylene.

Optionally, the substituent(s) of L₂ are each independently selected from a deuterium, a fluorine, a cyano, a methyl, an ethyl, an isopropyl, a tert-butyl, a trideuteromethyl, a trimethylsilyl, a trifluoromethyl, a cyclopentyl, a cyclohexyl, or a phenyl.

Optionally, L₂ is selected from a single bond or the group consisting of the following groups:

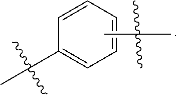

Further optionally, L₂ is selected from a single bond or the group consisting of the following groups:

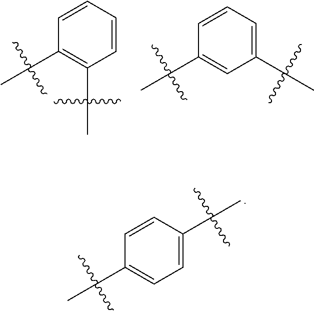 [SMILES: *c1ccccc1* ; *c1cccc(*)c1 ; *c1ccc(*)cc1]

L₁ and L₃ are each independently selected from a single bond, a substituted or unsubstituted arylene having 6, 7, 8, 9, 10, 11, 12, 13, 14, 15, 16, 17, or 18 carbon atoms, or a substituted or unsubstituted heteroarylene having 3, 4, 5, 6, 7, 8, 9, 10, 11, 12, 13, 14, 15, 16, 17, or 18 carbon atoms.

Optionally, the substituent(s) of L₁ and L₃ are each independently selected from a deuterium, a halogen group, a cyano, a trialkylsilyl having 3 to 12 carbon atoms, a cycloalkyl having 3 to 10 carbon atoms, an aryl having 6 to 12 carbon atoms, or a heteroaryl having 3 to 12 carbon atoms.

L₁ and L₃ are each independently selected from a single bond, a substituted or unsubstituted phenylene, a substituted or unsubstituted naphthylene, a substituted or unsubstituted biphenylene, a terphenylene, a substituted or unsubstituted fluorenylene, a substituted or unsubstituted dibenzofuranylene, a substituted or unsubstituted dibenzothienylene, or a substituted or unsubstituted carbazolylene.

the substituent(s) of L₁ and L₃ are each independently selected from a deuterium, a fluorine, a cyano, a trimethylsilyl, a cyclopentyl, a cyclohexyl, a phenyl, a naphthyl, a biphenyl, a dibenzofuranyl, a dibenzothienyl, or a carbazolyl.

Optionally, L₁ is selected from a single bond or the group consisting of the following groups:

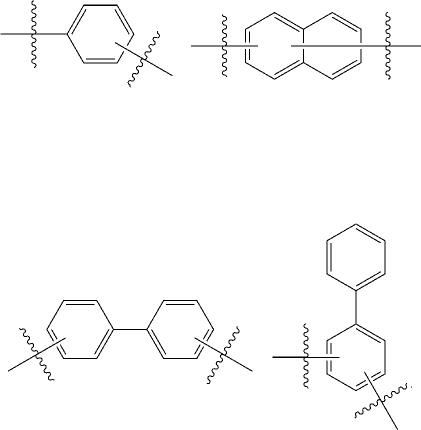

Further optionally, L₁ is selected from a single bond or the group consisting of the following groups:

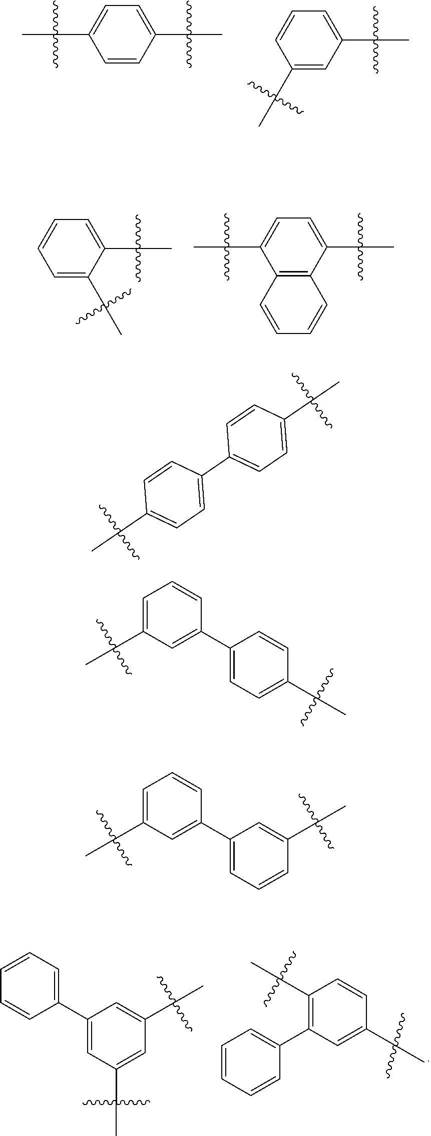

Optionally, L₃ is selected from a single bond or the group consisting of the following groups:

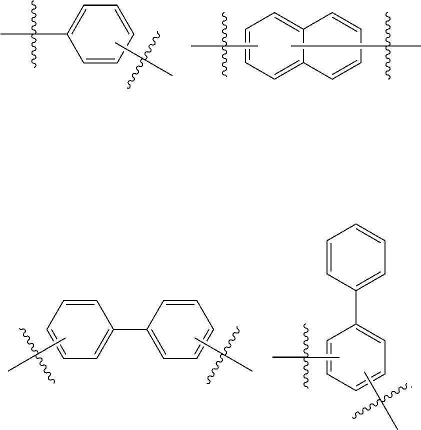

Further optionally, L₃ is selected from a single bond or the group consisting of the following groups:

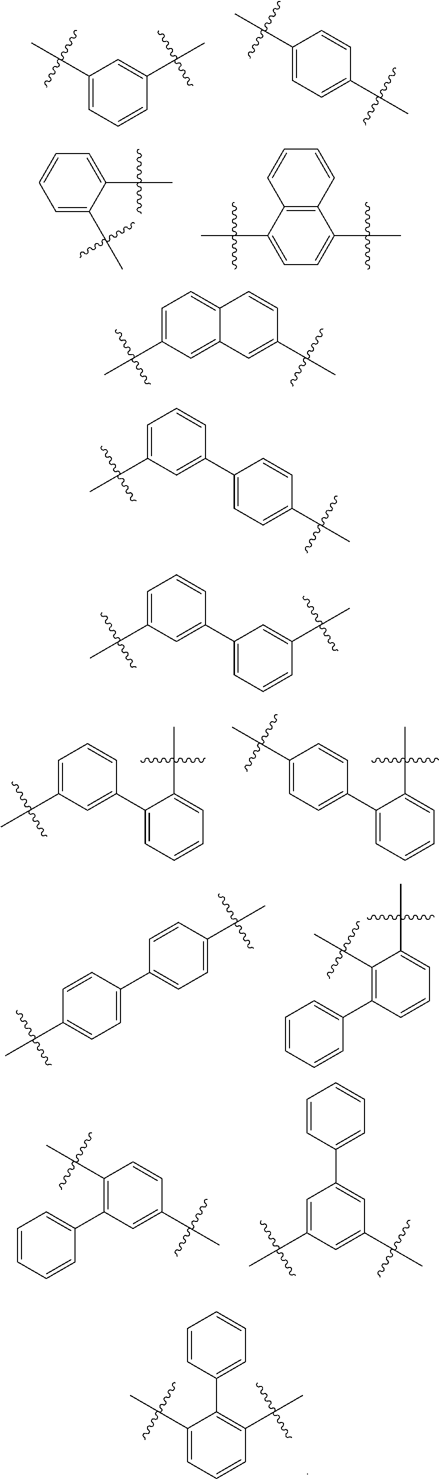

In some embodiments of the present disclosure, Ar₁ is selected from a substituted or unsubstituted aryl having 6 to 25 carbon atoms, or a substituted or unsubstituted heteroaryl having to 24 carbon atoms; Ar₁ is selected from a substituted or unsubstituted aryl having 6, 7, 8, 9, 10, 11, 12, 13, 14, 15, 16, 17, 18, 19, 20, 21, 22, 23, 24, or 25 carbon atoms, or a substituted or unsubstituted heteroaryl having 5, 6, 7, 8, 9, 10, 12, 13, 14, 15, 16, 17, 18, 19, 20, 21, 22, 23, or 24 carbon atoms.

Optionally, the substituent(s) of Ar₁ are each independently selected from a deuterium, a fluorine, a cyano, an alkyl having 1 to 5 carbon atoms, a deuteroalkyl having 1 to 5 carbon atoms, a trialkylsilyl, a haloalkyl having 1 to 5 carbon atoms, a cycloalkyl having 3 to 10 carbon atoms, a deuteroaryl having 6 to 12 carbon atoms, an aryl having 6 to 12 carbon atoms, or a heteroaryl having 5 to 12 carbon atoms; optionally, any two adjacent substituents of Ar₁ form a saturated or unsaturated 5- to 13-membered ring.

In some embodiments, Ar₁ is selected from a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted spirodifluorenyl, a substituted or unsubstituted phenanthryl, a substituted or unsubstituted dibenzofuranyl, a substituted or unsubstituted dibenzothienyl, or a substituted or unsubstituted carbazolyl.

Optionally, the substituent(s) of Ar₁ are each independently selected from a deuterium, a fluorine, a cyano, a methyl, an ethyl, an isopropyl, a tert-butyl, a trimethylsilyl, a trifluoromethyl, a trideuteromethyl, a cyclopentyl, a cyclohexyl, a pentadeuterophenyl, a phenyl, a naphthyl, a biphenyl, a dibenzofuranyl, a dibenzothienyl, or a carbazolyl; optionally, any two adjacent substituents of Ar₁ form a cyclopentane, a cyclohexane, or a fluorene ring.

In some embodiments, Ar₁ is selected from a substituted or unsubstituted group V, and the unsubstituted group V is selected from the group consisting of the following groups:

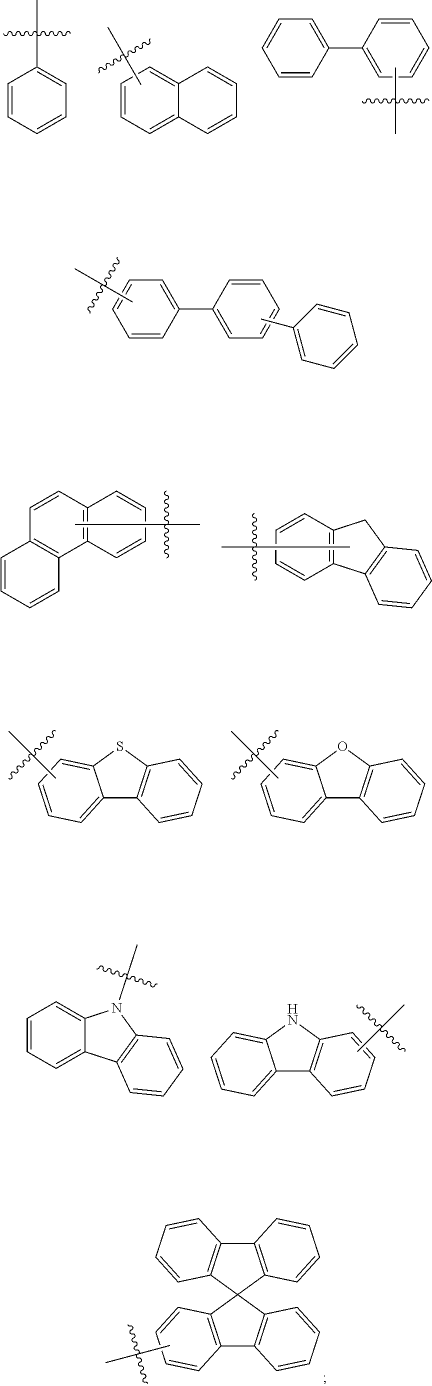

the substituted group V has one or more substituent(s), and each substituent is independently selected from a deuterium, a fluorine, a cyano, a methyl, an ethyl, an isopropyl, a tert-butyl, a trimethylsilyl, a trifluoromethyl, a trideuteromethyl, a cyclopentyl, a cyclohexyl, a pentadeuterophenyl, a phenyl, a naphthyl, a biphenyl, a dibenzofuranyl, a dibenzothienyl, or a carbazolyl, and when the number of the substituents of the group V is greater than 1, the substituents are identical or different.

In some embodiments, Ar₁ is selected from the group consisting of the following groups:

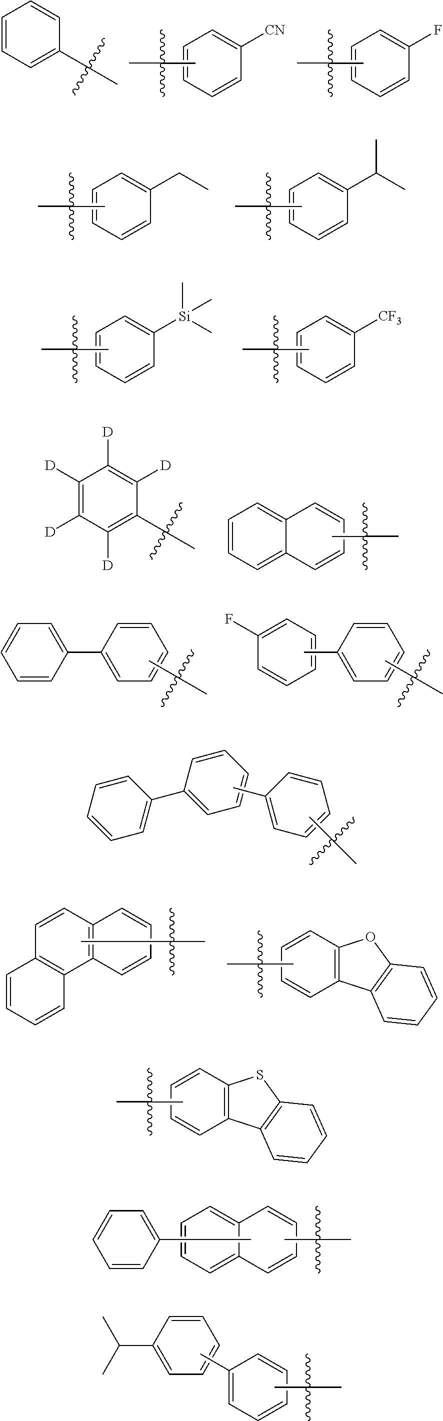

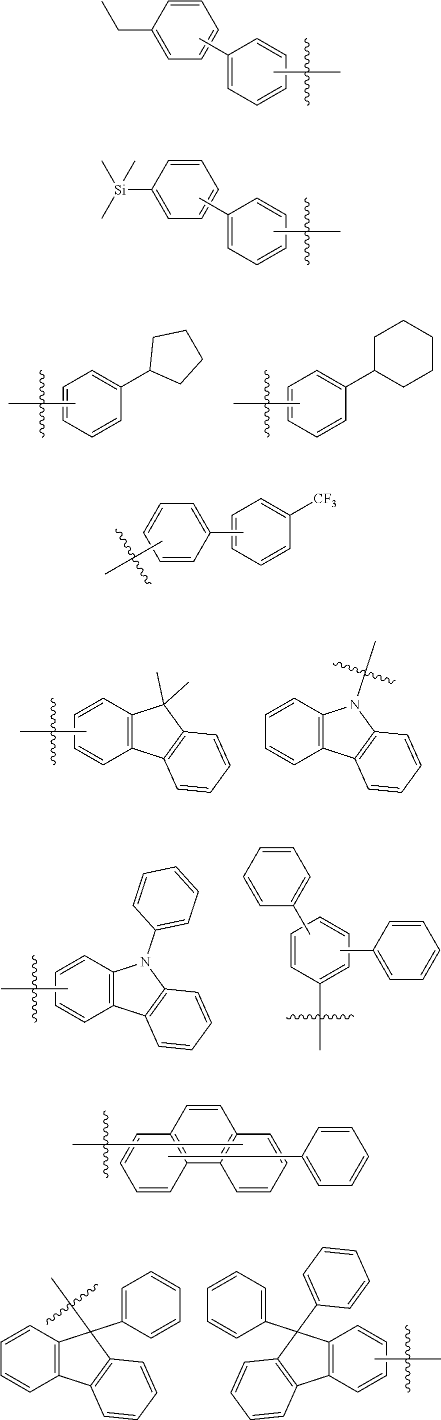

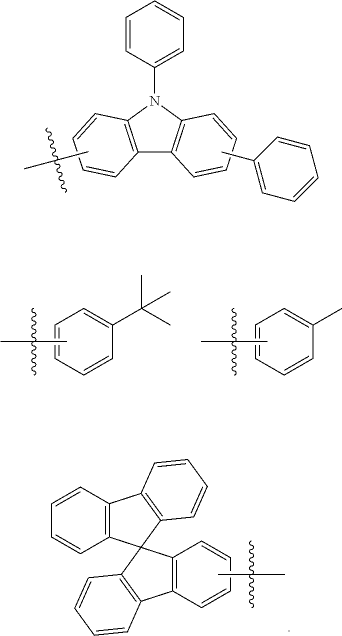

In some embodiments, Ar₁ is selected from the group consisting of the following groups:

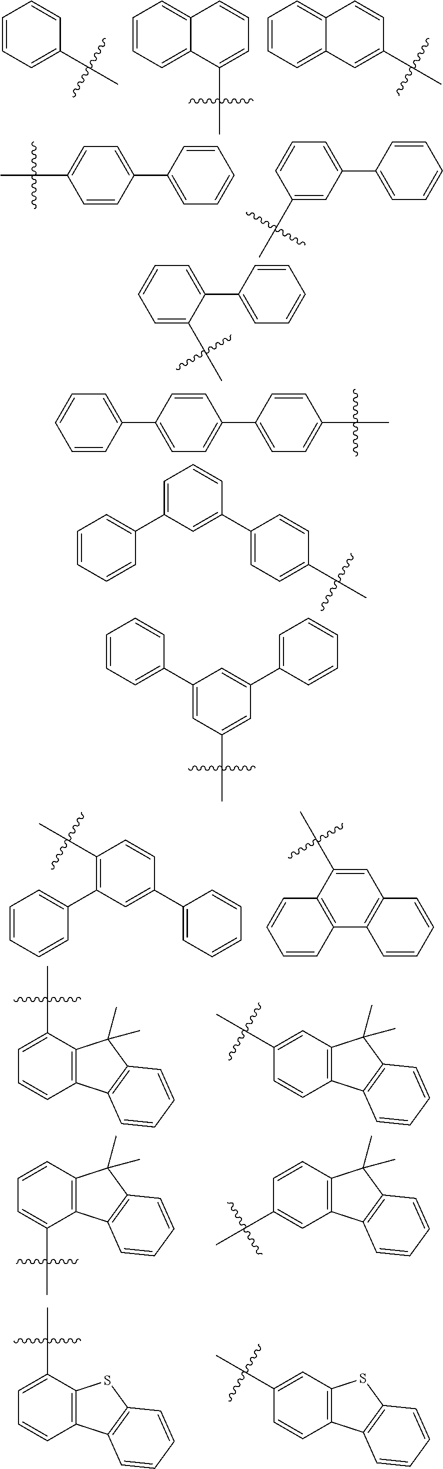

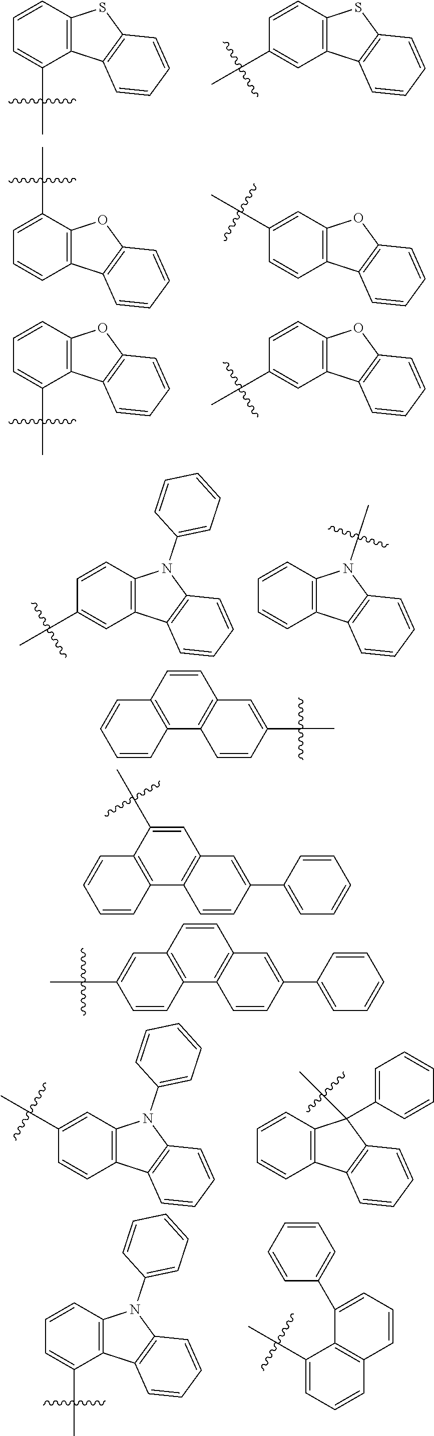

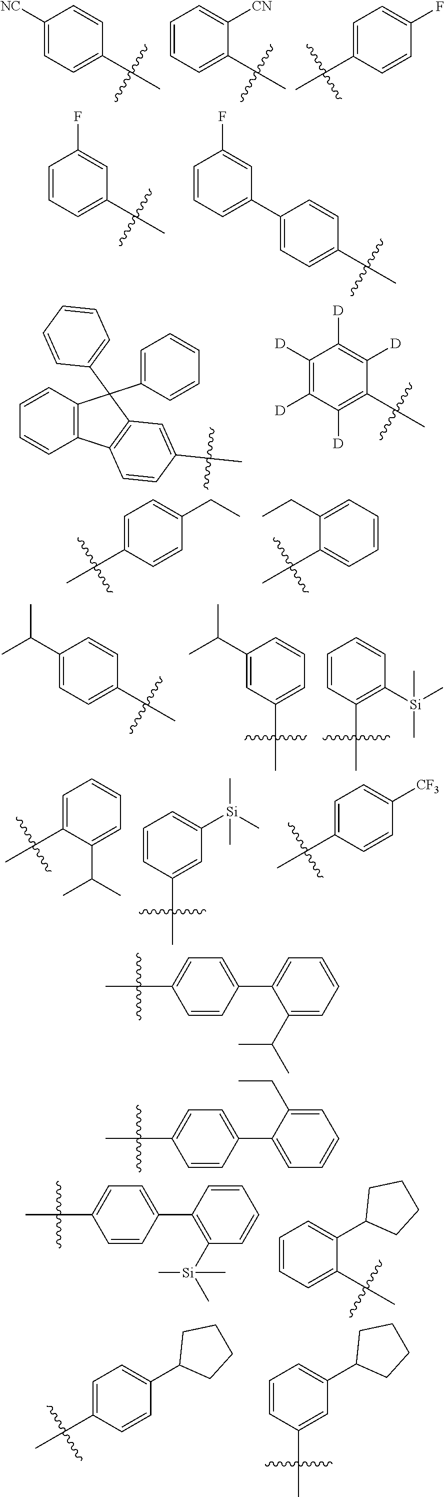

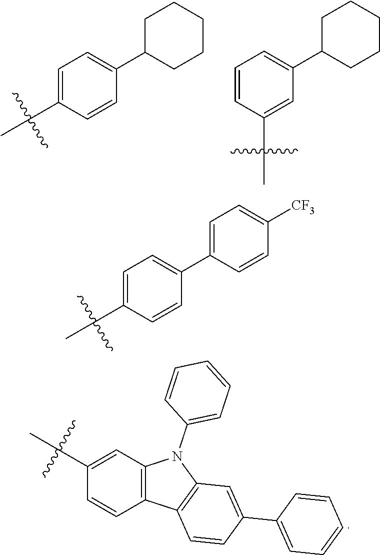

In some embodiments of the present disclosure, Ar₂ is selected from a substituted or unsubstituted aryl having 6, 7, 8, 9, 10, 11, 12, 13, 14, 15, 16, 17, or 18 carbon atoms, a substituted or unsubstituted dibenzofuranyl, or a substituted or unsubstituted dibenzothienyl.

Optionally, the substituent(s) of Ar₂ are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 10 carbon atoms, an aryl having 6 to 12 carbon atoms, a dibenzofuranyl, or a dibenzothienyl.

In some embodiments, Ar₂ is selected from a substituted or unsubstituted group Q, and the unsubstituted group O is selected from the group consisting of the following groups:

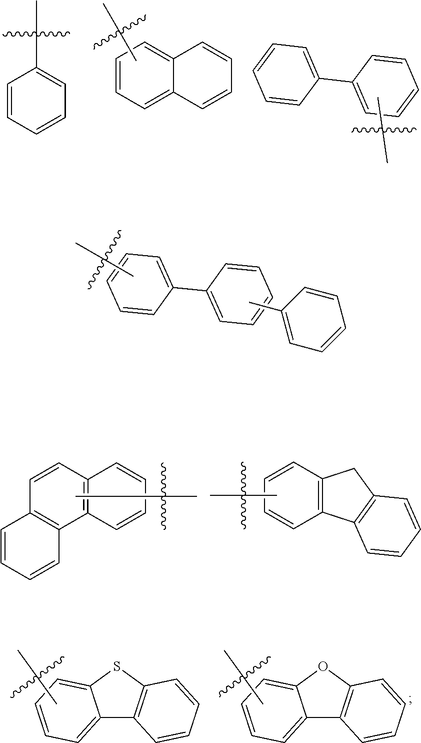

the substituted group Q has one or more substituent(s), and each substituent is independently selected from a deuterium, a fluorine, a cyano, a methyl, an ethyl, an isopropyl, a tert-butyl, a trideuteromethyl, a trimethylsilyl, a trifluoromethyl, a cyclopentyl, a cyclohexyl, a phenyl, a naphthyl, a biphenyl, a dibenzofuranyl, or a dibenzothienyl, and when the number of the substituents of the group Q is greater than 1, the substituents are identical or different.

In some embodiments, Ar₂ is selected from the group consisting of the following groups:

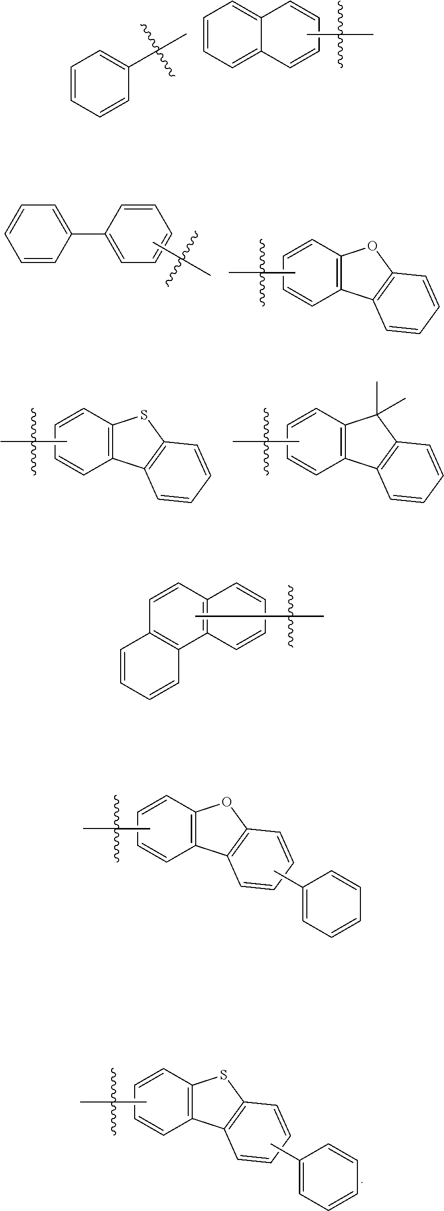

In some embodiments, Ar₂ is selected from the group consisting of the following groups:

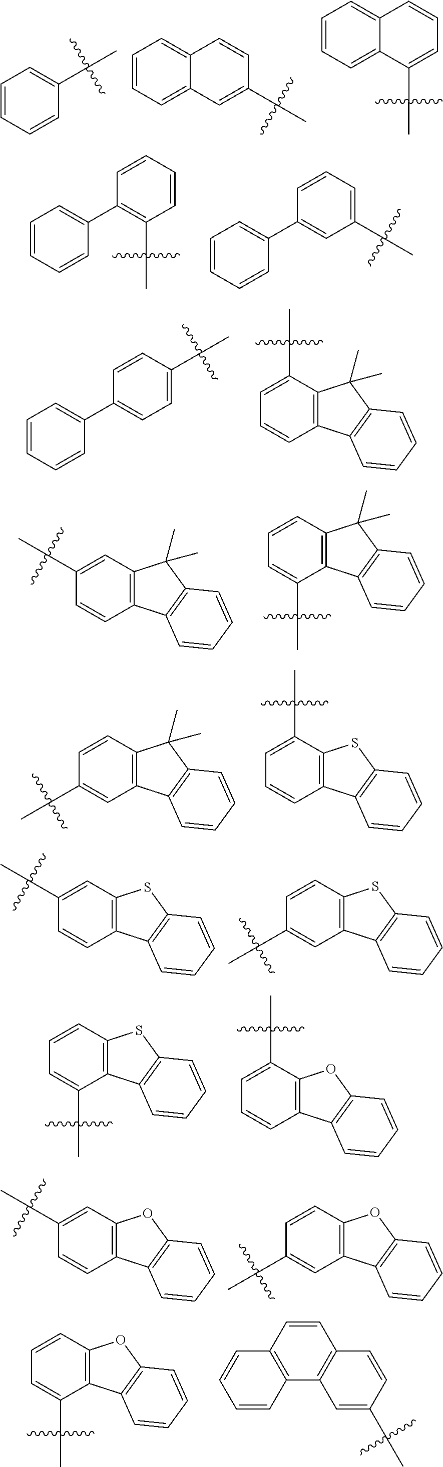

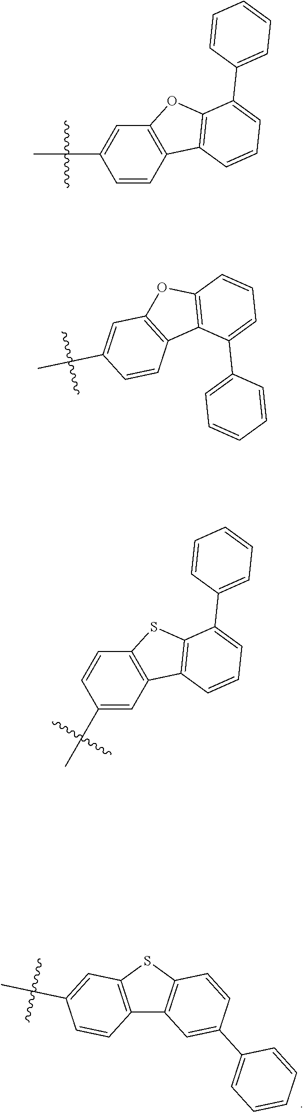 [SMILES: *c1ccc2c(c1)oc1c(-c3ccccc3)cccc12 ; *c1ccc2c(c1)oc1cccc(-c3ccccc3)c12 ; *c1ccc2sc3c(-c4ccccc4)cccc3c2c1 ; *c1ccc2c(c1)sc1ccc(-c3ccccc3)cc12]

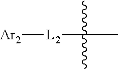

In some embodiments, in Formula I is selected from the group consisting of the following groups:

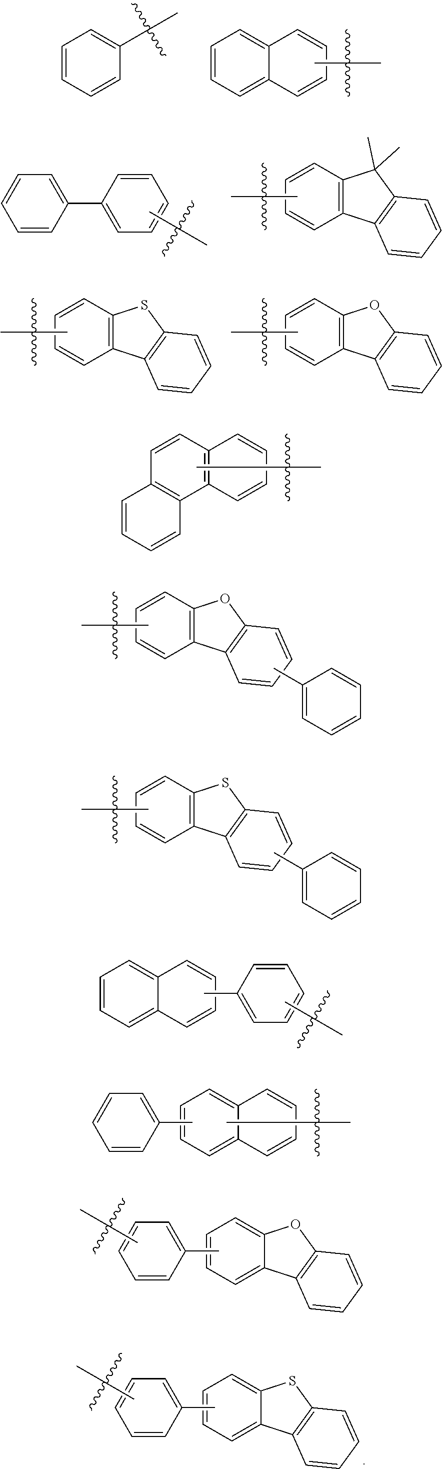

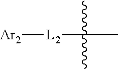

Optionally, in Formula I is selected from the group consisting of the following groups:

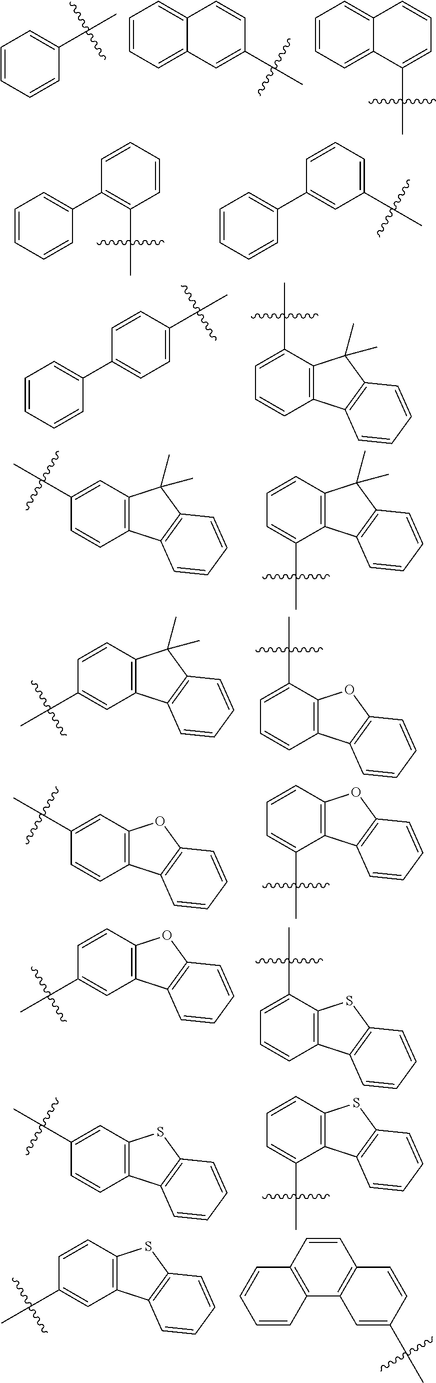

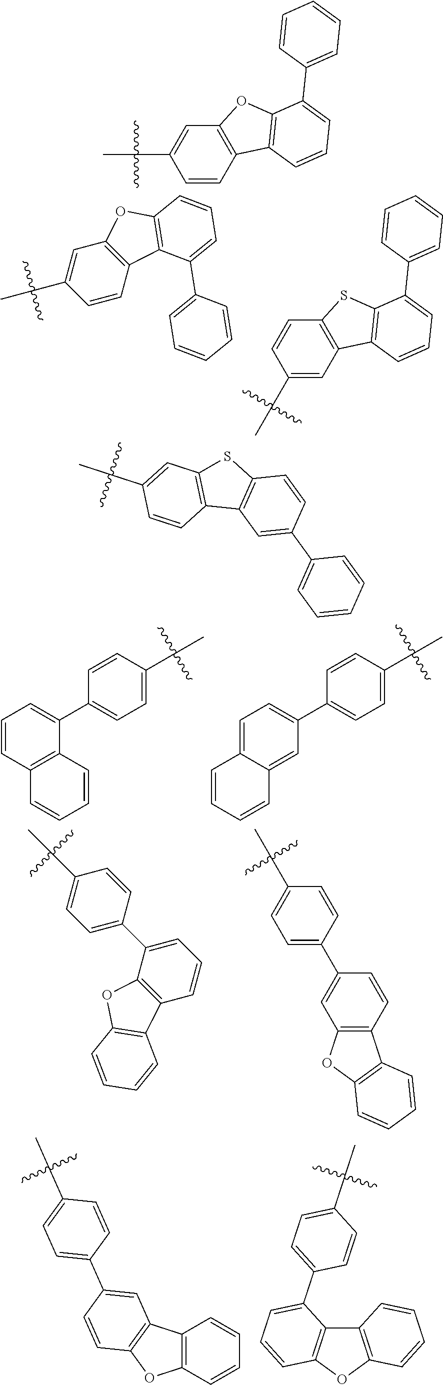

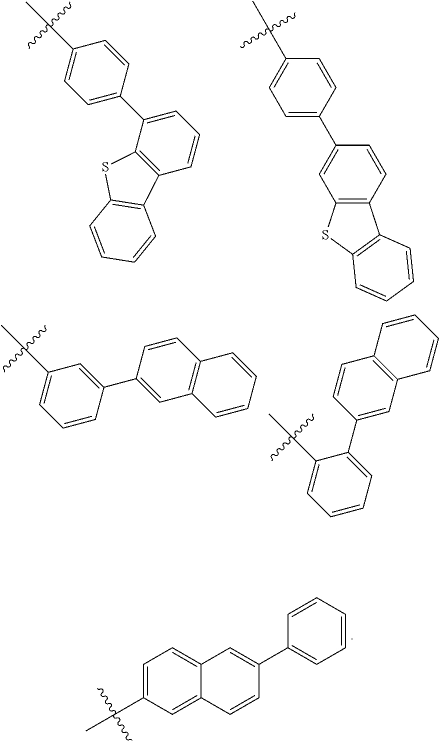

In some embodiments of the present disclosure, R₁, R₂, and R₃ are each independently selected from a methyl, a trideuteromethyl, an ethyl, an isopropyl, a tert-butyl, a phenyl, a pentadeuterophenyl, a naphthyl, a biphenyl, a pyridyl, a dibenzofuranyl, a dibenzothienyl, or a carbazolyl; or R₁ and R₂ are interconnected together with the atoms to which they are commonly connected to form a cyclopentyl, a cyclohexyl, an adamantyl, or a fluorene ring.

In some embodiments,

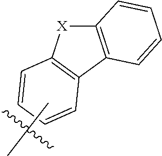

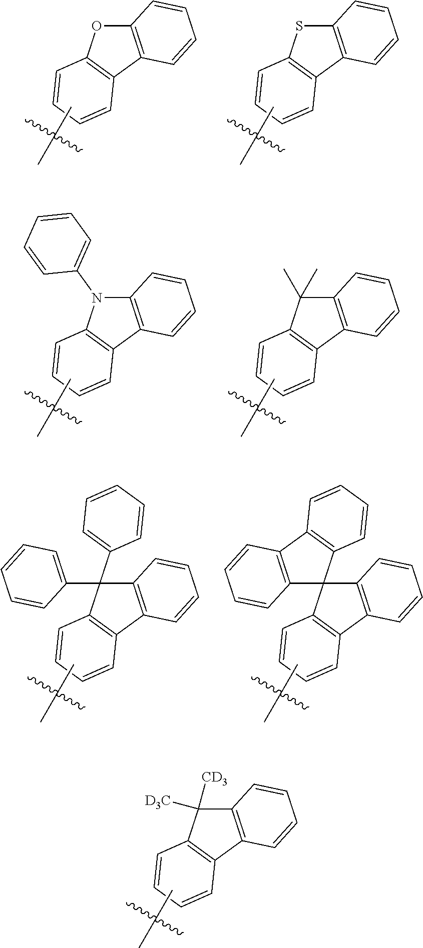

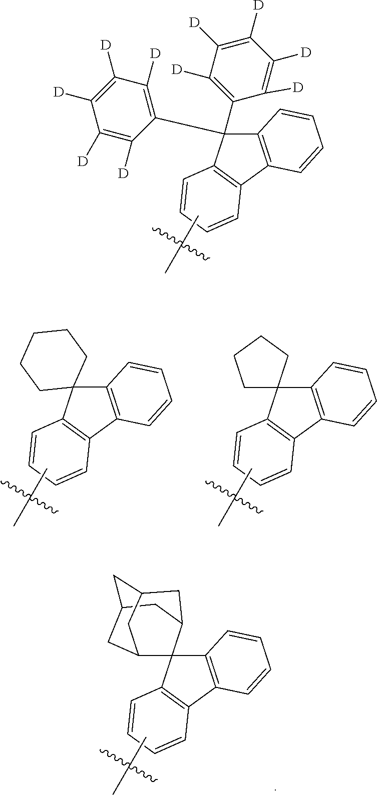

in Formula I is selected from the following groups:

In some embodiments of the present disclosure, each R is independently selected from a deuterium, a methyl, an ethyl, an isopropyl, a tert-butyl, or a trideuteromethyl.

Optionally, each R is identical, and all are selected from a methyl or a trideuteromethyl.

In some embodiments of the present disclosure, R₀ is selected from a hydrogen, a methyl, an ethyl, an isopropyl, a tert-butyl, a trideuteromethyl, a trifluoromethyl, a pentadeuterophenyl, a phenyl, a naphthyl, a biphenyl, a dibenzofuranyl, a dibenzothienyl, or a carbazolyl.

Specifically, the organic compound is selected from the group consisting of the following compounds:

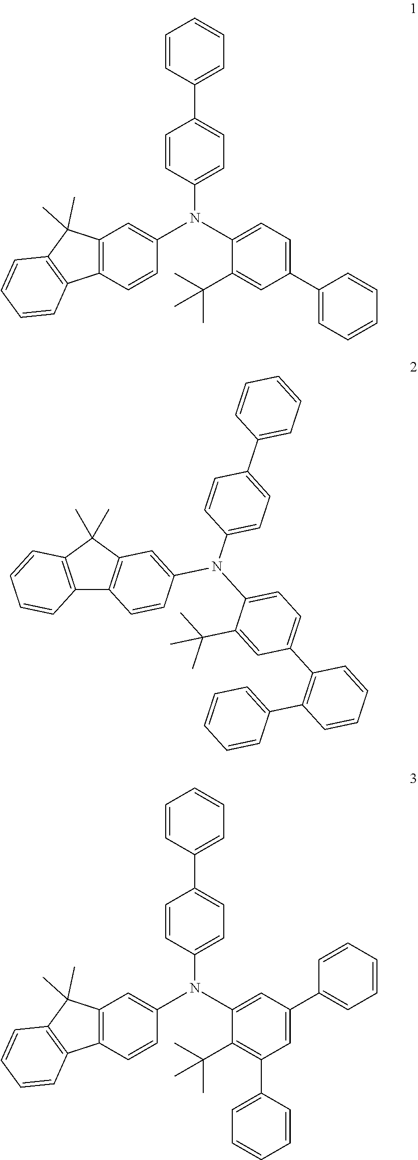 [SMILES: CC(C)(C)c1cc(-c2ccccc2)ccc1N(c1ccc(-c2ccccc2)cc1)c1ccc2c(c1)C(C)(C)c1ccccc1-2 ; CC(C)(C)c1cc(-c2ccccc2-c2ccccc2)ccc1N(c1ccc(-c2ccccc2)cc1)c1ccc2c(c1)C(C)(C)c1ccccc1-2 ; CC(C)(C)c1c(-c2ccccc2)cc(-c2ccccc2)cc1N(c1ccc(-c2ccccc2)cc1)c1ccc2c(c1)C(C)(C)c1ccccc1-2]

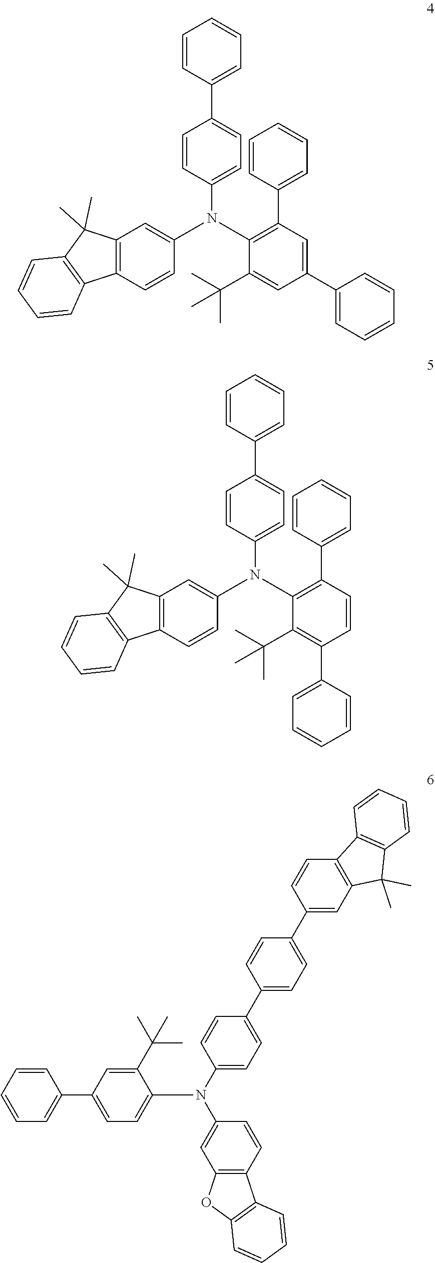 [SMILES: CC(C)(C)c1cc(-c2ccccc2)cc(-c2ccccc2)c1N(c1ccc(-c2ccccc2)cc1)c1ccc2c(c1)C(C)(C)c1ccccc1-2 ; CC(C)(C)c1c(-c2ccccc2)ccc(-c2ccccc2)c1N(c1ccc(-c2ccccc2)cc1)c1ccc2c(c1)C(C)(C)c1ccccc1-2 ; CC(C)(C)c1cc(-c2ccccc2)ccc1N(c1ccc(-c2ccc(-c3ccc4c(c3)C(C)(C)c3ccccc3-4)cc2)cc1)c1ccc2c(c1)oc1ccccc12]

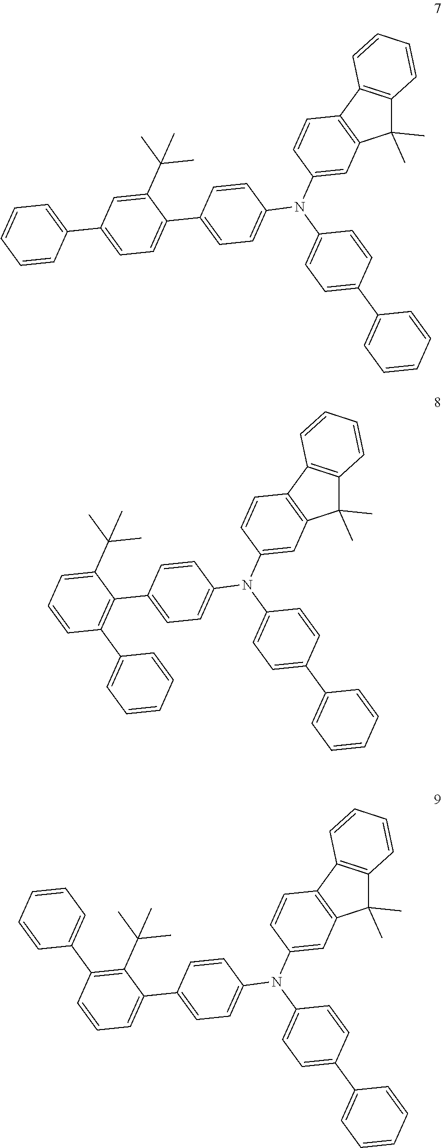 [SMILES: CC(C)(C)c1cc(-c2ccccc2)ccc1-c1ccc(N(c2ccc(-c3ccccc3)cc2)c2ccc3c(c2)C(C)(C)c2ccccc2-3)cc1 ; CC(C)(C)c1cccc(-c2ccccc2)c1-c1ccc(N(c2ccc(-c3ccccc3)cc2)c2ccc3c(c2)C(C)(C)c2ccccc2-3)cc1 ; CC(C)(C)c1c(-c2ccccc2)cccc1-c1ccc(N(c2ccc(-c3ccccc3)cc2)c2ccc3c(c2)C(C)(C)c2ccccc2-3)cc1]

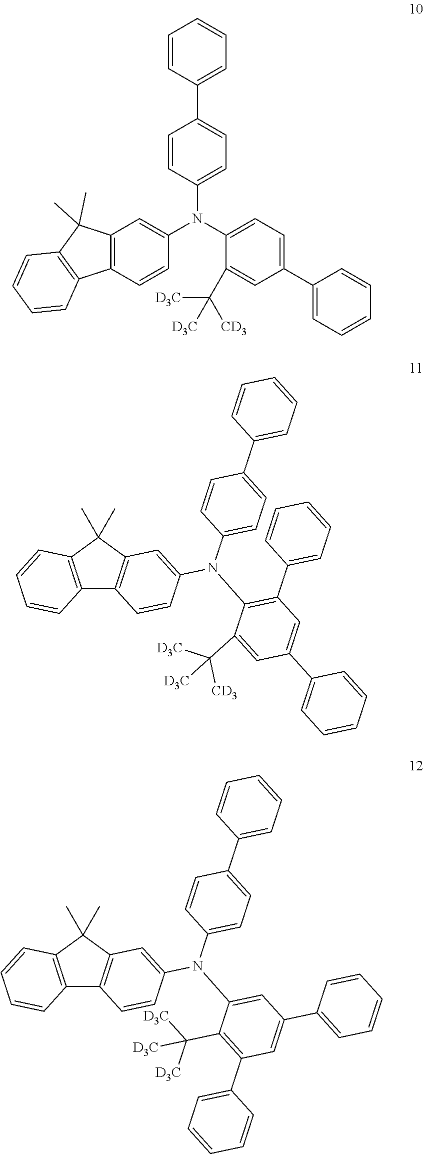 [SMILES: [2H]C([2H])([2H])C(c1cc(-c2ccccc2)ccc1N(c1ccc(-c2ccccc2)cc1)c1ccc2c(c1)C(C)(C)c1ccccc1-2)(C([2H])([2H])[2H])C([2H])([2H])[2H] ; [2H]C([2H])([2H])C(c1cc(-c2ccccc2)cc(-c2ccccc2)c1N(c1ccc(-c2ccccc2)cc1)c1ccc2c(c1)C(C)(C)c1ccccc1-2)(C([2H])([2H])[2H])C([2H])([2H])[2H] ; [2H]C([2H])([2H])C(c1c(-c2ccccc2)cc(-c2ccccc2)cc1N(c1ccc(-c2ccccc2)cc1)c1ccc2c(c1)C(C)(C)c1ccccc1-2)(C([2H])([2H])[2H])C([2H])([2H])[2H]]

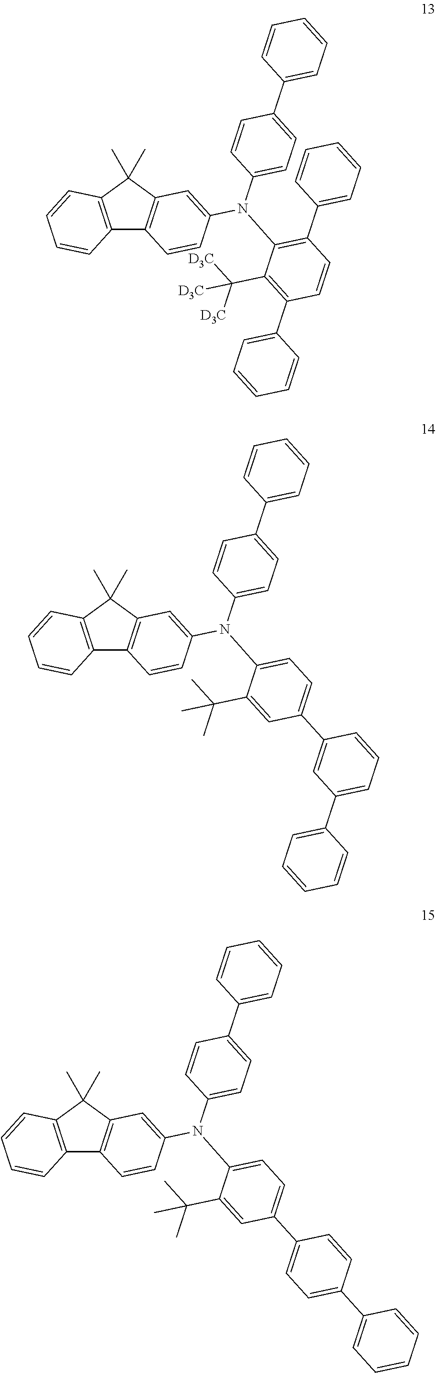 [SMILES: [2H]C([2H])([2H])C(c1c(-c2ccccc2)ccc(-c2ccccc2)c1N(c1ccc(-c2ccccc2)cc1)c1ccc2c(c1)C(C)(C)c1ccccc1-2)(C([2H])([2H])[2H])C([2H])([2H])[2H] ; CC(C)(C)c1cc(-c2cccc(-c3ccccc3)c2)ccc1N(c1ccc(-c2ccccc2)cc1)c1ccc2c(c1)C(C)(C)c1ccccc1-2 ; CC(C)(C)c1cc(-c2ccc(-c3ccccc3)cc2)ccc1N(c1ccc(-c2ccccc2)cc1)c1ccc2c(c1)C(C)(C)c1ccccc1-2]

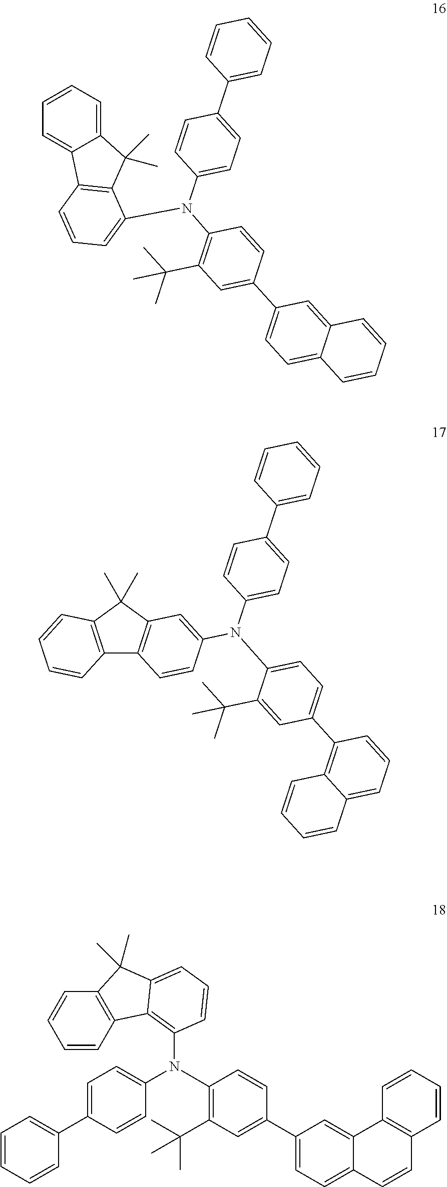 [SMILES: CC(C)(C)c1cc(-c2ccc3ccccc3c2)ccc1N(c1ccc(-c2ccccc2)cc1)c1cccc2c1C(C)(C)c1ccccc1-2 ; CC(C)(C)c1cc(-c2cccc3ccccc23)ccc1N(c1ccc(-c2ccccc2)cc1)c1ccc2c(c1)C(C)(C)c1ccccc1-2 ; CC(C)(C)c1cc(-c2ccc3ccc4ccccc4c3c2)ccc1N(c1ccc(-c2ccccc2)cc1)c1cccc2c1-c1ccccc1C2(C)C]

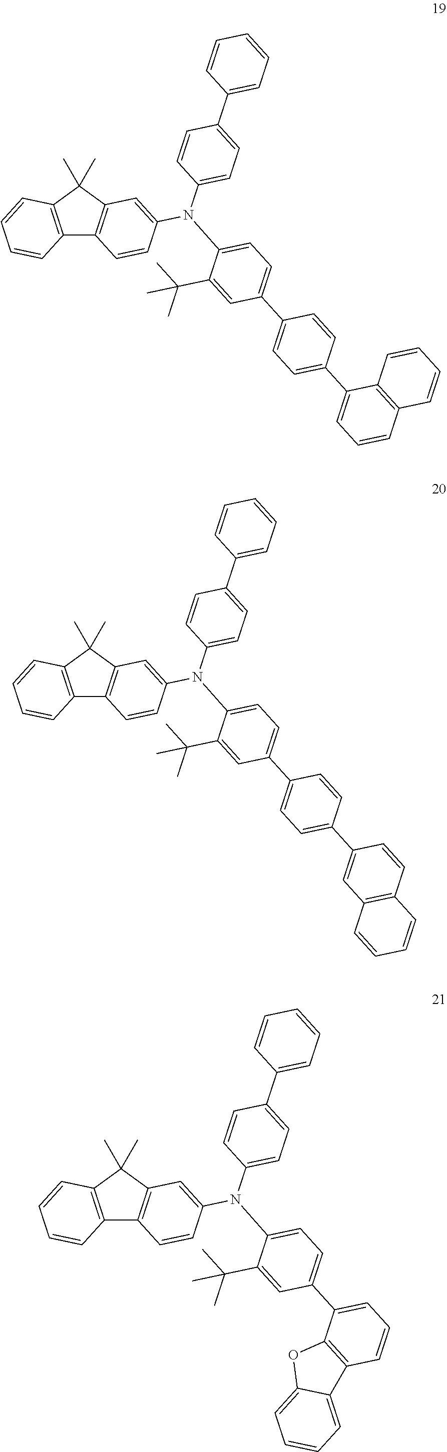 [SMILES: CC(C)(C)c1cc(-c2ccc(-c3cccc4ccccc34)cc2)ccc1N(c1ccc(-c2ccccc2)cc1)c1ccc2c(c1)C(C)(C)c1ccccc1-2 ; CC(C)(C)c1cc(-c2ccc(-c3ccc4ccccc4c3)cc2)ccc1N(c1ccc(-c2ccccc2)cc1)c1ccc2c(c1)C(C)(C)c1ccccc1-2 ; CC(C)(C)c1cc(-c2cccc3c2oc2ccccc23)ccc1N(c1ccc(-c2ccccc2)cc1)c1ccc2c(c1)C(C)(C)c1ccccc1-2]

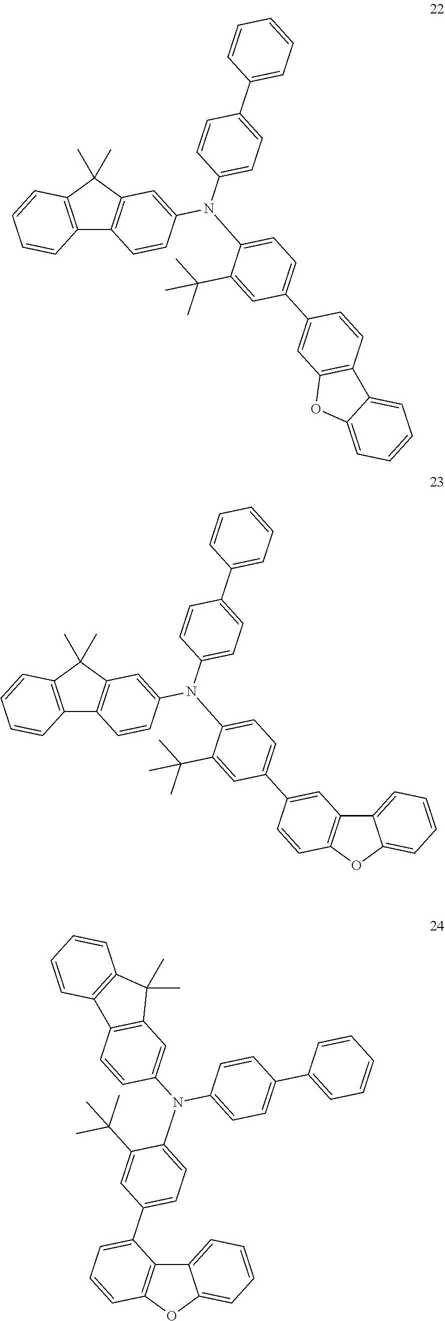 [SMILES: CC(C)(C)c1cc(-c2ccc3c(c2)oc2ccccc23)ccc1N(c1ccc(-c2ccccc2)cc1)c1ccc2c(c1)C(C)(C)c1ccccc1-2 ; CC(C)(C)c1cc(-c2ccc3oc4ccccc4c3c2)ccc1N(c1ccc(-c2ccccc2)cc1)c1ccc2c(c1)C(C)(C)c1ccccc1-2 ; CC(C)(C)c1cc(-c2cccc3oc4ccccc4c23)ccc1N(c1ccc(-c2ccccc2)cc1)c1ccc2c(c1)C(C)(C)c1ccccc1-2]

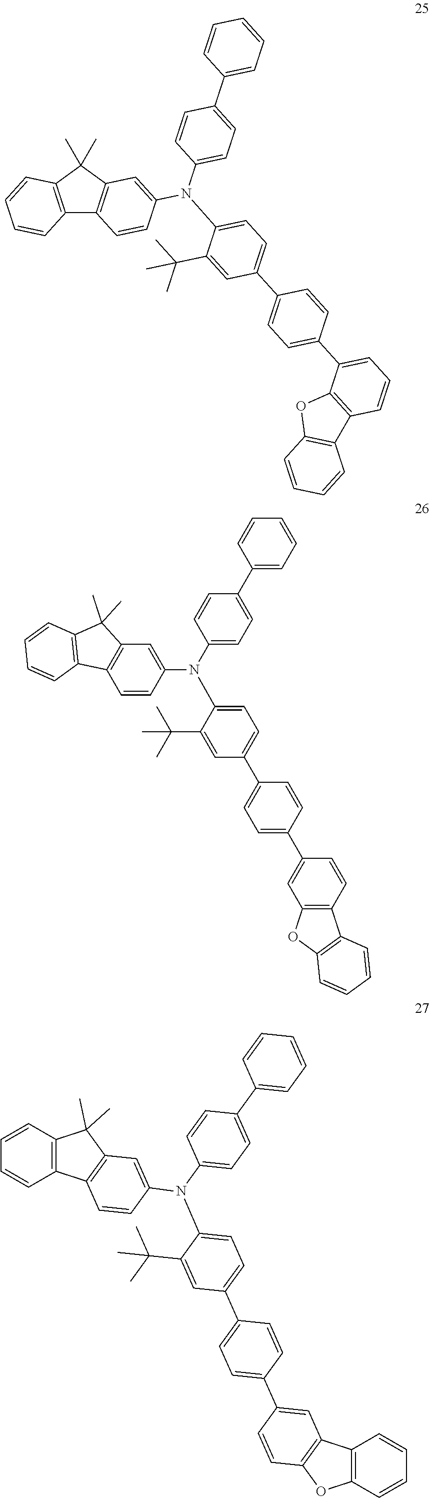

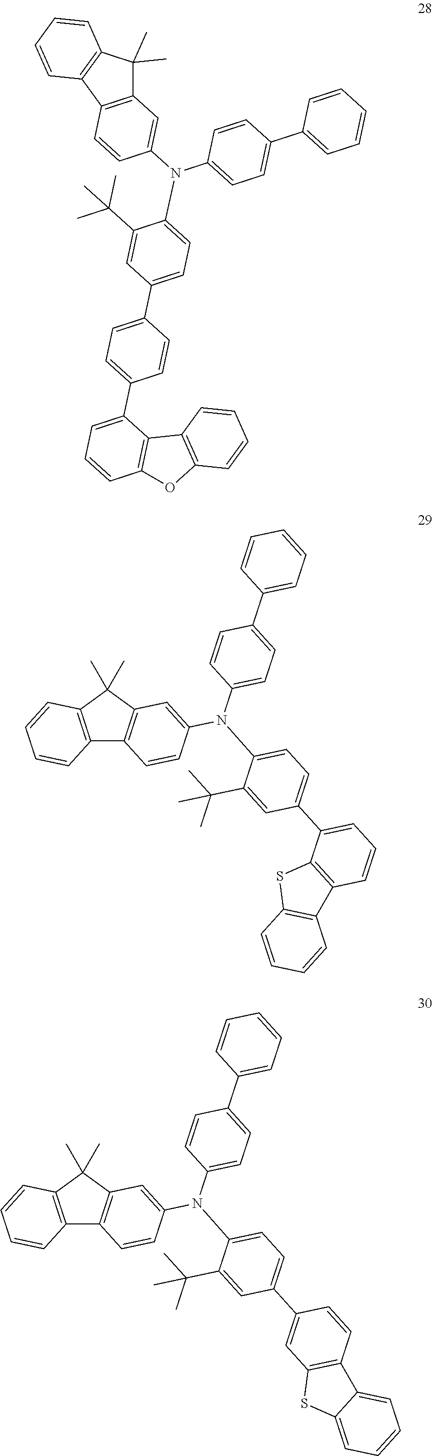 [SMILES: CC(C)(C)c1cc(-c2ccc(-c3cccc4oc5ccccc5c34)cc2)ccc1N(c1ccc(-c2ccccc2)cc1)c1ccc2c(c1)C(C)(C)c1ccccc1-2 ; CC(C)(C)c1cc(-c2cccc3c2sc2ccccc23)ccc1N(c1ccc(-c2ccccc2)cc1)c1ccc2c(c1)C(C)(C)c1ccccc1-2 ; CC(C)(C)c1cc(-c2ccc3c(c2)sc2ccccc23)ccc1N(c1ccc(-c2ccccc2)cc1)c1ccc2c(c1)C(C)(C)c1ccccc1-2]

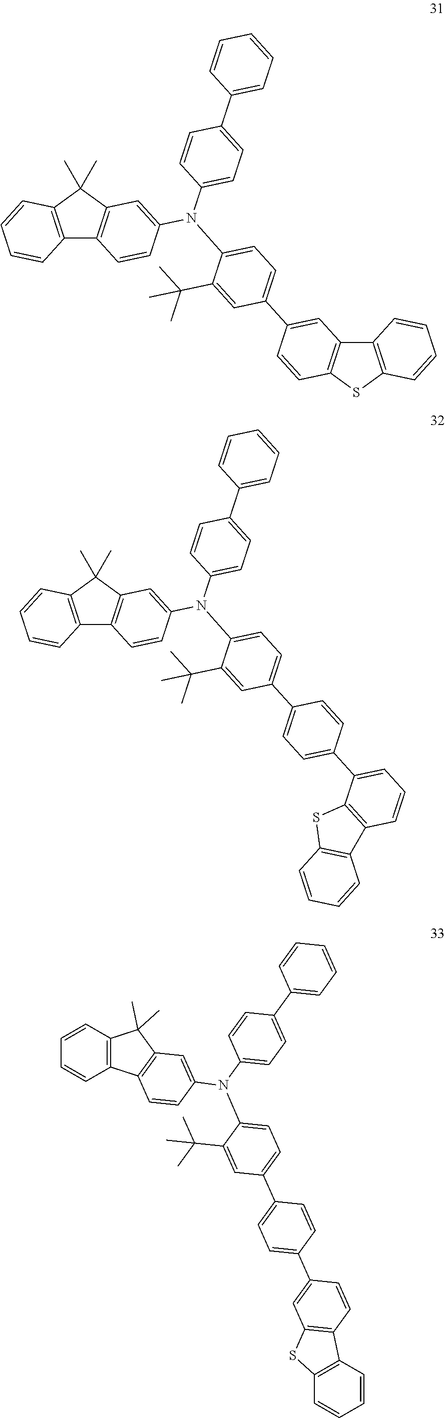

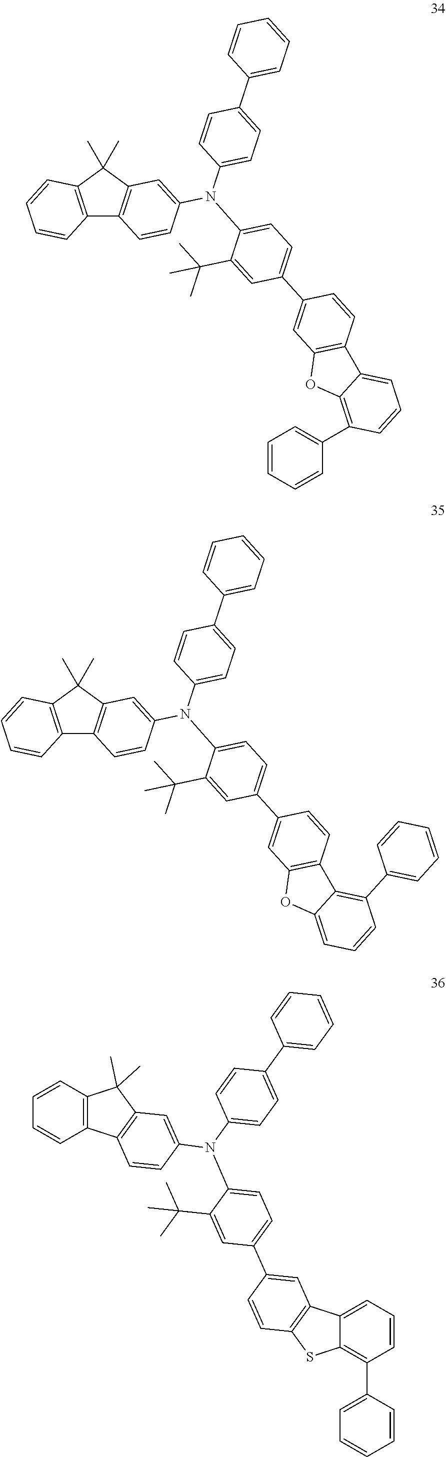 [SMILES: CC(C)(C)c1cc(-c2ccc3c(c2)oc2c(-c4ccccc4)cccc23)ccc1N(c1ccc(-c2ccccc2)cc1)c1ccc2c(c1)C(C)(C)c1ccccc1-2 ; CC(C)(C)c1cc(-c2ccc3c(c2)oc2cccc(-c4ccccc4)c23)ccc1N(c1ccc(-c2ccccc2)cc1)c1ccc2c(c1)C(C)(C)c1ccccc1-2 ; CC(C)(C)c1cc(-c2ccc3sc4c(-c5ccccc5)cccc4c3c2)ccc1N(c1ccc(-c2ccccc2)cc1)c1ccc2c(c1)C(C)(C)c1ccccc1-2]

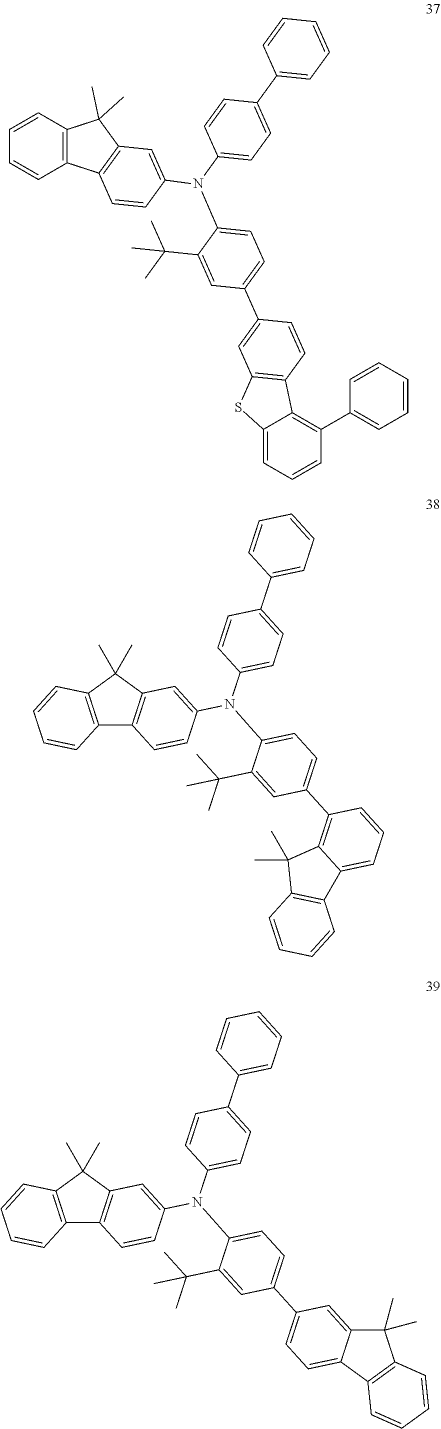

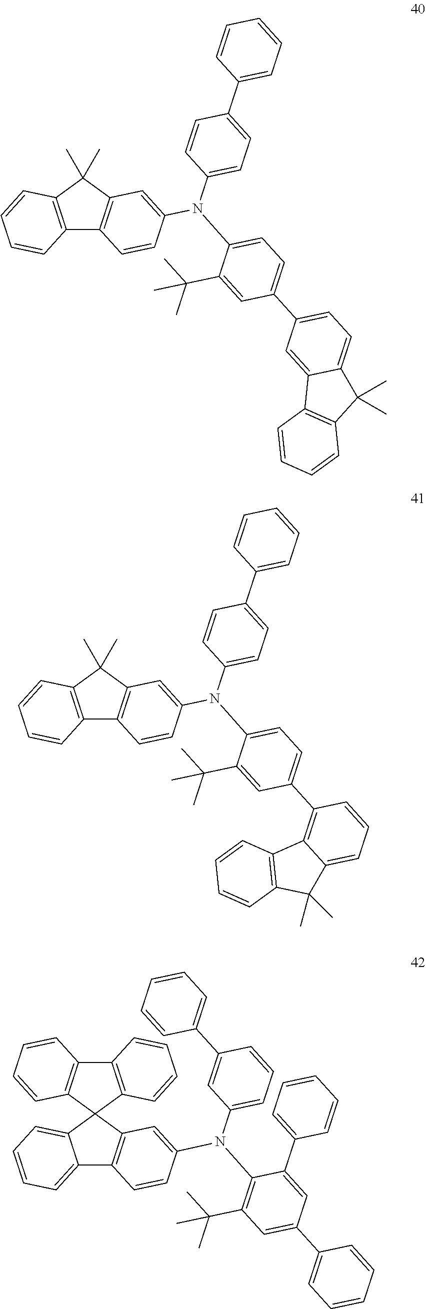 [SMILES: CC(C)(C)c1cc(-c2ccc3c(c2)-c2ccccc2C3(C)C)ccc1N(c1ccc(-c2ccccc2)cc1)c1ccc2c(c1)C(C)(C)c1ccccc1-2 ; CC(C)(C)c1cc(-c2cccc3c2-c2ccccc2C3(C)C)ccc1N(c1ccc(-c2ccccc2)cc1)c1ccc2c(c1)C(C)(C)c1ccccc1-2 ; CC(C)(C)c1cc(-c2ccccc2)cc(-c2ccccc2)c1N(c1cccc(-c2ccccc2)c1)c1ccc2c(c1)C1(c3ccccc3-c3ccccc31)c1ccccc1-2]

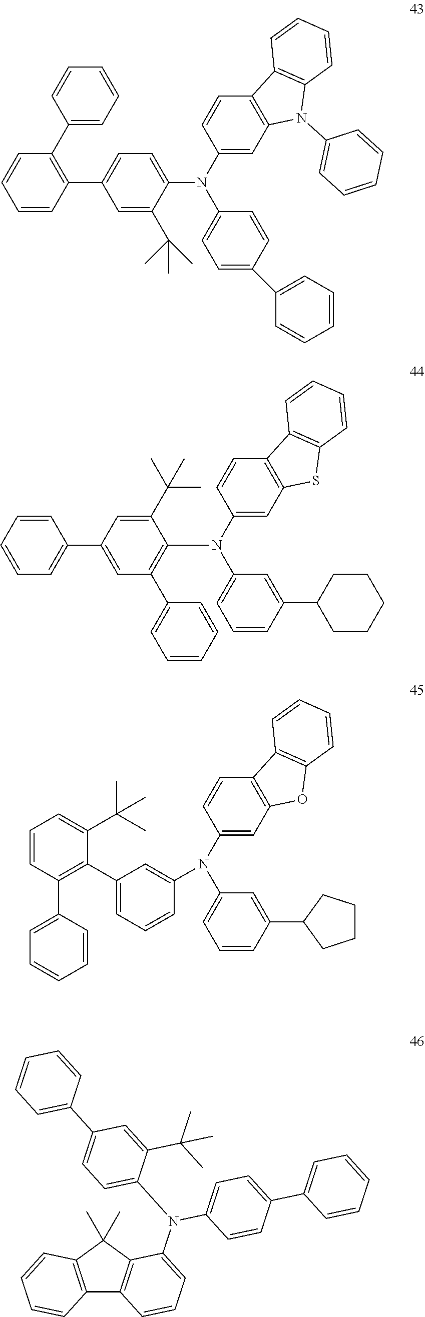

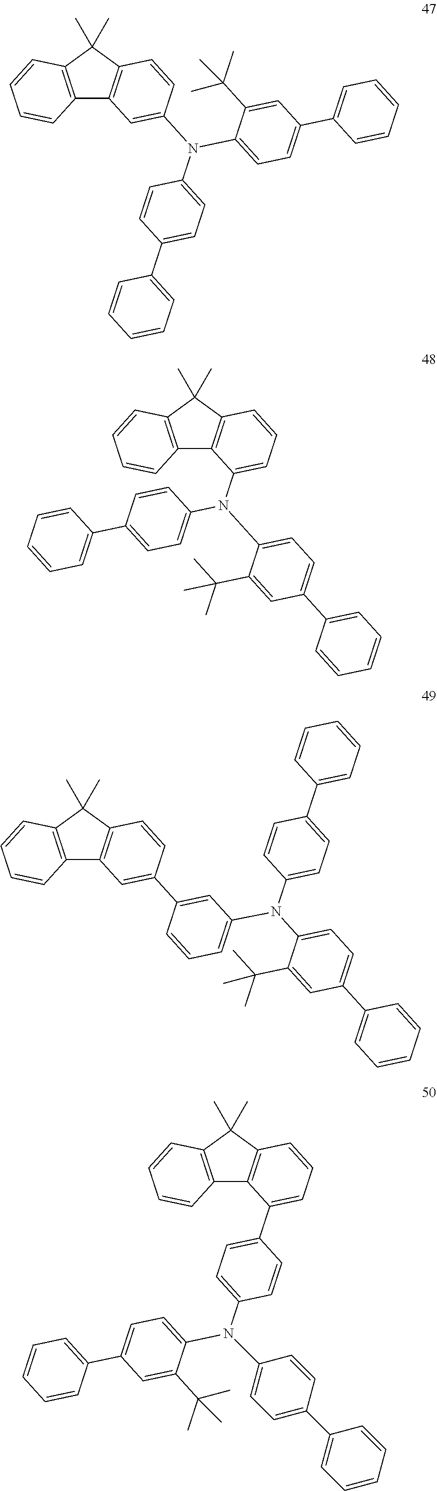

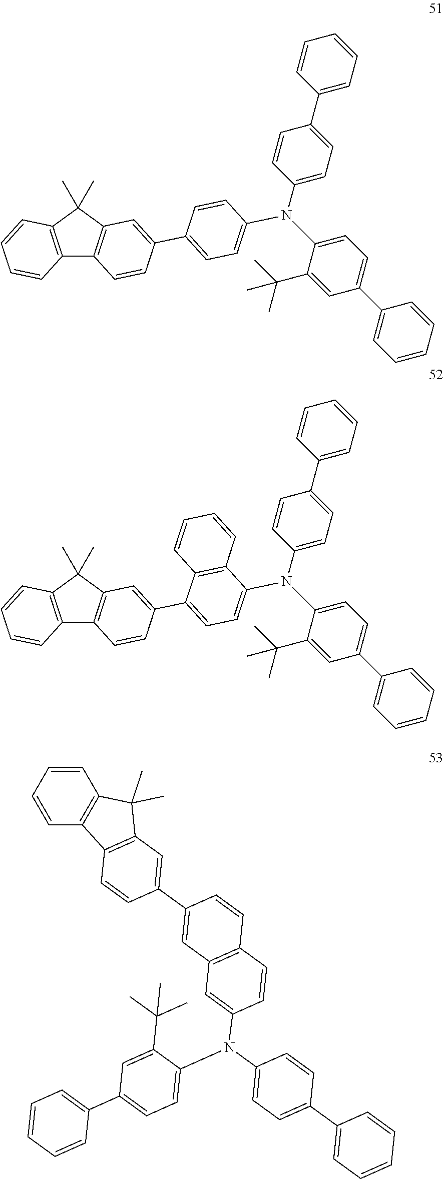 [SMILES: CC(C)(C)c1cc(-c2ccccc2)ccc1N(c1ccc(-c2ccccc2)cc1)c1ccc(-c2ccc3c(c2)C(C)(C)c2ccccc2-3)cc1 ; CC(C)(C)c1cc(-c2ccccc2)ccc1N(c1ccc(-c2ccccc2)cc1)c1ccc(-c2ccc3c(c2)C(C)(C)c2ccccc2-3)c2ccccc12 ; CC(C)(C)c1cc(-c2ccccc2)ccc1N(c1ccc(-c2ccccc2)cc1)c1ccc2ccc(-c3ccc4c(c3)C(C)(C)c3ccccc3-4)cc2c1]

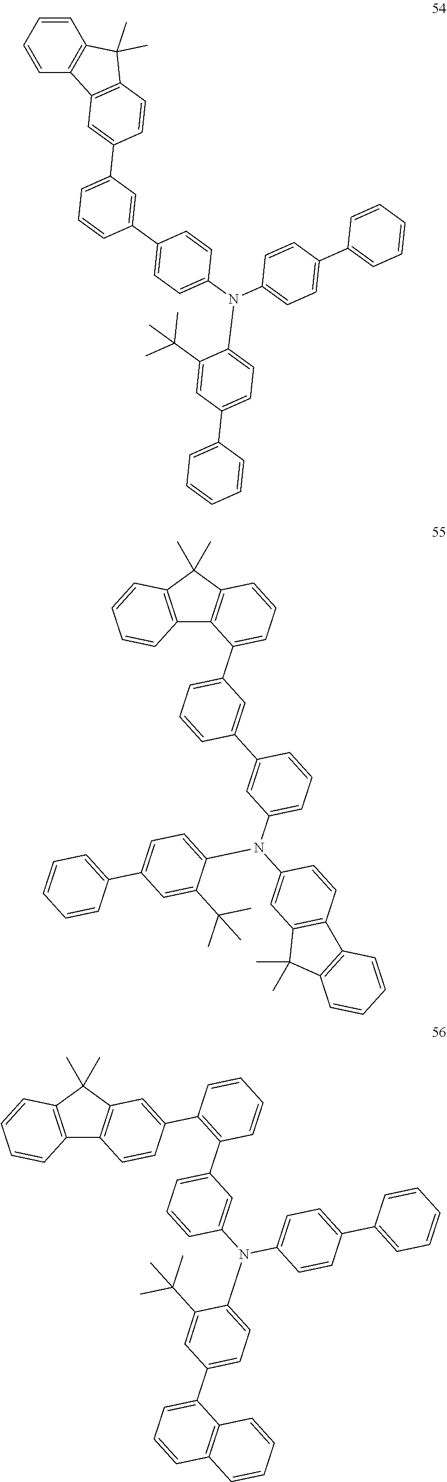

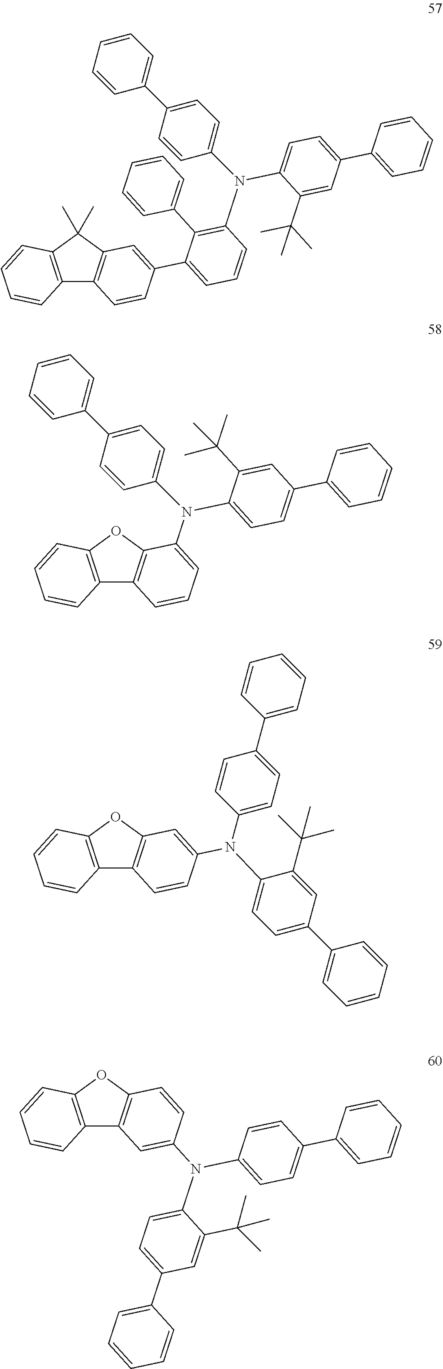 [SMILES: CC(C)(C)c1cc(-c2ccccc2)ccc1N(c1ccc(-c2ccccc2)cc1)c1cccc(-c2ccc3c(c2)C(C)(C)c2ccccc2-3)c1-c1ccccc1 ; CC(C)(C)c1cc(-c2ccccc2)ccc1N(c1ccc(-c2ccccc2)cc1)c1cccc2c1oc1ccccc12 ; CC(C)(C)c1cc(-c2ccccc2)ccc1N(c1ccc(-c2ccccc2)cc1)c1ccc2c(c1)oc1ccccc12 ; CC(C)(C)c1cc(-c2ccccc2)ccc1N(c1ccc(-c2ccccc2)cc1)c1ccc2oc3ccccc3c2c1]

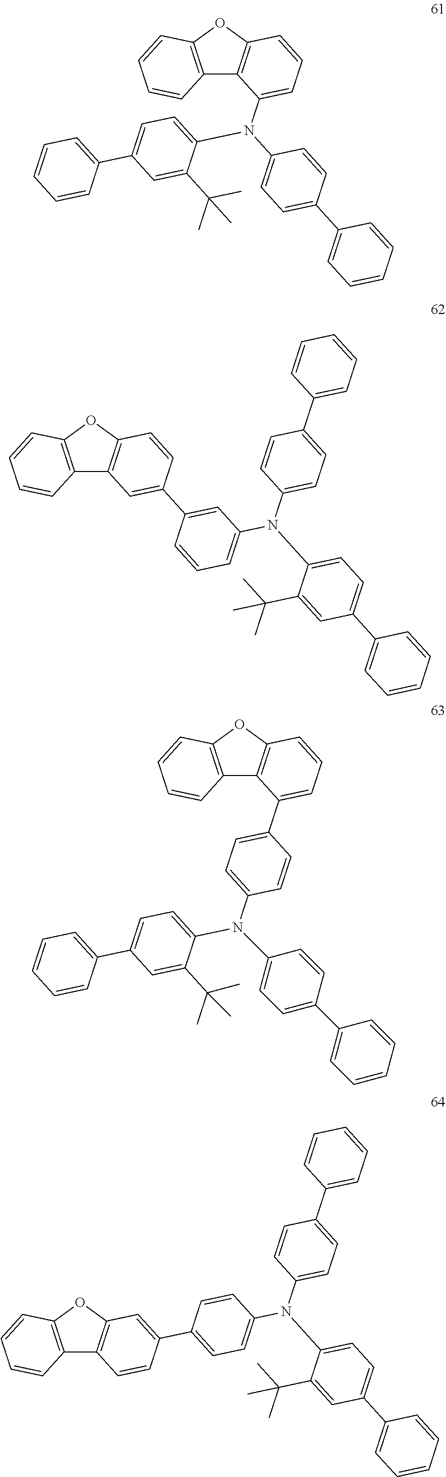

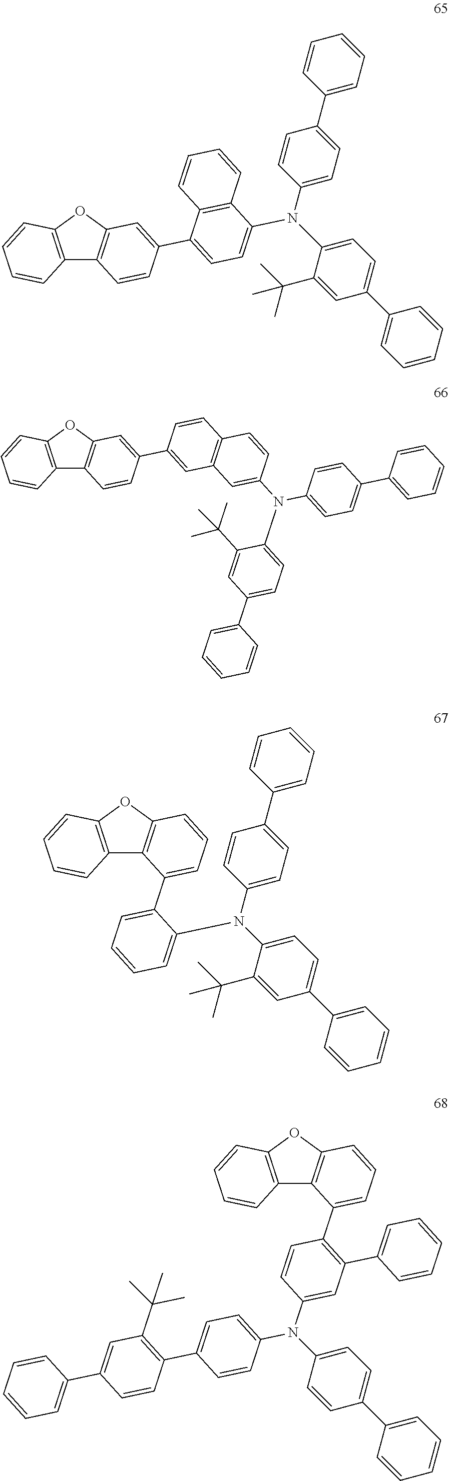 [SMILES: CC(C)(C)c1cc(-c2ccccc2)ccc1N(c1ccc(-c2ccccc2)cc1)c1ccc(-c2ccc3c(c2)oc2ccccc23)c2ccccc12 ; CC(C)(C)c1cc(-c2ccccc2)ccc1N(c1ccc(-c2ccccc2)cc1)c1ccc2ccc(-c3ccc4c(c3)oc3ccccc34)cc2c1 ; CC(C)(C)c1cc(-c2ccccc2)ccc1N(c1ccc(-c2ccccc2)cc1)c1ccccc1-c1cccc2oc3ccccc3c12 ; CC(C)(C)c1cc(-c2ccccc2)ccc1-c1ccc(N(c2ccc(-c3ccccc3)cc2)c2ccc(-c3cccc4oc5ccccc5c34)c(-c3ccccc3)c2)cc1]

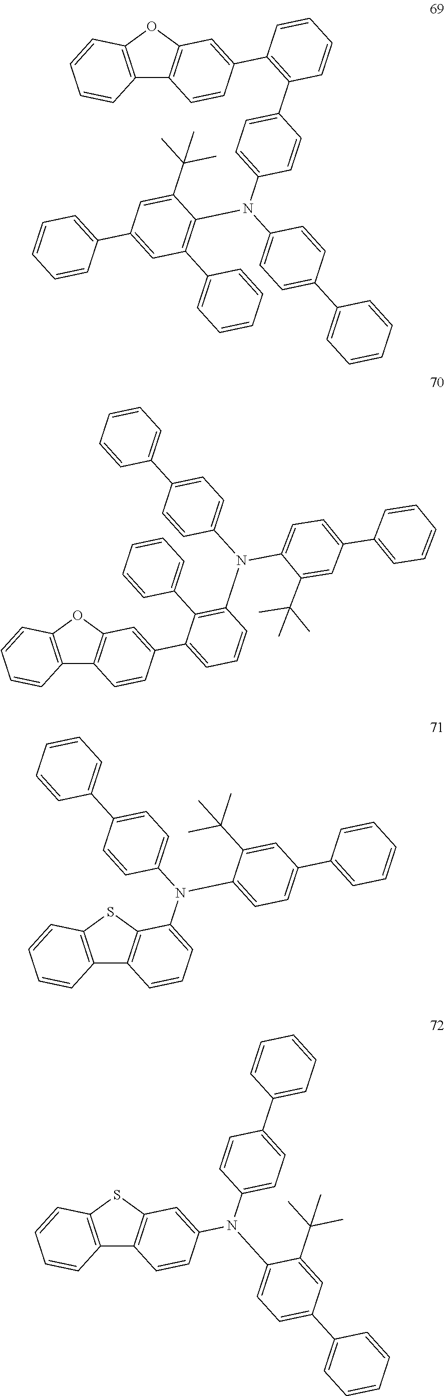 [SMILES: CC(C)(C)c1cc(-c2ccccc2)cc(-c2ccccc2)c1N(c1ccc(-c2ccccc2)cc1)c1ccc(-c2ccccc2-c2ccc3c(c2)oc2ccccc23)cc1 ; CC(C)(C)c1cc(-c2ccccc2)ccc1N(c1ccc(-c2ccccc2)cc1)c1cccc(-c2ccc3c(c2)oc2ccccc23)c1-c1ccccc1 ; CC(C)(C)c1cc(-c2ccccc2)ccc1N(c1ccc(-c2ccccc2)cc1)c1cccc2c1sc1ccccc12 ; CC(C)(C)c1cc(-c2ccccc2)ccc1N(c1ccc(-c2ccccc2)cc1)c1ccc2c(c1)sc1ccccc12]

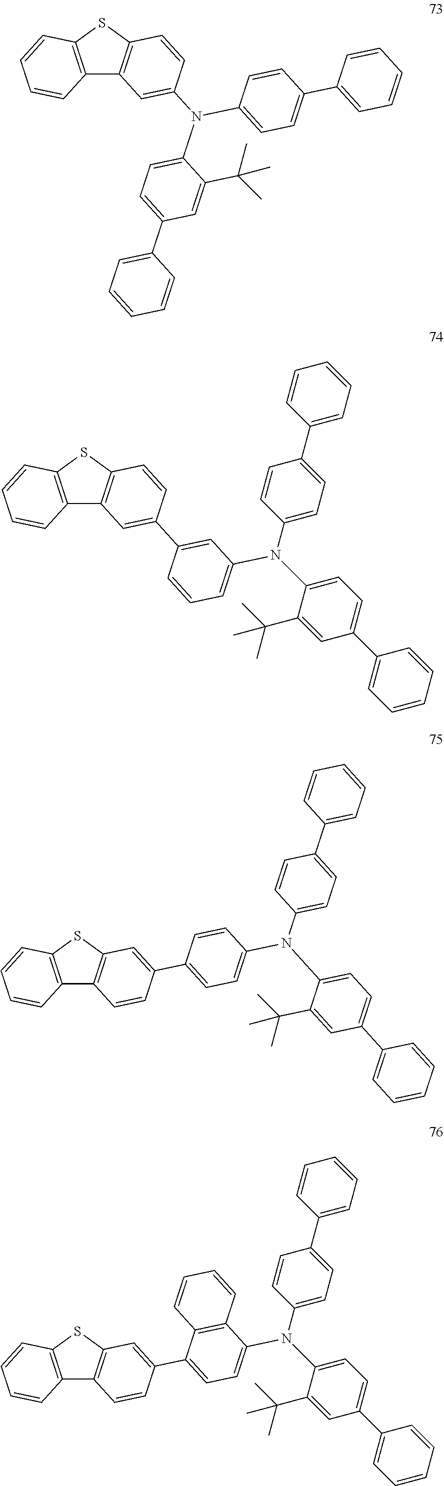 [SMILES: CC(C)(C)c1cc(-c2ccccc2)ccc1N(c1ccc(-c2ccccc2)cc1)c1ccc2sc3ccccc3c2c1 ; CC(C)(C)c1cc(-c2ccccc2)ccc1N(c1ccc(-c2ccccc2)cc1)c1cccc(-c2ccc3sc4ccccc4c3c2)c1 ; CC(C)(C)c1cc(-c2ccccc2)ccc1N(c1ccc(-c2ccccc2)cc1)c1ccc(-c2ccc3c(c2)sc2ccccc23)cc1 ; CC(C)(C)c1cc(-c2ccccc2)ccc1N(c1ccc(-c2ccccc2)cc1)c1ccc(-c2ccc3c(c2)sc2ccccc23)c2ccccc12]

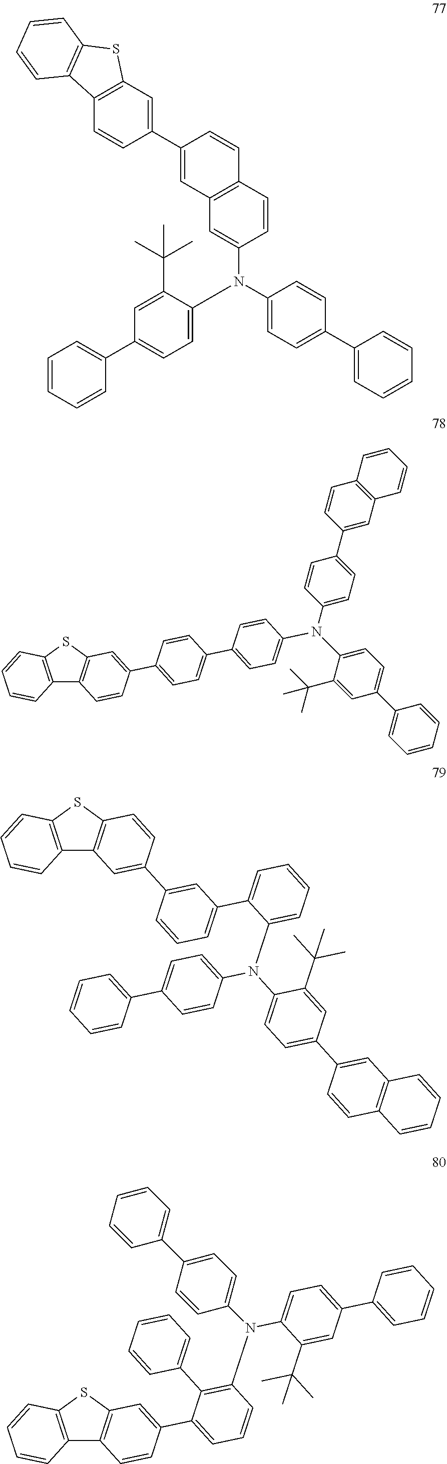 [SMILES: CC(C)(C)c1cc(-c2ccccc2)ccc1N(c1ccc(-c2ccccc2)cc1)c1ccc2ccc(-c3ccc4c(c3)sc3ccccc34)cc2c1 ; CC(C)(C)c1cc(-c2ccccc2)ccc1N(c1ccc(-c2ccc(-c3ccc4c(c3)sc3ccccc34)cc2)cc1)c1ccc(-c2ccc3ccccc3c2)cc1 ; CC(C)(C)c1cc(-c2ccc3ccccc3c2)ccc1N(c1ccc(-c2ccccc2)cc1)c1ccccc1-c1cccc(-c2ccc3sc4ccccc4c3c2)c1 ; CC(C)(C)c1cc(-c2ccccc2)ccc1N(c1ccc(-c2ccccc2)cc1)c1cccc(-c2ccc3c(c2)sc2ccccc23)c1-c1ccccc1]

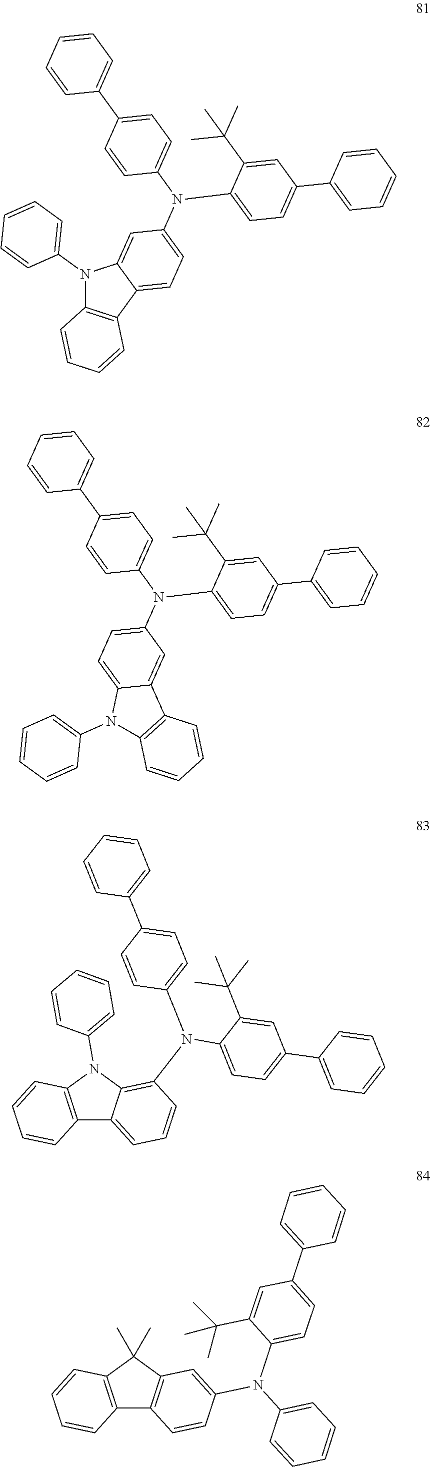 [SMILES: CC(C)(C)c1cc(-c2ccccc2)ccc1N(c1ccc(-c2ccccc2)cc1)c1ccc2c3ccccc3n(-c3ccccc3)c2c1 ; CC(C)(C)c1cc(-c2ccccc2)ccc1N(c1ccc(-c2ccccc2)cc1)c1ccc2c(c1)c1ccccc1n2-c1ccccc1 ; CC(C)(C)c1cc(-c2ccccc2)ccc1N(c1ccc(-c2ccccc2)cc1)c1cccc2c3ccccc3n(-c3ccccc3)c12 ; CC(C)(C)c1cc(-c2ccccc2)ccc1N(c1ccccc1)c1ccc2c(c1)C(C)(C)c1ccccc1-2]

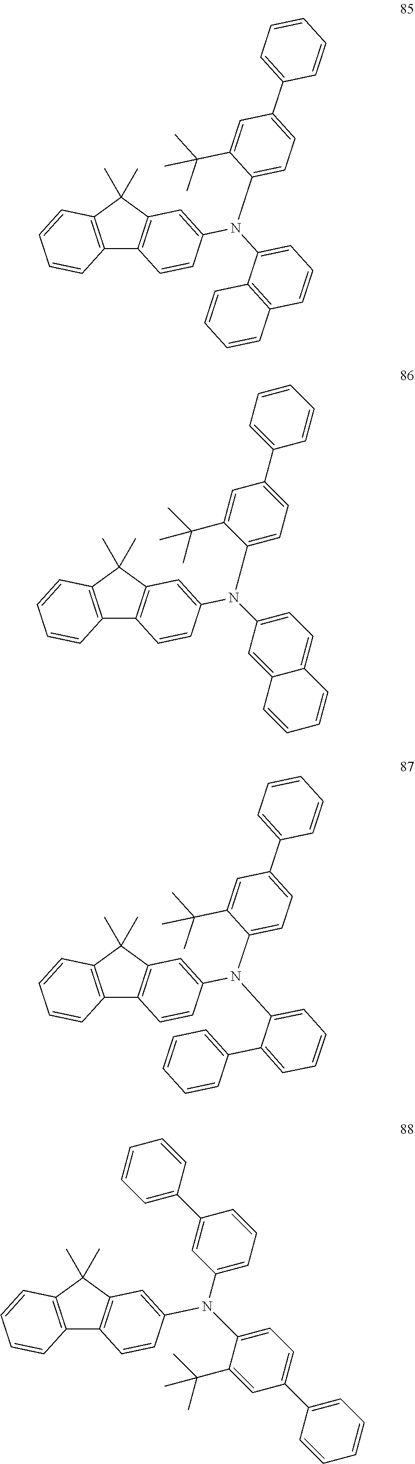

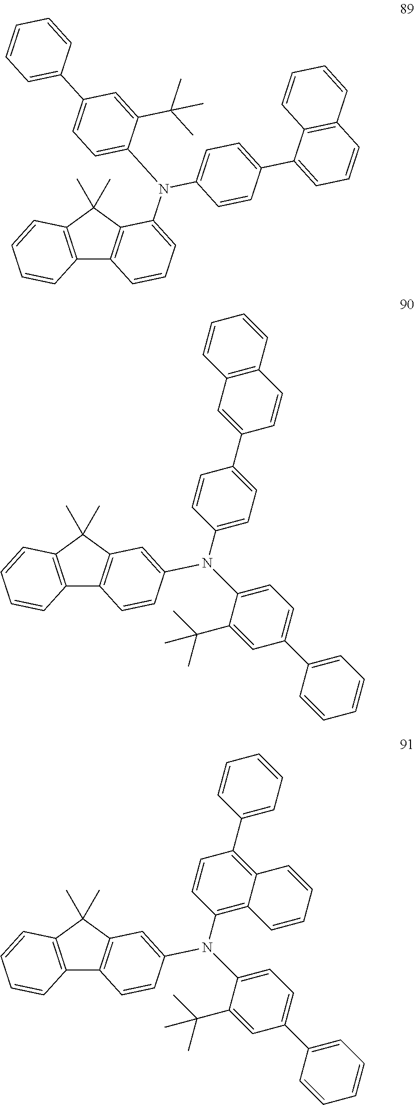 [SMILES: CC(C)(C)c1cc(-c2ccccc2)ccc1N(c1ccc(-c2cccc3ccccc23)cc1)c1cccc2c1C(C)(C)c1ccccc1-2 ; CC(C)(C)c1cc(-c2ccccc2)ccc1N(c1ccc(-c2ccc3ccccc3c2)cc1)c1ccc2c(c1)C(C)(C)c1ccccc1-2 ; CC(C)(C)c1cc(-c2ccccc2)ccc1N(c1ccc2c(c1)C(C)(C)c1ccccc1-2)c1ccc(-c2ccccc2)c2ccccc12]

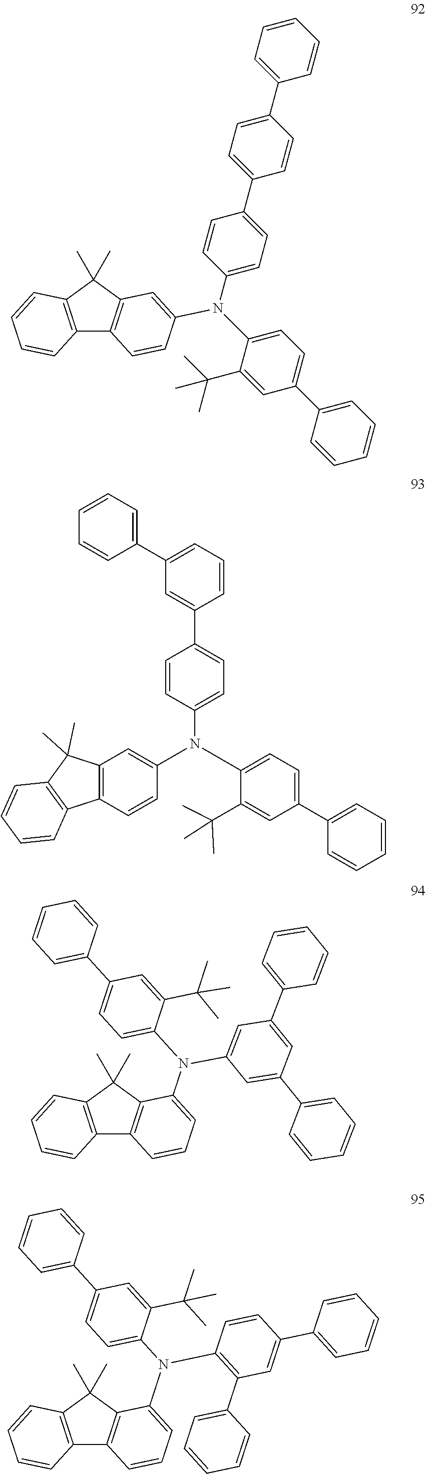 [SMILES: CC(C)(C)c1cc(-c2ccccc2)ccc1N(c1ccc(-c2ccc(-c3ccccc3)cc2)cc1)c1ccc2c(c1)C(C)(C)c1ccccc1-2 ; CC(C)(C)c1cc(-c2ccccc2)ccc1N(c1ccc(-c2cccc(-c3ccccc3)c2)cc1)c1ccc2c(c1)C(C)(C)c1ccccc1-2 ; CC(C)(C)c1cc(-c2ccccc2)ccc1N(c1cc(-c2ccccc2)cc(-c2ccccc2)c1)c1cccc2c1C(C)(C)c1ccccc1-2 ; CC(C)(C)c1cc(-c2ccccc2)ccc1N(c1ccc(-c2ccccc2)cc1-c1ccccc1)c1cccc2c1C(C)(C)c1ccccc1-2]

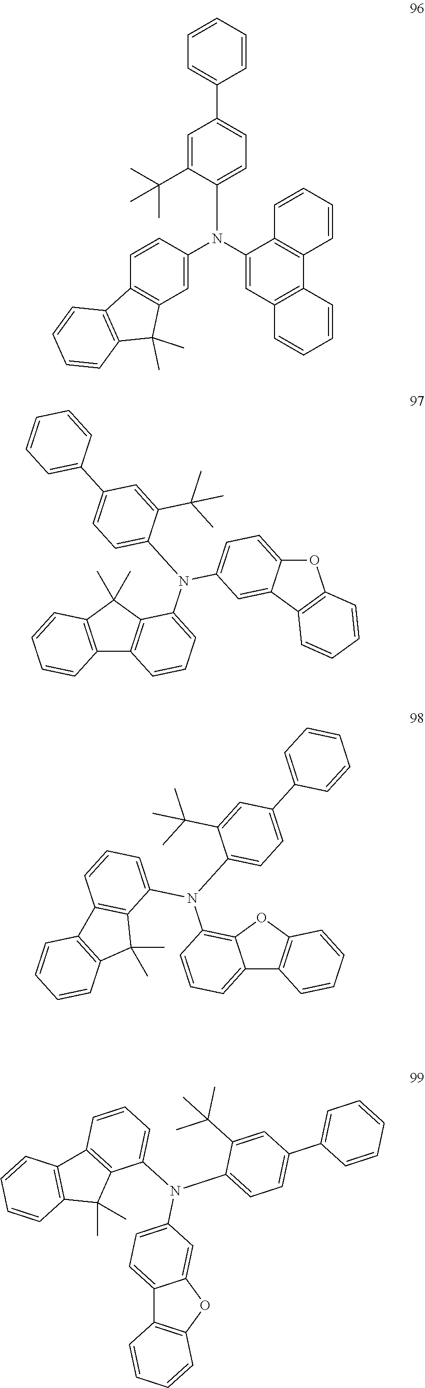

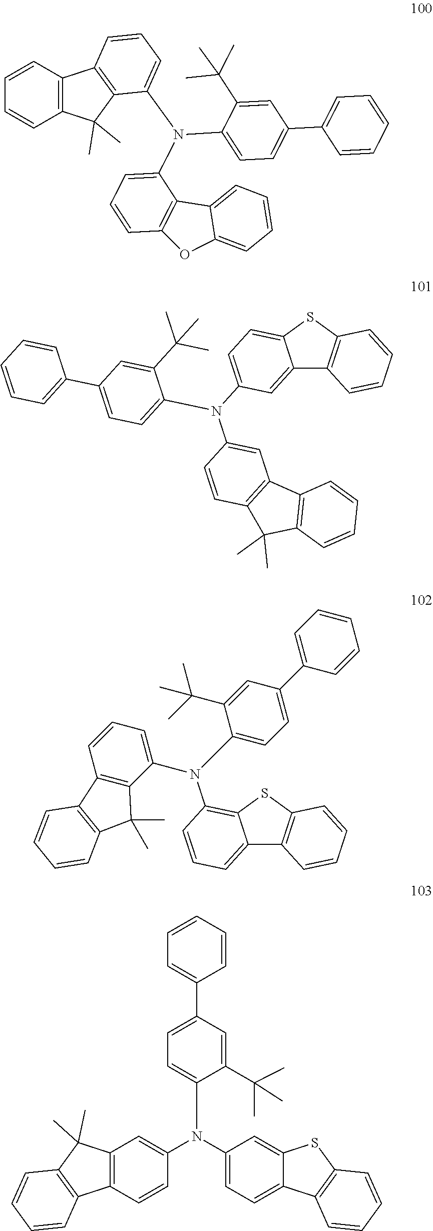 [SMILES: CC(C)(C)c1cc(-c2ccccc2)ccc1N(c1cccc2c1C(C)(C)c1ccccc1-2)c1cccc2oc3ccccc3c12 ; CC(C)(C)c1cc(-c2ccccc2)ccc1N(c1ccc2c(c1)-c1ccccc1C2(C)C)c1ccc2sc3ccccc3c2c1 ; CC(C)(C)c1cc(-c2ccccc2)ccc1N(c1cccc2c1C(C)(C)c1ccccc1-2)c1cccc2c1sc1ccccc12 ; CC(C)(C)c1cc(-c2ccccc2)ccc1N(c1ccc2c(c1)C(C)(C)c1ccccc1-2)c1ccc2c(c1)sc1ccccc12]

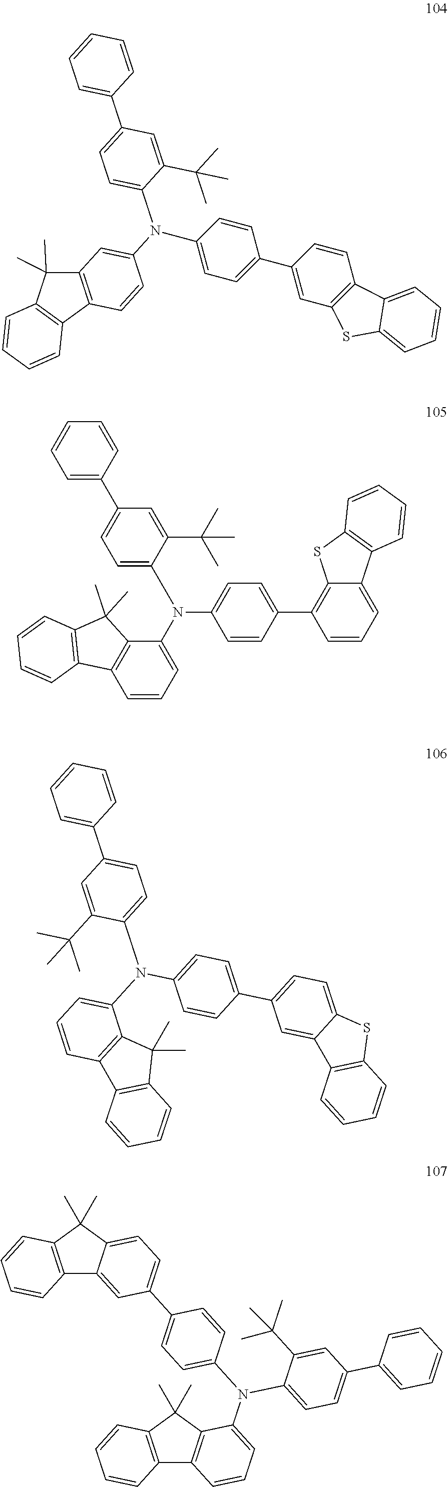 [SMILES: CC(C)(C)c1cc(-c2ccccc2)ccc1N(c1ccc(-c2ccc3c(c2)sc2ccccc23)cc1)c1ccc2c(c1)C(C)(C)c1ccccc1-2 ; CC(C)(C)c1cc(-c2ccccc2)ccc1N(c1ccc(-c2cccc3c2sc2ccccc23)cc1)c1cccc2c1C(C)(C)c1ccccc1-2 ; CC(C)(C)c1cc(-c2ccccc2)ccc1N(c1ccc(-c2ccc3sc4ccccc4c3c2)cc1)c1cccc2c1C(C)(C)c1ccccc1-2 ; CC(C)(C)c1cc(-c2ccccc2)ccc1N(c1ccc(-c2ccc3c(c2)-c2ccccc2C3(C)C)cc1)c1cccc2c1C(C)(C)c1ccccc1-2]

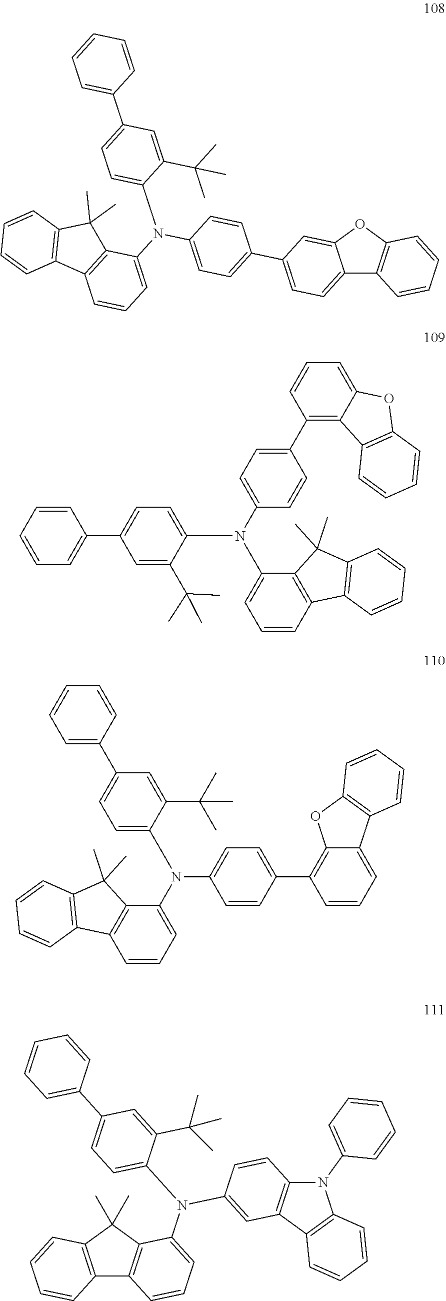 [SMILES: CC(C)(C)c1cc(-c2ccccc2)ccc1N(c1ccc(-c2ccc3c(c2)oc2ccccc23)cc1)c1cccc2c1C(C)(C)c1ccccc1-2 ; CC(C)(C)c1cc(-c2ccccc2)ccc1N(c1ccc(-c2cccc3oc4ccccc4c23)cc1)c1cccc2c1C(C)(C)c1ccccc1-2 ; CC(C)(C)c1cc(-c2ccccc2)ccc1N(c1ccc(-c2cccc3c2oc2ccccc23)cc1)c1cccc2c1C(C)(C)c1ccccc1-2 ; CC(C)(C)c1cc(-c2ccccc2)ccc1N(c1ccc2c(c1)c1ccccc1n2-c1ccccc1)c1cccc2c1C(C)(C)c1ccccc1-2]

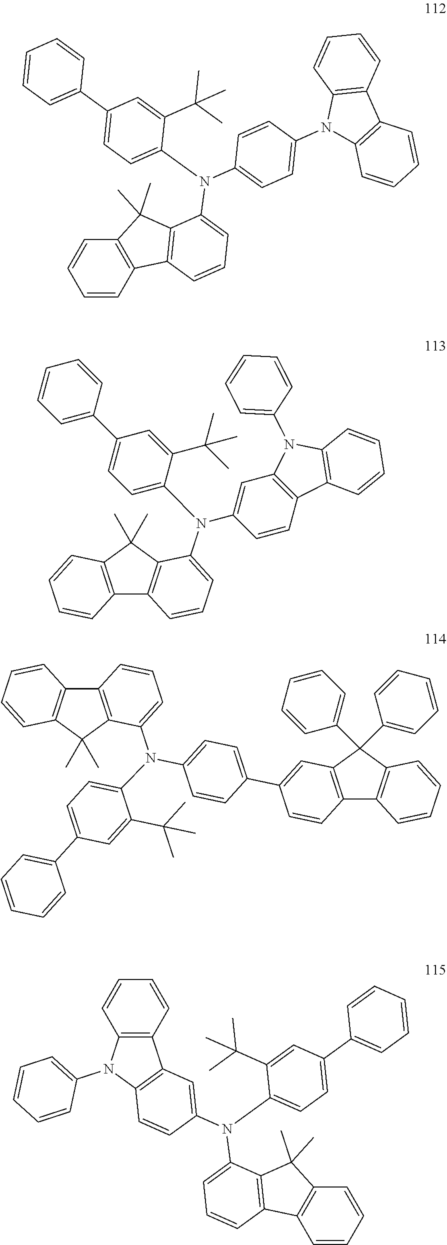 [SMILES: CC(C)(C)c1cc(-c2ccccc2)ccc1N(c1ccc(-n2c3ccccc3c3ccccc32)cc1)c1cccc2c1C(C)(C)c1ccccc1-2 ; CC(C)(C)c1cc(-c2ccccc2)ccc1N(c1ccc2c3ccccc3n(-c3ccccc3)c2c1)c1cccc2c1C(C)(C)c1ccccc1-2 ; CC(C)(C)c1cc(-c2ccccc2)ccc1N(c1ccc(-c2ccc3c(c2)C(c2ccccc2)(c2ccccc2)c2ccccc2-3)cc1)c1cccc2c1C(C)(C)c1ccccc1-2 ; CC(C)(C)c1cc(-c2ccccc2)ccc1N(c1ccc2c(c1)c1ccccc1n2-c1ccccc1)c1cccc2c1C(C)(C)c1ccccc1-2]

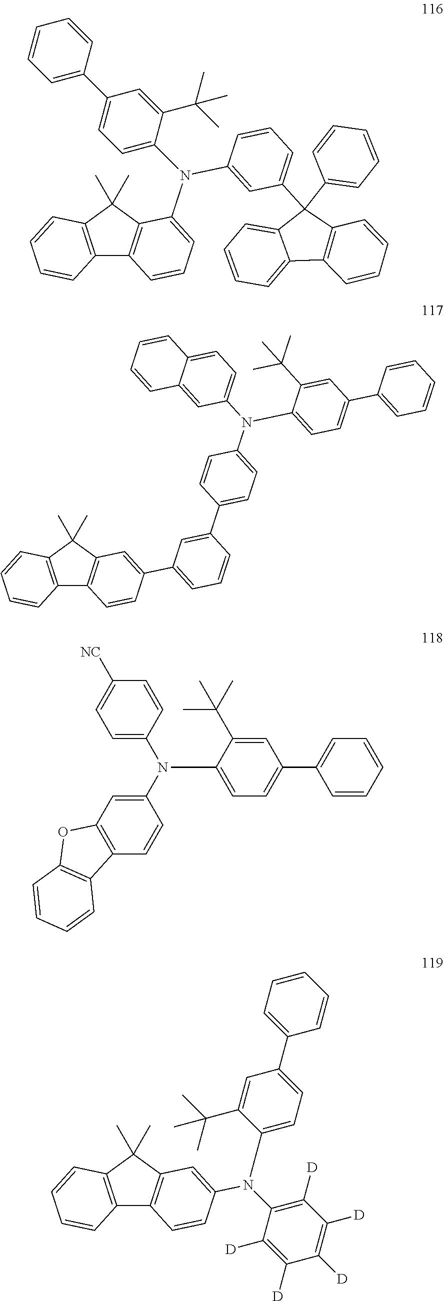

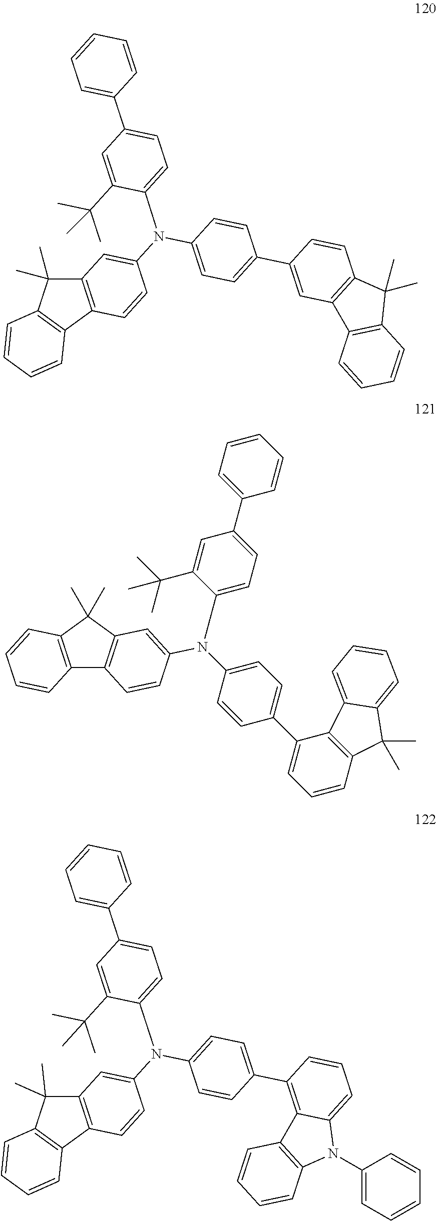 [SMILES: CC(C)(C)c1cc(-c2ccccc2)ccc1N(c1ccc(-c2ccc3c(c2)-c2ccccc2C3(C)C)cc1)c1ccc2c(c1)C(C)(C)c1ccccc1-2 ; CC(C)(C)c1cc(-c2ccccc2)ccc1N(c1ccc(-c2cccc3c2-c2ccccc2C3(C)C)cc1)c1ccc2c(c1)C(C)(C)c1ccccc1-2 ; CC(C)(C)c1cc(-c2ccccc2)ccc1N(c1ccc(-c2cccc3c2c2ccccc2n3-c2ccccc2)cc1)c1ccc2c(c1)C(C)(C)c1ccccc1-2]

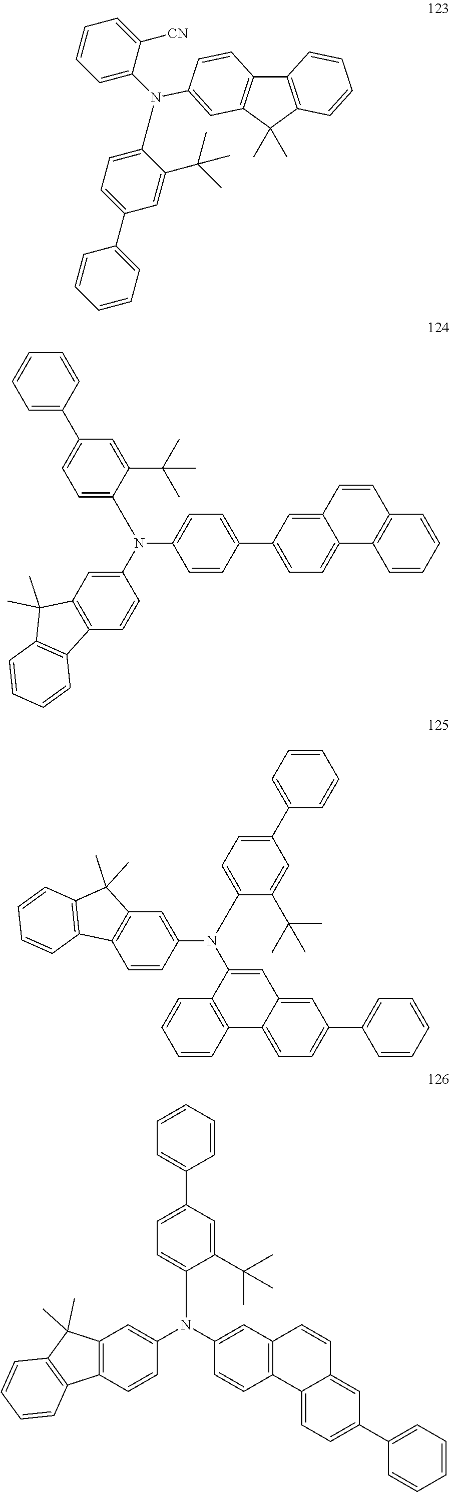 [SMILES: CC(C)(C)c1cc(-c2ccccc2)ccc1N(c1ccc2c(c1)C(C)(C)c1ccccc1-2)c1ccccc1C#N ; CC(C)(C)c1cc(-c2ccccc2)ccc1N(c1ccc(-c2ccc3c(ccc4ccccc43)c2)cc1)c1ccc2c(c1)C(C)(C)c1ccccc1-2 ; CC(C)(C)c1cc(-c2ccccc2)ccc1N(c1ccc2c(c1)C(C)(C)c1ccccc1-2)c1cc2cc(-c3ccccc3)ccc2c2ccccc12 ; CC(C)(C)c1cc(-c2ccccc2)ccc1N(c1ccc2c(c1)C(C)(C)c1ccccc1-2)c1ccc2c(ccc3cc(-c4ccccc4)ccc32)c1]

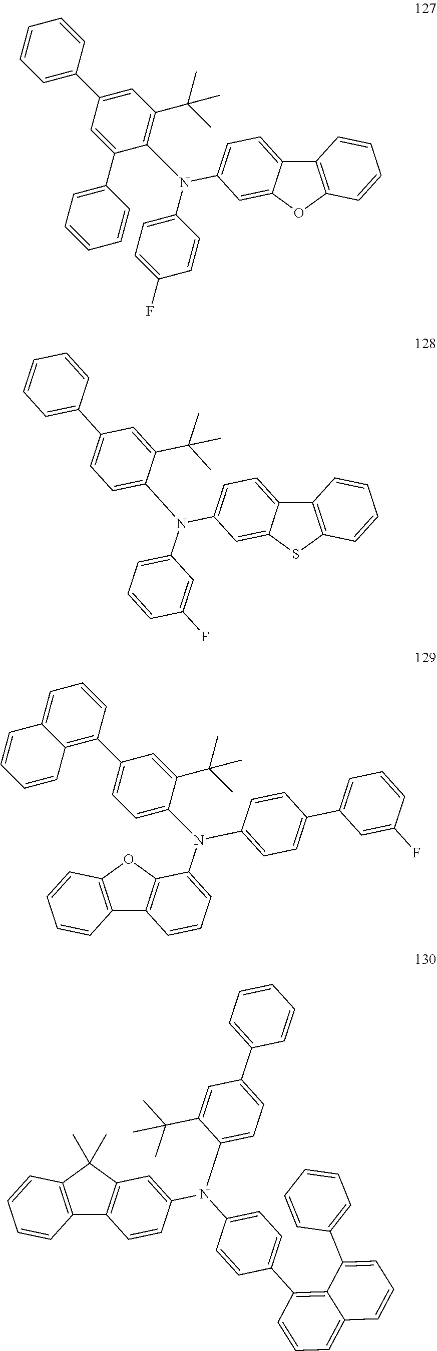 [SMILES: CC(C)(C)c1cc(-c2ccccc2)cc(-c2ccccc2)c1N(c1ccc(F)cc1)c1ccc2c(c1)oc1ccccc12 ; CC(C)(C)c1cc(-c2ccccc2)ccc1N(c1cccc(F)c1)c1ccc2c(c1)sc1ccccc12 ; CC(C)(C)c1cc(-c2cccc3ccccc23)ccc1N(c1ccc(-c2cccc(F)c2)cc1)c1cccc2c1oc1ccccc12 ; CC(C)(C)c1cc(-c2ccccc2)ccc1N(c1ccc(-c2cccc3cccc(-c4ccccc4)c23)cc1)c1ccc2c(c1)C(C)(C)c1ccccc1-2]

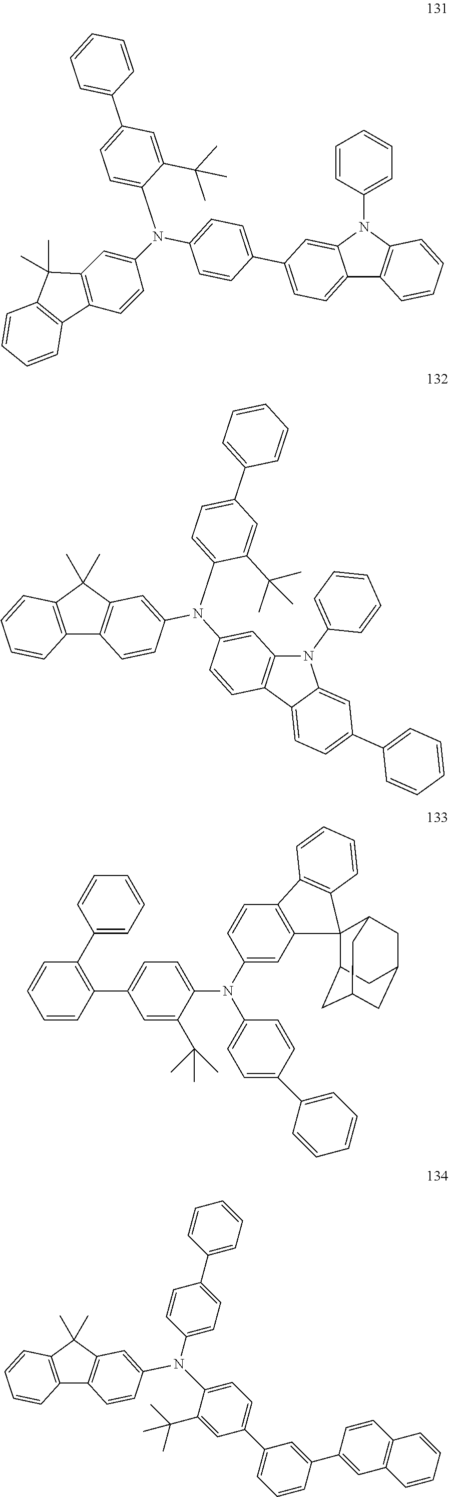 [SMILES: CC(C)(C)c1cc(-c2ccccc2)ccc1N(c1ccc(-c2ccc3c4ccccc4n(-c4ccccc4)c3c2)cc1)c1ccc2c(c1)C(C)(C)c1ccccc1-2 ; CC(C)(C)c1cc(-c2ccccc2)ccc1N(c1ccc2c(c1)C(C)(C)c1ccccc1-2)c1ccc2c3ccc(-c4ccccc4)cc3n(-c3ccccc3)c2c1 ; CC(C)(C)c1cc(-c2ccccc2-c2ccccc2)ccc1N(c1ccc(-c2ccccc2)cc1)c1ccc2c(c1)C1(c3ccccc3-2)C2CC3CC(C2)CC1C3 ; CC(C)(C)c1cc(-c2cccc(-c3ccc4ccccc4c3)c2)ccc1N(c1ccc(-c2ccccc2)cc1)c1ccc2c(c1)C(C)(C)c1ccccc1-2]

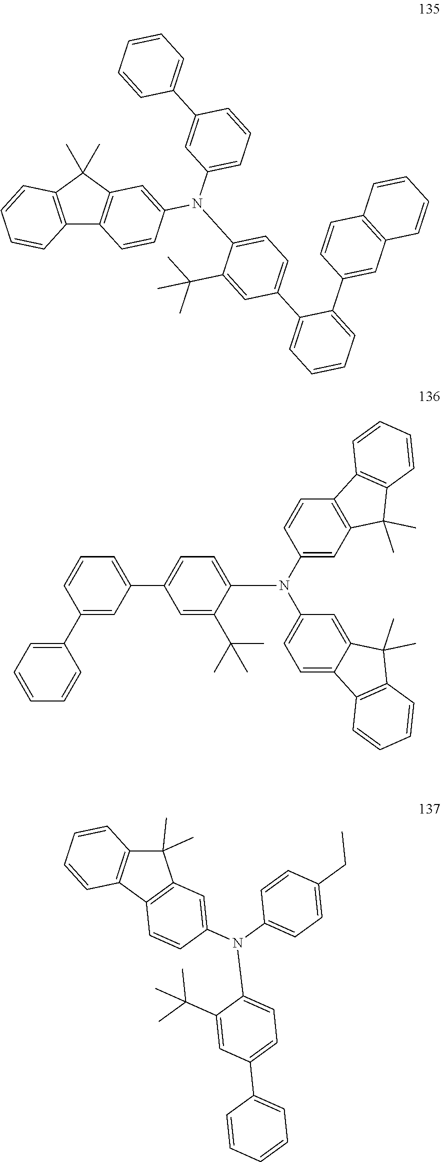 [SMILES: CC(C)(C)c1cc(-c2ccccc2-c2ccc3ccccc3c2)ccc1N(c1cccc(-c2ccccc2)c1)c1ccc2c(c1)C(C)(C)c1ccccc1-2 ; CC(C)(C)c1cc(-c2cccc(-c3ccccc3)c2)ccc1N(c1ccc2c(c1)C(C)(C)c1ccccc1-2)c1ccc2c(c1)C(C)(C)c1ccccc1-2 ; CCc1ccc(N(c2ccc3c(c2)C(C)(C)c2ccccc2-3)c2ccc(-c3ccccc3)cc2C(C)(C)C)cc1]

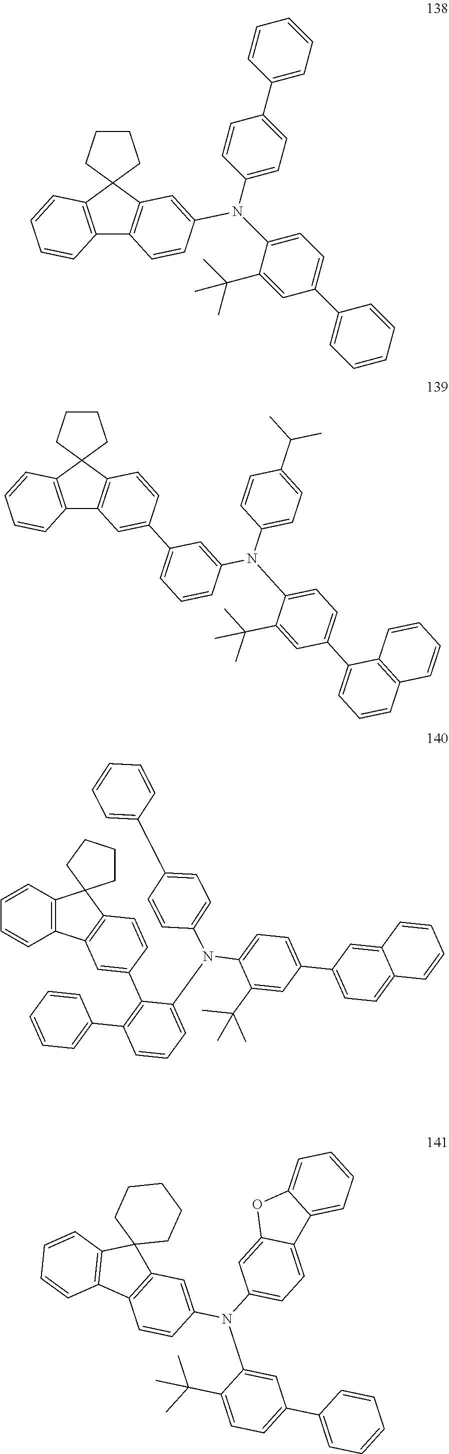 [SMILES: CC(C)(C)c1cc(-c2ccccc2)ccc1N(c1ccc(-c2ccccc2)cc1)c1ccc2c(c1)C1(CCCC1)c1ccccc1-2 ; CC(C)c1ccc(N(c2cccc(-c3ccc4c(c3)-c3ccccc3C43CCCC3)c2)c2ccc(-c3cccc4ccccc34)cc2C(C)(C)C)cc1 ; CC(C)(C)c1cc(-c2ccc3ccccc3c2)ccc1N(c1ccc(-c2ccccc2)cc1)c1cccc(-c2ccccc2)c1-c1ccc2c(c1)-c1ccccc1C21CCCC1 ; CC(C)(C)c1ccc(-c2ccccc2)cc1N(c1ccc2c(c1)C1(CCCCC1)c1ccccc1-2)c1ccc2c(c1)oc1ccccc12]

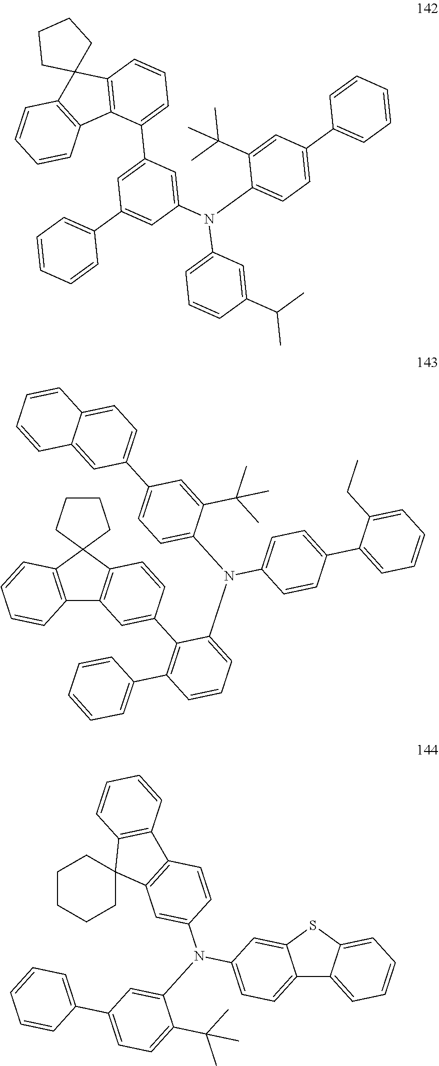 [SMILES: CC(C)c1cccc(N(c2cc(-c3ccccc3)cc(-c3cccc4c3-c3ccccc3C43CCCC3)c2)c2ccc(-c3ccccc3)cc2C(C)(C)C)c1 ; CCc1ccccc1-c1ccc(N(c2ccc(-c3ccc4ccccc4c3)cc2C(C)(C)C)c2cccc(-c3ccccc3)c2-c2ccc3c(c2)-c2ccccc2C32CCCC2)cc1 ; CC(C)(C)c1ccc(-c2ccccc2)cc1N(c1ccc2c(c1)C1(CCCCC1)c1ccccc1-2)c1ccc2c(c1)sc1ccccc12]

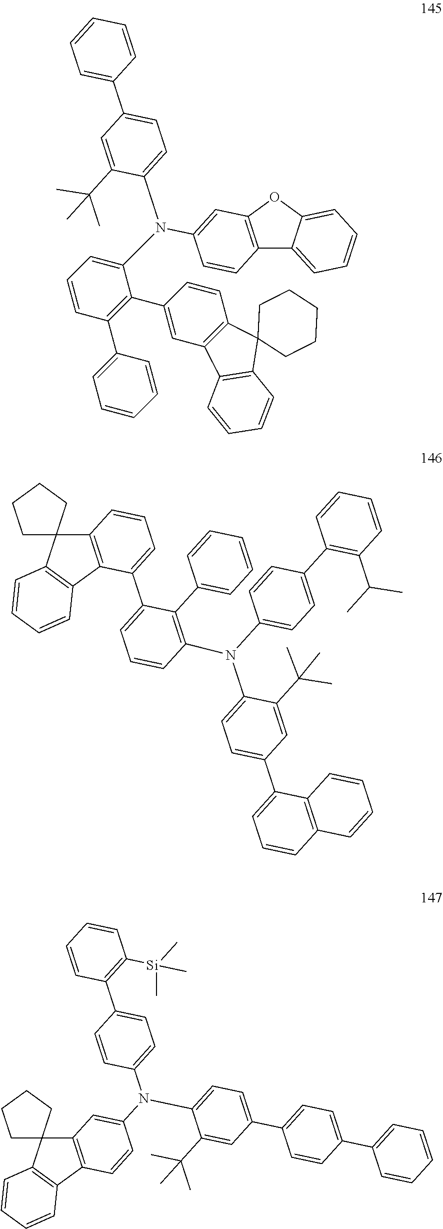 [SMILES: CC(C)(C)c1cc(-c2ccccc2)ccc1N(c1ccc2c(c1)oc1ccccc12)c1cccc(-c2ccccc2)c1-c1ccc2c(c1)-c1ccccc1C21CCCCC1 ; CC(C)c1ccccc1-c1ccc(N(c2ccc(-c3cccc4ccccc34)cc2C(C)(C)C)c2cccc(-c3cccc4c3-c3ccccc3C43CCCC3)c2-c2ccccc2)cc1 ; CC(C)(C)c1cc(-c2ccc(-c3ccccc3)cc2)ccc1N(c1ccc(-c2ccccc2[Si](C)(C)C)cc1)c1ccc2c(c1)C1(CCCC1)c1ccccc1-2]

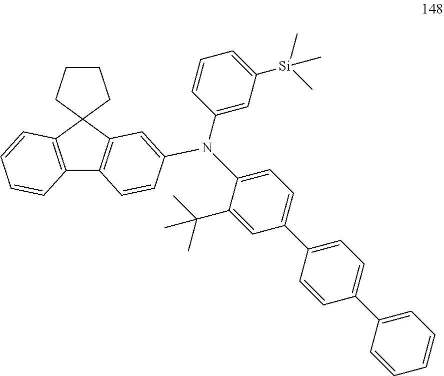 [SMILES: CC(C)(C)c1cc(-c2ccc(-c3ccccc3)cc2)ccc1N(c1cccc([Si](C)(C)C)c1)c1ccc2c(c1)C1(CCCC1)c1ccccc1-2]

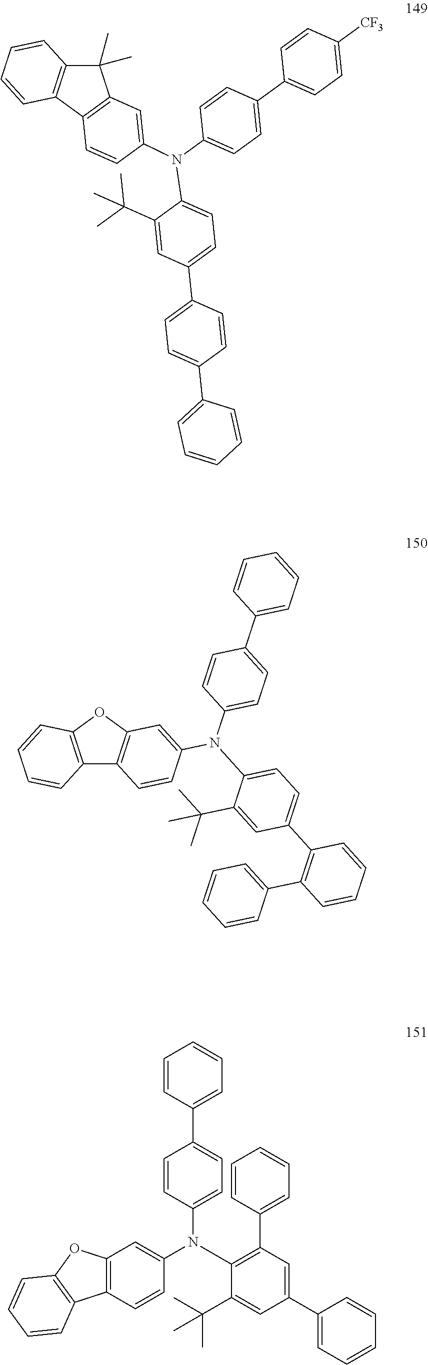 [SMILES: CC(C)(C)c1cc(-c2ccc(-c3ccccc3)cc2)ccc1N(c1ccc(-c2ccc(C(F)(F)F)cc2)cc1)c1ccc2c(c1)C(C)(C)c1ccccc1-2 ; CC(C)(C)c1cc(-c2ccccc2-c2ccccc2)ccc1N(c1ccc(-c2ccccc2)cc1)c1ccc2c(c1)oc1ccccc12 ; CC(C)(C)c1cc(-c2ccccc2)cc(-c2ccccc2)c1N(c1ccc(-c2ccccc2)cc1)c1ccc2c(c1)oc1ccccc12]

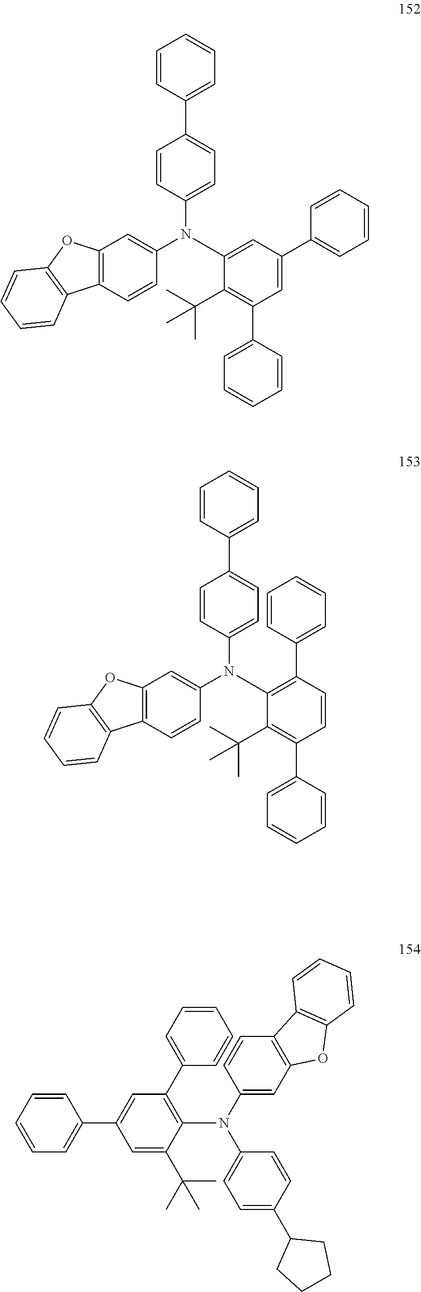 [SMILES: CC(C)(C)c1c(-c2ccccc2)cc(-c2ccccc2)cc1N(c1ccc(-c2ccccc2)cc1)c1ccc2c(c1)oc1ccccc12 ; CC(C)(C)c1c(-c2ccccc2)ccc(-c2ccccc2)c1N(c1ccc(-c2ccccc2)cc1)c1ccc2c(c1)oc1ccccc12 ; CC(C)(C)c1cc(-c2ccccc2)cc(-c2ccccc2)c1N(c1ccc(C2CCCC2)cc1)c1ccc2c(c1)oc1ccccc12]

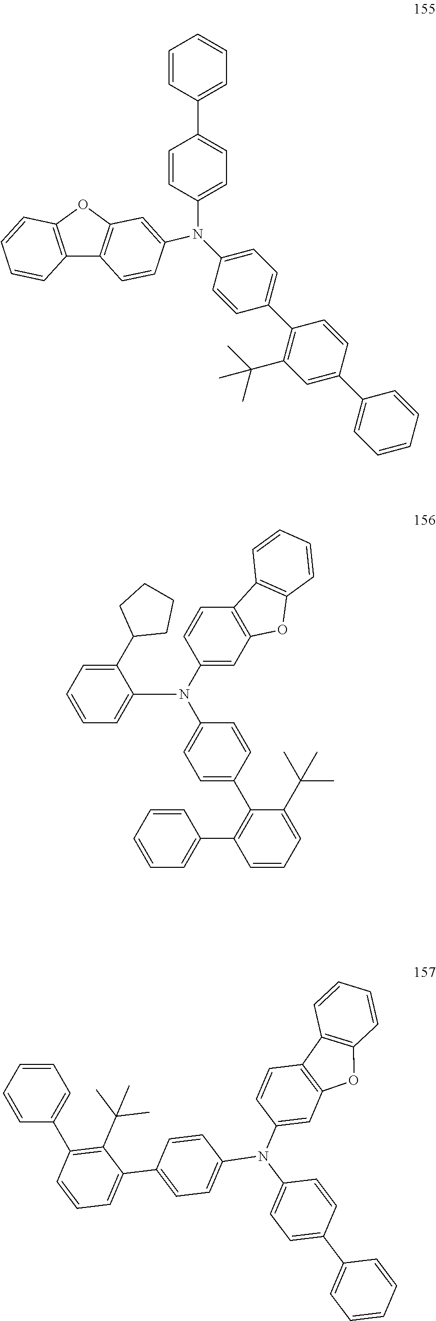 [SMILES: CC(C)(C)c1cc(-c2ccccc2)ccc1-c1ccc(N(c2ccc(-c3ccccc3)cc2)c2ccc3c(c2)oc2ccccc23)cc1 ; CC(C)(C)c1cccc(-c2ccccc2)c1-c1ccc(N(c2ccc3c(c2)oc2ccccc23)c2ccccc2C2CCCC2)cc1 ; CC(C)(C)c1c(-c2ccccc2)cccc1-c1ccc(N(c2ccc(-c3ccccc3)cc2)c2ccc3c(c2)oc2ccccc23)cc1]

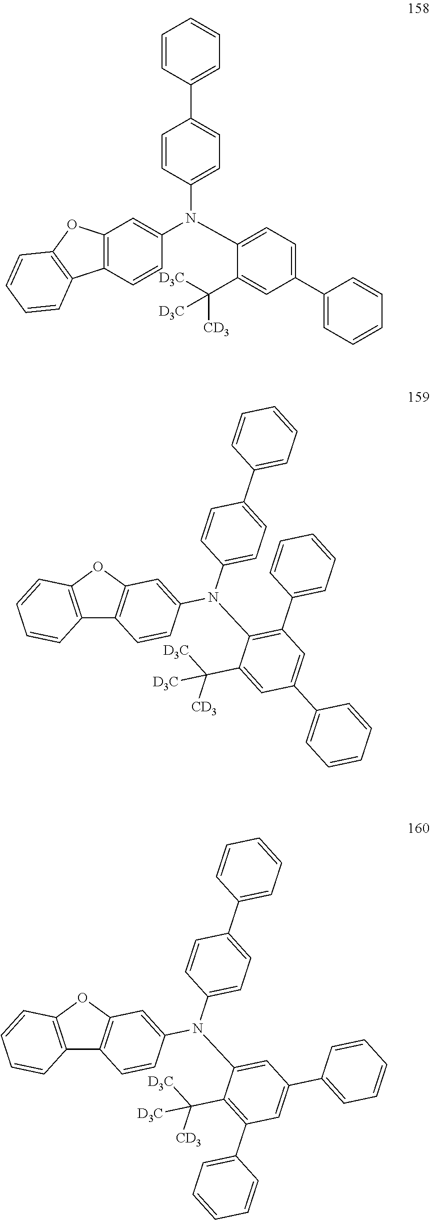 [SMILES: [2H]C([2H])([2H])C(c1cc(-c2ccccc2)ccc1N(c1ccc(-c2ccccc2)cc1)c1ccc2c(c1)oc1ccccc12)(C([2H])([2H])[2H])C([2H])([2H])[2H] ; [2H]C([2H])([2H])C(c1cc(-c2ccccc2)cc(-c2ccccc2)c1N(c1ccc(-c2ccccc2)cc1)c1ccc2c(c1)oc1ccccc12)(C([2H])([2H])[2H])C([2H])([2H])[2H] ; [2H]C([2H])([2H])C(c1c(-c2ccccc2)cc(-c2ccccc2)cc1N(c1ccc(-c2ccccc2)cc1)c1ccc2c(c1)oc1ccccc12)(C([2H])([2H])[2H])C([2H])([2H])[2H]]

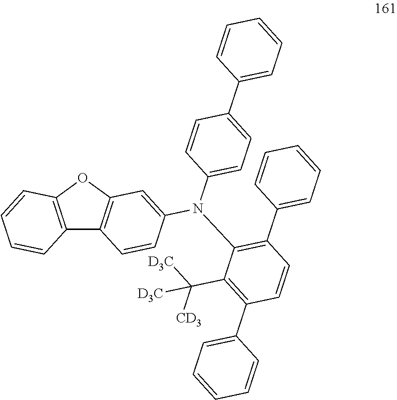 [SMILES: [2H]C([2H])([2H])C(c1c(-c2ccccc2)ccc(-c2ccccc2)c1N(c1ccc(-c2ccccc2)cc1)c1ccc2c(c1)oc1ccccc12)(C([2H])([2H])[2H])C([2H])([2H])[2H]]

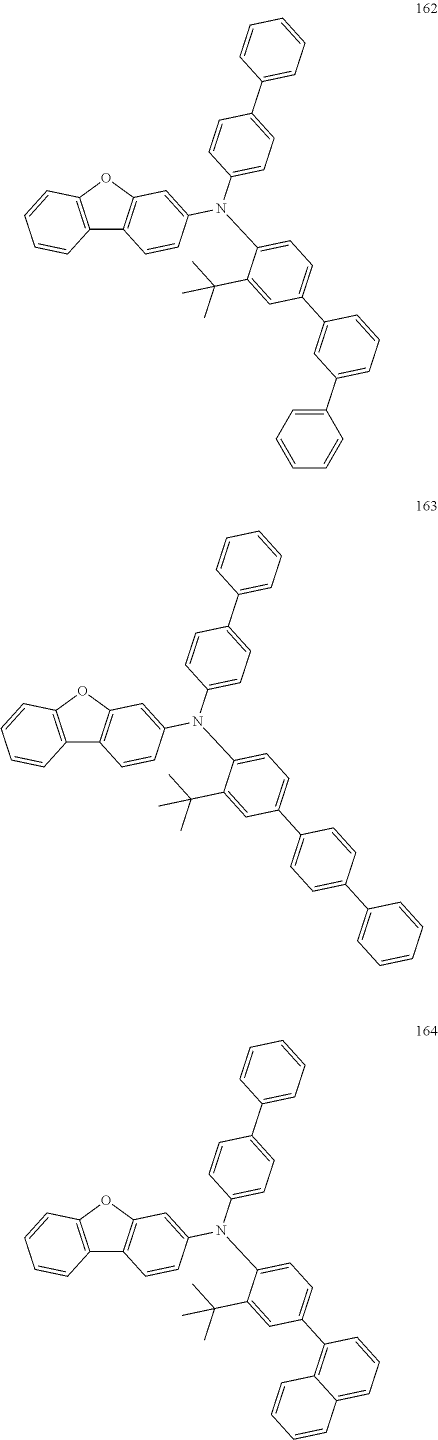 [SMILES: CC(C)(C)c1cc(-c2cccc(-c3ccccc3)c2)ccc1N(c1ccc(-c2ccccc2)cc1)c1ccc2c(c1)oc1ccccc12 ; CC(C)(C)c1cc(-c2ccc(-c3ccccc3)cc2)ccc1N(c1ccc(-c2ccccc2)cc1)c1ccc2c(c1)oc1ccccc12 ; CC(C)(C)c1cc(-c2cccc3ccccc23)ccc1N(c1ccc(-c2ccccc2)cc1)c1ccc2c(c1)oc1ccccc12]

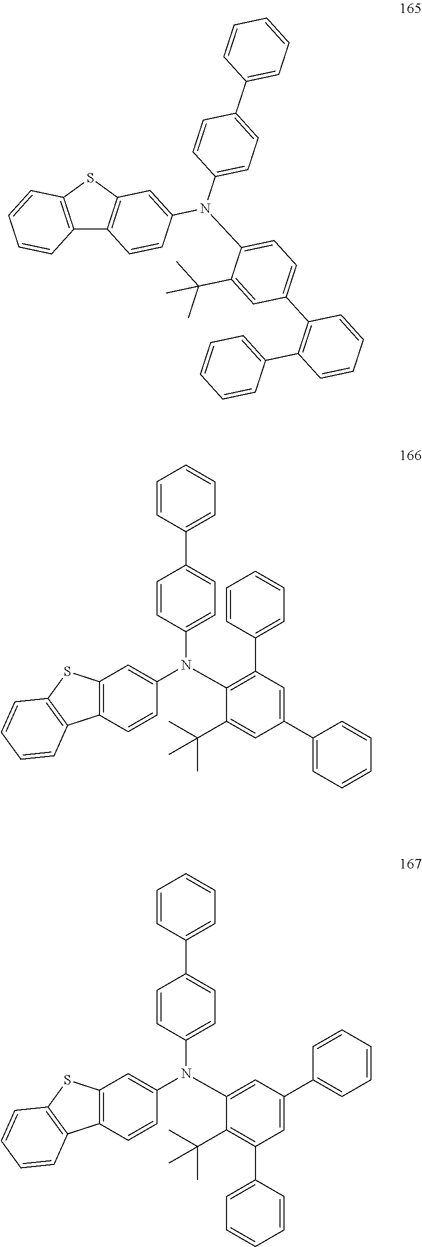 [SMILES: CC(C)(C)c1cc(-c2ccccc2-c2ccccc2)ccc1N(c1ccc(-c2ccccc2)cc1)c1ccc2c(c1)sc1ccccc12 ; CC(C)(C)c1cc(-c2ccccc2)cc(-c2ccccc2)c1N(c1ccc(-c2ccccc2)cc1)c1ccc2c(c1)sc1ccccc12 ; CC(C)(C)c1c(-c2ccccc2)cc(-c2ccccc2)cc1N(c1ccc(-c2ccccc2)cc1)c1ccc2c(c1)sc1ccccc12]

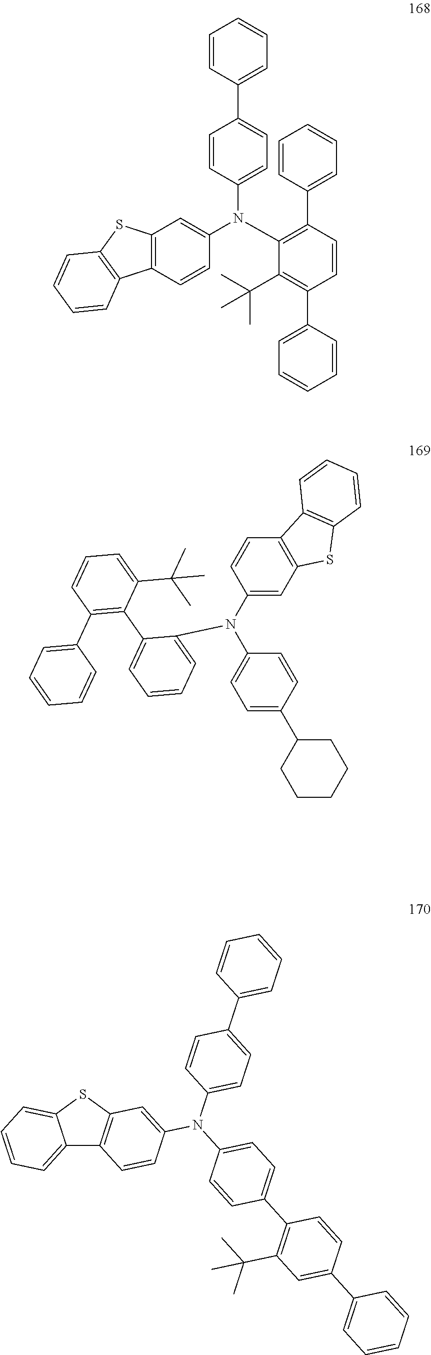 [SMILES: CC(C)(C)c1c(-c2ccccc2)ccc(-c2ccccc2)c1N(c1ccc(-c2ccccc2)cc1)c1ccc2c(c1)sc1ccccc12 ; CC(C)(C)c1cccc(-c2ccccc2)c1-c1ccccc1N(c1ccc(C2CCCCC2)cc1)c1ccc2c(c1)sc1ccccc12 ; CC(C)(C)c1cc(-c2ccccc2)ccc1-c1ccc(N(c2ccc(-c3ccccc3)cc2)c2ccc3c(c2)sc2ccccc23)cc1]

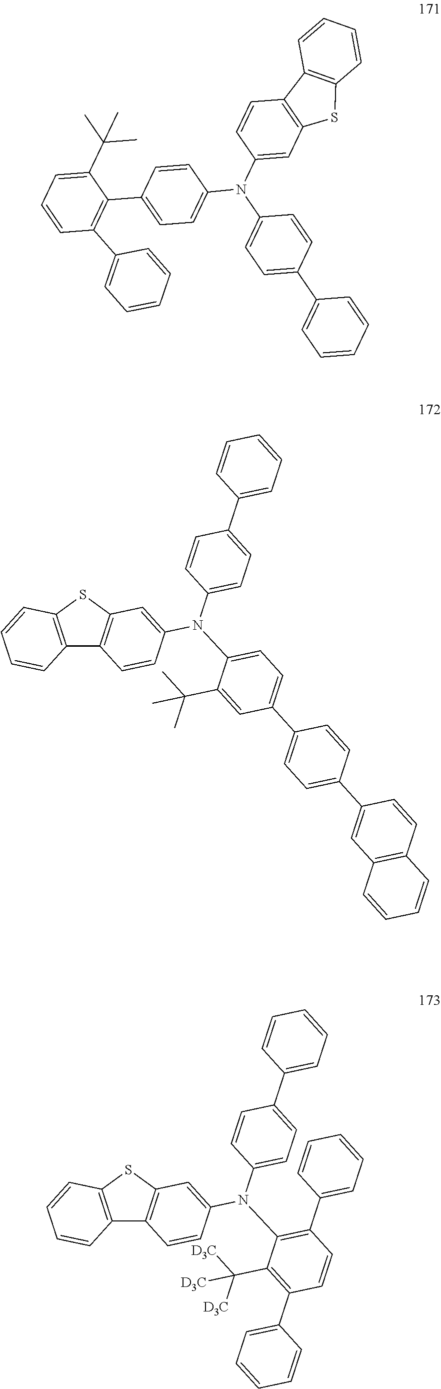 [SMILES: CC(C)(C)c1cccc(-c2ccccc2)c1-c1ccc(N(c2ccc(-c3ccccc3)cc2)c2ccc3c(c2)sc2ccccc23)cc1 ; CC(C)(C)c1cc(-c2ccc(-c3ccc4ccccc4c3)cc2)ccc1N(c1ccc(-c2ccccc2)cc1)c1ccc2c(c1)sc1ccccc12 ; [2H]C([2H])([2H])C(c1c(-c2ccccc2)ccc(-c2ccccc2)c1N(c1ccc(-c2ccccc2)cc1)c1ccc2c(c1)sc1ccccc12)(C([2H])([2H])[2H])C([2H])([2H])[2H]]

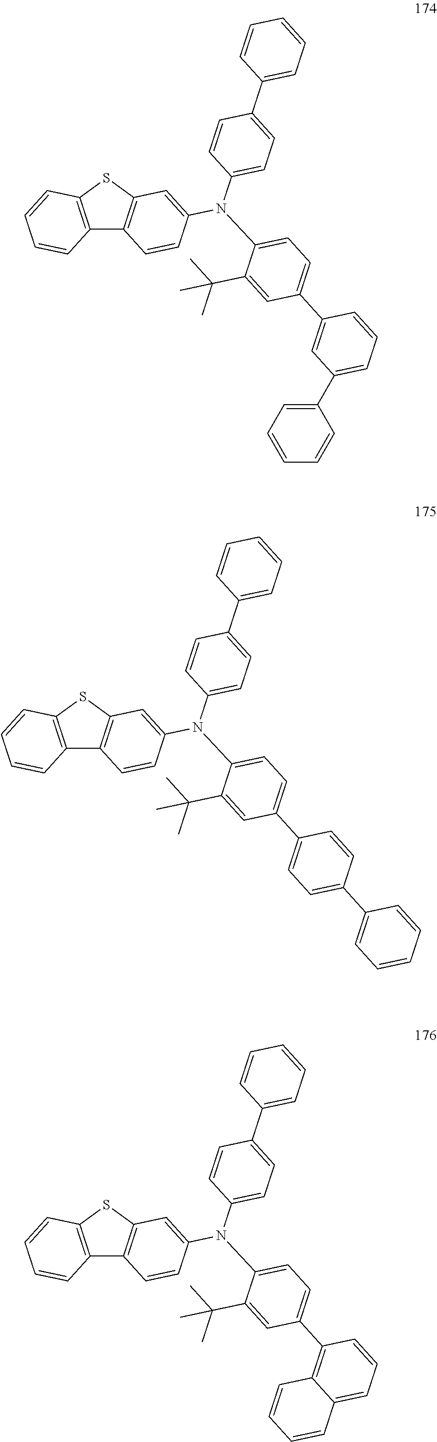 [SMILES: CC(C)(C)c1cc(-c2cccc(-c3ccccc3)c2)ccc1N(c1ccc(-c2ccccc2)cc1)c1ccc2c(c1)sc1ccccc12 ; CC(C)(C)c1cc(-c2ccc(-c3ccccc3)cc2)ccc1N(c1ccc(-c2ccccc2)cc1)c1ccc2c(c1)sc1ccccc12 ; CC(C)(C)c1cc(-c2cccc3ccccc23)ccc1N(c1ccc(-c2ccccc2)cc1)c1ccc2c(c1)sc1ccccc12]

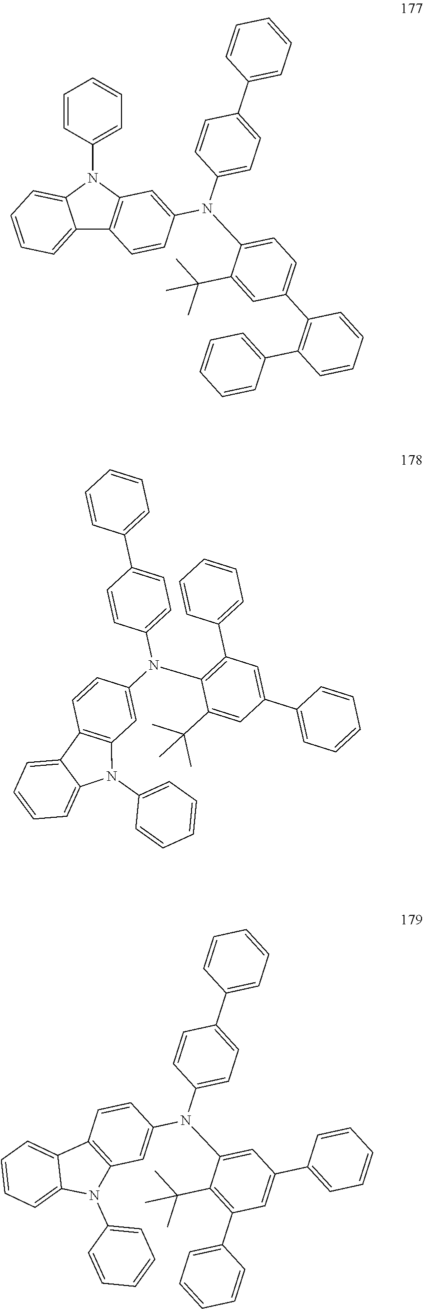 [SMILES: CC(C)(C)c1cc(-c2ccccc2-c2ccccc2)ccc1N(c1ccc(-c2ccccc2)cc1)c1ccc2c3ccccc3n(-c3ccccc3)c2c1 ; CC(C)(C)c1cc(-c2ccccc2)cc(-c2ccccc2)c1N(c1ccc(-c2ccccc2)cc1)c1ccc2c3ccccc3n(-c3ccccc3)c2c1 ; CC(C)(C)c1c(-c2ccccc2)cc(-c2ccccc2)cc1N(c1ccc(-c2ccccc2)cc1)c1ccc2c3ccccc3n(-c3ccccc3)c2c1]

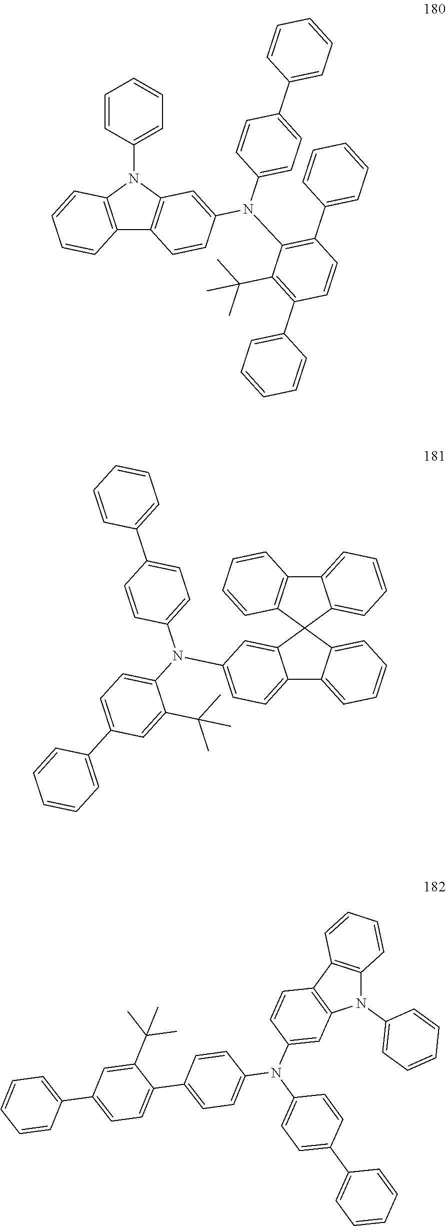 [SMILES: CC(C)(C)c1c(-c2ccccc2)ccc(-c2ccccc2)c1N(c1ccc(-c2ccccc2)cc1)c1ccc2c3ccccc3n(-c3ccccc3)c2c1 ; CC(C)(C)c1cc(-c2ccccc2)ccc1N(c1ccc(-c2ccccc2)cc1)c1ccc2c(c1)C1(c3ccccc3-c3ccccc31)c1ccccc1-2 ; CC(C)(C)c1cc(-c2ccccc2)ccc1-c1ccc(N(c2ccc(-c3ccccc3)cc2)c2ccc3c4ccccc4n(-c4ccccc4)c3c2)cc1]

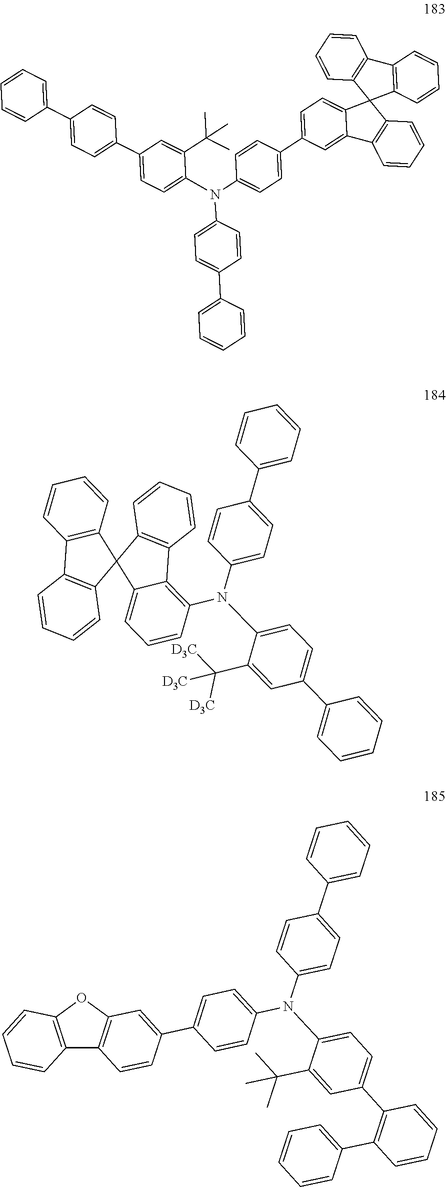 [SMILES: CC(C)(C)c1cc(-c2ccc(-c3ccccc3)cc2)ccc1N(c1ccc(-c2ccccc2)cc1)c1ccc(-c2ccc3c(c2)-c2ccccc2C32c3ccccc3-c3ccccc32)cc1 ; [2H]C([2H])([2H])C(c1cc(-c2ccccc2)ccc1N(c1ccc(-c2ccccc2)cc1)c1cccc2c1-c1ccccc1C21c2ccccc2-c2ccccc21)(C([2H])([2H])[2H])C([2H])([2H])[2H] ; CC(C)(C)c1cc(-c2ccccc2-c2ccccc2)ccc1N(c1ccc(-c2ccccc2)cc1)c1ccc(-c2ccc3c(c2)oc2ccccc23)cc1]

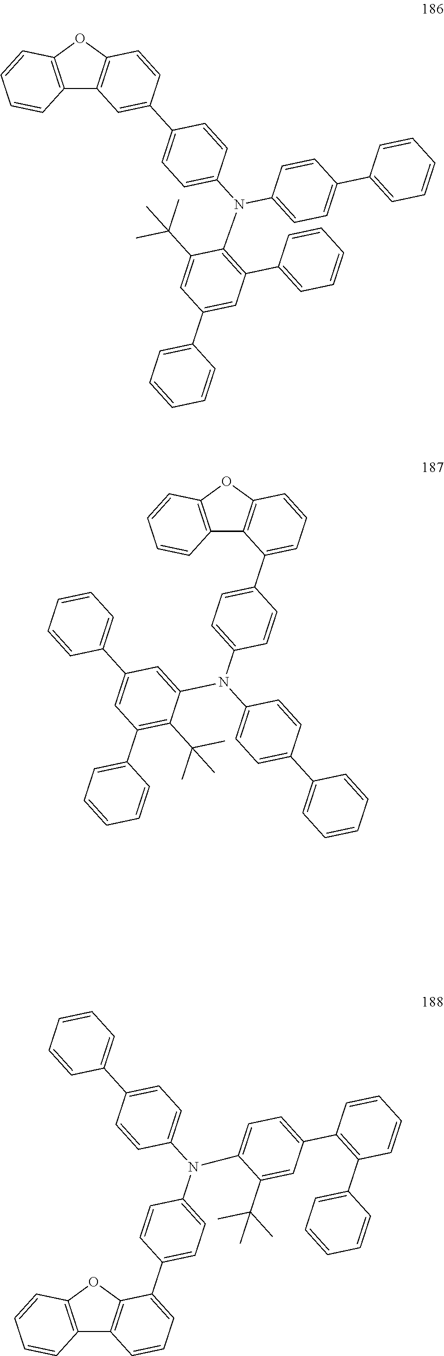 [SMILES: CC(C)(C)c1cc(-c2ccccc2)cc(-c2ccccc2)c1N(c1ccc(-c2ccccc2)cc1)c1ccc(-c2ccc3oc4ccccc4c3c2)cc1 ; CC(C)(C)c1c(-c2ccccc2)cc(-c2ccccc2)cc1N(c1ccc(-c2ccccc2)cc1)c1ccc(-c2cccc3oc4ccccc4c23)cc1 ; CC(C)(C)c1cc(-c2ccccc2-c2ccccc2)ccc1N(c1ccc(-c2ccccc2)cc1)c1ccc(-c2cccc3c2oc2ccccc23)cc1]

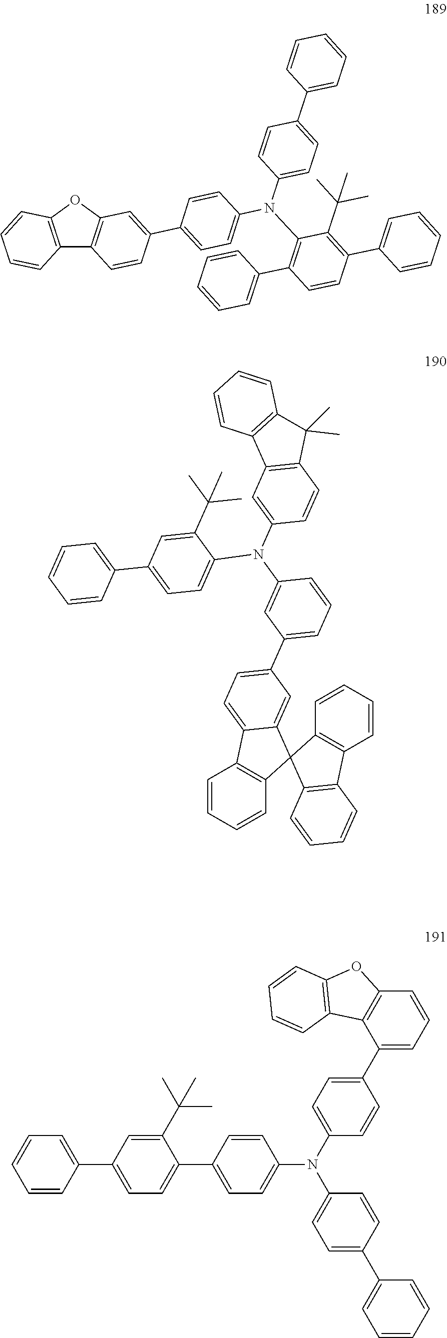 [SMILES: CC(C)(C)c1c(-c2ccccc2)ccc(-c2ccccc2)c1N(c1ccc(-c2ccccc2)cc1)c1ccc(-c2ccc3c(c2)oc2ccccc23)cc1 ; CC(C)(C)c1cc(-c2ccccc2)ccc1N(c1cccc(-c2ccc3c(c2)C2(c4ccccc4-c4ccccc42)c2ccccc2-3)c1)c1ccc2c(c1)-c1ccccc1C2(C)C ; CC(C)(C)c1cc(-c2ccccc2)ccc1-c1ccc(N(c2ccc(-c3ccccc3)cc2)c2ccc(-c3cccc4oc5ccccc5c34)cc2)cc1]

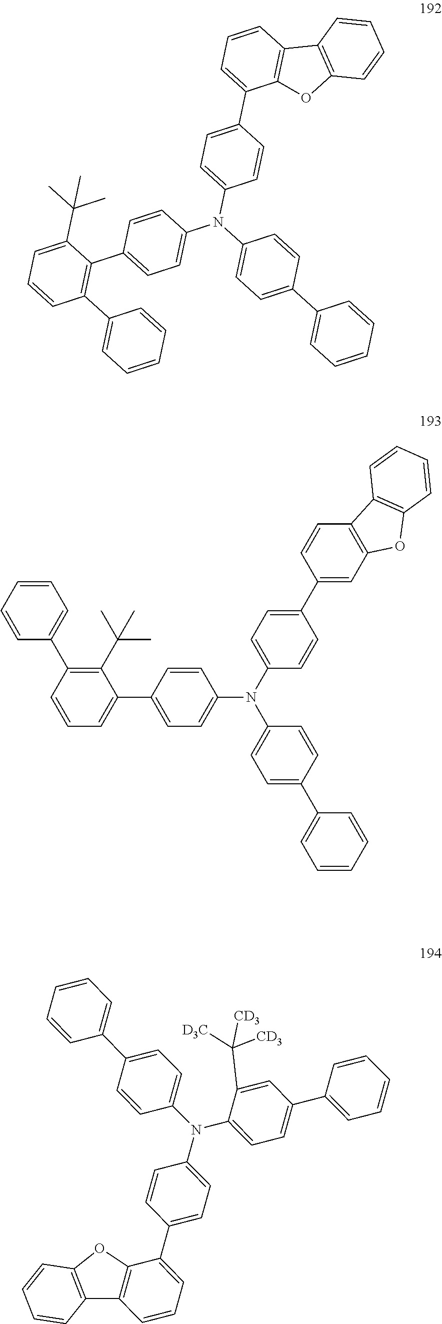 [SMILES: CC(C)(C)c1cccc(-c2ccccc2)c1-c1ccc(N(c2ccc(-c3ccccc3)cc2)c2ccc(-c3cccc4c3oc3ccccc34)cc2)cc1 ; CC(C)(C)c1c(-c2ccccc2)cccc1-c1ccc(N(c2ccc(-c3ccccc3)cc2)c2ccc(-c3ccc4c(c3)oc3ccccc34)cc2)cc1 ; [2H]C([2H])([2H])C(c1cc(-c2ccccc2)ccc1N(c1ccc(-c2ccccc2)cc1)c1ccc(-c2cccc3c2oc2ccccc23)cc1)(C([2H])([2H])[2H])C([2H])([2H])[2H]]

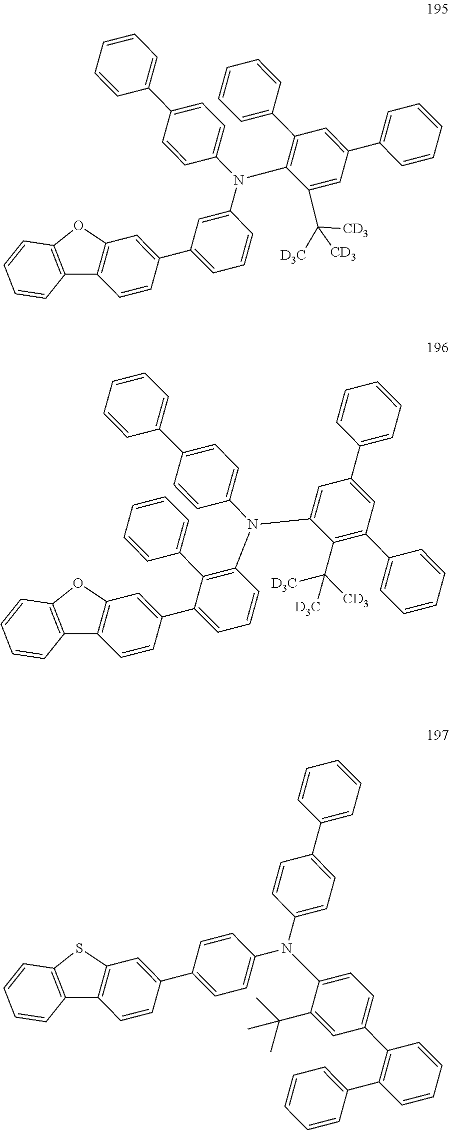 [SMILES: [2H]C([2H])([2H])C(c1cc(-c2ccccc2)cc(-c2ccccc2)c1N(c1ccc(-c2ccccc2)cc1)c1cccc(-c2ccc3c(c2)oc2ccccc23)c1)(C([2H])([2H])[2H])C([2H])([2H])[2H] ; [2H]C([2H])([2H])C(c1c(-c2ccccc2)cc(-c2ccccc2)cc1N(c1ccc(-c2ccccc2)cc1)c1cccc(-c2ccc3c(c2)oc2ccccc23)c1-c1ccccc1)(C([2H])([2H])[2H])C([2H])([2H])[2H] ; CC(C)(C)c1cc(-c2ccccc2-c2ccccc2)ccc1N(c1ccc(-c2ccccc2)cc1)c1ccc(-c2ccc3c(c2)sc2ccccc23)cc1]

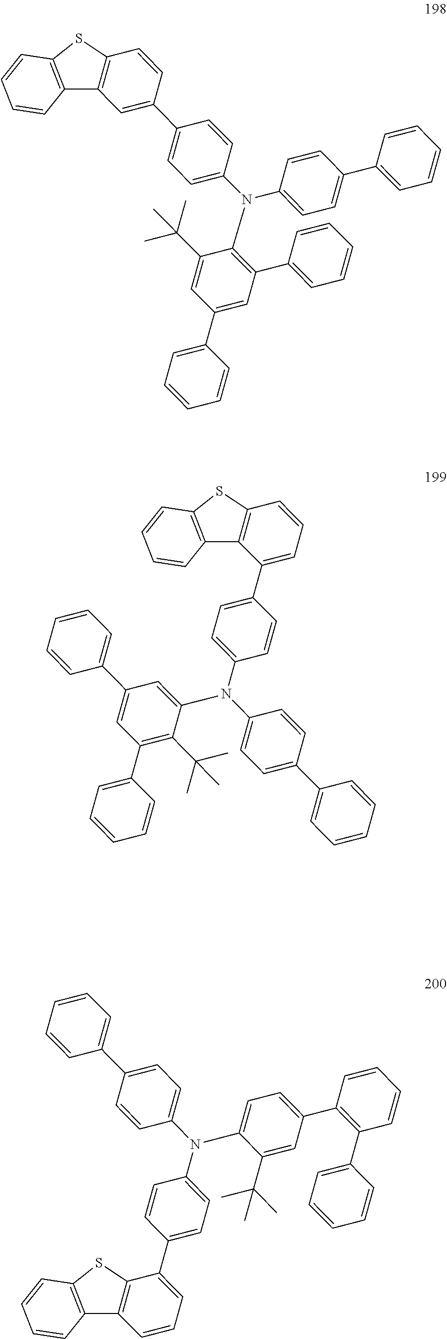 [SMILES: CC(C)(C)c1cc(-c2ccccc2)cc(-c2ccccc2)c1N(c1ccc(-c2ccccc2)cc1)c1ccc(-c2ccc3sc4ccccc4c3c2)cc1 ; CC(C)(C)c1c(-c2ccccc2)cc(-c2ccccc2)cc1N(c1ccc(-c2ccccc2)cc1)c1ccc(-c2cccc3sc4ccccc4c23)cc1 ; CC(C)(C)c1cc(-c2ccccc2-c2ccccc2)ccc1N(c1ccc(-c2ccccc2)cc1)c1ccc(-c2cccc3c2sc2ccccc23)cc1]

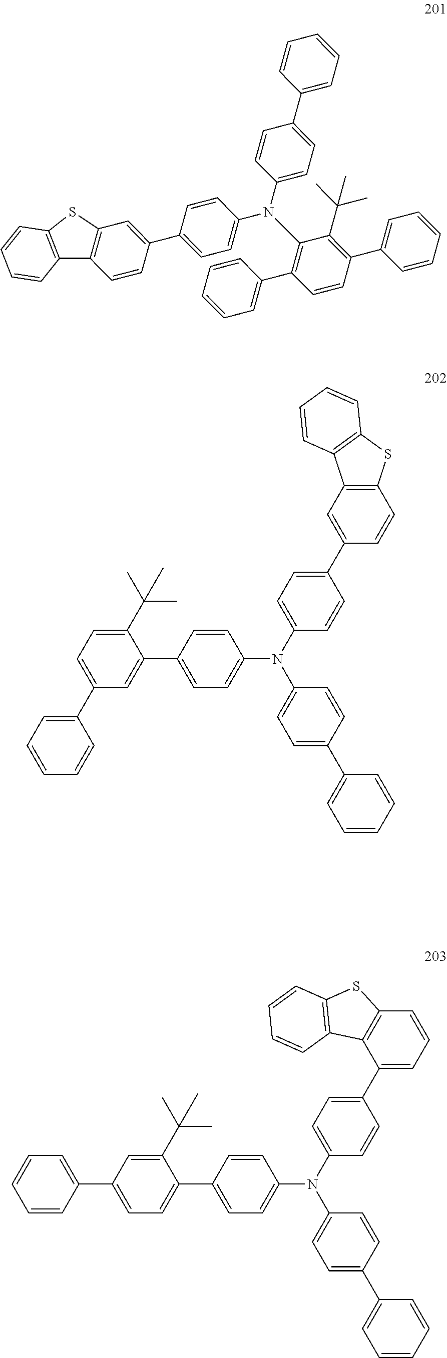 [SMILES: CC(C)(C)c1c(-c2ccccc2)ccc(-c2ccccc2)c1N(c1ccc(-c2ccccc2)cc1)c1ccc(-c2ccc3c(c2)sc2ccccc23)cc1 ; CC(C)(C)c1ccc(-c2ccccc2)cc1-c1ccc(N(c2ccc(-c3ccccc3)cc2)c2ccc(-c3ccc4sc5ccccc5c4c3)cc2)cc1 ; CC(C)(C)c1cc(-c2ccccc2)ccc1-c1ccc(N(c2ccc(-c3ccccc3)cc2)c2ccc(-c3cccc4sc5ccccc5c34)cc2)cc1]

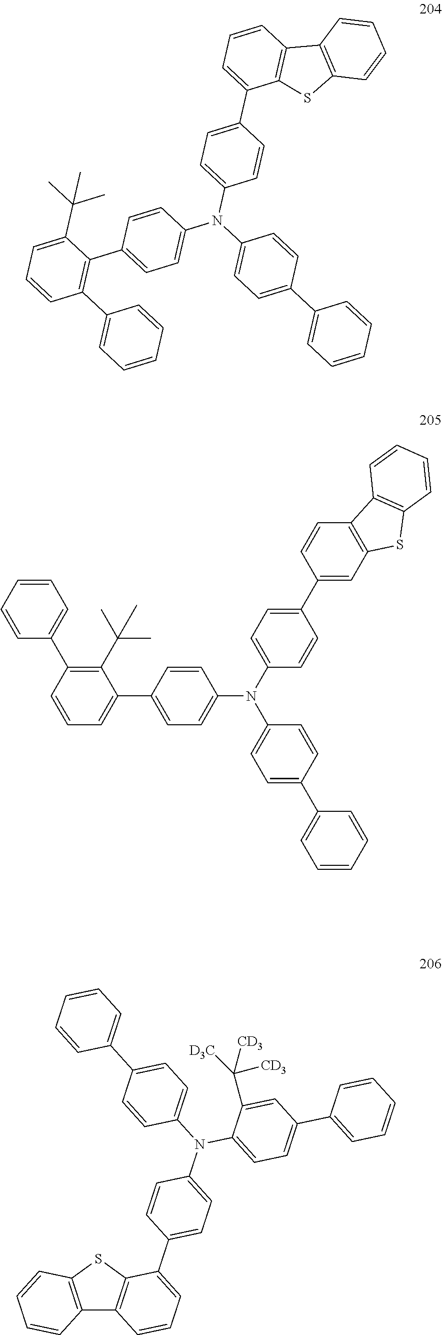 [SMILES: CC(C)(C)c1cccc(-c2ccccc2)c1-c1ccc(N(c2ccc(-c3ccccc3)cc2)c2ccc(-c3cccc4c3sc3ccccc34)cc2)cc1 ; CC(C)(C)c1c(-c2ccccc2)cccc1-c1ccc(N(c2ccc(-c3ccccc3)cc2)c2ccc(-c3ccc4c(c3)sc3ccccc34)cc2)cc1 ; [2H]C([2H])([2H])C(c1cc(-c2ccccc2)ccc1N(c1ccc(-c2ccccc2)cc1)c1ccc(-c2cccc3c2sc2ccccc23)cc1)(C([2H])([2H])[2H])C([2H])([2H])[2H]]

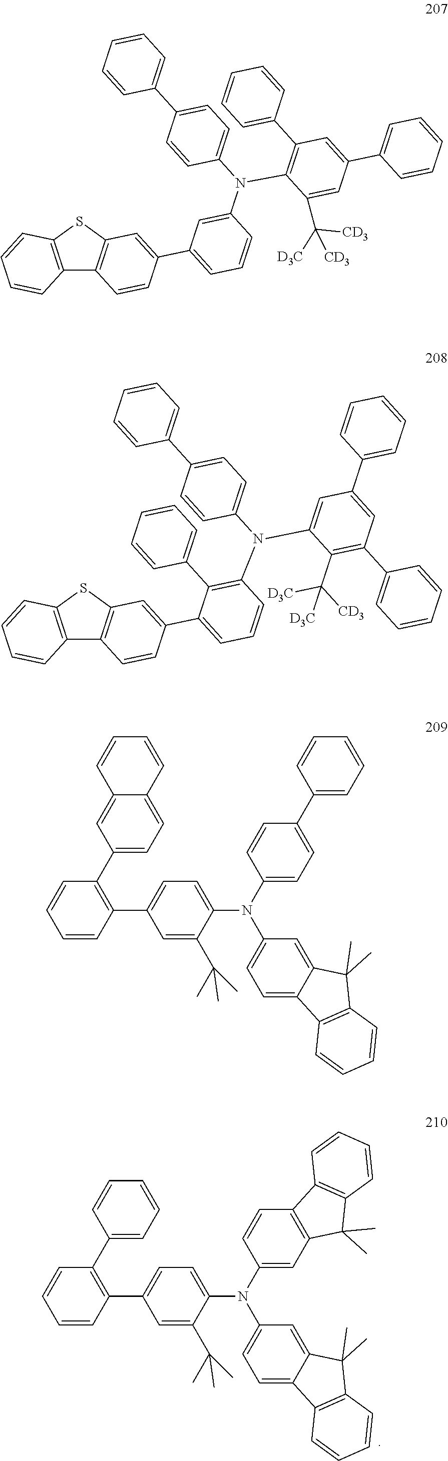 [SMILES: [2H]C([2H])([2H])C(c1cc(-c2ccccc2)cc(-c2ccccc2)c1N(c1ccc(-c2ccccc2)cc1)c1cccc(-c2ccc3c(c2)sc2ccccc23)c1)(C([2H])([2H])[2H])C([2H])([2H])[2H] ; [2H]C([2H])([2H])C(c1c(-c2ccccc2)cc(-c2ccccc2)cc1N(c1ccc(-c2ccccc2)cc1)c1cccc(-c2ccc3c(c2)sc2ccccc23)c1-c1ccccc1)(C([2H])([2H])[2H])C([2H])([2H])[2H] ; CC(C)(C)c1cc(-c2ccccc2-c2ccc3ccccc3c2)ccc1N(c1ccc(-c2ccccc2)cc1)c1ccc2c(c1)C(C)(C)c1ccccc1-2 ; CC(C)(C)c1cc(-c2ccccc2-c2ccccc2)ccc1N(c1ccc2c(c1)C(C)(C)c1ccccc1-2)c1ccc2c(c1)C(C)(C)c1ccccc1-2]

In a second aspect, the present disclosure provides an electronic element, comprising an anode and a cathode disposed opposite to each other, and a functional layer disposed between the anode and the cathode; the functional layer contains the organic compound of the present disclosure.

Optionally, the electronic element is an organic electroluminescent device or a photoelectric conversion device. Further optionally, the electronic element is a red organic electroluminescent device.

Optionally, the functional layer comprises a hole-transport layer, which contains the organic compound of the present disclosure.

Further optionally, the hole transport layer comprises a first hole transport layer and a second hole transport layer. The first hole transport layer is closer to the anode compared to the second hole transport layer, wherein the second hole transport layer comprises the organic compound of the present disclosure.

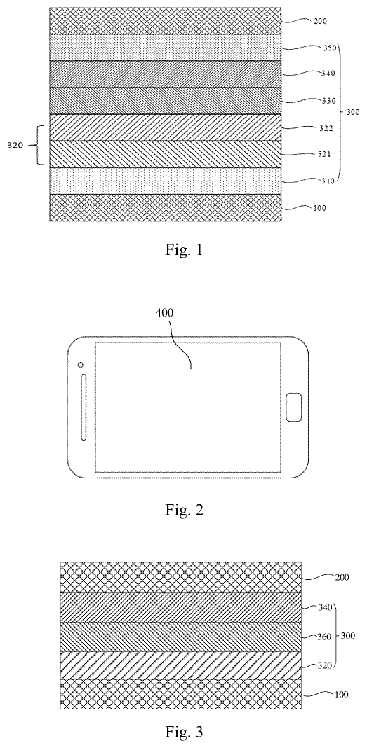

In an embodiment, the electronic element is an organic electroluminescent device. As shown in FIG. 1, the organic electroluminescent device may comprise an anode 100, a first hole transport layer 321, a second hole transport layer 322, an organic light-emitting layer 330, an electron transport layer 340, and a cathode 200 that are stacked. Among them, the first hole transport layer 321 and the second hole transport layer constitute a hole transport layer 320.

Optionally, the anode 100 comprises an anode material as follows, which is preferably a high work function material contributing to injection of holes into the functional layer. Specific examples of the anode material include: metals such as nickel, platinum, vanadium, chromium, copper, zinc, gold, or alloys thereof; metal oxides such as zinc oxide, indium oxide, indium tin oxide (ITO), and indium zinc oxide (IZO); combinations of metals and oxides, such as ZnO:Al or SnO₂:Sb; or conductive polymers such as poly(3-methylthiophene), poly [3,4-(ethylene-1,2-dioxy) thiophene] (PEDT), polypyrrole, and polyaniline, but are not limited thereto. Preferably, a transparent electrode comprising indium tin oxide (ITO) as the anode is included.

Optionally, the hole transport layer includes one or more hole transport material(s). The hole transport materials may be selected from carbazole polymers, carbazole-connected triarylamine compounds, and other types of compounds. It is not particularly defined in the present disclosure. For example, the material of the first hole transport layer is selected from the group consisting of the following compounds:

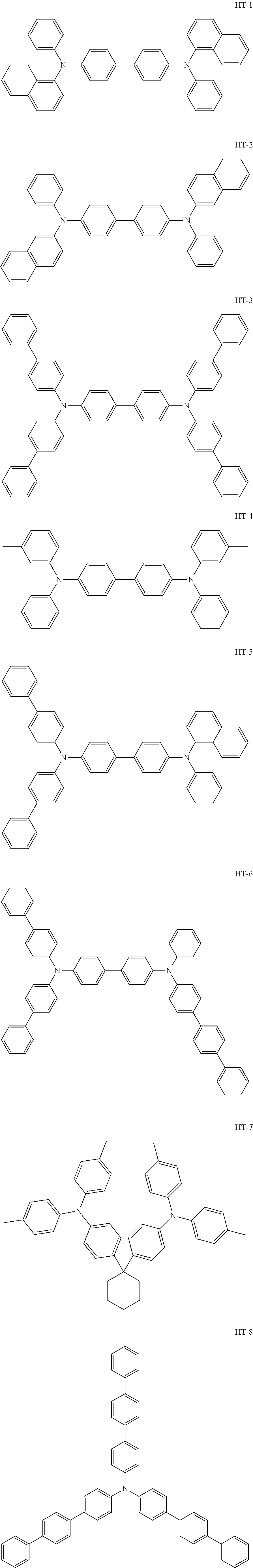

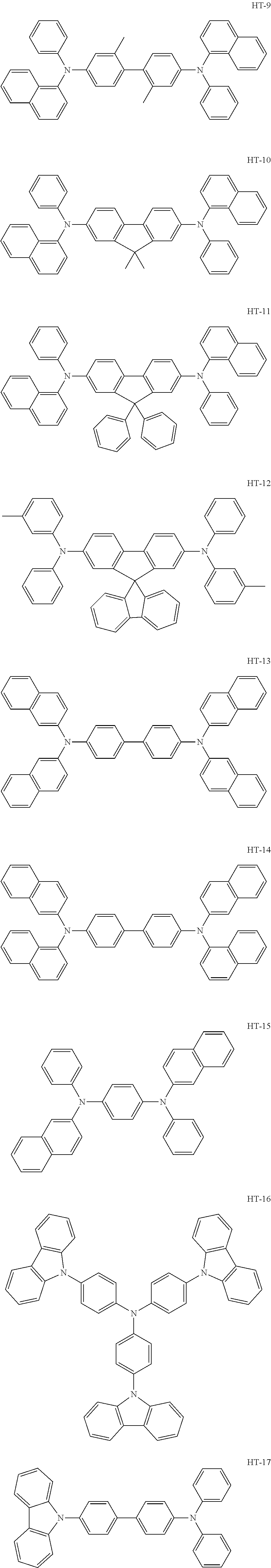

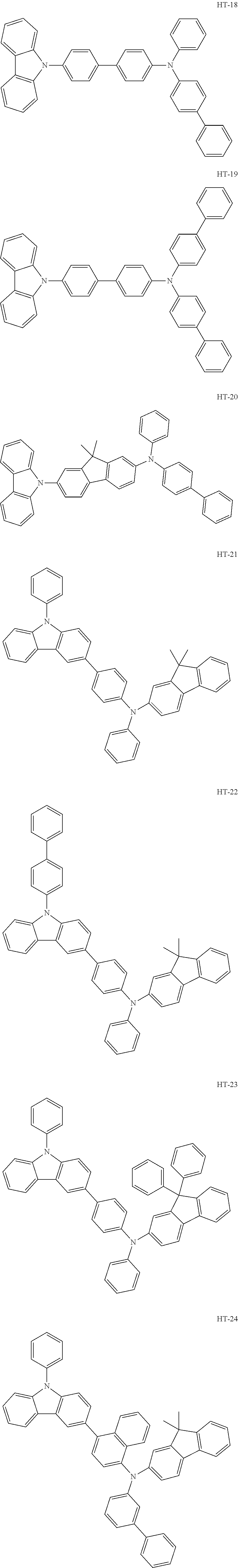

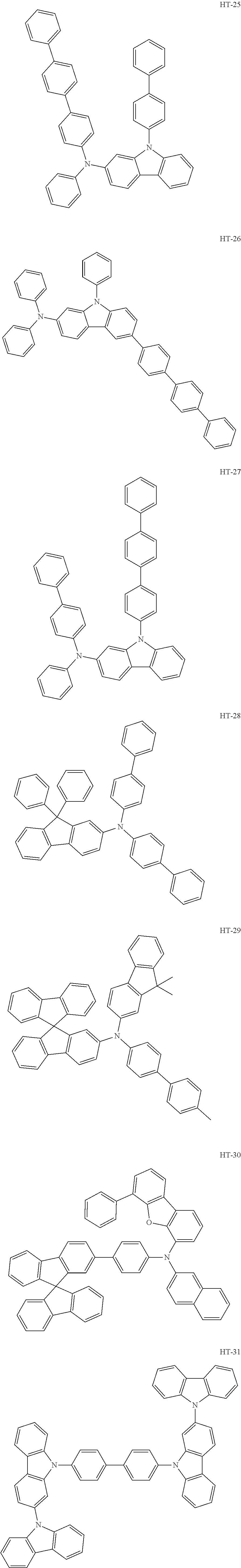

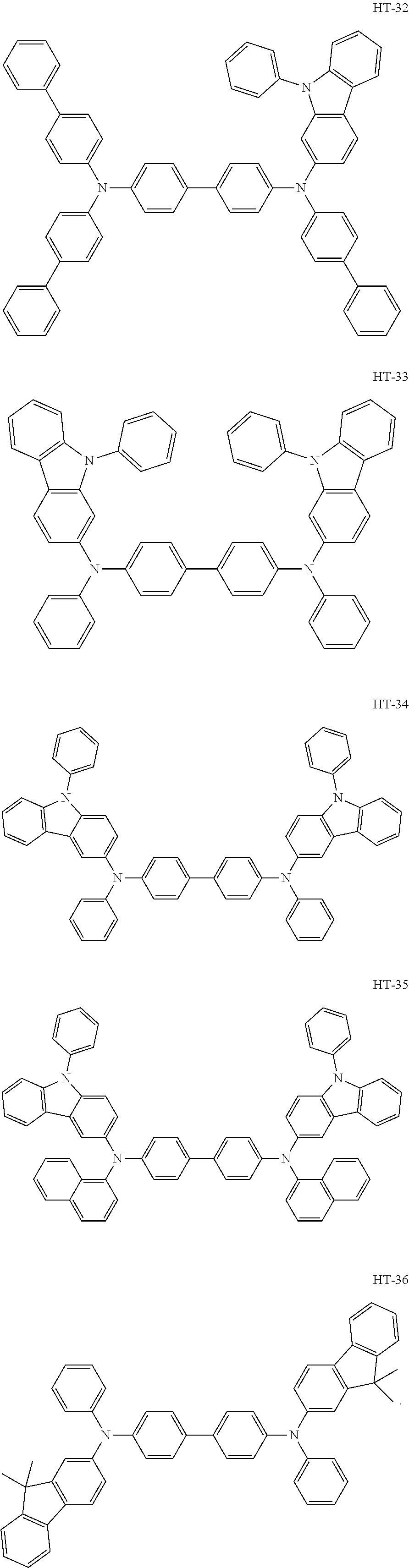

In a specific embodiment, the first hole transport layer 321 is compound HT-36.

Optionally, the second hole transport layer 322 is the compound of the present disclosure.

Optionally, the organic light-emitting layer 330 may be composed of a single light-emitting layer material or may comprise a host material and a dopant material. Optionally, the organic light-emitting layer 330 is composed of a host material and a dopant material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the dopant material, thereby enabling the dopant material to emit light.

The host material of the organic light-emitting layer 330 may be a metal chelating compound, a stilbene-based derivative, an aromatic amine derivative, a dibenzofuran derivative, or other types of material. It is not particularly defined in the present disclosure. The host material may be a single host material, or a mixed host material. In an embodiment of the present disclosure, the host material of the organic light-emitting layer 330 is RH.

The dopant material of the organic light-emitting layer 330 can be selected with reference to the existing technology, and for example, may be selected from an iridium (III) organometallic complex, a platinum (II) organometallic complex, a ruthenium (II) complex, etc. The specific examples of the dopant material include but are not limited to,

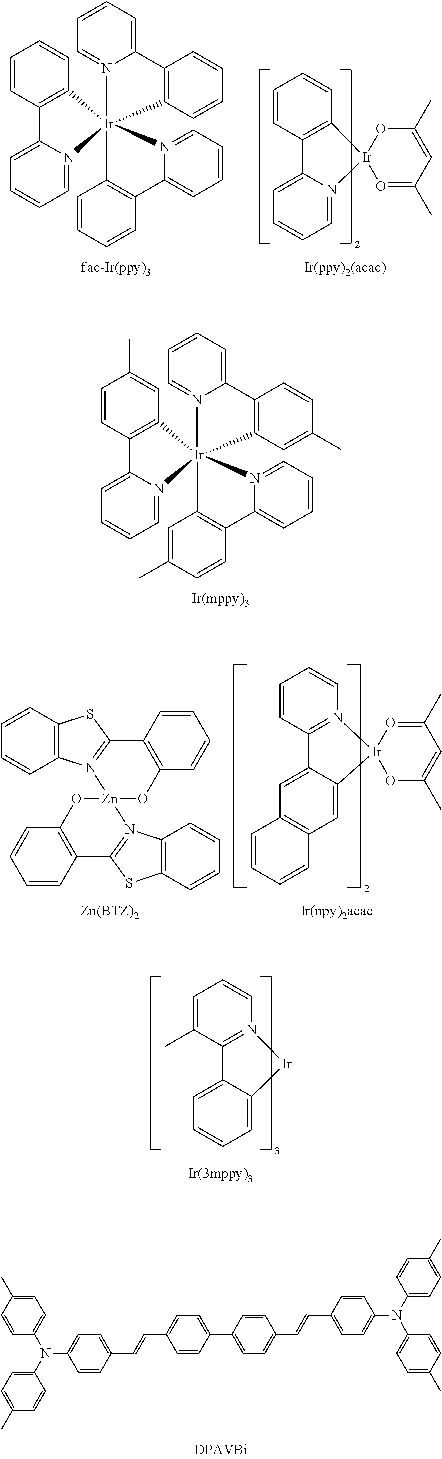

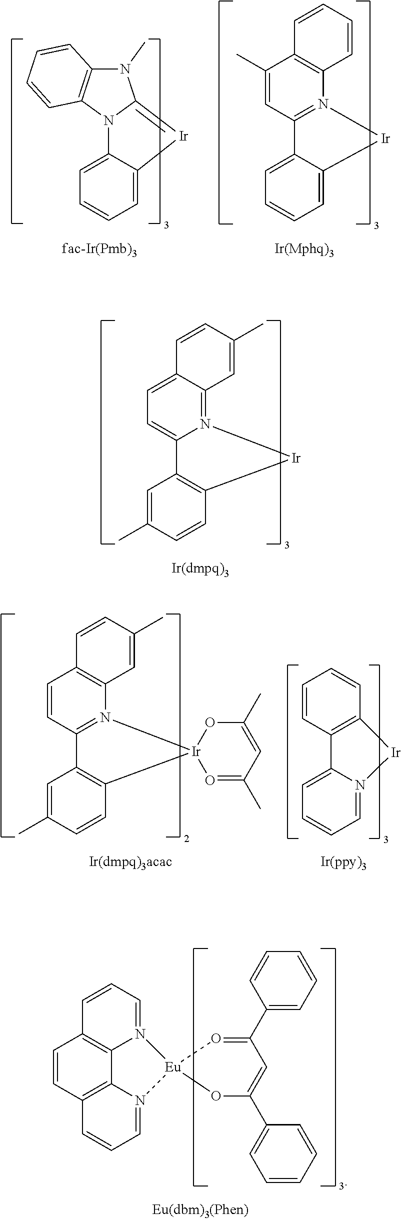

In an embodiment of the present disclosure, the dopant material of the organic light-emitting layer 330 is Ir(dmpq)₃acac.

Optionally, the electron transport layer 340 may be a single-layer structure or a multi-layer structure, which may comprise one or more electron transport material(s). The electron transport material typically includes a metal complex or/or a nitrogen-containing heterocyclic derivative, in which the metal complex material may be selected from LiQ, Alq₃, etc; The nitrogen-containing heterocyclic derivative may be an aromatic ring with a nitrogen-containing 6- or 5-membered ring skeleton, a fused aromatic ring compound with a nitrogen-containing 6- or 5-membered ring skeleton, etc. Specific examples include but are not limited to 1,10-phenanthroline compounds such as Bphen, NBphen, ET-20, BimiBphen, or anthracene compounds, triazine compounds, or pyrimidine compounds with nitrogen-containing heteroaryl as shown below. In an embodiment of the present disclosure, the electron transport layer 340 is composed of ET-9 and LiQ.

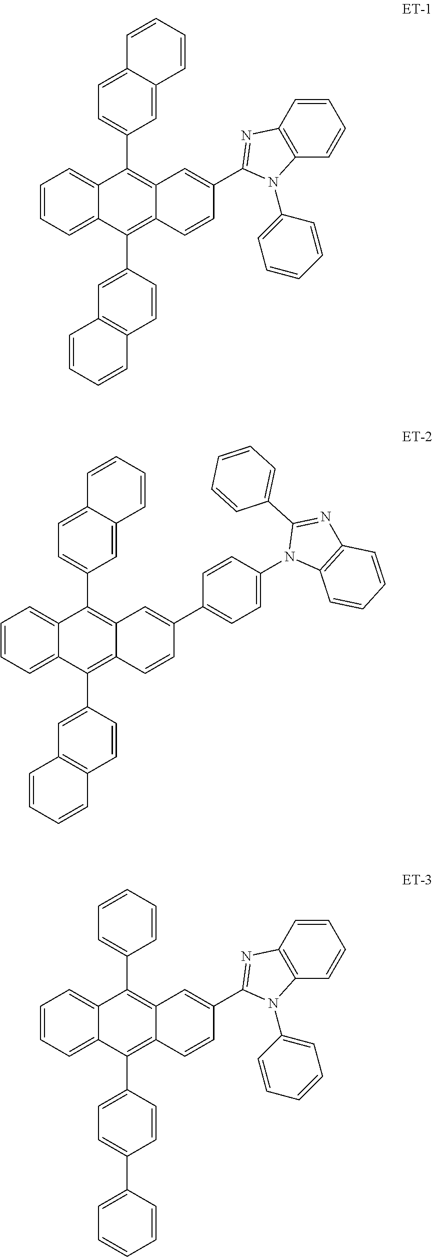 [SMILES: c1ccc(-n2c(-c3ccc4c(-c5ccc6ccccc6c5)c5ccccc5c(-c5ccc6ccccc6c5)c4c3)nc3ccccc32)cc1 ; c1ccc(-c2nc3ccccc3n2-c2ccc(-c3ccc4c(-c5ccc6ccccc6c5)c5ccccc5c(-c5ccc6ccccc6c5)c4c3)cc2)cc1 ; c1ccc(-c2ccc(-c3c4ccccc4c(-c4ccccc4)c4cc(-c5nc6ccccc6n5-c5ccccc5)ccc34)cc2)cc1]

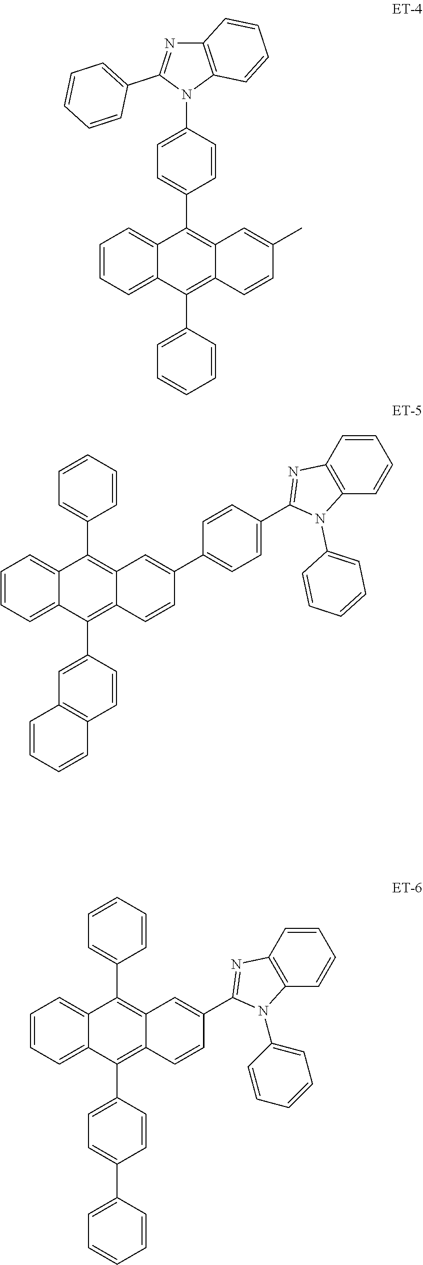 [SMILES: Cc1ccc2c(-c3ccccc3)c3ccccc3c(-c3ccc(-n4c(-c5ccccc5)nc5ccccc54)cc3)c2c1 ; c1ccc(-c2c3ccccc3c(-c3ccc4ccccc4c3)c3ccc(-c4ccc(-c5nc6ccccc6n5-c5ccccc5)cc4)cc23)cc1 ; c1ccc(-c2ccc(-c3c4ccccc4c(-c4ccccc4)c4cc(-c5nc6ccccc6n5-c5ccccc5)ccc34)cc2)cc1]

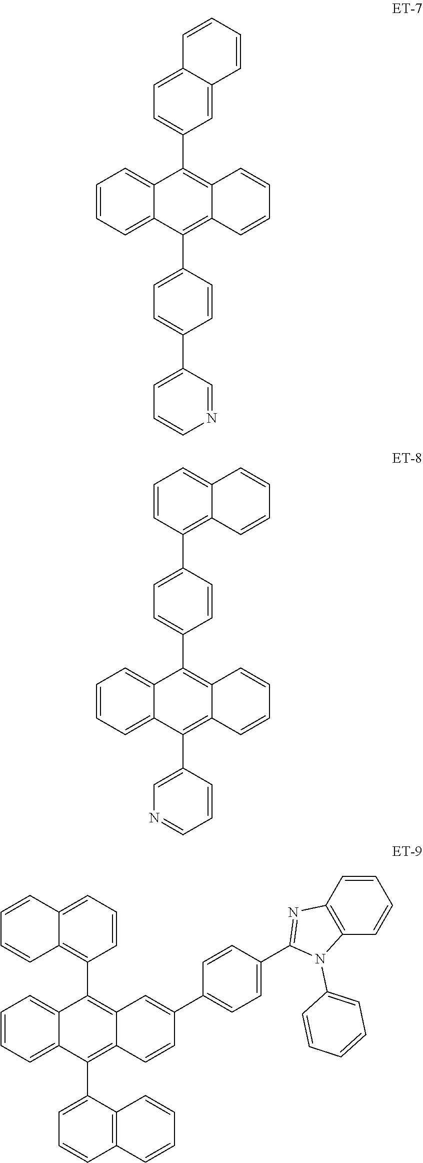

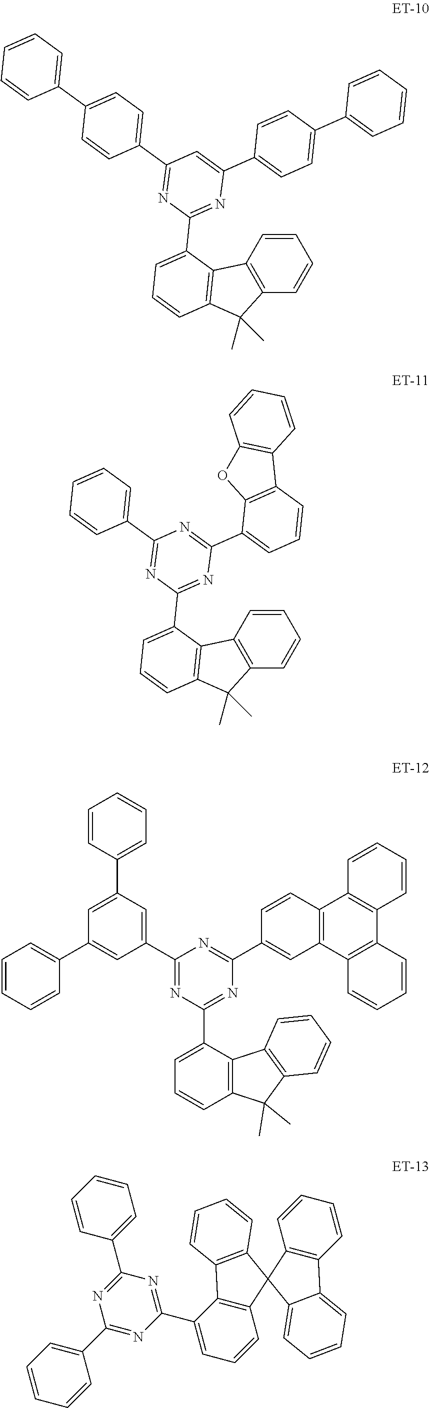

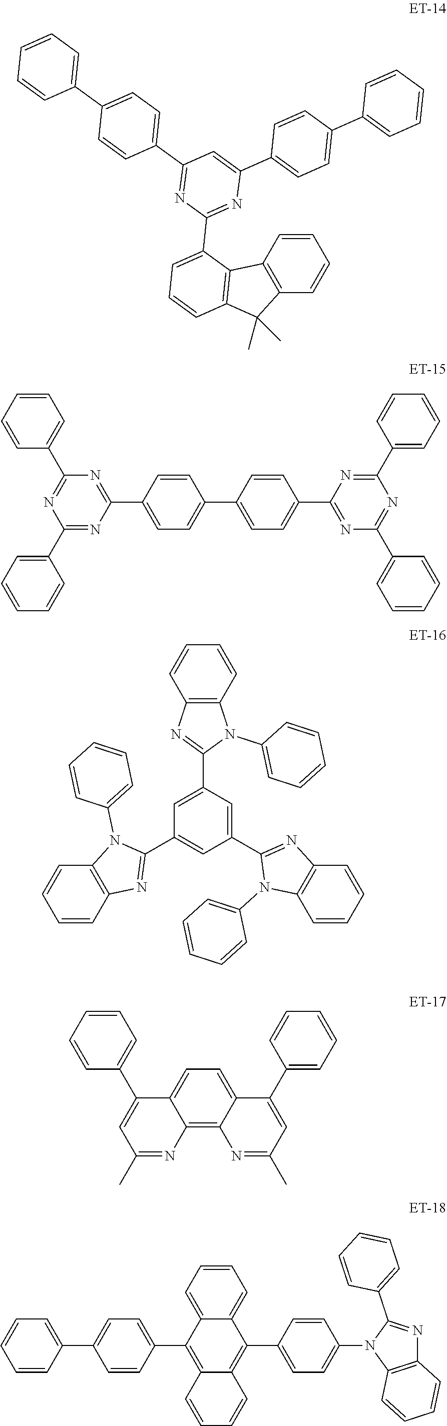 [SMILES: CC1(C)c2ccccc2-c2c(-c3nc(-c4ccc(-c5ccccc5)cc4)cc(-c4ccc(-c5ccccc5)cc4)n3)cccc21 ; c1ccc(-c2nc(-c3ccccc3)nc(-c3ccc(-c4ccc(-c5nc(-c6ccccc6)nc(-c6ccccc6)n5)cc4)cc3)n2)cc1 ; c1ccc(-n2c(-c3cc(-c4nc5ccccc5n4-c4ccccc4)cc(-c4nc5ccccc5n4-c4ccccc4)c3)nc3ccccc32)cc1 ; Cc1cc(-c2ccccc2)c2ccc3c(-c4ccccc4)cc(C)nc3c2n1 ; c1ccc(-c2ccc(-c3c4ccccc4c(-c4ccc(-n5c(-c6ccccc6)nc6ccccc65)cc4)c4ccccc34)cc2)cc1]

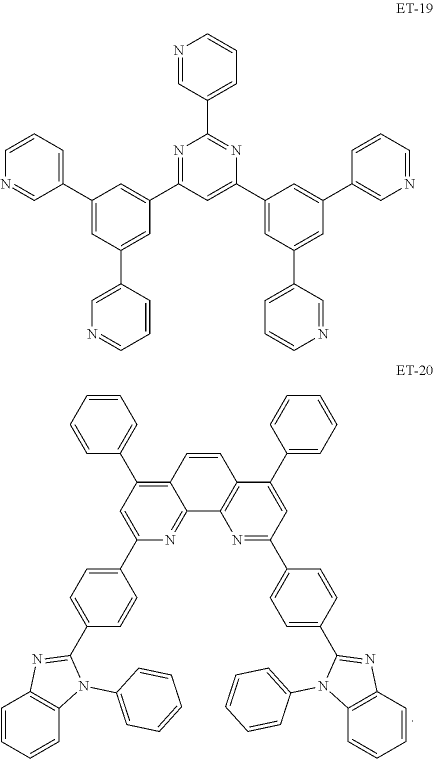

In an embodiment, the cathode 200 may comprise a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, lead, and alloys thereof; or multi-layer materials such as LiF/Al, Liq/Al, LiO₂/Al, LiF/Ca, LiF/Al, and BaF₂/Ca. Preferably, a metal electrode comprising magnesium and silver as the cathode is included.

Alternatively, as shown in FIG. 1, a hole injection layer 310 is further provided between the anode 100 and the first hole transport layer 321 to enhance the ability to inject holes into the first hole transport layer 321. The hole injection layer 310 may choose to use a benzidine derivative, a starburst arylamine-based compound, a phthalocyanine derivative, or other materials. It is not particularly defined in the present disclosure. For example, the compound contained in the hole injection layer 310 is selected from the group consisting of the following compounds:

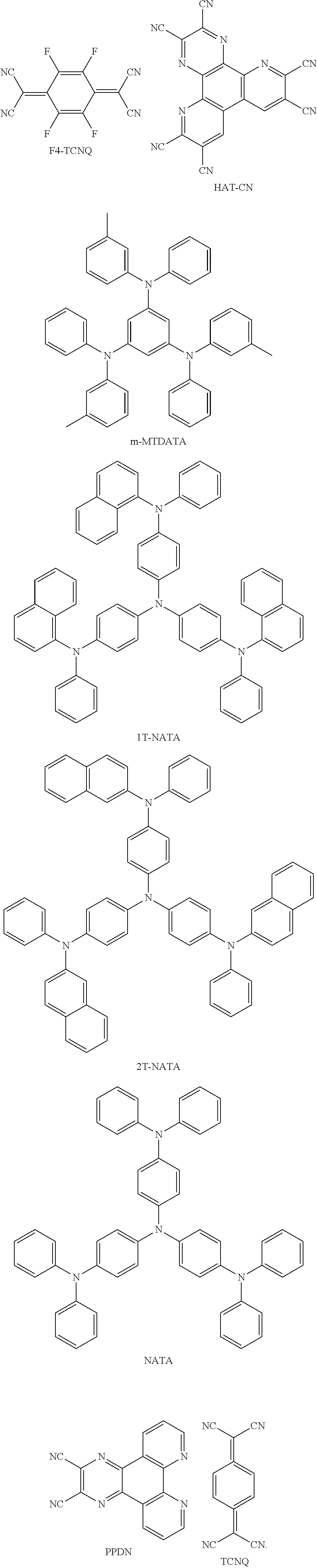

In a specific embodiment of the present disclosure, the hole injection layer 310 is F4-TCNQ and HT-36.

Optionally, as shown in FIG. 1, an electron injection layer 350 is further provided between the cathode 200 and the electron transport layer 340 to enhance the ability to inject electrons into the electron transport layer 340. The electron injection layer 350 may comprise an inorganic material such as an alkali metal sulfide, an alkali metal halide, or may comprise a complex of an alkali metal and an organic compound. For example, the electron injection layer 350 comprises Yb.

According to another embodiment, the electronic element is a photoelectric conversion device. As shown in FIG. 3, the photoelectric conversion device may include an anode 100 and a cathode 200 disposed opposite to each other, and a functional layer 300 disposed between the anode 100 and the cathode 200; the functional layer 300 comprises the organic compound provided in the present disclosure.

According to a specific embodiment, as shown in FIG. 3, the photoelectric conversion device includes an anode 100, a hole transport layer 320, a photoelectric conversion layer 360, an electron transport layer 340, and a cathode 200 sequentially stacked. Optionally, the hole transport layer 320 comprises the organic compound of the present disclosure.

Optionally, the photoelectric conversion device may be a solar cell, especially an organic thin film solar cell. For example, in an embodiment of the present disclosure, the solar cell comprises an anode, a hole transport layer, a photoelectric conversion layer, an electron transport layer, and a cathode sequentially stacked, wherein the hole transport layer comprises the organic compound of the present disclosure.

In a third aspect, the present disclosure provides an electronic apparatus, comprising the electronic element described in the second aspect of the present disclosure.

According to an embodiment, as shown in FIG. 2, the electronic apparatus is a first electronic apparatus 400 comprising the above-described organic electroluminescent device. The first electronic apparatus 400 may be, for example, a display apparatus, a lighting apparatus, an optical communication apparatus, or other types of electronic apparatus, examples of which may include, but are not limited to, a computer screen, a mobile phone screen, a television, an electronic paper, an emergency lamp, an optical module, etc.

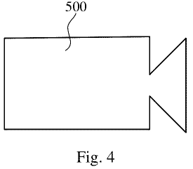

According to another embodiment, as shown in FIG. 4, the electronic apparatus is a second electronic apparatus 500 comprising the above-described organic electroluminescent device. The second electronic apparatus 500 may be, for example, a solar power generation equipment, a photodetector, a fingerprint recognition equipment, an optical module, a CCD camera, or other types of electronic apparatus.

The synthesis method of the organic compound in the present disclosure will be demonstrated in detail with the following synthesis examples, but the present disclosure is not limited in any way by this.

The compounds of the synthetic methods not mentioned in the present disclosure are all raw material products commercially available.

### Synthesis Example

Synthesis of IM L1:

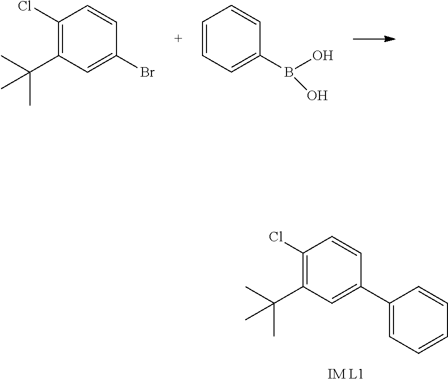 [SMILES: CC(C)(C)c1cc(Br)ccc1Cl ; OB(O)c1ccccc1 ; CC(C)(C)c1cc(-c2ccccc2)ccc1Cl]

To a three-necked flask was added 2-tert-butyl-4-bromo-1-chlorobenzene (50.0 g, 201.97 mmol), phenylboronic acid (27.1 g, 222.17 mmol), bis(triphenylphosphine) palladium dichloride (0.7 g, 1.01 mmol), potassium carbonate (56.2 g, 403.94 mmol), ethylene glycol, dimethyl ether (400 mL) and water (100 mL), and the mixture was stirred and heated to 65° C. to 70° C. for 15 hours of the reaction under nitrogen protection; then the reaction mixture was cooled down to room temperature and washed with water, followed by separation of the organic phase, drying on anhydrous magnesium sulfate and then removal of the solvent under reduced pressure, obtaining a crude product. The crude product was purified by silica gel column chromatography using dichloromethane/ethanol as an eluent, to obtain IM L1 (30.7 g, yield 62.1%).

The IM Lx listed in Table 1 were synthesized with reference to the synthesis method for IM L1, except that Raw material 1 was used instead of 2-tert-butyl-4-bromo-1-chlorobenzene, and Raw material 2 was used instead of phenylboronic acid. The main raw materials used, the synthesized intermediates, and their yields are shown in Table 1.

TABLE 1

| Raw material 1 | Raw material 2 | IM Lx | Yield/% |
|---|---|---|---|
| 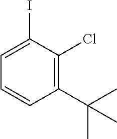 CAS:1369952-20-4 | 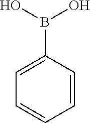 | 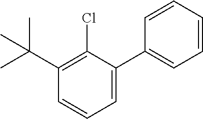 IM L2 | 67.3 |
| 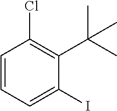 CAS:1369919-05-0 | 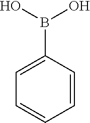 | 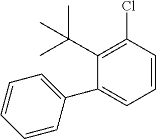 IM L3 | 66.5 |
| 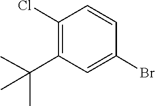 CAS:1020717-87-6 | 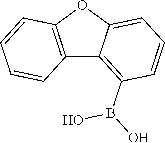 [SMILES: OB(O)c1cccc2oc3ccccc3c12] | 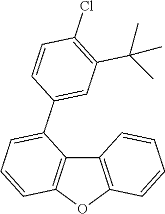 IM L4 | 64.8 |
| 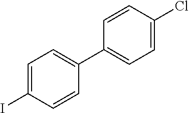 [SMILES: Clc1ccc(-c2ccc(I)cc2)cc1] CAS:60200-91-1 | 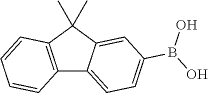 [SMILES: CC1(C)c2ccccc2-c2ccc(B(O)O)cc21] | 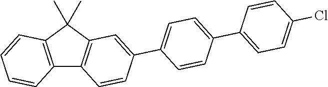 IM L5 | 65.6 |

### Synthesis of IM B1:

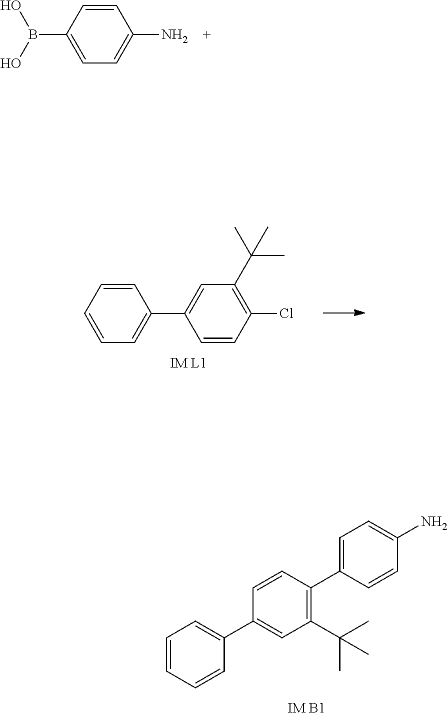 [SMILES: Nc1ccc(B(O)O)cc1 ; CC(C)(C)c1cc(-c2ccccc2)ccc1Cl ; CC(C)(C)c1cc(-c2ccccc2)ccc1-c1ccc(N)cc1]

To a three-necked flask was added 4-aminophenylboronic acid (30.0 g, 219.1 mmol), IM L1 (59.0 g, 241.0 mmol), bis(triphenylphosphine) palladium dichloride (0.77 g, 1.1 mmol), potassium carbonate (60.49 g, 438.2 mmol), ethylene glycol, dimethyl ether (400 mL) and water (60 mL), and the mixture was stirred and heated to 75° C. to 78° C. for 32 hours of the reaction under nitrogen protection; the reaction mixture was cooled down to room temperature and washed with water, followed by separation of the organic phase, drying on anhydrous magnesium sulfate and then removal of the solvent under reduced pressure, obtaining a crude product. The crude product is purified by silica gel column chromatography using dichloromethane/n-heptane as an eluent, to obtain IM B1 as an oily (42.8 g, yield 64.8%)

The IM Bx listed in Table 2 were synthesized with reference to the synthesis method for IM B1, except that Raw material 3 was used instead of IM L1. The main raw materials used, the synthesized intermediates, and their yields are shown in Table 2.

TABLE 2

| Raw material 3 | IM Bx | Yield/% |
|---|---|---|
| 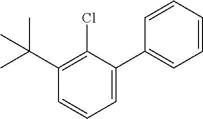 IM L2 | 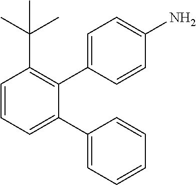 IM B2 | 60.2 |
| 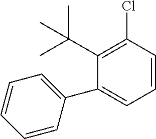 IM L3 | 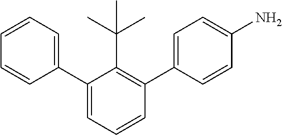 IM B3 | 62.8 |

### Synthesis of IM K1:

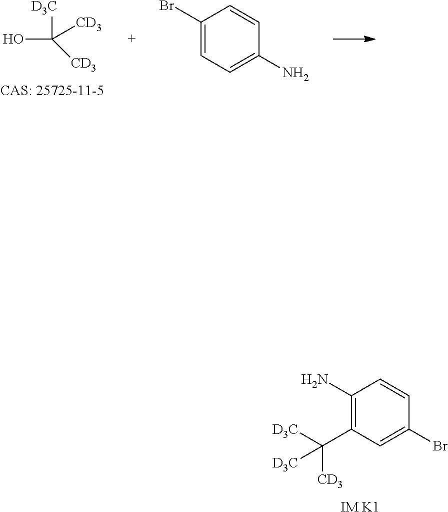

To a three necked flask was added dichloromethane (350 mL), tert butanol-D9 (50 g, 601.1 mmol), 4-bromoaniline (103.4 g, 601.1 mmol), and tert butoxycarbonyl (60 g, 601.1 mmol) with stirring, and the mixture was stirred under nitrogen protection, and cooled down to 15° C. by circulating refrigeration; deuterated sulfuric acid (60.2 g, 601.1 mmol) was added dropwise and reacted for 10 hours; the reaction mixture was placed in ice water to cool down to room temperature and washed with water, followed by separation of the organic phase, extraction with dichloromethane, drying on anhydrous magnesium sulfate, and removal of the solvent under reduced pressure, obtaining a crude product. The crude product was purified by silica gel column chromatography using dichloromethane/n-hexane as eluent to obtain IM K1 (60 g, yield 62.1%).

### Synthesis of IM A1:

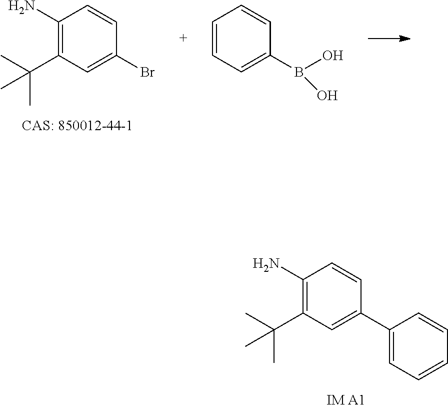

To a three-necked flask was added 4-bromo-2-tert-butylaniline (40.0 g, 175.3 mmol), phenylboronic acid (22.6 g, 184.1 mmol), tetrakis(triphenylphosphine) palladium (4.07 g, 3.5 mmol), potassium carbonate (48.7 g, 350.7 mmol), tetrabutyl ammonium bromide (11.36 g, 35.1 mmol), toluene (400 mL), ethanol (100 mL), and deionized water, and the mixture was stirred and heated to 75° C. to 80° C. for 26 hours of the reaction under nitrogen protection; then the reaction mixture was cooled down to room temperature and washed with water, followed by separation of the organic phase, drying on anhydrous magnesium sulfate and then removal of the solvent under reduced pressure, obtaining a crude product. The crude product is purified by silica gel column chromatography using dichloromethane/ethanol as an eluent, to obtain IM A1 as an oily (25.6 g, yield 64.8%).

The IM Ax listed in Table 3 were synthesized with reference to the synthesis method for IM A1, except that Raw material 4 was used instead of phenylboronic acid. The main raw materials used, the synthesized intermediates, and their yields are shown in Table 3.

TABLE 3

| Raw material 4 | IM Ax | Yield/% |
|---|---|---|
| 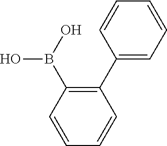 [SMILES: OB(O)c1ccccc1-c1ccccc1] CAS:4688-76-0 | 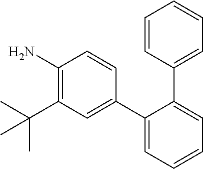 IM A2 | 62.3 |
| 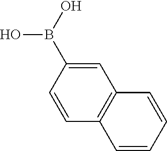 [SMILES: OB(O)c1ccc2ccccc2c1] CAS:32316-92-0 | 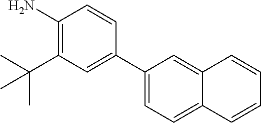 IM A3 | 63.5 |
| 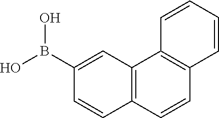 [SMILES: OB(O)c1ccc2ccc3ccccc3c2c1] | 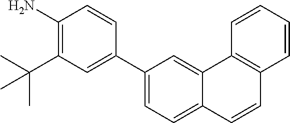 IM A4 | 59.7 |
| 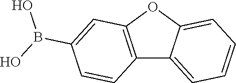 [SMILES: OB(O)c1ccc2c(c1)oc1ccccc12] CAS:395087-89-5 | 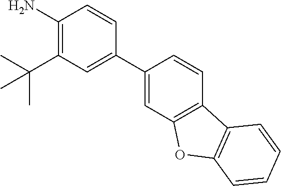 IM A5 | 60.4 |
| 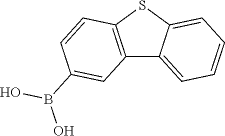 [SMILES: OB(O)c1ccc2sc3ccccc3c2c1] CAS:668983-97-9 | 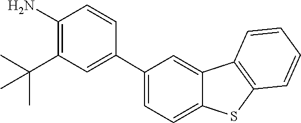 IM A6 | 59.5 |
| 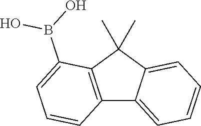 [SMILES: CC1(C)c2ccccc2-c2cccc(B(O)O)c21] CAS:1251825-71-4 | 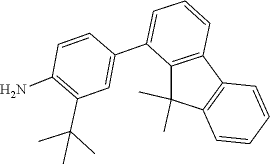 IM A7 | 61.2 |
| 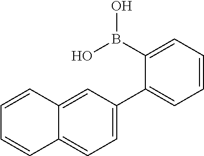 [SMILES: OB(O)c1ccccc1-c1ccc2ccccc2c1] | 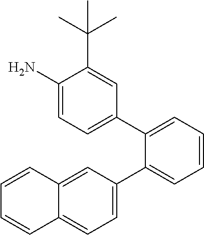 IM A8 | 53.5 |
| 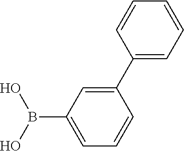 [SMILES: OB(O)c1cccc(-c2ccccc2)c1] CAS:5122-95-2 | 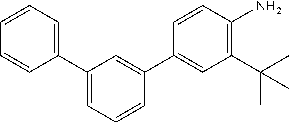 IM A9 | 63.5 |
| 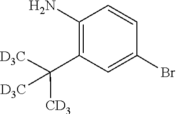 [SMILES: [2H]C([2H])([2H])C(c1cc(Br)ccc1N)(C([2H])([2H])[2H])C([2H])([2H])[2H]] LM K1 | 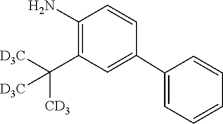 IM A10 | 64.8 |
| 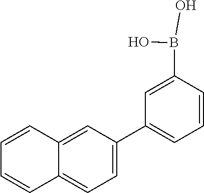 [SMILES: OB(O)c1cccc(-c2ccc3ccccc3c2)c1] | 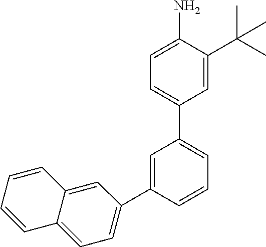 IM A11 | 58.7 |

### Synthesis of IM A1-1:

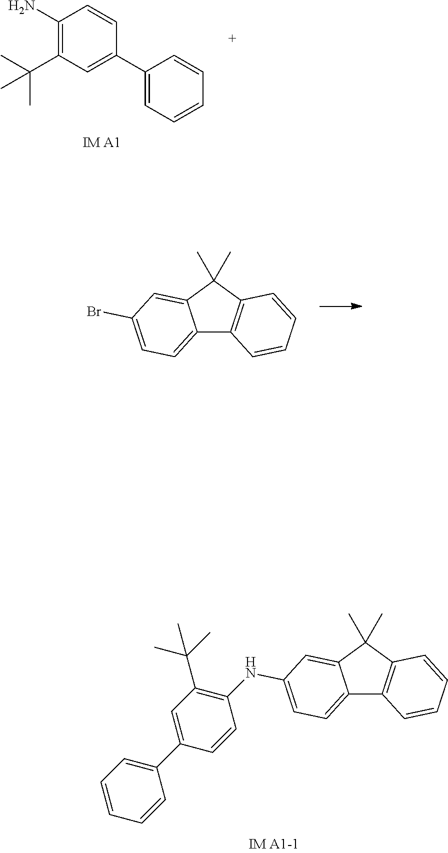

To a nitrogen-protected round bottom flask was added IM A1 (25.6 g, 113.6 mmol), 2-bromo-9,9-dimethylfluorene (31.0 g, 113.6 mmol), tris(dibenzylidene acetone) dipalladium (1.0 g, 1.1 mmol), 2-dicyclohexanephosphino-2′,4′,6′-triisopropylbiphenyl (0.9 g, 2.3 mmol), sodium tert-butoxide (16.38 g, 170.4 mmol), and toluene (240 mL), and the mixture was stirred and heated to 105° C. to 110° C. for 16 hours of reaction; the reaction solution was cooled down to room temperature and washed with water, followed by separation of the organic phase, drying on anhydrous magnesium sulfate, and removal of the solvent under reduced pressure, obtaining a crude product. The crude product was purified by silica gel column chromatography using dichloromethane/n-heptane, and then purified by recrystallization from toluene/n-heptane, to obtain IM A1-1 as a white solid (30.84 g, yield 65.0%).

The intermediates listed in Table 4 were synthesized with reference to the method for IM A1-1, except that Raw material 5 was used instead of IM A1, and Raw material 6 was used instead of 2-bromo-9,9-dimethylfluorene. The main raw materials used, the synthesized intermediates, and their yields are shown in Table 4.

TABLE 4

| Raw material 5 | Raw material 6 | Intermediate | Yield/% |
|---|---|---|---|
| 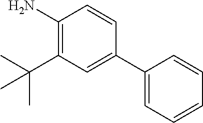 IM A1 | 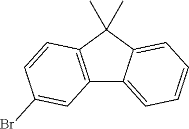 [SMILES: CC1(C)c2ccccc2-c2cc(Br)ccc21] | 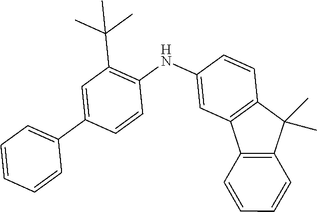 [SMILES: CC(C)(C)c1cc(-c2ccccc2)ccc1Nc1ccc2c(c1)-c1ccccc1C2(C)C] IM A9-1 | 67.1 |
| 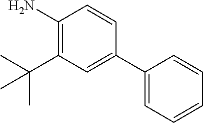 IM A1 | 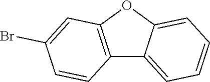 [SMILES: Brc1ccc2c(c1)oc1ccccc12] | 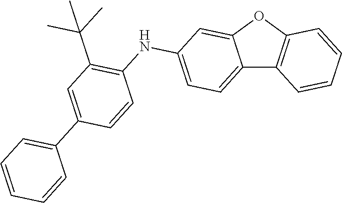 [SMILES: CC(C)(C)c1cc(-c2ccccc2)ccc1Nc1ccc2c(c1)oc1ccccc12] IM A10-1 | 68.3 |
| 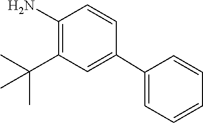 IM A1 | 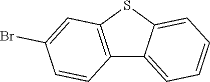 [SMILES: Brc1ccc2c(c1)sc1ccccc12] | 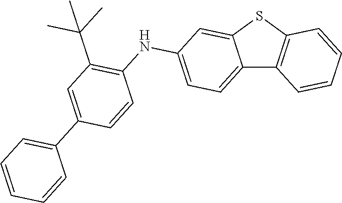 [SMILES: CC(C)(C)c1cc(-c2ccccc2)ccc1Nc1ccc2c(c1)sc1ccccc12] IM A11-1 | 66.5 |
| 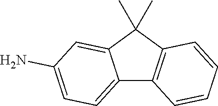 | 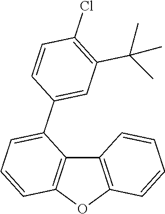 [SMILES: CC(C)(C)c1cc(-c2cccc3oc4ccccc4c23)ccc1Cl] LM L4 | 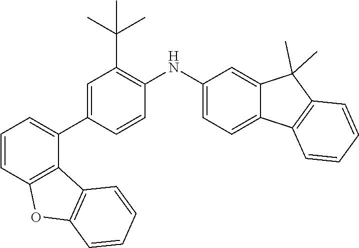 [SMILES: CC(C)(C)c1cc(-c2cccc3oc4ccccc4c23)ccc1Nc1ccc2c(c1)C(C)(C)c1ccccc1-2] IM A12-1 | 58.7 |
| 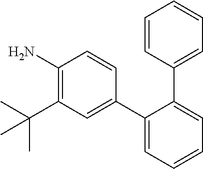 IM A2 | 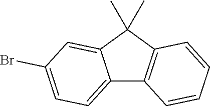 [SMILES: CC1(C)c2ccccc2-c2ccc(Br)cc21] | 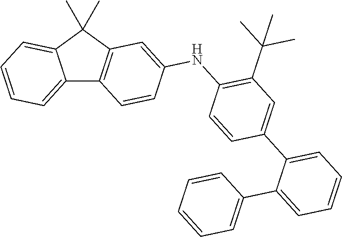 [SMILES: CC(C)(C)c1cc(-c2ccccc2-c2ccccc2)ccc1Nc1ccc2c(c1)C(C)(C)c1ccccc1-2] IM A2-1 | 64.2 |
| 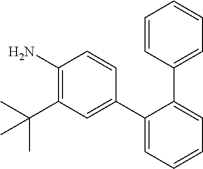 IM A2 | 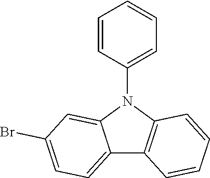 [SMILES: Brc1ccc2c3ccccc3n(-c3ccccc3)c2c1] | 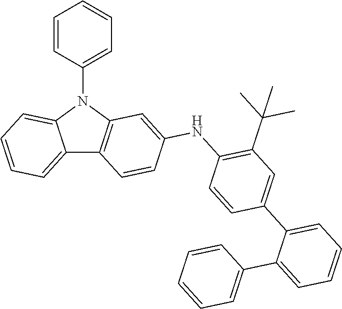 [SMILES: CC(C)(C)c1cc(-c2ccccc2-c2ccccc2)ccc1Nc1ccc2c3ccccc3n(-c3ccccc3)c2c1] IM A17-1 | 59.3 |
| 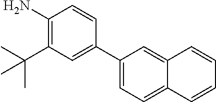 IM A3 | 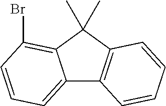 [SMILES: CC1(C)c2ccccc2-c2cccc(Br)c21] | 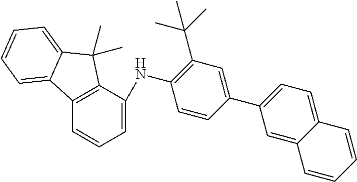 [SMILES: CC(C)(C)c1cc(-c2ccc3ccccc3c2)ccc1Nc1cccc2c1C(C)(C)c1ccccc1-2] IM A3-1 | 62.6 |
| 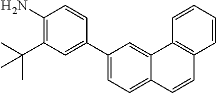 IM A4 | 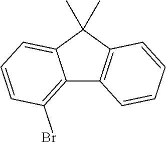 [SMILES: CC1(C)c2ccccc2-c2c(Br)cccc21] | 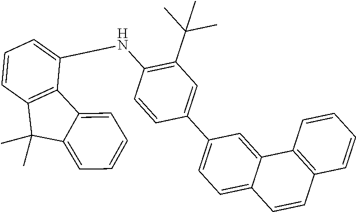 [SMILES: CC(C)(C)c1cc(-c2ccc3ccc4ccccc4c3c2)ccc1NCc1cccc2c1-c1ccccc1C2(C)C] IM A4-1 | 60.5 |
| 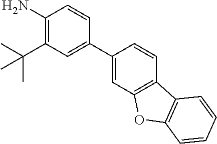 IM A5 | 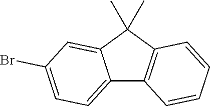 [SMILES: CC1(C)c2ccccc2-c2ccc(Br)cc21] | 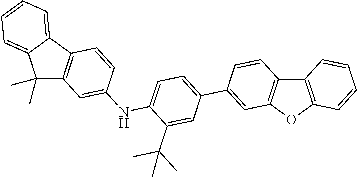 [SMILES: CC(C)(C)c1cc(-c2ccc3c(c2)oc2ccccc23)ccc1Nc1ccc2c(c1)C(C)(C)c1ccccc1-2] IM A5-1 | 59.8 |
| 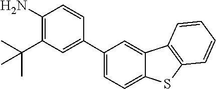 IM A6 | 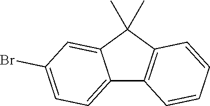 [SMILES: CC1(C)c2ccccc2-c2ccc(Br)cc21] | 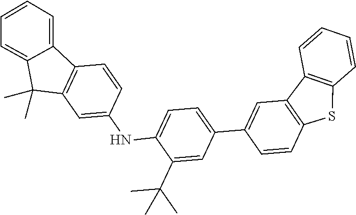 [SMILES: CC(C)(C)c1cc(-c2ccc3sc4ccccc4c3c2)ccc1Nc1ccc2c(c1)C(C)(C)c1ccccc1-2] IM A6-1 | 58.5 |
| 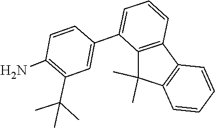 IM A7 | 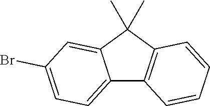 [SMILES: CC1(C)c2ccccc2-c2ccc(Br)cc21] | 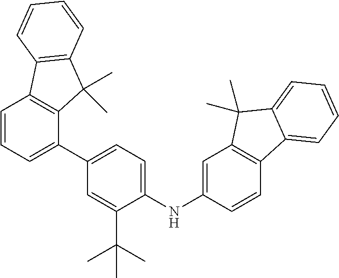 [SMILES: CC(C)(C)c1cc(-c2cccc3c2C(C)(C)c2ccccc2-3)ccc1Nc1ccc2c(c1)C(C)(C)c1ccccc1-2] IM A7-1 | 57.2 |
| 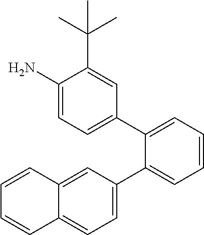 LM A8 | 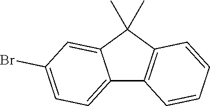 [SMILES: CC1(C)c2ccccc2-c2ccc(Br)cc21] | 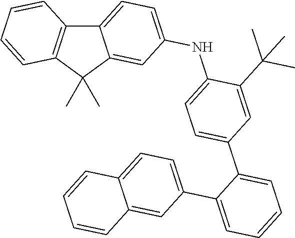 [SMILES: CC(C)(C)c1cc(-c2ccccc2-c2ccc3ccccc3c2)ccc1Nc1ccc2c(c1)C(C)(C)c1ccccc1-2] LMA8-1 | 60.8 |
| 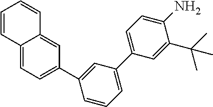 IM A11 | 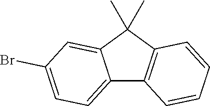 [SMILES: CC1(C)c2ccccc2-c2ccc(Br)cc21] | 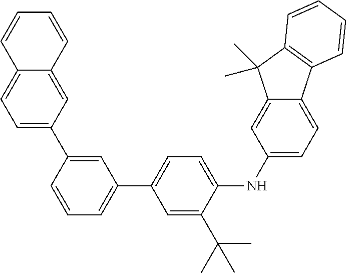 [SMILES: CC(C)(C)c1cc(-c2cccc(-c3ccc4ccccc4c3)c2)ccc1Nc1ccc2c(c1)C(C)(C)c1ccccc1-2] IM A16-1 | 56.7 |
| 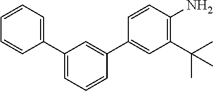 LM A9 | 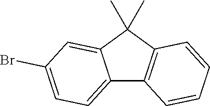 [SMILES: CC1(C)c2ccccc2-c2ccc(Br)cc21] | 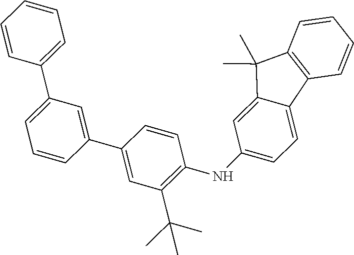 [SMILES: CC(C)(C)c1cc(-c2cccc(-c3ccccc3)c2)ccc1Nc1ccc2c(c1)C(C)(C)c1ccccc1-2] IM A14-1 | 62.5 |
| 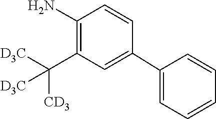 LM A10 | 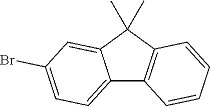 [SMILES: CC1(C)c2ccccc2-c2ccc(Br)cc21] | 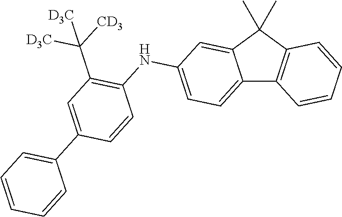 [SMILES: [2H]C([2H])([2H])C(c1cc(-c2ccccc2)ccc1Nc1ccc2c(c1)C(C)(C)c1ccccc1-2)(C([2H])([2H])[2H])C([2H])([2H])[2H]] LM A15-1 | 64.5 |
| 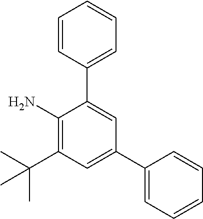 CAS:173282-40-1 | 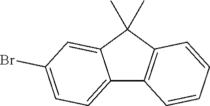 [SMILES: CC1(C)c2ccccc2-c2ccc(Br)cc21] | 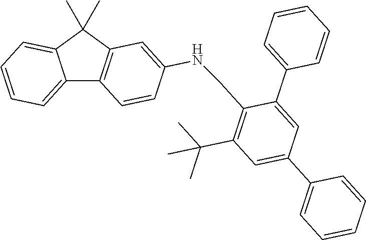 [SMILES: CC(C)(C)c1cc(-c2ccccc2)cc(-c2ccccc2)c1CNc1ccc2c(c1)C(C)(C)c1ccccc1-2] IM A13-1 | 63.4 |
| 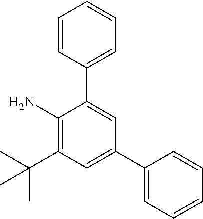 CAS:173282-40-1 | 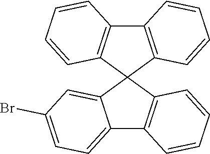 [SMILES: Brc1ccc2c(c1)C1(c3ccccc3-c3ccccc31)c1ccccc1-2] | 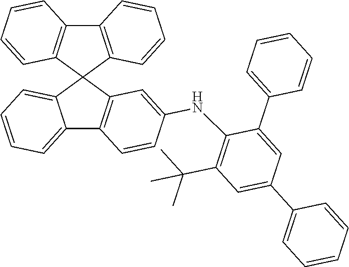 [SMILES: CC(C)(C)c1cc(-c2ccccc2)cc(-c2ccccc2)c1Nc1ccc2c(c1)C1(c3ccccc3-c3ccccc31)c1ccccc1-2] IM A18-1 | 59.2 |
| 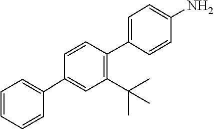 IM B1 | 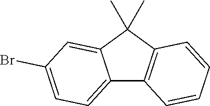 [SMILES: CC1(C)c2ccccc2-c2ccc(Br)cc21] | 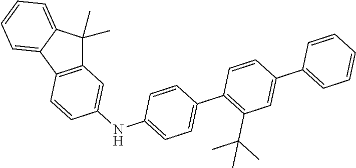 [SMILES: CC(C)(C)c1cc(-c2ccccc2)ccc1-c1ccc(Nc2ccc3c(c2)C(C)(C)c2ccccc2-3)cc1] IM B1-1 | 62.8 |
| 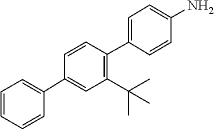 IM B1 | 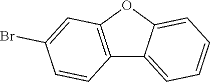 [SMILES: Brc1ccc2c(c1)oc1ccccc12] | 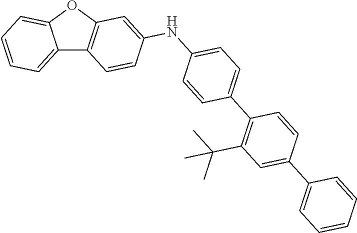 [SMILES: CC(C)(C)c1cc(-c2ccccc2)ccc1-c1ccc(Nc2ccc3c(c2)oc2ccccc23)cc1] IM B7-1 | 62.5 |
| 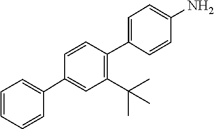 IM B1 | 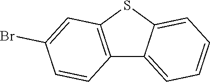 [SMILES: Brc1ccc2c(c1)sc1ccccc12] | 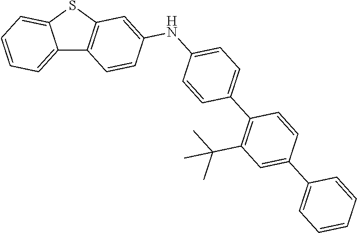 [SMILES: CC(C)(C)c1cc(-c2ccccc2)ccc1-c1ccc(Nc2ccc3c(c2)sc2ccccc23)cc1] IM B8-1 | 60.0 |
| 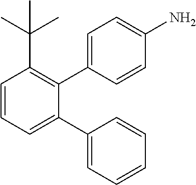 IM B2 | 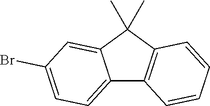 [SMILES: CC1(C)c2ccccc2-c2ccc(Br)cc21] | 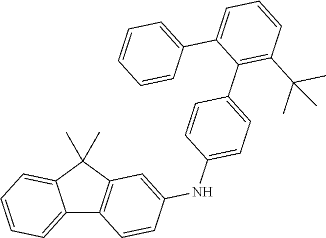 [SMILES: CC(C)(C)c1cccc(-c2ccccc2)c1-c1ccc(Nc2ccc3c(c2)C(C)(C)c2ccccc2-3)cc1] IM B2-1 | 64.5 |
| 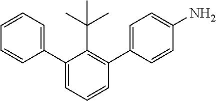 IM B3 | 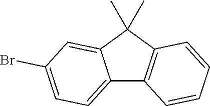 [SMILES: CC1(C)c2ccccc2-c2ccc(Br)cc21] | 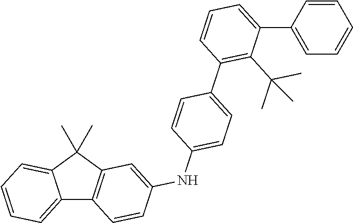 [SMILES: CC(C)(C)c1c(-c2ccccc2)cccc1-c1ccc(Nc2ccc3c(c2)C(C)(C)c2ccccc2-3)cc1] IM B3-1 | 63.1 |

### Synthesis of Compound 1

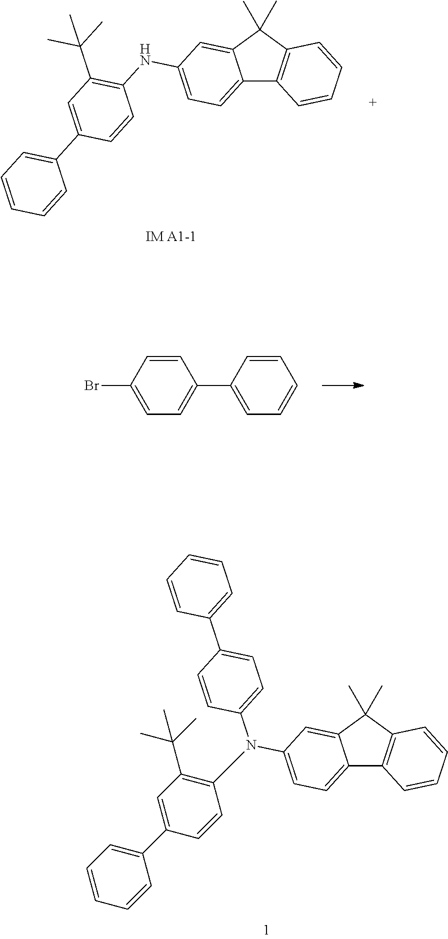 [SMILES: CC(C)(C)c1cc(-c2ccccc2)ccc1Nc1ccc2c(c1)C(C)(C)c1ccccc1-2 ; Brc1ccc(-c2ccccc2)cc1 ; CC(C)(C)c1cc(-c2ccccc2)ccc1N(c1ccc(-c2ccccc2)cc1)c1ccc2c(c1)C(C)(C)c1ccccc1-2]

To a nitrogen-protected round bottom flask was added IM A1-1 (30 g, 71.8 mmol), 4-bromobiphenyl (16.7 g, 71.8 mmol), tris(dibenzylidene acetone) dipalladium (0.3 g, 0.7 mmol), 2-dicyclohexanephosphino-2,6-dimethoxybiphenyl (0.6 g, 1.4 mmol), sodium tert-butoxide (10.4 g, 107.7 mmol) and toluene (280 mL), and the mixture was stirred and heated to 105° C. to 110° C. for 18 hours of reaction; the reaction solution was cooled down to room temperature and washed with water, followed by separation of the organic phase, drying on anhydrous magnesium sulfate, and removal of the solvent under reduced pressure, obtaining a crude product. The crude product was purified by silica gel column chromatography using dichloromethane/n-heptane, and then purified by recrystallization from toluene/n-heptane, to obtain Compound 1 as a white solid (26.65 g, yield 65.1%). Mass Spectra (m/z)=570.3 [M+H]⁺.

The compounds listed in Table 5 were synthesized with reference to the method for Compound 1, except that Raw material 7 was used instead of IM A1-1 and Raw material 8 was used instead of 4-bromobiphenyl. The main raw materials used, the synthesized intermediates, and their mass spectra are shown in Table 5.

TABLE 5

| | | | | Mass |
|---|---|---|---|---|
| | | | | Spectra |
| | | | Yield/ | (m/z)/ |
| Raw material 7 | Raw material 8 | Compound | % | [M + H]⁺ |
| 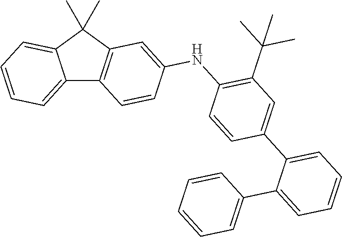 [SMILES: CC(C)(C)c1cc(-c2ccccc2-c2ccccc2)ccc1Nc1ccc2c(c1)C(C)(C)c1ccccc1-2] IM A2-1 | 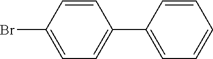 [SMILES: Brc1ccc(-c2ccccc2)cc1] | 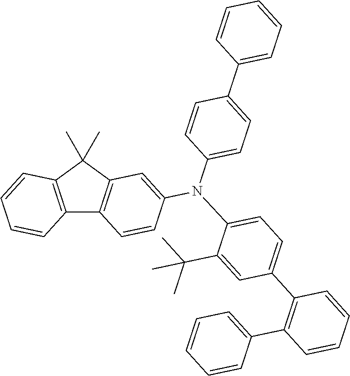 [SMILES: CC(C)(C)c1cc(-c2ccccc2-c2ccccc2)ccc1N(c1ccc(-c2ccccc2)cc1)c1ccc2c(c1)C(C)(C)c1ccccc1-2] 2 | 63.2 | 646.3 |
| 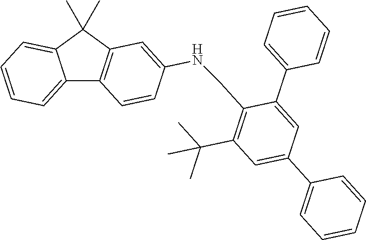 [SMILES: CC(C)(C)c1cc(-c2ccccc2)cc(-c2ccccc2)c1CNc1ccc2c(c1)C(C)(C)c1ccccc1-2] IM A13-1 | 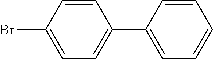 [SMILES: Brc1ccc(-c2ccccc2)cc1] | 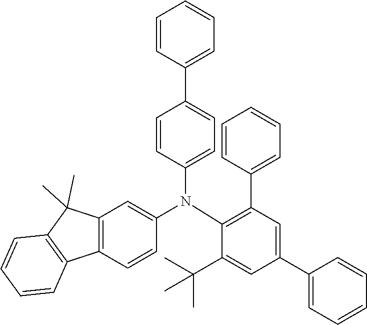 [SMILES: CC(C)(C)c1cc(-c2ccccc2)cc(-c2ccccc2)c1N(c1ccc(-c2ccccc2)cc1)c1ccc2c(c1)C(C)(C)c1ccccc1-2] 4 | 57.0 | 646.3 |
| 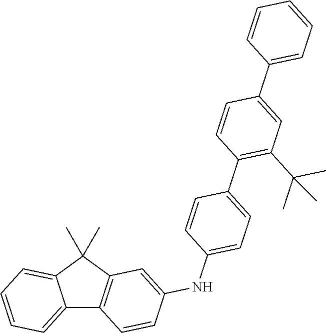 [SMILES: CC(C)(C)c1cc(-c2ccccc2)ccc1-c1ccc(Nc2ccc3c(c2)C(C)(C)c2ccccc2-3)cc1] IM B1-1 | 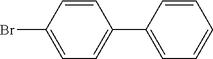 [SMILES: Brc1ccc(-c2ccccc2)cc1] | 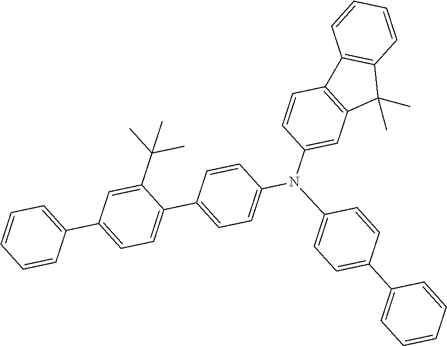 [SMILES: CC(C)(C)c1cc(-c2ccccc2)ccc1-c1ccc(N(c2ccc(-c3ccccc3)cc2)c2ccc3c(c2)C(C)(C)c2ccccc2-3)cc1] 7 | 60.3 | 646.3 |
| 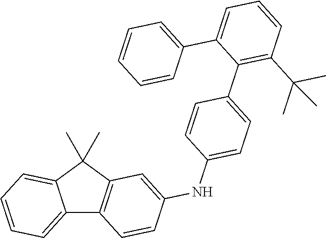 [SMILES: CC(C)(C)c1cccc(-c2ccccc2)c1-c1ccc(Nc2ccc3c(c2)C(C)(C)c2ccccc2-3)cc1] IM B2-1 | 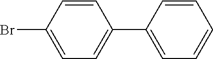 [SMILES: Brc1ccc(-c2ccccc2)cc1] | 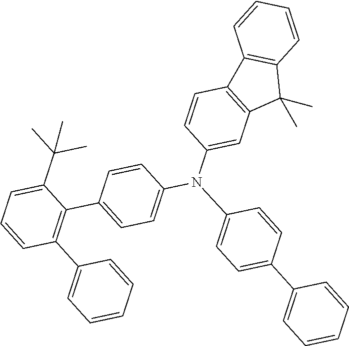 [SMILES: CC(C)(C)c1cccc(-c2ccccc2)c1-c1ccc(N(c2ccc(-c3ccccc3)cc2)c2ccc3c(c2)C(C)(C)c2ccccc2-3)cc1] 8 | 59.4 | 646.3 |
| 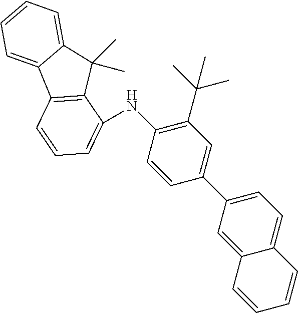 [SMILES: CC(C)(C)c1cc(-c2ccc3ccccc3c2)ccc1Nc1cccc2c1C(C)(C)c1ccccc1-2] IM A3-1 | 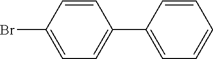 [SMILES: Brc1ccc(-c2ccccc2)cc1] | 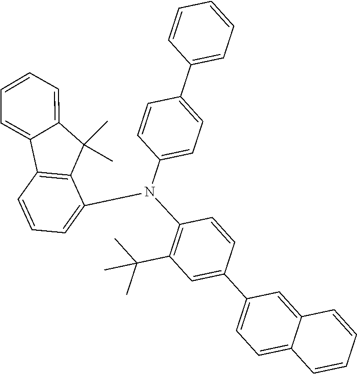 [SMILES: CC(C)(C)c1cc(-c2ccc3ccccc3c2)ccc1N(Cc1cccc2c1C(C)(C)c1ccccc1-2)c1ccc(-c2ccccc2)cc1] 16 | 62.1 | 620.3 |
| 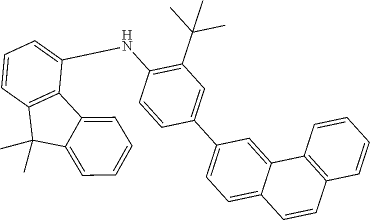 [SMILES: CC(C)(C)c1cc(-c2ccc3ccc4ccccc4c3c2)ccc1NCc1cccc2c1-c1ccccc1C2(C)C] IM A4-1 | 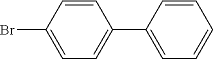 [SMILES: Brc1ccc(-c2ccccc2)cc1] | 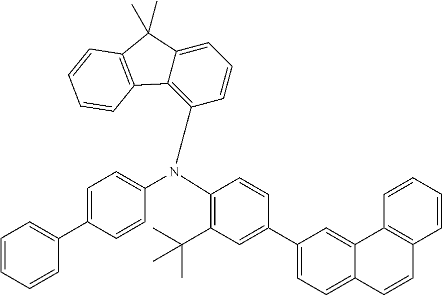 [SMILES: CC(C)(C)c1cc(-c2ccc3ccc4ccccc4c3c2)ccc1N(Cc1cccc2c1-c1ccccc1C2(C)C)c1ccc(-c2ccccc2)cc1] 18 | 58.5 | 670.3 |
| 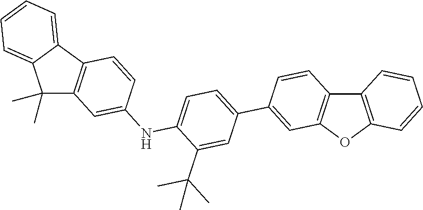 [SMILES: CC(C)(C)c1cc(-c2ccc3c(c2)oc2ccccc23)ccc1Nc1ccc2c(c1)C(C)(C)c1ccccc1-2] IM A5-1 | 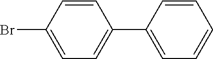 [SMILES: Brc1ccc(-c2ccccc2)cc1] | 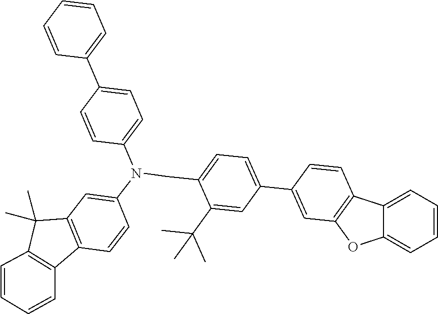 [SMILES: CC(C)(C)c1cc(-c2ccc3c(c2)oc2ccccc23)ccc1CN(c1ccc(-c2ccccc2)cc1)c1ccc2c(c1)C(C)(C)c1ccccc1-2] 22 | 56.7 | 660.3 |
| 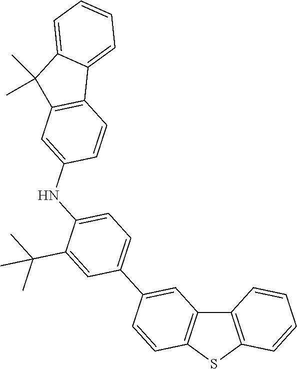 [SMILES: CC(C)(C)c1cc(-c2ccc3sc4ccccc4c3c2)ccc1Nc1ccc2c(c1)C(C)(C)c1ccccc1-2] IM A6-1 | 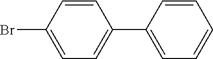 [SMILES: Brc1ccc(-c2ccccc2)cc1] | 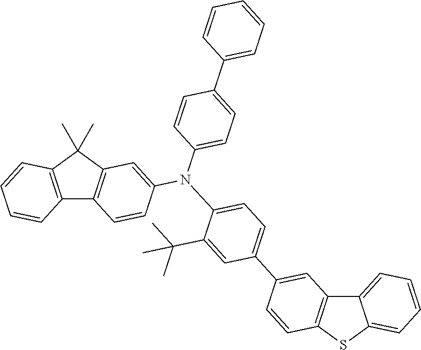 [SMILES: CC(C)(C)c1cc(-c2ccc3sc4ccccc4c3c2)ccc1N(c1ccc(-c2ccccc2)cc1)c1ccc2c(c1)C(C)(C)c1ccccc1-2] 31 | 56.0 | 676.3 |
| 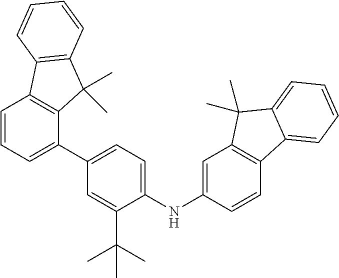 [SMILES: CC(C)(C)c1cc(-c2cccc3c2C(C)(C)c2ccccc2-3)ccc1Nc1ccc2c(c1)C(C)(C)c1ccccc1-2] IM A7-1 | 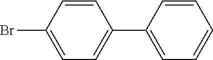 [SMILES: Brc1ccc(-c2ccccc2)cc1] | 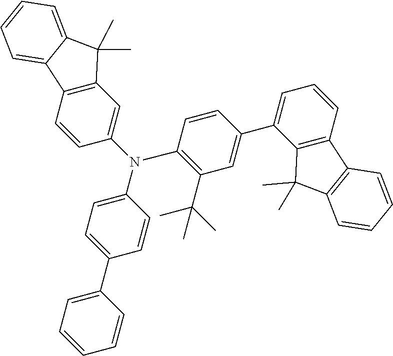 [SMILES: CC(C)(C)c1cc(-c2cccc3c2C(C)(C)c2ccccc2-3)ccc1N(c1ccc(-c2ccccc2)cc1)c1ccc2c(c1)C(C)(C)c1ccccc1-2] 38 | 55.3 | 686.4 |
| 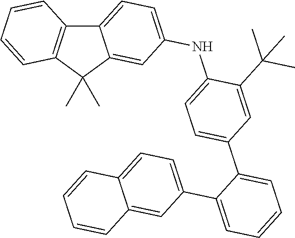 [SMILES: CC(C)(C)c1cc(-c2ccccc2-c2ccc3ccccc3c2)ccc1Nc1ccc2c(c1)C(C)(C)c1ccccc1-2] LM A8-1 | 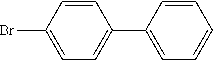 [SMILES: Brc1ccc(-c2ccccc2)cc1] | 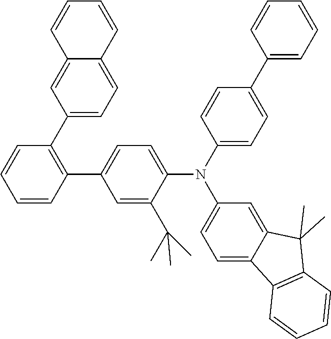 [SMILES: CC(C)(C)c1cc(-c2ccccc2-c2ccc3ccccc3c2)ccc1N(c1ccc(-c2ccccc2)cc1)c1ccc2c(c1)C(C)(C)c1ccccc1-2] 209 | 53.2 | 696.4 |
| 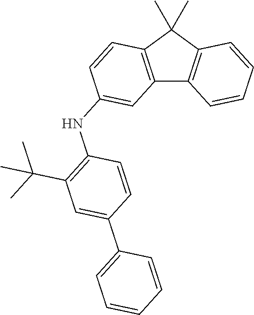 [SMILES: CC(C)(C)c1cc(-c2ccccc2)ccc1Nc1ccc2c(c1)-c1ccccc1C2(C)C] IM A9-1 | 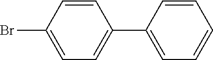 [SMILES: Brc1ccc(-c2ccccc2)cc1] | 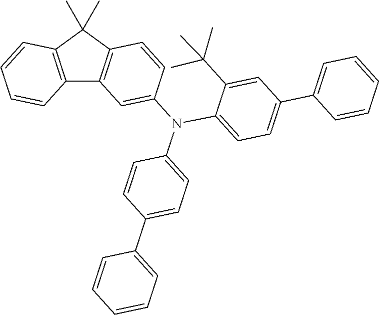 [SMILES: CC(C)(C)c1cc(-c2ccccc2)ccc1N(c1ccc(-c2ccccc2)cc1)c1ccc2c(c1)-c1ccccc1C2(C)C] 47 | 63.5 | 570.3 |
| 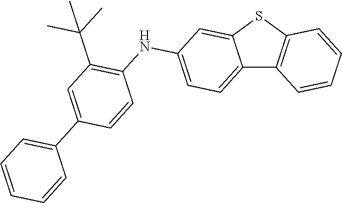 [SMILES: CC(C)(C)c1cc(-c2ccccc2)ccc1Nc1ccc2c(c1)sc1ccccc12] IM A11-1 | 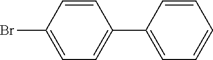 [SMILES: Brc1ccc(-c2ccccc2)cc1] | 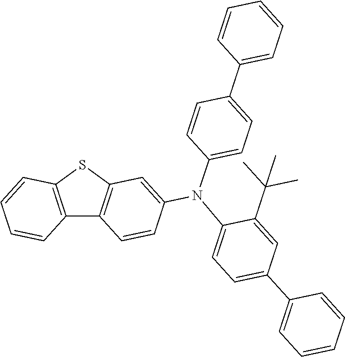 [SMILES: CC(C)(C)c1cc(-c2ccccc2)ccc1N(c1ccc(-c2ccccc2)cc1)c1ccc2c(c1)sc1ccccc12] 72 | 60.1 | 560.2 |
| 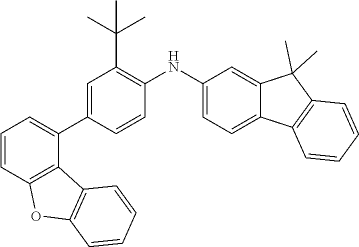 [SMILES: CC(C)(C)c1cc(-c2cccc3oc4ccccc4c23)ccc1Nc1ccc2c(c1)C(C)(C)c1ccccc1-2] IM A12-1 | 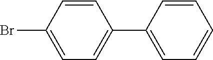 [SMILES: Brc1ccc(-c2ccccc2)cc1] | 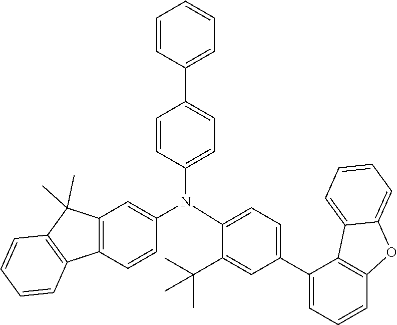 [SMILES: CC(C)(C)c1cc(-c2cccc3oc4ccccc4c23)ccc1N(c1ccc(-c2ccccc2)cc1)c1ccc2c(c1)C(C)(C)c1ccccc1-2] 24 | 61.3 | 660.3 |
| 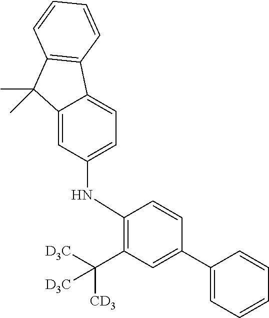 [SMILES: [2H]C([2H])([2H])C(c1cc(-c2ccccc2)ccc1Nc1ccc2c(c1)C(C)(C)c1ccccc1-2)(C([2H])([2H])[2H])C([2H])([2H])[2H]] LM A15-1 | 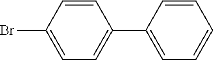 [SMILES: Brc1ccc(-c2ccccc2)cc1] | 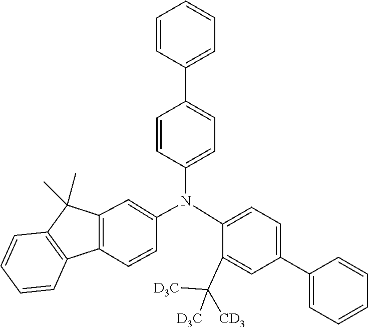 [SMILES: [2H]C([2H])([2H])C(c1cc(-c2ccccc2)ccc1N(c1ccc(-c2ccccc2)cc1)c1ccc2c(c1)C(C)(C)c1ccccc1-2)(C([2H])([2H])[2H])C([2H])([2H])[2H]] 10 | 62.5 | 579.4 |
| 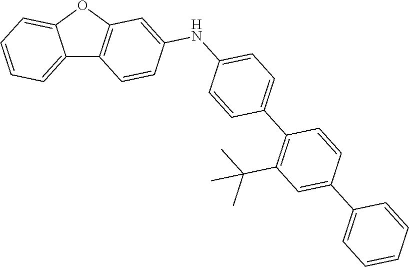 [SMILES: CC(C)(C)c1cc(-c2ccccc2)ccc1-c1ccc(Nc2ccc3c(c2)oc2ccccc23)cc1] IM B7-1 | 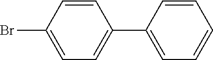 [SMILES: Brc1ccc(-c2ccccc2)cc1] | 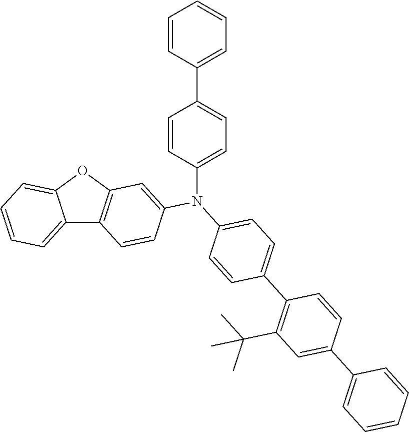 [SMILES: CC(C)(C)c1cc(-c2ccccc2)ccc1-c1ccc(N(c2ccc(-c3ccccc3)cc2)c2ccc3c(c2)oc2ccccc23)cc1] 155 | 59.7 | 620.3 |
| 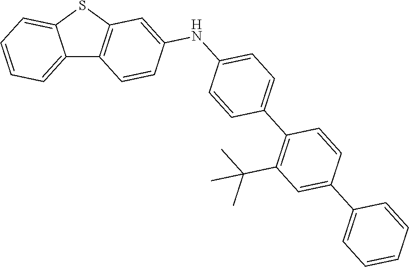 [SMILES: CC(C)(C)c1cc(-c2ccccc2)ccc1-c1ccc(Nc2ccc3c(c2)sc2ccccc23)cc1] IM B8-1 | 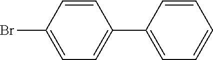 [SMILES: Brc1ccc(-c2ccccc2)cc1] | 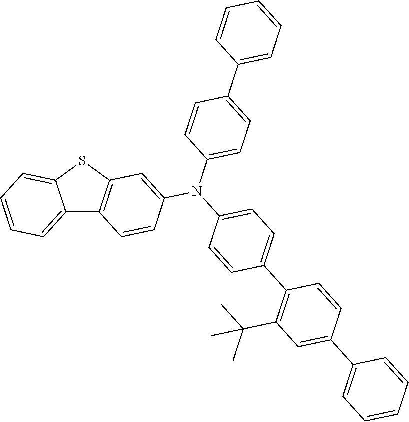 [SMILES: CC(C)(C)c1cc(-c2ccccc2)ccc1-c1ccc(N(c2ccc(-c3ccccc3)cc2)c2ccc3c(c2)sc2ccccc23)cc1] 170 | 58.8 | 636.3 |
| 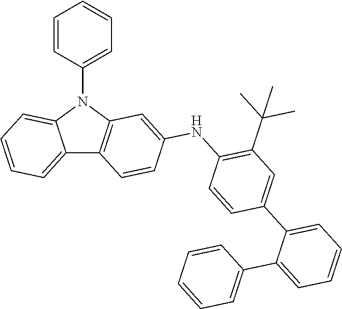 [SMILES: CC(C)(C)c1cc(-c2ccccc2-c2ccccc2)ccc1Nc1ccc2c3ccccc3n(-c3ccccc3)c2c1] IM A17-1 | 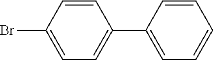 [SMILES: Brc1ccc(-c2ccccc2)cc1] | 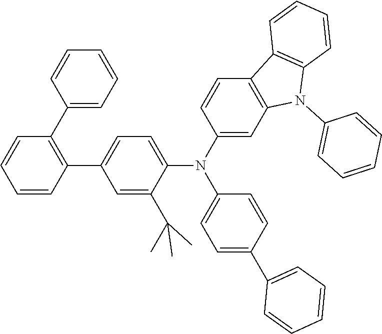 [SMILES: CC(C)(C)c1cc(-c2ccccc2-c2ccccc2)ccc1N(c1ccc(-c2ccccc2)cc1)c1ccc2c3ccccc3n(-c3ccccc3)c2c1] 42 | 49.9 | 695.3 |
| 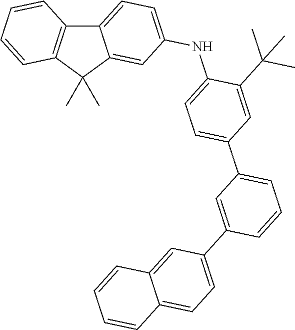 [SMILES: CC(C)(C)c1cc(-c2cccc(-c3ccc4ccccc4c3)c2)ccc1Nc1ccc2c(c1)C(C)(C)c1ccccc1-2] IM A16-1 | 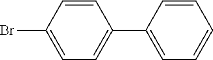 [SMILES: Brc1ccc(-c2ccccc2)cc1] | 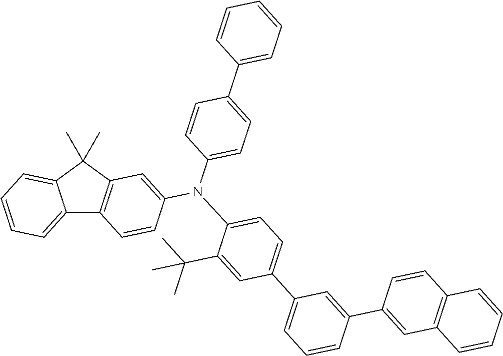 [SMILES: CC(C)(C)c1cc(-c2cccc(-c3ccc4ccccc4c3)c2)ccc1N(c1ccc(-c2ccccc2)cc1)c1ccc2c(c1)C(C)(C)c1ccccc1-2] 134 | 54.7 | 696.4 |
| 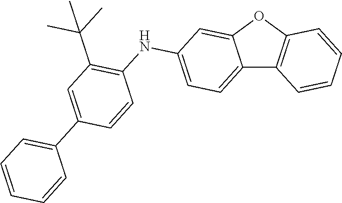 [SMILES: CC(C)(C)c1cc(-c2ccccc2)ccc1Nc1ccc2c(c1)oc1ccccc12] IM A10-1 | 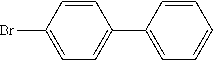 [SMILES: Brc1ccc(-c2ccccc2)cc1] | 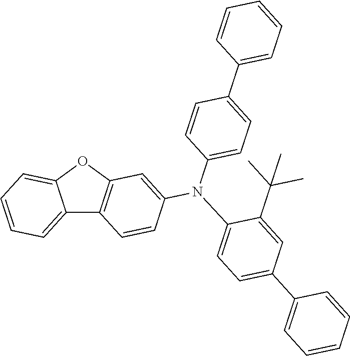 [SMILES: CC(C)(C)c1cc(-c2ccccc2)ccc1N(c1ccc(-c2ccccc2)cc1)c1ccc2c(c1)oc1ccccc12] 59 | 62.0 | 544.3 |
| 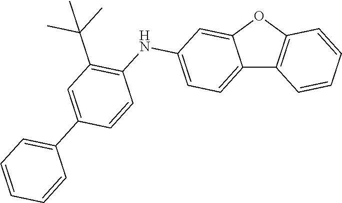 [SMILES: CC(C)(C)c1cc(-c2ccccc2)ccc1Nc1ccc2c(c1)oc1ccccc12] IM A10-1 | 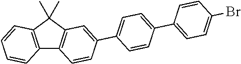 [SMILES: CC1(C)c2ccccc2-c2ccc(-c3ccc(-c4ccc(Br)cc4)cc3)cc21] IM L5 | 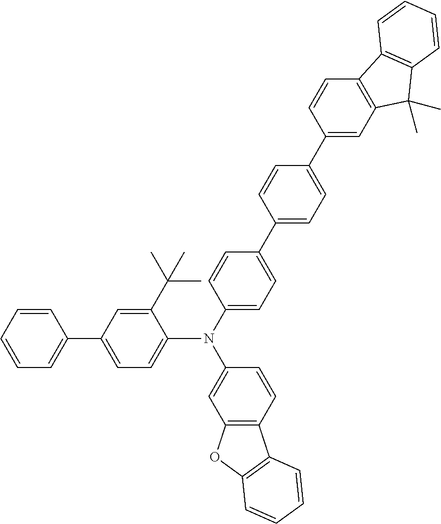 [SMILES: CC(C)(C)c1cc(-c2ccccc2)ccc1N(c1ccc(-c2ccc(-c3ccc4c(c3)C(C)(C)c3ccccc3-4)cc2)cc1)c1ccc2c(c1)oc1ccccc12] 6 | 56.3 | 736.4 |
| 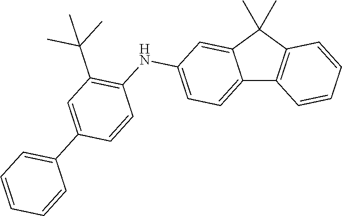 [SMILES: CC(C)(C)c1cc(-c2ccccc2)ccc1Nc1ccc2c(c1)C(C)(C)c1ccccc1-2] IM A1-1 | 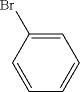 | 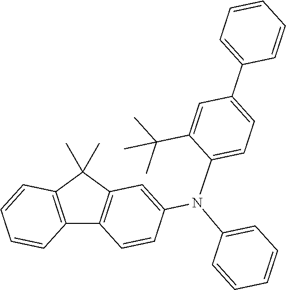 [SMILES: CC(C)(C)c1cc(-c2ccccc2)ccc1N(c1ccccc1)c1ccc2c(c1)C(C)(C)c1ccccc1-2] 84 | 68.5 | 494.3 |
| 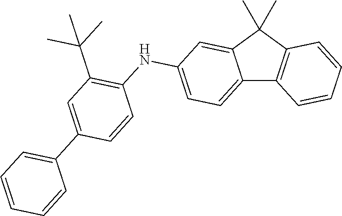 [SMILES: CC(C)(C)c1cc(-c2ccccc2)ccc1Nc1ccc2c(c1)C(C)(C)c1ccccc1-2] IM A1-1 | 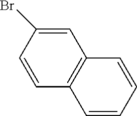 [SMILES: Brc1ccc2ccccc2c1] | 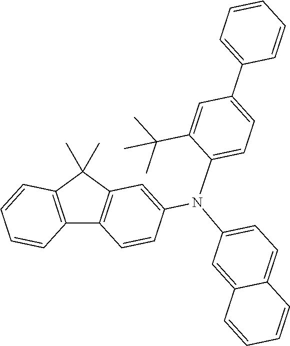 [SMILES: CC(C)(C)c1cc(-c2ccccc2)ccc1N(c1ccc2c(c1)C(C)(C)c1ccccc1-2)c1ccc2ccccc2c1] 86 | 65.4 | 544.3 |
| 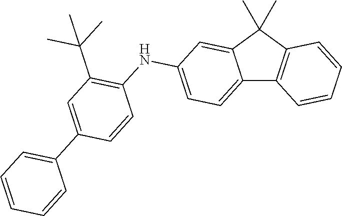 [SMILES: CC(C)(C)c1cc(-c2ccccc2)ccc1Nc1ccc2c(c1)C(C)(C)c1ccccc1-2] IM A1-1 | 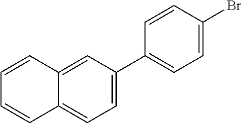 [SMILES: Brc1ccc(-c2ccc3ccccc3c2)cc1] | 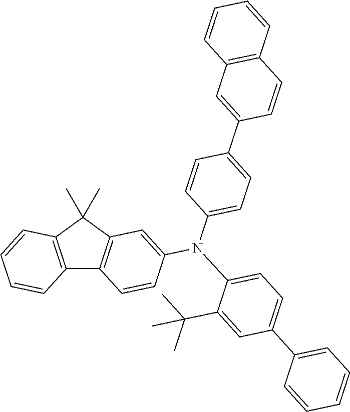 [SMILES: CC(C)(C)c1cc(-c2ccccc2)ccc1N(c1ccc(-c2ccc3ccccc3c2)cc1)c1ccc2c(c1)C(C)(C)c1ccccc1-2] 90 | 64.9 | 620.3 |
| 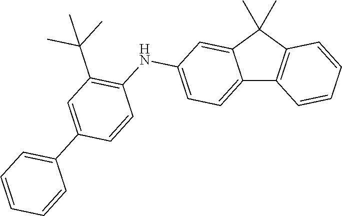 [SMILES: CC(C)(C)c1cc(-c2ccccc2)ccc1Nc1ccc2c(c1)C(C)(C)c1ccccc1-2] IM A1-1 | 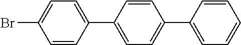 [SMILES: Brc1ccc(-c2ccc(-c3ccccc3)cc2)cc1] | 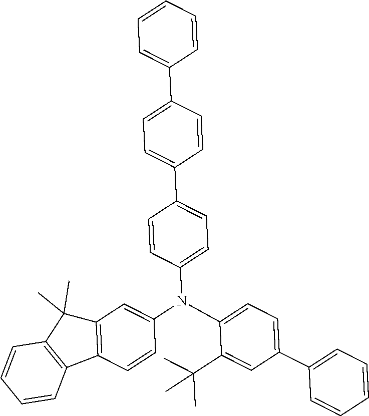 [SMILES: CC(C)(C)c1cc(-c2ccccc2)ccc1N(c1ccc(-c2ccc(-c3ccccc3)cc2)cc1)c1ccc2c(c1)C(C)(C)c1ccccc1-2] 92 | 65.3 | 646.3 |
| 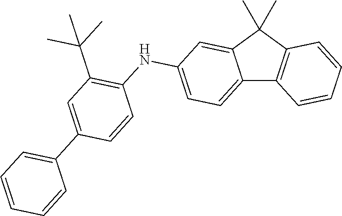 [SMILES: CC(C)(C)c1cc(-c2ccccc2)ccc1Nc1ccc2c(c1)C(C)(C)c1ccccc1-2] IM A1-1 | 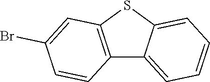 [SMILES: Brc1ccc2c(c1)sc1ccccc12] | 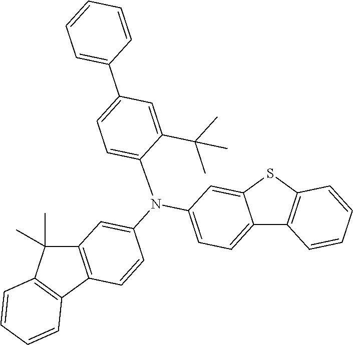 [SMILES: CC(C)(C)c1cc(-c2ccccc2)ccc1N(c1ccc2c(c1)C(C)(C)c1ccccc1-2)c1ccc2c(c1)sc1ccccc12] 103 | 66.0 | 600.3 |
| 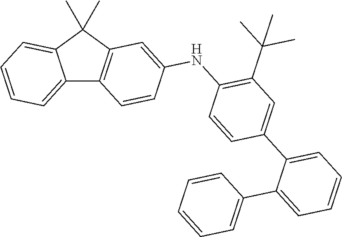 [SMILES: CC(C)(C)c1cc(-c2ccccc2-c2ccccc2)ccc1Nc1ccc2c(c1)C(C)(C)c1ccccc1-2] IM A2-1 | 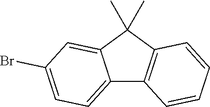 [SMILES: CC1(C)c2ccccc2-c2ccc(Br)cc21] | 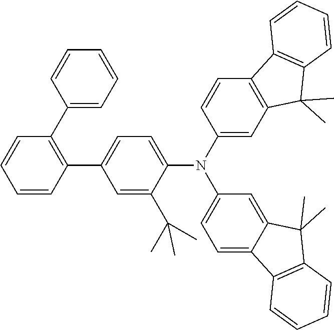 [SMILES: CC(C)(C)c1cc(-c2ccccc2-c2ccccc2)ccc1N(c1ccc2c(c1)C(C)(C)c1ccccc1-2)c1ccc2c(c1)C(C)(C)c1ccccc1-2] 210 | 62.7 | 686.4 |
| 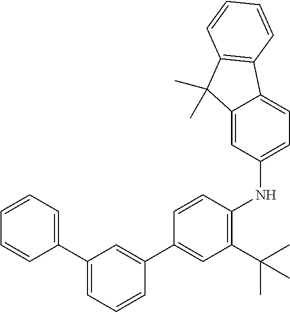 [SMILES: CC(C)(C)c1cc(-c2cccc(-c3ccccc3)c2)ccc1Nc1ccc2c(c1)C(C)(C)c1ccccc1-2] IM A14-1 | 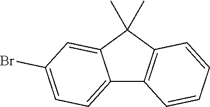 [SMILES: CC1(C)c2ccccc2-c2ccc(Br)cc21] | 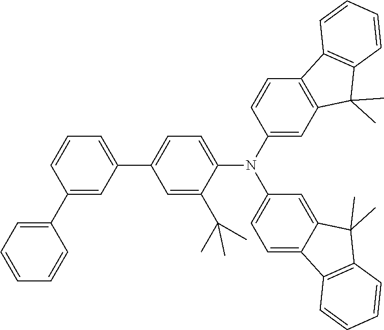 [SMILES: CC(C)(C)c1cc(-c2cccc(-c3ccccc3)c2)ccc1N(c1ccc2c(c1)C(C)(C)c1ccccc1-2)c1ccc2c(c1)C(C)(C)c1ccccc1-2] 136 | 63.5 | 686.4 |
| 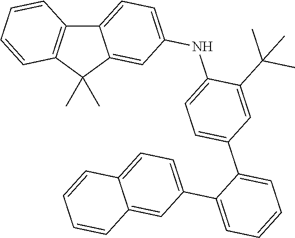 [SMILES: CC(C)(C)c1cc(-c2ccccc2-c2ccc3ccccc3c2)ccc1Nc1ccc2c(c1)C(C)(C)c1ccccc1-2] LM A8-1 | 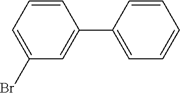 [SMILES: Brc1cccc(-c2ccccc2)c1] | 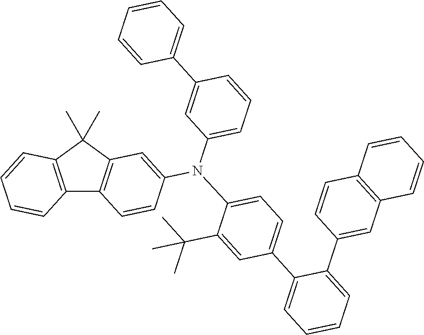 [SMILES: CC(C)(C)c1cc(-c2ccccc2-c2ccc3ccccc3c2)ccc1N(c1cccc(-c2ccccc2)c1)c1ccc2c(c1)C(C)(C)c1ccccc1-2] 135 | 59.1 | 696.4 |
| 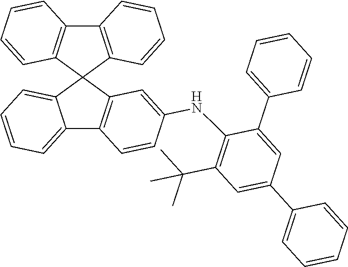 [SMILES: CC(C)(C)c1cc(-c2ccccc2)cc(-c2ccccc2)c1Nc1ccc2c(c1)C1(c3ccccc3-c3ccccc31)c1ccccc1-2] IM A18-1 | 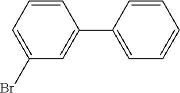 [SMILES: Brc1cccc(-c2ccccc2)c1] | 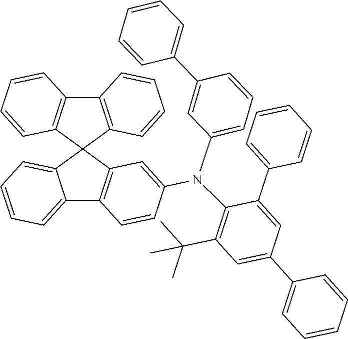 [SMILES: CC(C)(C)c1cc(-c2ccccc2)cc(-c2ccccc2)c1N(c1cccc(-c2ccccc2)c1)c1ccc2c(c1)C1(c3ccccc3-c3ccccc31)c1ccccc1-2] 42 | 65.1 | 768.4 |

NMR data of some Compounds are shown in Table 6 below.

TABLE 6

| Compound | NMR data |
|---|---|
| Compound 1 | ¹H-NMR (CD₂Cl₂, 400 Mhz): 8.24(d, 1H), 7.99(s, 1H), 7.97 − 7.90(m, 8H), 7.87 − 7.79(m, 7H), 7.53 − |
| | 7.46(m, 2H), 7.15(d, 2H), 7.08(d, 1H), 6.89(d, 1H), 6.67(s, 1H), 1.31(s, 6H), 1.01(s, 9H). |
| Compound 90 | ¹H-NMR (CD₂Cl₂, 400 Mhz): 8.29 − 8.22(m, 5H), 7.94(d, 1H), 7.99(s, 1H), 7.97 − 7.88(m, 12H), |
| | 7.53 − 7.46(m, 2H), 7.14(d, 2H), 7.08(d, 1H), 6.89(d, 1H), 6.67(s, 1H), 1.35(s, 6H), 1.11(s, 9H). |

### Fabrication and Performance Evaluation of Organic Electroluminescent Device

### Example 1

An ITO/Ag/ITO substrate with a thickness of 100 Å, 1000 Å, and 100 Å in an order was cut into a size of 40 mm (length)×40 mm (width)×0.7 mm (height), and prepared into a test substrate having cathode, anode, and insulation layer patterns with photolithography process. Ultraviolet ozone and O₂:N₂ plasma were used to perform surface treatment to remove surface floating debris and improve the anode work function of the substrate.

F4-TCNQ and HT-36 were co-vapor deposited on the test substrate (anode) at a vapor deposition rate ratio of 2%:98% to form a hole injection layer with a thickness of 100 Å.

HT-36 was vapor deposited on the hole injection layer to form a first hole transport layer with a thickness of 1024 Å.

The Compound 1 was vapor deposited on the first hole transport layer to form a second hole transport layer with a thickness of 720 Å.

RH and Ir(dmpq)₃acac were co-vapor deposited on the second hole transport layer at a vapor deposition rate ratio of 97:3 to form an organic light-emitting layer with a thickness of 400 Å.

ET-9 and LiQ were co-vapor deposited on the organic emitting-light layer at a vapor deposition rate ratio of 1:1 to form an electron transport layer with a thickness of 310 Å. Yb was vapor deposited on the electron transport layer to form an electron injection layer with a thickness of 10 Å. Then, magnesium (Mg) and silver (Ag) were co-vapor deposited on the electron injection layer at a vapor deposition rate ratio of 1:9 to form a cathode with a thickness of 120 Å.

Finally, Compound CP-1 was vapor deposited on the cathode to form an organic capling layer (CPL) with a thickness of 680 Å, thus completing the fabrication of the organic electroluminescent device.

### Examples 2 to 30

Organic electroluminescent devices were fabricated using the same method as Example 1, except that Compound 1 was replaced with the compound shown in Table 7 when forming the second hole transport layer. 5 Comparative Examples 1 to 6

Organic electroluminescent devices were fabricated using the same method as Example 1, except that Compound 1 was replaced with the compounds A, B, C, D, E, or F, when forming the second hole transport layer.

The main material structures used in the above Examples and Comparative Examples are shown in Table 7 below.

TABLE 7 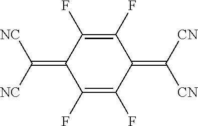 [SMILES: N#CC(C#N)=c1c(F)c(F)c(=C(C#N)C#N)c(F)c1F]
F4-TCNQ 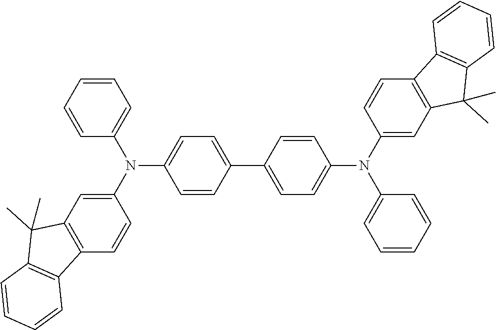 [SMILES: CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21]
HT-36 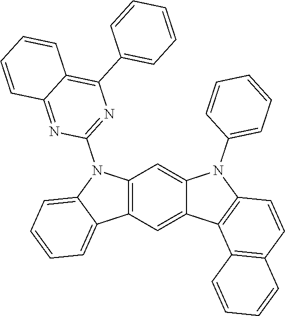 [SMILES: c1ccc(-c2nc(-n3c4ccccc4c4cc5c6c7ccccc7ccc6n(-c6ccccc6)c5cc43)nc3ccccc23)cc1]
RH 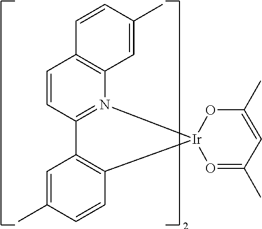
Ir(dmpq)₃acac 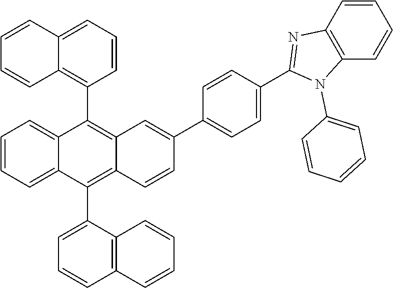 [SMILES: c1ccc(-n2c(-c3ccc(-c4ccc5c(-c6cccc7ccccc67)c6ccccc6c(-c6cccc7ccccc67)c5c4)cc3)nc3ccccc32)cc1]
ET-9 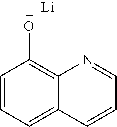 [SMILES: [Li+] ; Oc1cccc2cccnc12]
LiQ 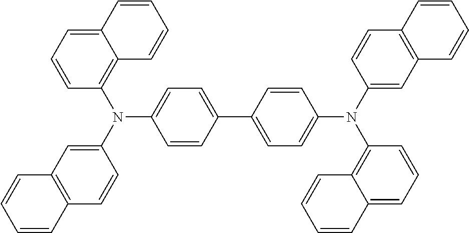 [SMILES: c1ccc2cc(N(c3ccc(-c4ccc(N(c5ccc6ccccc6c5)c5cccc6ccccc56)cc4)cc3)c3cccc4ccccc34)ccc2c1]
CP-1 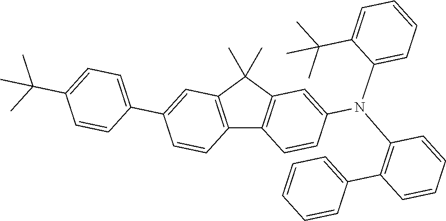 [SMILES: CC(C)(C)c1ccc(-c2ccc3c(c2)C(C)(C)c2cc(N(c4ccccc4-c4ccccc4)c4ccccc4C(C)(C)C)ccc2-3)cc1]
Compound A 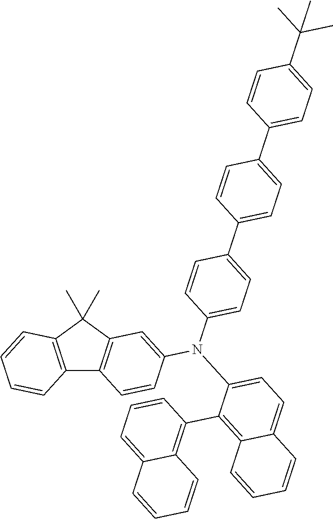 [SMILES: CC(C)(C)c1ccc(-c2ccc(-c3ccc(N(c4ccc5c(c4)C(C)(C)c4ccccc4-5)c4ccc5ccccc5c4-c4cccc5ccccc45)cc3)cc2)cc1]
Compound B 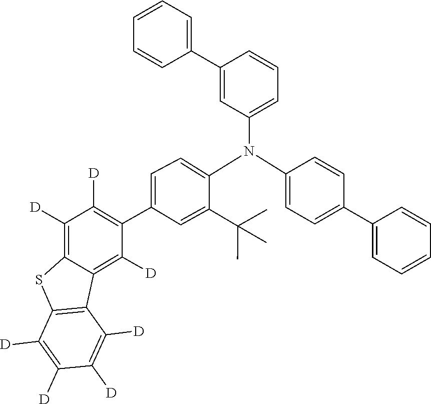 [SMILES: [2H]c1c([2H])c([2H])c2c(sc3c([2H])c([2H])c(-c4ccc(N(c5ccc(-c6ccccc6)cc5)c5cccc(-c6ccccc6)c5)c(C(C)(C)C)c4)c([2H])c32)c1[2H]]
Compound C 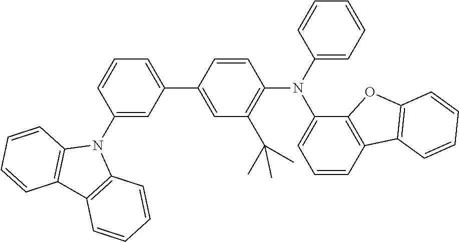 [SMILES: CC(C)(C)c1cc(-c2cccc(-n3c4ccccc4c4ccccc43)c2)ccc1N(c1ccccc1)c1cccc2c1oc1ccccc12]
Compound D 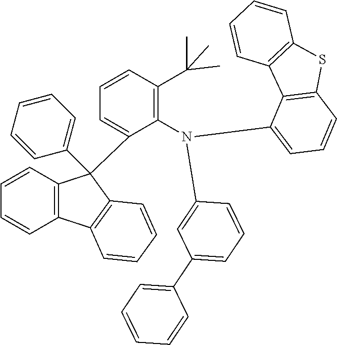 [SMILES: CC(C)(C)c1cccc(C2(c3ccccc3)c3ccccc3-c3ccccc32)c1N(c1cccc(-c2ccccc2)c1)c1cccc2sc3ccccc3c12]
Compound E 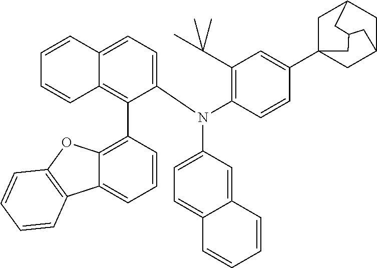 [SMILES: CC(C)(C)c1cc(C23CC4CC(CC(C4)C2)C3)ccc1N(c1ccc2ccccc2c1)c1ccc2ccccc2c1-c1cccc2c1oc1ccccc12]
Compound F

Performance tests were conducted on the devices fabricated in the Examples and Comparative Examples, in which IVL (driving voltage, current efficiency, color coordinates) data were tested at a current density of 10 mA/cm², and T95 lifetime was tested at a current density of 20 mA/cm². The results are shown in Table 8.

TABLE 8

| | | | | Color | |
|---|---|---|---|---|---|
| | | | | Coor- | |
| | Second Hole | Driving | Current | dinates | T95 |
| Example | Transport | Voltage | Efficiency | CIEx, | Lifetime |
| No. | Layer | (V) | (Cd/A) | CIEy | (h) |
| Example 1 | Compound 1 | 3.76 | 62.31 | 0.68, 0.32 | 488 |
| Example 2 | Compound 2 | 3.80 | 62.62 | 0.68, 0.32 | 496 |
| Example 3 | Compound 4 | 3.81 | 62.54 | 0.68, 0.32 | 494 |
| Example 4 | Compound 7 | 4.05 | 58.63 | 0.68, 0.32 | 457 |
| Example 5 | Compound 8 | 4.07 | 58.49 | 0.68, 0.32 | 451 |
| Example 6 | Compound | 3.68 | 62.03 | 0.68, 0.32 | 483 |
| | 16 | | | | |
| Example 7 | Compound | 3.79 | 62.34 | 0.68, 0.32 | 490 |
| | 18 | | | | |
| Example 8 | Compound | 3.71 | 63.75 | 0.68, 0.32 | 500 |
| | 22 | | | | |
| Example 9 | Compound | 3.66 | 63.14 | 0.68, 0.32 | 497 |
| | 31 | | | | |
| Example 10 | Compound | 3.64 | 63.05 | 0.68, 0.32 | 495 |
| | 38 | | | | |
| Example 11 | Compound | 3.80 | 62.56 | 0.68, 0.32 | 493 |
| | 209 | | | | |
| Example 12 | Compound | 3.67 | 62.07 | 0.68, 0.32 | 484 |
| | 47 | | | | |
| Example 13 | Compound | 3.69 | 62.01 | 0.68, 0.32 | 481 |
| | 72 | | | | |
| Example 14 | Compound | 3.72 | 63.24 | 0.68, 0.32 | 499 |
| | 24 | | | | |
| Example 15 | Compound | 3.63 | 62.65 | 0.68, 0.32 | 486 |
| | 10 | | | | |
| Example 16 | Compound | 4.08 | 58.57 | 0.68, 0.32 | 456 |
| | 155 | | | | |
| Example 17 | Compound | 4.09 | 58.51 | 0.68, 0.32 | 454 |
| | 170 | | | | |
| Example 18 | Compound | 3.74 | 61.91 | 0.68, 0.32 | 480 |
| | 42 | | | | |
| Example 19 | Compound | 3.78 | 62.87 | 0.68, 0.32 | 492 |
| | 134 | | | | |
| Example 20 | Compound | 3.73 | 62.13 | 0.68, 0.32 | 485 |
| | 59 | | | | |
| Example 21 | Compound 6 | 3.62 | 63.66 | 0.68, 0.32 | 504 |
| Example 22 | Compound | 3.77 | 63.57 | 0.68, 0.32 | 501 |
| | 84 | | | | |
| Example 23 | Compound | 3.86 | 63.78 | 0.68, 0.32 | 506 |
| | 86 | | | | |
| Example 24 | Compound | 3.65 | 62.94 | 0.68, 0.32 | 498 |
| | 90 | | | | |
| Example 25 | Compound | 3.85 | 63.17 | 0.68, 0.32 | 502 |
| | 92 | | | | |
| Example 26 | Compound | 3.75 | 63.59 | 0.68, 0.32 | 505 |
| | 103 | | | | |
| Example 27 | Compound | 3.83 | 62.27 | 0.68, 0.32 | 489 |
| | 210 | | | | |
| Example 28 | Compound | 3.84 | 63.88 | 0.68, 0.32 | 503 |
| | 136 | | | | |
| Example 29 | Compound | 3.82 | 62.76 | 0.68, 0.32 | 491 |
| | 135 | | | | |
| Example 30 | Compound | 3.61 | 61.97 | 0.68, 0.32 | 482 |
| | 42 | | | | |
| Compar- | Compound | 4.21 | 50.63 | 0.68, 0.32 | 384 |
| ative | A | | | | |
| Example 1 | | | | | |
| Compar- | Compound | 4.24 | 50.84 | 0.68, 0.32 | 380 |
| ative | B | | | | |
| Example 2 | | | | | |
| Compar- | Compound | 4.19 | 52.43 | 0.68, 0.32 | 391 |
| ative | C | | | | |
| Example 3 | | | | | |
| Compar- | Compound | 4.11 | 52.71 | 0.68, 0.33 | 409 |
| ative | D | | | | |
| Example 4 | | | | | |
| Compar- | Compound | 4.17 | 52.59 | 0.68, 0.34 | 398 |
| ative | E | | | | |
| Example 5 | | | | | |
| Compar- | Compound | 4.15 | 52.66 | 0.68, 0.35 | 402 |
| ative | F | | | | |
| Example 6 | | | | | |

According to the results in Table 8, it can be seen that Examples 1 to 30 of compounds as the second hole transport layer have improved the current efficiency and lifetime of the organic electroluminescent devices respectively by at least 11% and at least 11% compared to Comparative Examples 1 to 6 of devices corresponding to known compounds.

The preferred embodiments of the present disclosure are described in detail above in conjunction with the accompanying drawings. However, the present disclosure is not limited to the specific details of the above embodiments. Within the scope of the technical concept of the present disclosure, various simple modifications can be made to the technical solutions of the present disclosure, and all of these simple modifications fall within the protection scope of the present disclosure.

1. An organic compound, having a structure shown in a Formula I: 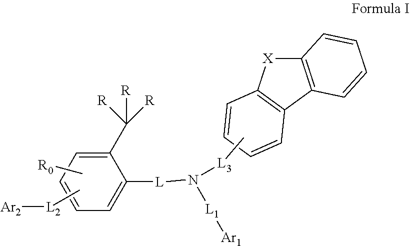
wherein X is selected from O, S, C(R₁R₂), or N(R₃);
R₁, R₂, and R₃ are identical or different, and are each independently selected from an alkyl having 1 to 5 carbon atoms, a deuteroalkyl having 1 to 5 carbon atoms, a deuteroaryl having 6 to carbon atoms, an aryl having 6 to 20 carbon atoms, or a heteroaryl having 3 to 20 carbon atoms; or R₁ and R₂ are interconnected together with the atoms to which they are commonly connected to form a 5- to 13-membered saturated or unsaturated ring;
each R is independently selected from a deuterium, an alkyl having 1 to 5 carbon atoms, or a deuteroalkyl having 1 to 5 carbon atoms;
R₀ is selected from a hydrogen, an alkyl having 1 to 5 carbon atoms, a deuteroalkyl having 1 to 5 carbon atoms, a haloalkyl having 1 to 5 carbon atoms, a deuteroaryl having 6 to 18 carbon atoms, an aryl having 6 to 18 carbon atoms, or a heteroaryl having 3 to 18 carbon atoms;
L is selected from a single bond, a substituted or unsubstituted arylene having 6 to 18 carbon atoms, or a substituted or unsubstituted heteroarylene having 3 to 18 carbon atoms;
the substituent(s) of L are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 10 carbon atoms, an aryl having 6 to 12 carbon atoms, or a heteroaryl having 3 to 12 carbon atoms;
Ar₂ is selected from a substituted or unsubstituted aryl having 6 to 18 carbon atoms, a substituted or unsubstituted dibenzofuranyl, or a substituted or unsubstituted dibenzothienyl;
the substituent(s) of Ar₂ are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 10 carbon atoms, an aryl having 6 to 12 carbon atoms, a dibenzofuranyl, or a dibenzothienyl;
L₂ is selected from a single bond, a substituted or unsubstituted arylene having 6 to 12 carbon atoms, or a substituted or unsubstituted heteroarylene having 3 to 18 carbon atoms;
the substituent(s) of L₂ are each independently selected from a deuterium, a halogen group, a cyano, an alkyl having 1 to 5 carbon atoms, a deuteroalkyl having 1 to 5 carbon atoms, a trialkylsilyl having 3 to 12 carbon atoms, a haloalkyl having 1 to 5 carbon atoms, a cycloalkyl having 3 to 6 carbon atoms, or a phenyl;
Ar₁ is selected from a substituted or unsubstituted aryl having 6 to 30 carbon atoms, or a substituted or unsubstituted heteroaryl having 3 to 30 carbon atoms;
the substituent(s) of Ar₁ are each independently selected from a deuterium, a halogen group, a cyano, an alkyl having 1 to 10 carbon atoms, a deuteroalkyl having 1 to 10 carbon atoms, a trialkylsilyl having 3 to 12 carbon atoms, a haloalkyl having 1 to 10 carbon atoms, a cycloalkyl having 3 to 12 carbon atoms, a deuteroaryl having 6 to 20 carbon atoms, an aryl having 6 to 20 carbon atoms, or a heteroaryl having 3 to 20 carbon atoms; optionally, any two adjacent substituents of Ar₁ form a saturated or unsaturated 3- to 15-membered ring;
L₁ and L₃ are identical or different, and are each independently selected from a single bond, a substituted or unsubstituted arylene having 6 to 18 carbon atoms, or a substituted or unsubstituted heteroarylene having 3 to 18 carbon atoms;
the substituent(s) of L₁ and L₃ are each independently selected from a deuterium, a halogen group, a cyano, a trialkylsilyl having 3 to 12 carbon atoms, a cycloalkyl having 3 to 10 carbon atoms, an aryl having 6 to 12 carbon atoms, or a heteroaryl having 3 to 12 carbon atoms.

2. The organic compound according to claim 1, wherein L is selected from a single bond, a substituted or unsubstituted phenylene, a substituted or unsubstituted naphthylene, a substituted or unsubstituted biphenylene, a substituted or unsubstituted fluorenylene, a substituted or unsubstituted dibenzofuranylene, a substituted or unsubstituted dibenzothienylene, or a substituted or unsubstituted carbazolylene;
optionally, the substituent(s) of L are each independently selected from a deuterium, a fluorine, a cyano, a methyl, an ethyl, an isopropyl, a tert-butyl, a trideuteromethyl, a trimethylsilyl, a trifluoromethyl, a cyclopentyl, a cyclohexyl, a phenyl, a naphthyl, a biphenyl, a dibenzofuranyl, a dibenzothienyl, or a carbazolyl.

3. The organic compound according to claim 1, wherein L₂ is selected from a single bond, a substituted or unsubstituted phenylene, a substituted or unsubstituted naphthylene, a biphenylene, a substituted or unsubstituted dibenzofuranylene, a substituted or unsubstituted dibenzothienylene, or a substituted or unsubstituted carbazolylene;
optionally, the substituent(s) of L₂ are each independently selected from a deuterium, a fluorine, a cyano, a methyl, an ethyl, an isopropyl, a tert-butyl, a trideuteromethyl, a trimethylsilyl, a trifluoromethyl, a cyclopentane, a cyclohexane, or a phenyl.

4. The organic compound according to claim 1, wherein L₁ and L₃ are each independently selected from a single bond, a substituted or unsubstituted phenylene, a substituted or unsubstituted naphthylene, a substituted or unsubstituted biphenylene, a terphenylene, a substituted or unsubstituted fluorenylene, a substituted or unsubstituted dibenzofuranylene, a substituted or unsubstituted dibenzothienylene, or a substituted or unsubstituted carbazolylene;
the substituent(s) of L₁ and L₃ are each independently selected from a deuterium, a fluorine, a cyano, a trimethylsilyl, a cyclopentyl, a cyclohexyl, a phenyl, a naphthyl, a biphenyl, a dibenzofuranyl, a dibenzothienyl, or a carbazolyl.

5. The organic compound according to claim 1, wherein Ar₁ is selected from a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted spirodifluorenyl, a substituted or unsubstituted phenanthryl, a substituted or unsubstituted dibenzofuranyl, a substituted or unsubstituted dibenzothienyl, or a substituted or unsubstituted carbazolyl.
optionally, the substituent(s) of Ar₁ are each independently selected from a deuterium, a fluorine, a cyano, a methyl, an ethyl, an isopropyl, a tert-butyl, a trimethylsilyl, a trifluoromethyl, a trideuteromethyl, a cyclopentyl, a cyclohexyl, a pentadeuterophenyl, a phenyl, a naphthyl, a biphenyl, a dibenzofuranyl, a dibenzothienyl, or a carbazolyl; optionally, any two adjacent substituents of Ar₁ form a cyclopentane, cyclohexane or a fluorene ring.

6. The organic compound according to claim 1, wherein Ar₁ is selected from the following groups:

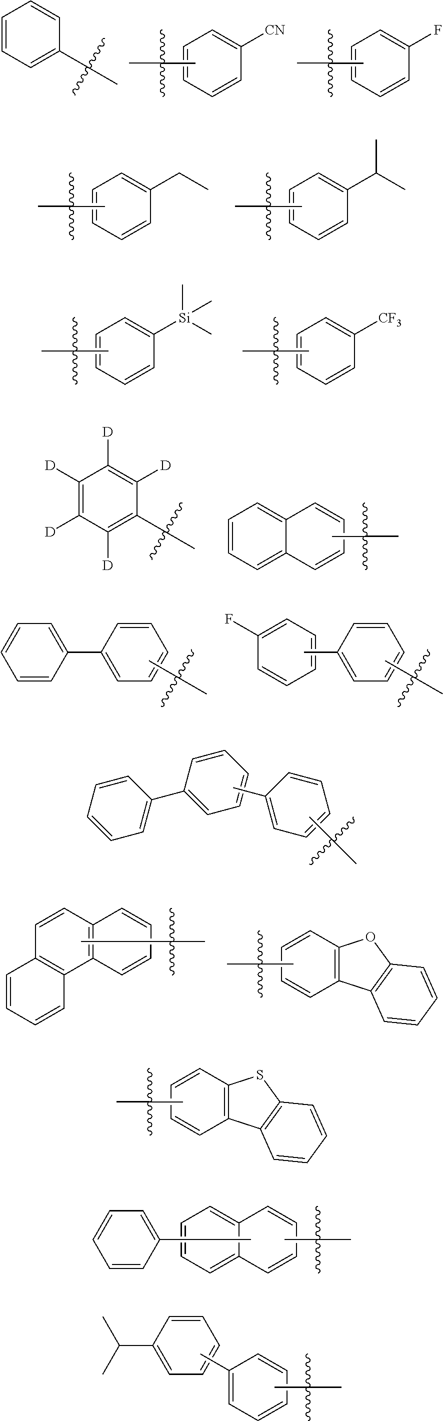

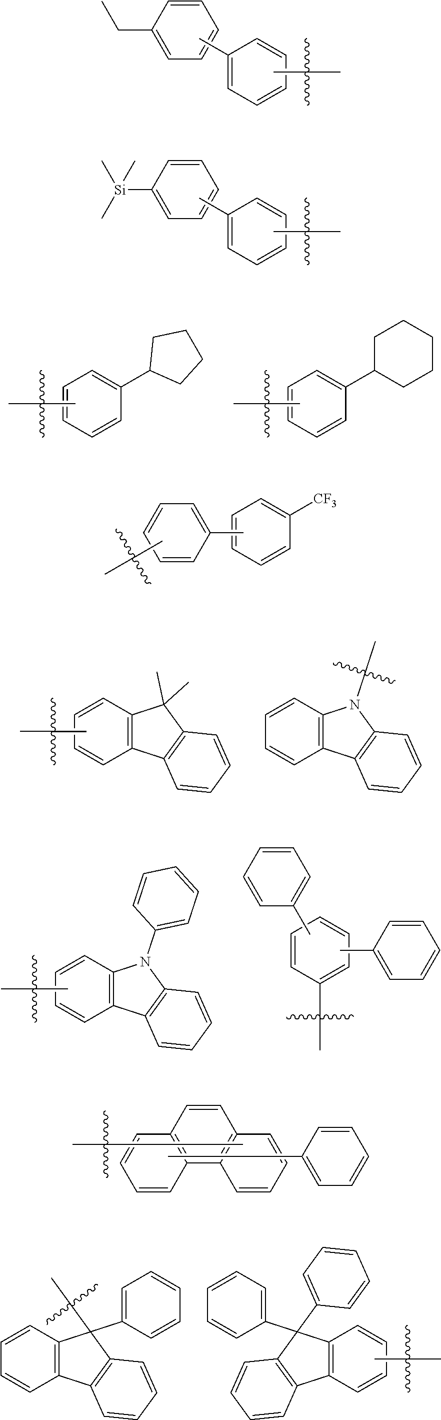

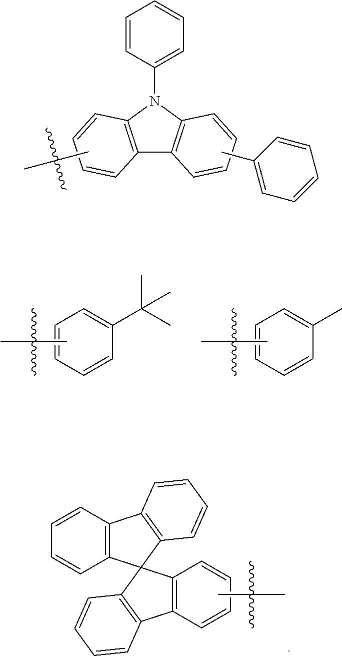

7. The organic compound according to claim 1, wherein Ar₂ is selected from a substituted or unsubstituted group Q, and the unsubstituted group Q is selected from the group consisting of the following groups: 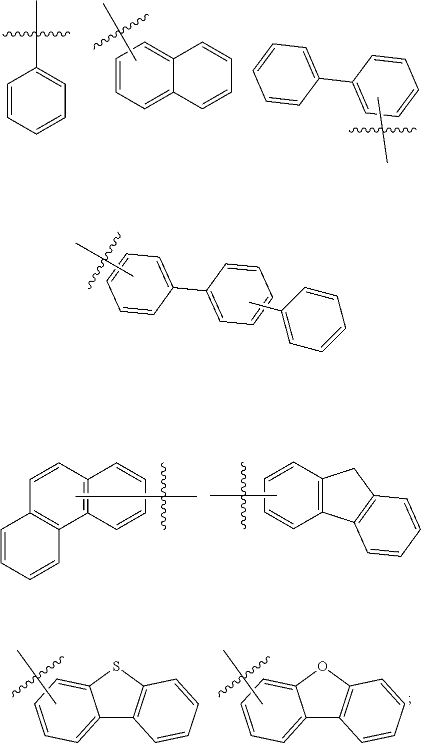
the substituted group Q has one or more substituent(s), and each substituent is independently selected from a deuterium, a fluorine, a cyano, a methyl, an ethyl, an isopropyl, a tert-butyl, a trideuteromethyl, a trimethylsilyl, a trifluoromethyl, a cyclopentyl, a cyclohexyl, a phenyl, a naphthyl, a biphenyl, a dibenzofuranyl, or a dibenzothienyl, and when the number of the substituents of the group Q is greater than 1, the substituents are identical or different.

8. The organic compound according to claim 1, wherein 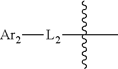
in Formula I is selected from the following groups:

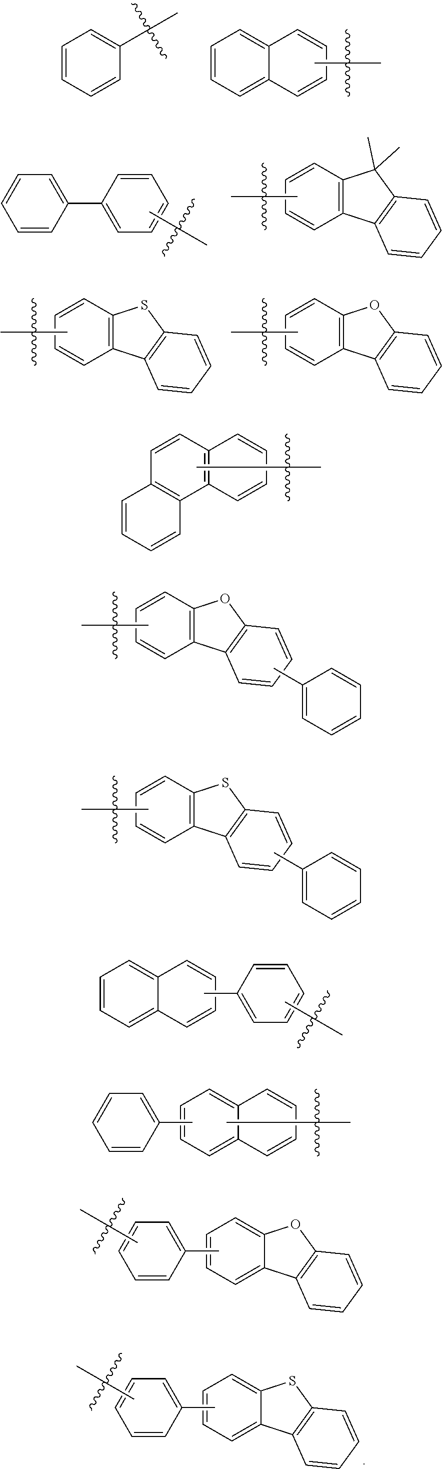

9. The organic compound according to claim 1, wherein R₁, R₂, and R₃ are each independently selected from a methyl, a trideuteromethyl, an ethyl, an isopropyl, a tert-butyl, a phenyl, a pentadeuterophenyl, a naphthyl, a biphenyl, a pyridyl, a dibenzofuranyl, a dibenzothienyl or a carbazolyl; or R₁ and R₂ are interconnected together with the atoms to which they are commonly connected to form a cyclopentane, a cyclohexane, an adamantane, or a fluorene ring.

10. The organic compound according to claim 1, wherein each R is independently selected from a deuterium, a methyl, an ethyl, an isopropyl, a tert-butyl, or a trideuteromethyl.

11. The organic compound according to claim 1, wherein R₀ is selected from a hydrogen, a methyl, an ethyl, an isopropyl, a tert-butyl, a trideuteromethyl, a trifluoromethyl, a pentadeuterophenyl, a phenyl, a naphthyl, a biphenyl, a dibenzofuranyl, a dibenzothienyl, or a carbazolyl.

12. The organic compound according to claim 1, wherein the organic compound is selected from the group consisting of the following compounds:

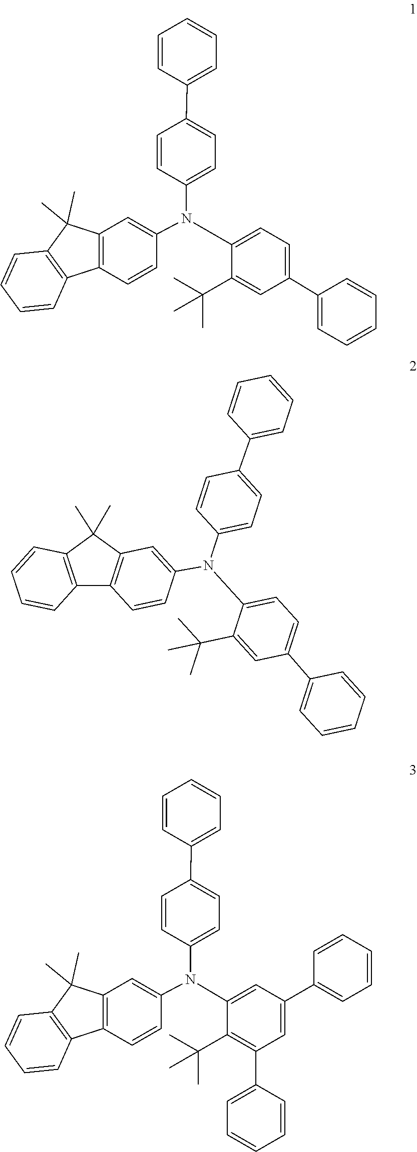 [SMILES: CC(C)(C)c1cc(-c2ccccc2)ccc1N(c1ccc(-c2ccccc2)cc1)c1ccc2c(c1)C(C)(C)c1ccccc1-2 ; CC(C)(C)c1cc(-c2ccccc2)ccc1N(c1ccc(-c2ccccc2)cc1)c1ccc2c(c1)C(C)(C)c1ccccc1-2 ; CC(C)(C)c1c(-c2ccccc2)cc(-c2ccccc2)cc1N(c1ccc(-c2ccccc2)cc1)c1ccc2c(c1)C(C)(C)c1ccccc1-2]

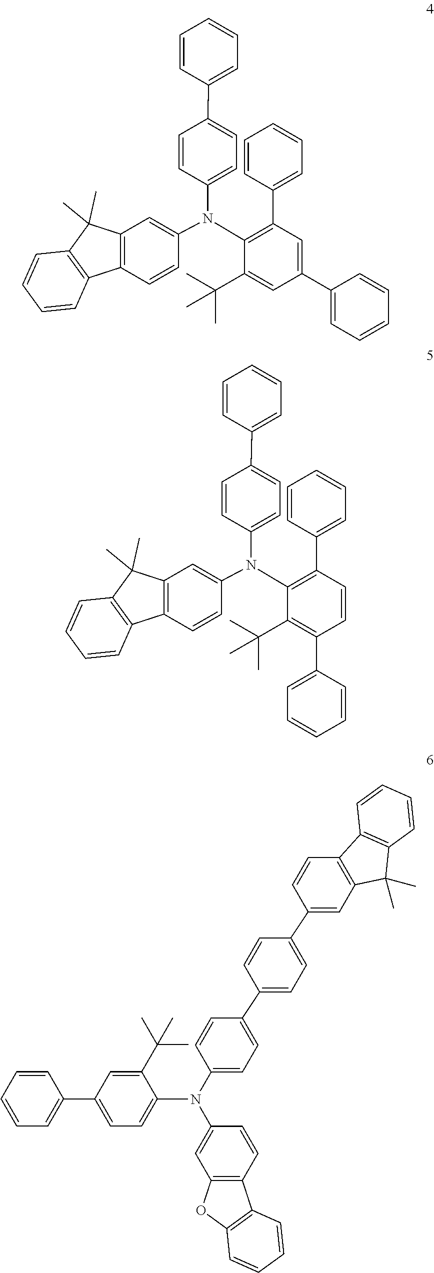 [SMILES: CC(C)(C)c1cc(-c2ccccc2)cc(-c2ccccc2)c1N(c1ccc(-c2ccccc2)cc1)c1ccc2c(c1)C(C)(C)c1ccccc1-2 ; CC(C)(C)c1c(-c2ccccc2)ccc(-c2ccccc2)c1N(c1ccc(-c2ccccc2)cc1)c1ccc2c(c1)C(C)(C)c1ccccc1-2 ; CC(C)(C)c1cc(-c2ccccc2)ccc1N(c1ccc(-c2ccc(-c3ccc4c(c3)C(C)(C)c3ccccc3-4)cc2)cc1)c1ccc2c(c1)oc1ccccc12]

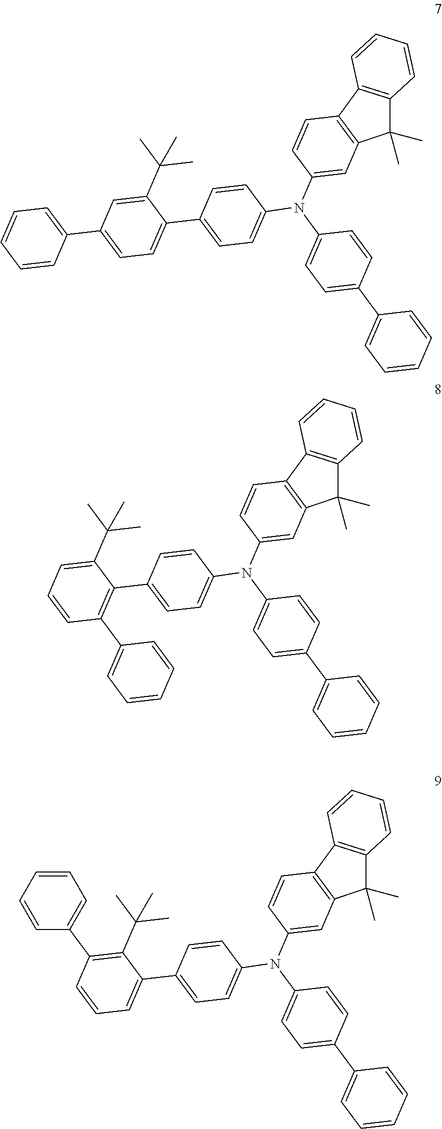 [SMILES: CC(C)(C)c1cc(-c2ccccc2)ccc1-c1ccc(N(c2ccc(-c3ccccc3)cc2)c2ccc3c(c2)C(C)(C)c2ccccc2-3)cc1 ; CC(C)(C)c1cccc(-c2ccccc2)c1-c1ccc(N(c2ccc(-c3ccccc3)cc2)c2ccc3c(c2)C(C)(C)c2ccccc2-3)cc1 ; CC(C)(C)c1c(-c2ccccc2)cccc1-c1ccc(N(c2ccc(-c3ccccc3)cc2)c2ccc3c(c2)C(C)(C)c2ccccc2-3)cc1]

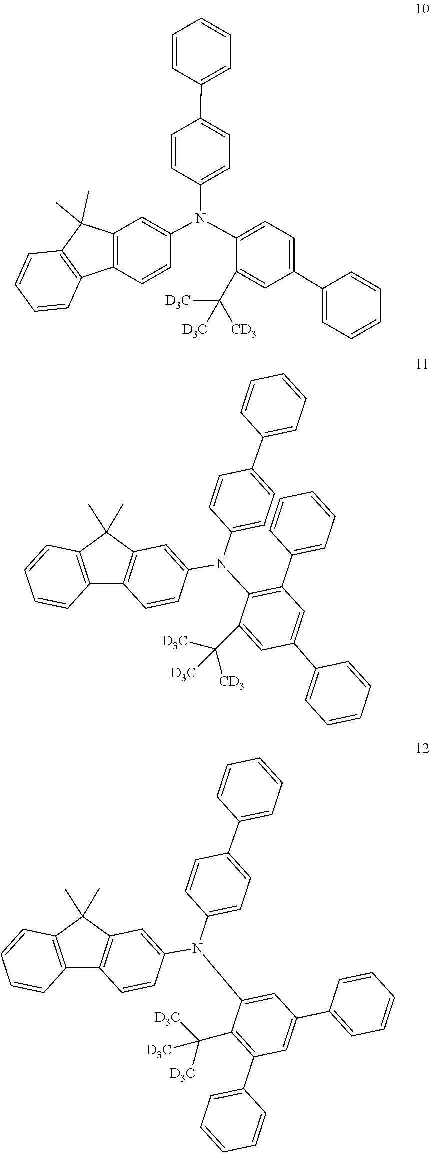 [SMILES: [2H]C([2H])([2H])C(c1cc(-c2ccccc2)ccc1N(c1ccc(-c2ccccc2)cc1)c1ccc2c(c1)C(C)(C)c1ccccc1-2)(C([2H])([2H])[2H])C([2H])([2H])[2H] ; [2H]C([2H])([2H])C(c1cc(-c2ccccc2)cc(-c2ccccc2)c1N(c1ccc(-c2ccccc2)cc1)c1ccc2c(c1)C(C)(C)c1ccccc1-2)(C([2H])([2H])[2H])C([2H])([2H])[2H] ; [2H]C([2H])([2H])C(c1c(-c2ccccc2)cc(-c2ccccc2)cc1N(c1ccc(-c2ccccc2)cc1)c1ccc2c(c1)C(C)(C)c1ccccc1-2)(C([2H])([2H])[2H])C([2H])([2H])[2H]]

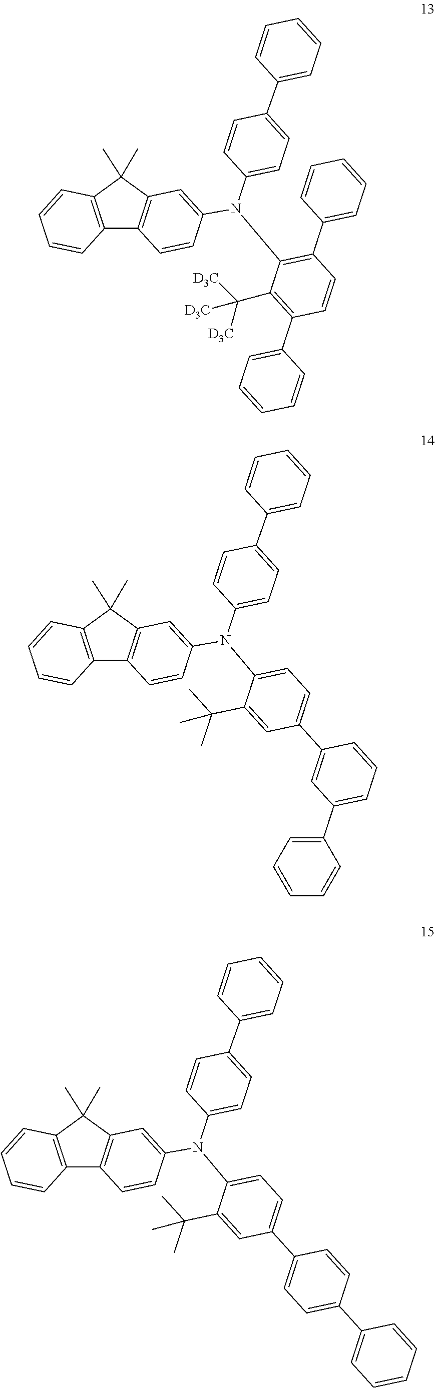

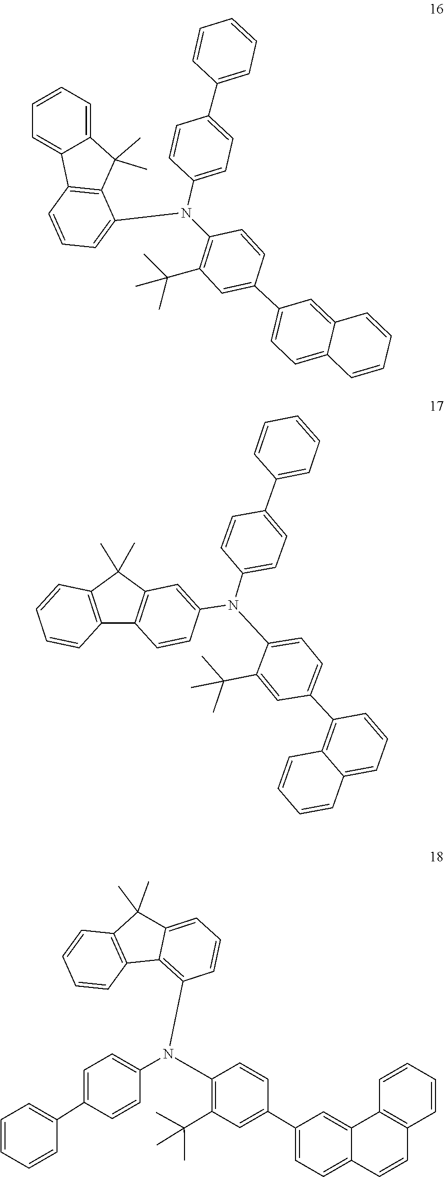 [SMILES: CC(C)(C)c1cc(-c2ccc3ccccc3c2)ccc1N(c1ccc(-c2ccccc2)cc1)c1cccc2c1C(C)(C)c1ccccc1-2 ; CC(C)(C)c1cc(-c2cccc3ccccc23)ccc1N(c1ccc(-c2ccccc2)cc1)c1ccc2c(c1)C(C)(C)c1ccccc1-2 ; CC(C)(C)c1cc(-c2ccc3ccc4ccccc4c3c2)ccc1N(c1ccc(-c2ccccc2)cc1)c1cccc2c1-c1ccccc1C2(C)C]

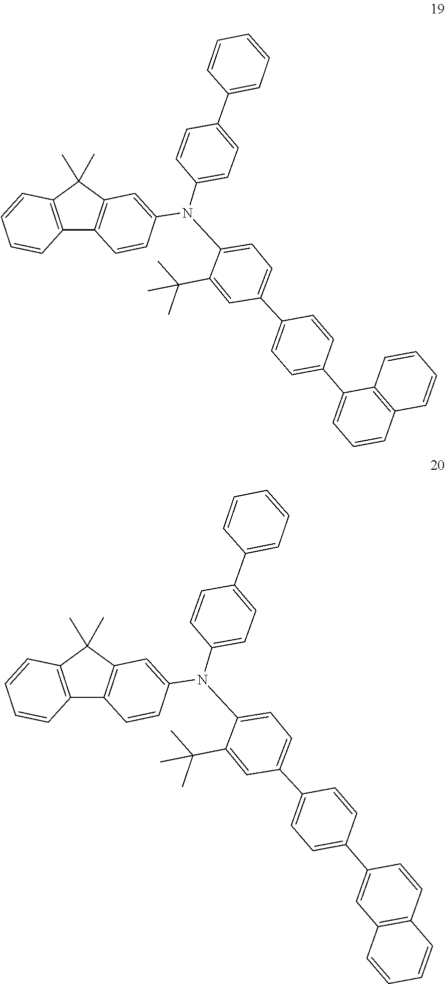 [SMILES: CC(C)(C)c1cc(-c2ccc(-c3cccc4ccccc34)cc2)ccc1N(c1ccc(-c2ccccc2)cc1)c1ccc2c(c1)C(C)(C)c1ccccc1-2 ; CC(C)(C)c1cc(-c2ccc(-c3ccc4ccccc4c3)cc2)ccc1N(c1ccc(-c2ccccc2)cc1)c1ccc2c(c1)C(C)(C)c1ccccc1-2]

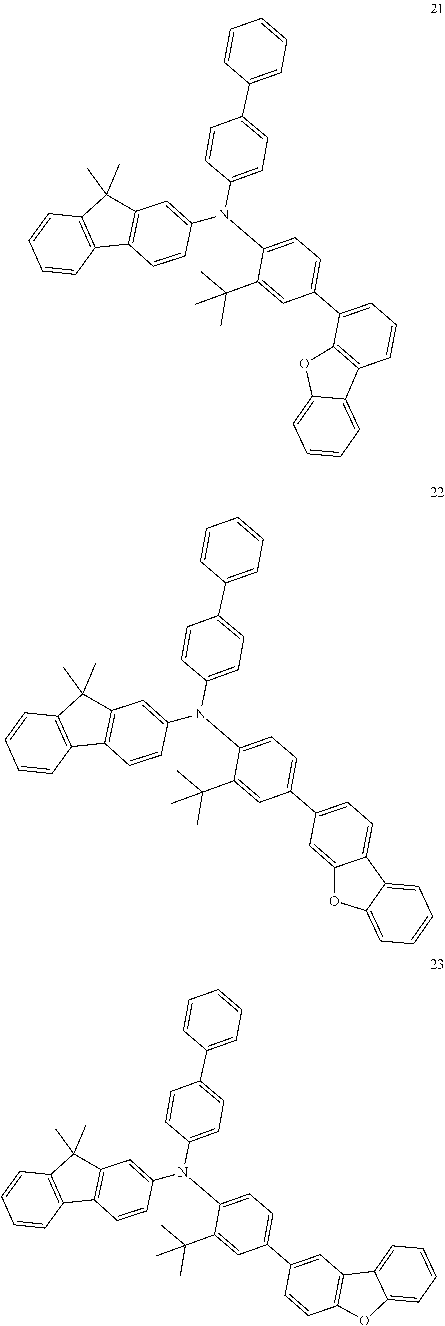 [SMILES: CC(C)(C)c1cc(-c2cccc3c2oc2ccccc23)ccc1N(c1ccc(-c2ccccc2)cc1)c1ccc2c(c1)C(C)(C)c1ccccc1-2 ; CC(C)(C)c1cc(-c2ccc3c(c2)oc2ccccc23)ccc1N(c1ccc(-c2ccccc2)cc1)c1ccc2c(c1)C(C)(C)c1ccccc1-2 ; CC(C)(C)c1cc(-c2ccc3oc4ccccc4c3c2)ccc1N(c1ccc(-c2ccccc2)cc1)c1ccc2c(c1)C(C)(C)c1ccccc1-2]

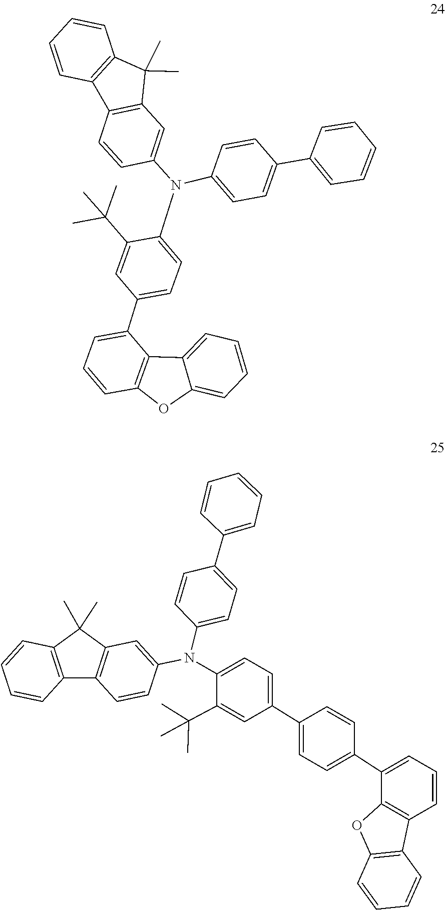 [SMILES: CC(C)(C)c1cc(-c2cccc3oc4ccccc4c23)ccc1N(c1ccc(-c2ccccc2)cc1)c1ccc2c(c1)C(C)(C)c1ccccc1-2 ; CC(C)(C)c1cc(-c2ccc(-c3cccc4c3oc3ccccc34)cc2)ccc1N(c1ccc(-c2ccccc2)cc1)c1ccc2c(c1)C(C)(C)c1ccccc1-2]

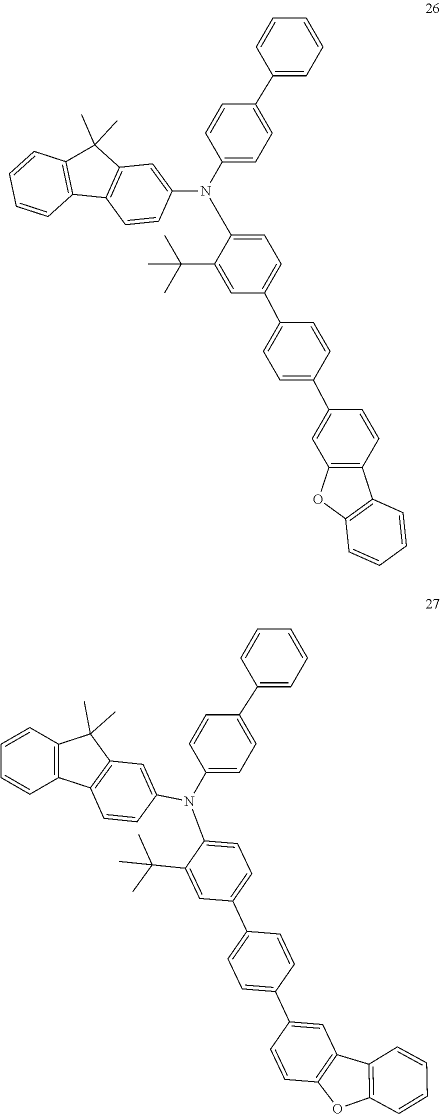 [SMILES: CC(C)(C)c1cc(-c2ccc(-c3ccc4c(c3)oc3ccccc34)cc2)ccc1N(c1ccc(-c2ccccc2)cc1)c1ccc2c(c1)C(C)(C)c1ccccc1-2 ; CC(C)(C)c1cc(-c2ccc(-c3ccc4oc5ccccc5c4c3)cc2)ccc1N(c1ccc(-c2ccccc2)cc1)c1ccc2c(c1)C(C)(C)c1ccccc1-2]

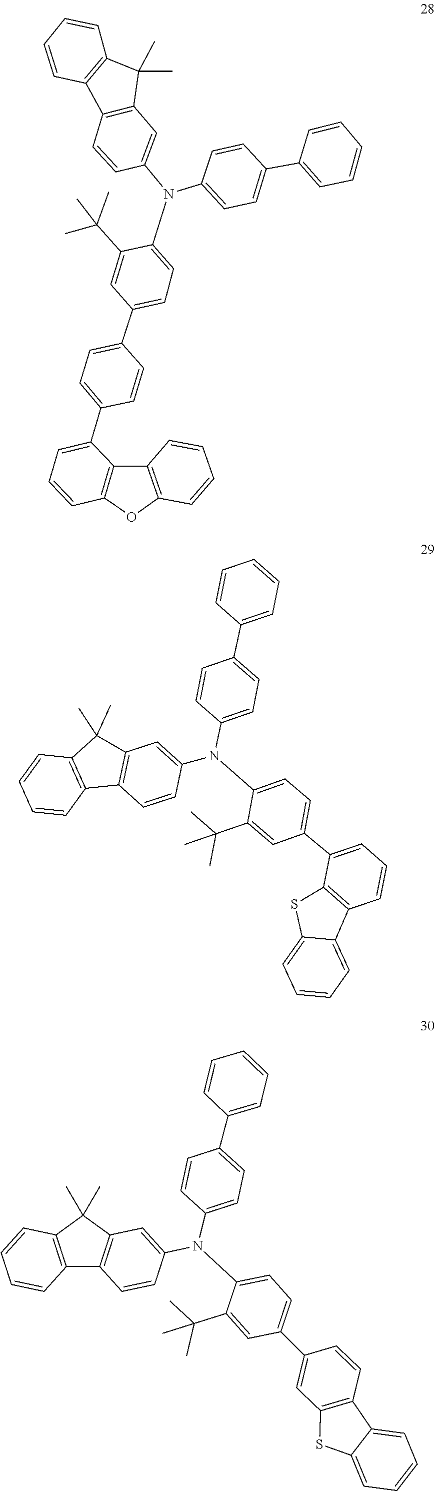 [SMILES: CC(C)(C)c1cc(-c2ccc(-c3cccc4oc5ccccc5c34)cc2)ccc1N(c1ccc(-c2ccccc2)cc1)c1ccc2c(c1)C(C)(C)c1ccccc1-2 ; CC(C)(C)c1cc(-c2cccc3c2sc2ccccc23)ccc1N(c1ccc(-c2ccccc2)cc1)c1ccc2c(c1)C(C)(C)c1ccccc1-2 ; CC(C)(C)c1cc(-c2ccc3c(c2)sc2ccccc23)ccc1N(c1ccc(-c2ccccc2)cc1)c1ccc2c(c1)C(C)(C)c1ccccc1-2]

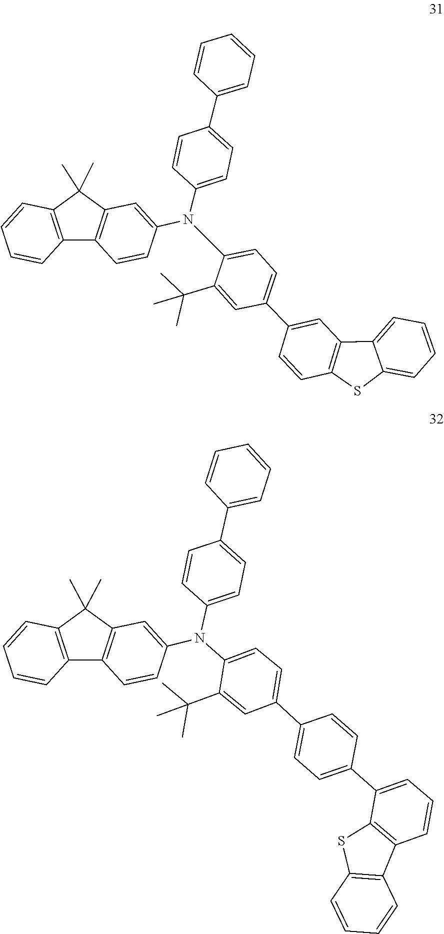 [SMILES: CC(C)(C)c1cc(-c2ccc3sc4ccccc4c3c2)ccc1N(c1ccc(-c2ccccc2)cc1)c1ccc2c(c1)C(C)(C)c1ccccc1-2 ; CC(C)(C)c1cc(-c2ccc(-c3cccc4c3sc3ccccc34)cc2)ccc1N(c1ccc(-c2ccccc2)cc1)c1ccc2c(c1)C(C)(C)c1ccccc1-2]

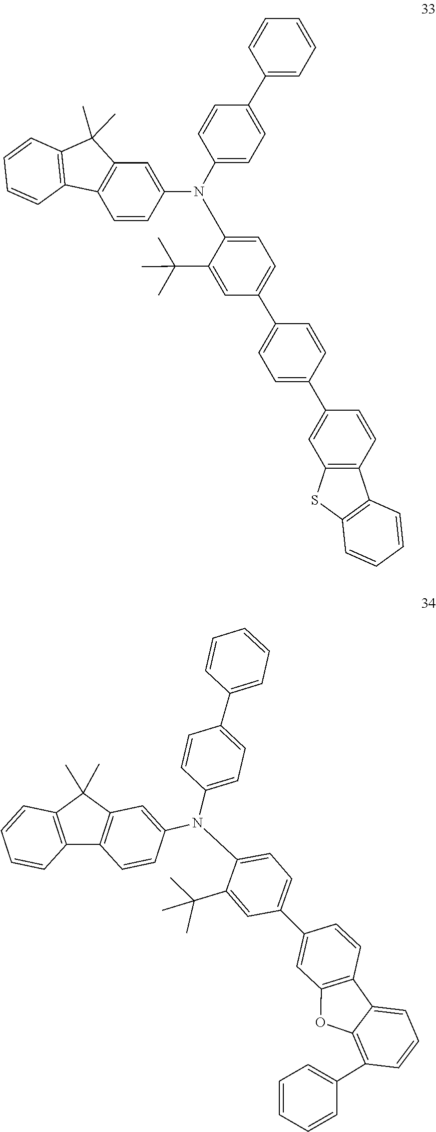 [SMILES: CC(C)(C)c1cc(-c2ccc(-c3ccc4c(c3)sc3ccccc34)cc2)ccc1N(c1ccc(-c2ccccc2)cc1)c1ccc2c(c1)C(C)(C)c1ccccc1-2 ; CC(C)(C)c1cc(-c2ccc3c(c2)oc2c(-c4ccccc4)cccc23)ccc1N(c1ccc(-c2ccccc2)cc1)c1ccc2c(c1)C(C)(C)c1ccccc1-2]

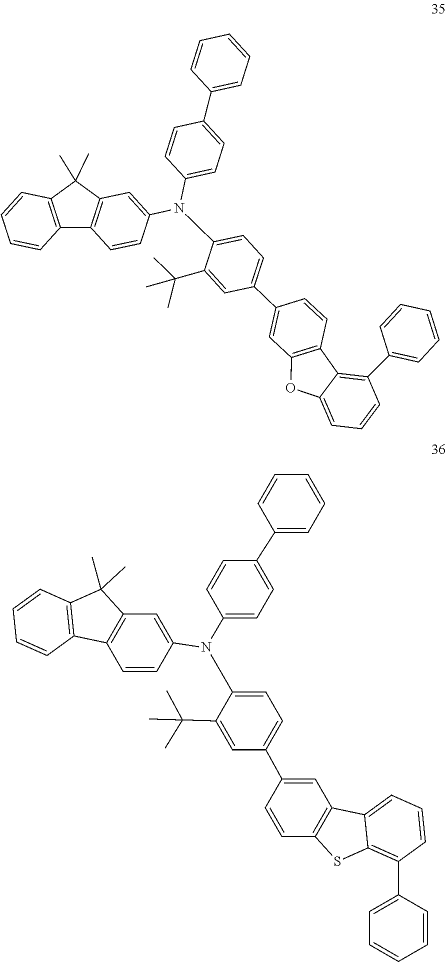 [SMILES: CC(C)(C)c1cc(-c2ccc3c(c2)oc2cccc(-c4ccccc4)c23)ccc1N(c1ccc(-c2ccccc2)cc1)c1ccc2c(c1)C(C)(C)c1ccccc1-2 ; CC(C)(C)c1cc(-c2ccc3sc4c(-c5ccccc5)cccc4c3c2)ccc1N(c1ccc(-c2ccccc2)cc1)c1ccc2c(c1)C(C)(C)c1ccccc1-2]

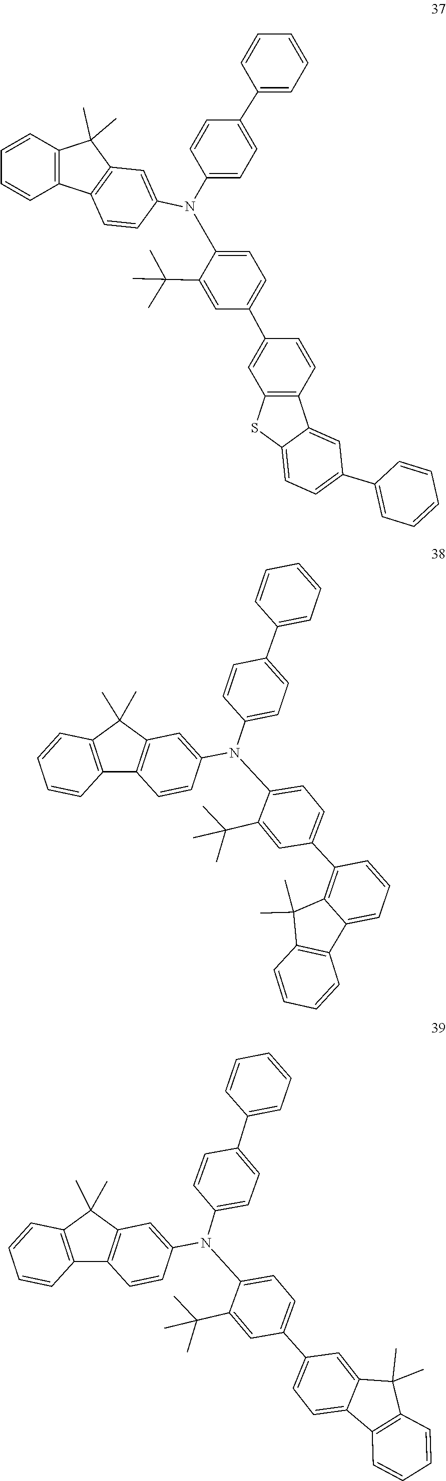 [SMILES: CC(C)(C)c1cc(-c2ccc3c(c2)sc2ccc(-c4ccccc4)cc23)ccc1N(c1ccc(-c2ccccc2)cc1)c1ccc2c(c1)C(C)(C)c1ccccc1-2 ; CC(C)(C)c1cc(-c2cccc3c2C(C)(C)c2ccccc2-3)ccc1N(c1ccc(-c2ccccc2)cc1)c1ccc2c(c1)C(C)(C)c1ccccc1-2 ; CC(C)(C)c1cc(-c2ccc3c(c2)C(C)(C)c2ccccc2-3)ccc1N(c1ccc(-c2ccccc2)cc1)c1ccc2c(c1)C(C)(C)c1ccccc1-2]

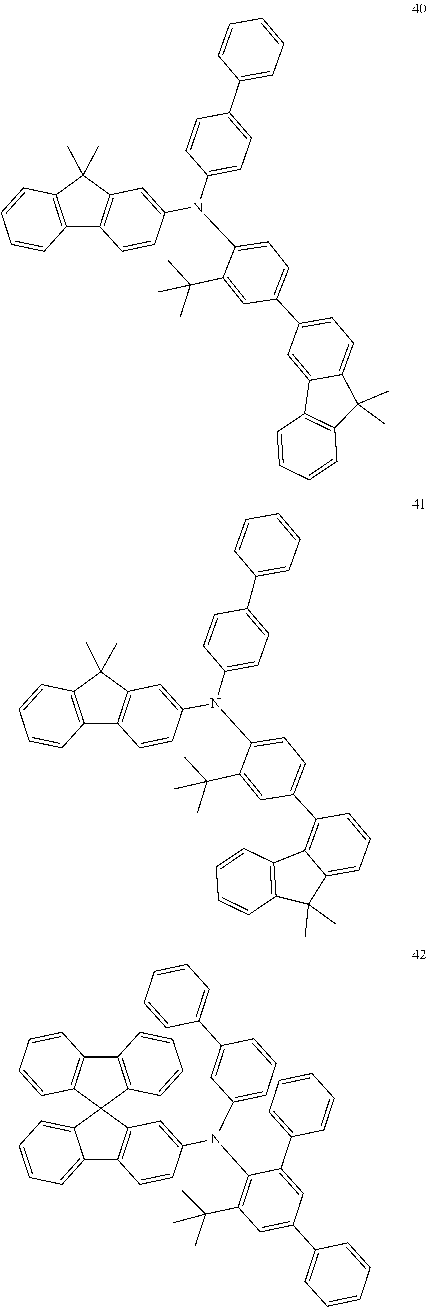

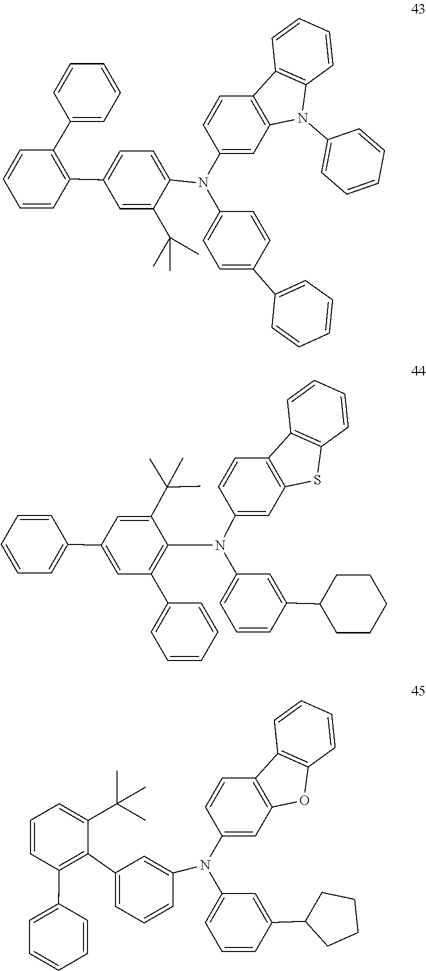

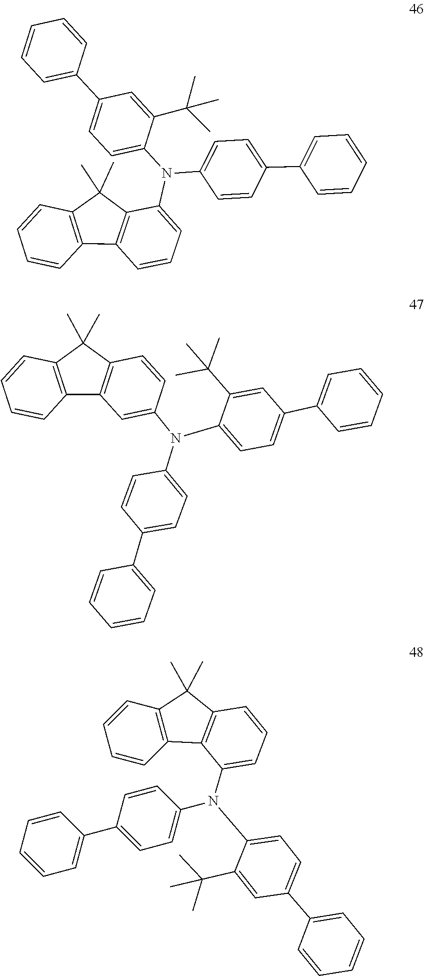

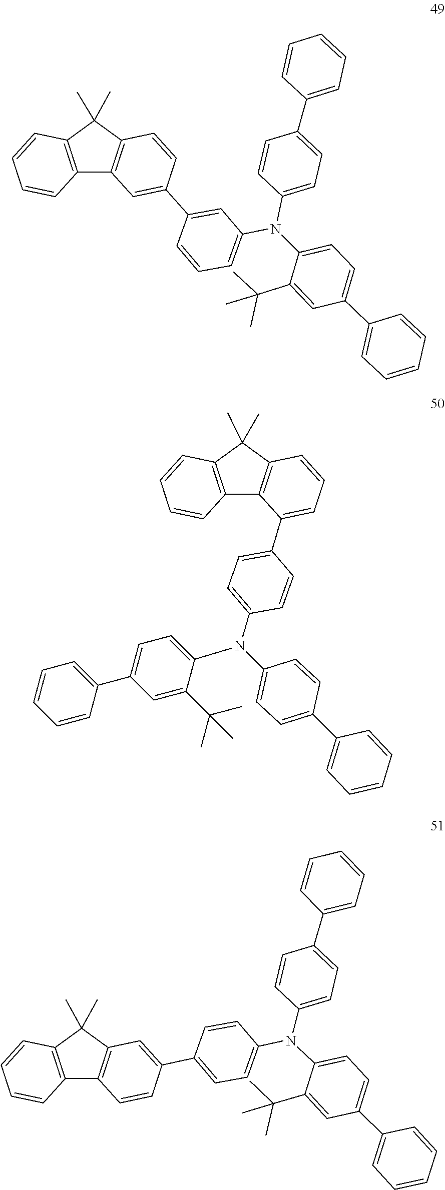 [SMILES: CC(C)(C)c1cc(-c2ccccc2)ccc1N(c1ccc(-c2ccccc2)cc1)c1cccc(-c2ccc3c(c2)-c2ccccc2C3(C)C)c1 ; CC(C)(C)c1cc(-c2ccccc2)ccc1N(c1ccc(-c2ccccc2)cc1)c1ccc(-c2cccc3c2-c2ccccc2C3(C)C)cc1 ; CC(C)(C)c1cc(-c2ccccc2)ccc1N(c1ccc(-c2ccccc2)cc1)c1ccc(-c2ccc3c(c2)C(C)(C)c2ccccc2-3)cc1]

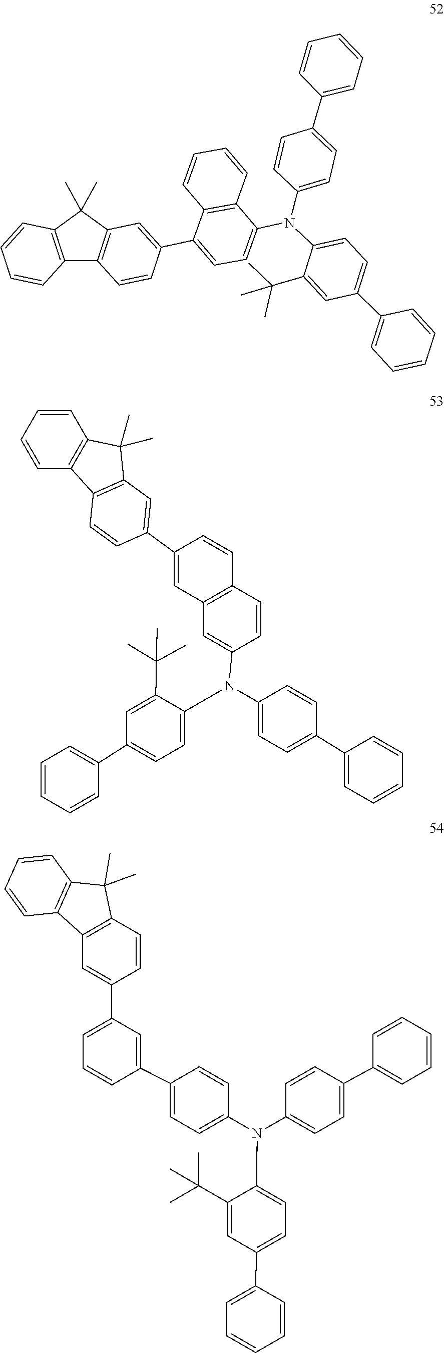

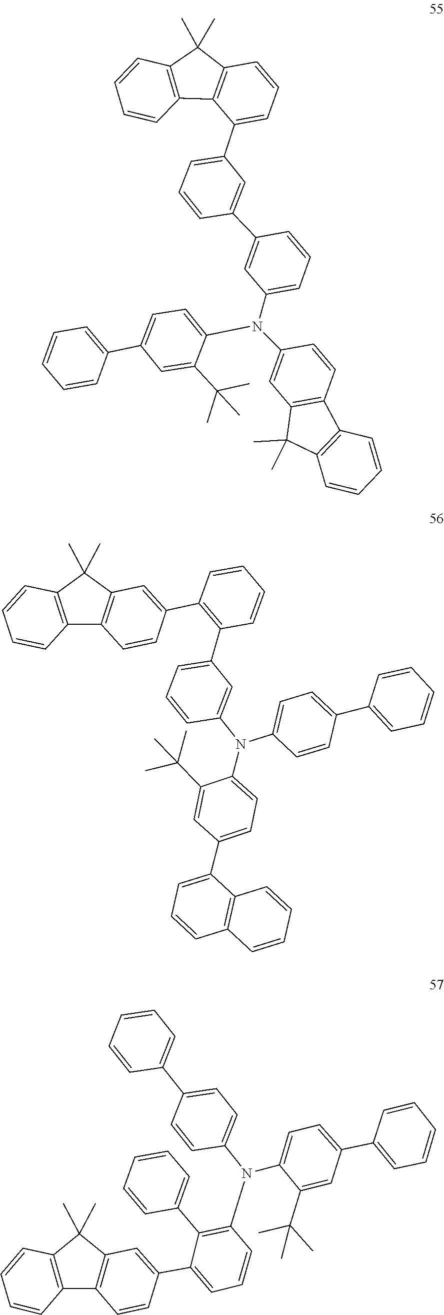 [SMILES: CC(C)(C)c1cc(-c2ccccc2)ccc1N(c1cccc(-c2cccc(-c3cccc4c3-c3ccccc3C4(C)C)c2)c1)c1ccc2c(c1)C(C)(C)c1ccccc1-2 ; CC(C)(C)c1cc(-c2cccc3ccccc23)ccc1N(c1ccc(-c2ccccc2)cc1)c1cccc(-c2ccccc2-c2ccc3c(c2)C(C)(C)c2ccccc2-3)c1 ; CC(C)(C)c1cc(-c2ccccc2)ccc1N(c1ccc(-c2ccccc2)cc1)c1cccc(-c2ccc3c(c2)C(C)(C)c2ccccc2-3)c1-c1ccccc1]

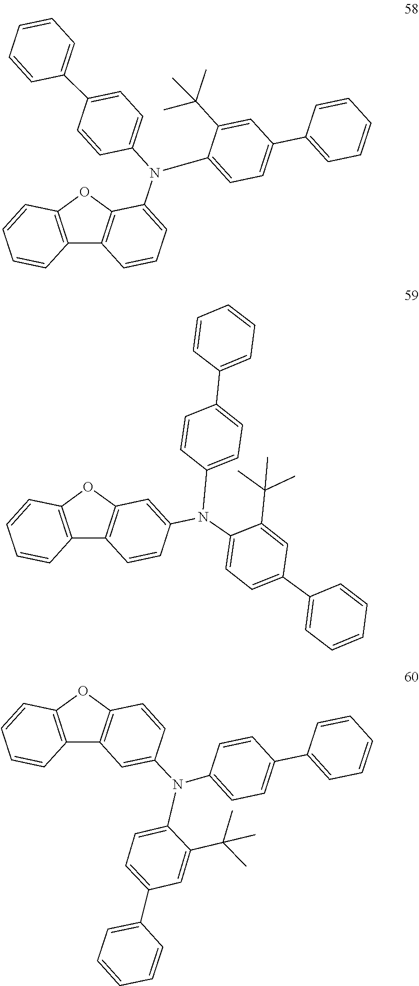 [SMILES: CC(C)(C)c1cc(-c2ccccc2)ccc1N(c1ccc(-c2ccccc2)cc1)c1cccc2c1oc1ccccc12 ; CC(C)(C)c1cc(-c2ccccc2)ccc1N(c1ccc(-c2ccccc2)cc1)c1ccc2c(c1)oc1ccccc12 ; CC(C)(C)c1cc(-c2ccccc2)ccc1N(c1ccc(-c2ccccc2)cc1)c1ccc2oc3ccccc3c2c1]

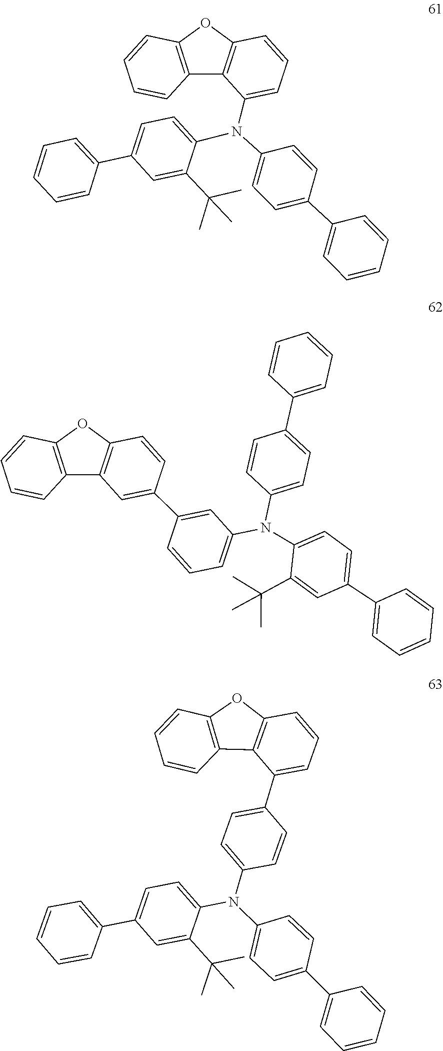 [SMILES: CC(C)(C)c1cc(-c2ccccc2)ccc1N(c1ccc(-c2ccccc2)cc1)c1cccc2oc3ccccc3c12 ; CC(C)(C)c1cc(-c2ccccc2)ccc1N(c1ccc(-c2ccccc2)cc1)c1cccc(-c2ccc3oc4ccccc4c3c2)c1 ; CC(C)(C)c1cc(-c2ccccc2)ccc1N(c1ccc(-c2ccccc2)cc1)c1ccc(-c2cccc3oc4ccccc4c23)cc1]

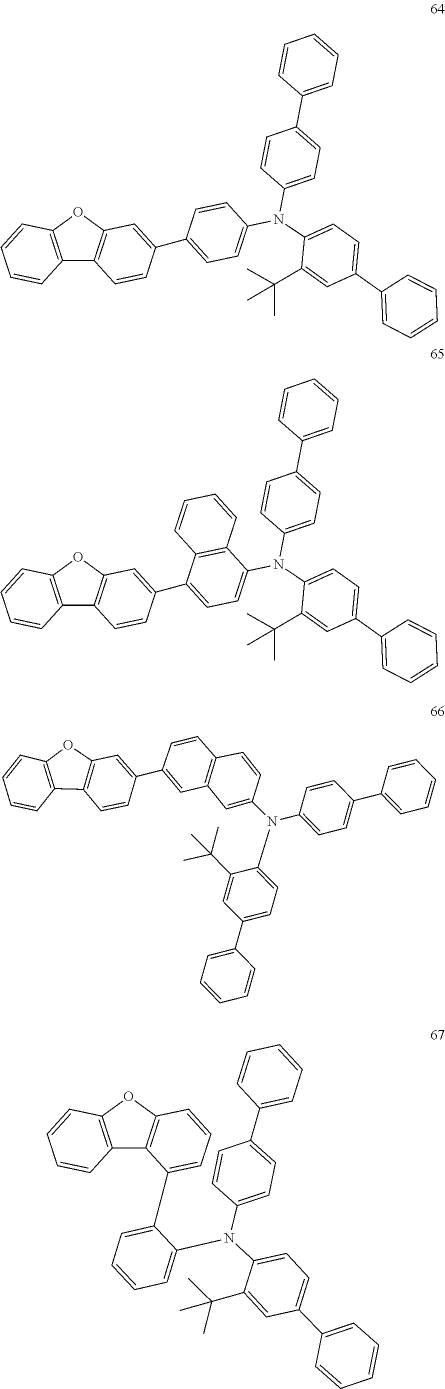 [SMILES: CC(C)(C)c1cc(-c2ccccc2)ccc1N(c1ccc(-c2ccccc2)cc1)c1ccc(-c2ccc3c(c2)oc2ccccc23)cc1 ; CC(C)(C)c1cc(-c2ccccc2)ccc1N(c1ccc(-c2ccccc2)cc1)c1ccc(-c2ccc3c(c2)oc2ccccc23)c2ccccc12 ; CC(C)(C)c1cc(-c2ccccc2)ccc1N(c1ccc(-c2ccccc2)cc1)c1ccc2ccc(-c3ccc4c(c3)oc3ccccc34)cc2c1 ; CC(C)(C)c1cc(-c2ccccc2)ccc1N(c1ccc(-c2ccccc2)cc1)c1ccccc1-c1cccc2oc3ccccc3c12]

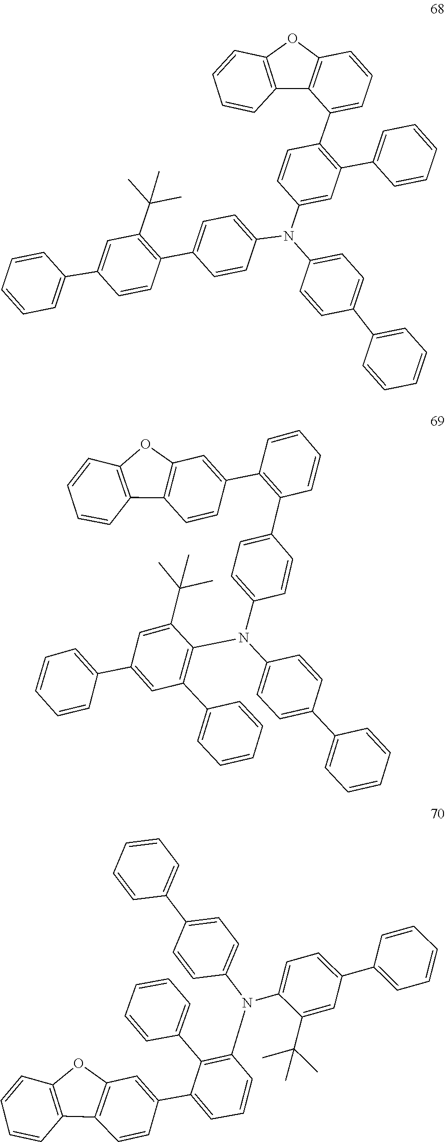 [SMILES: CC(C)(C)c1cc(-c2ccccc2)ccc1-c1ccc(N(c2ccc(-c3ccccc3)cc2)c2ccc(-c3cccc4oc5ccccc5c34)c(-c3ccccc3)c2)cc1 ; CC(C)(C)c1cc(-c2ccccc2)cc(-c2ccccc2)c1N(c1ccc(-c2ccccc2)cc1)c1ccc(-c2ccccc2-c2ccc3c(c2)oc2ccccc23)cc1 ; CC(C)(C)c1cc(-c2ccccc2)ccc1N(c1ccc(-c2ccccc2)cc1)c1cccc(-c2ccc3c(c2)oc2ccccc23)c1-c1ccccc1]

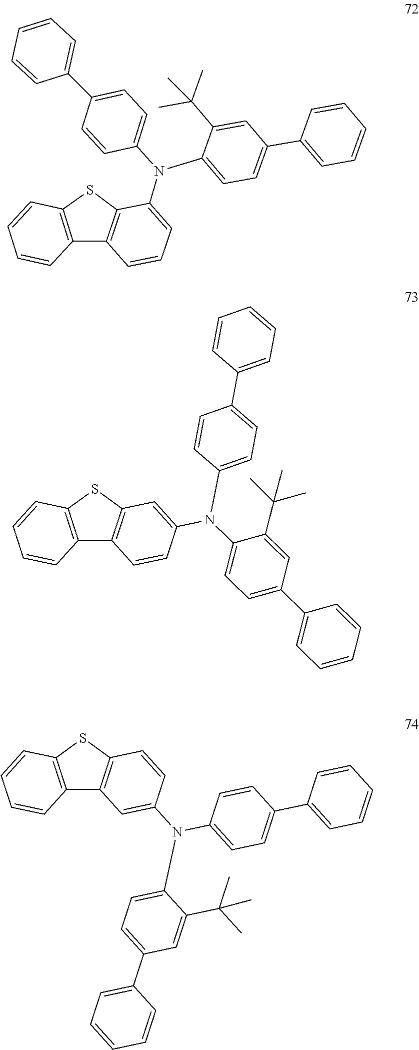 [SMILES: CC(C)(C)c1cc(-c2ccccc2)ccc1N(c1ccc(-c2ccccc2)cc1)c1cccc2c1sc1ccccc12 ; CC(C)(C)c1cc(-c2ccccc2)ccc1N(c1ccc(-c2ccccc2)cc1)c1ccc2c(c1)sc1ccccc12 ; CC(C)(C)c1cc(-c2ccccc2)ccc1N(c1ccc(-c2ccccc2)cc1)c1ccc2sc3ccccc3c2c1]

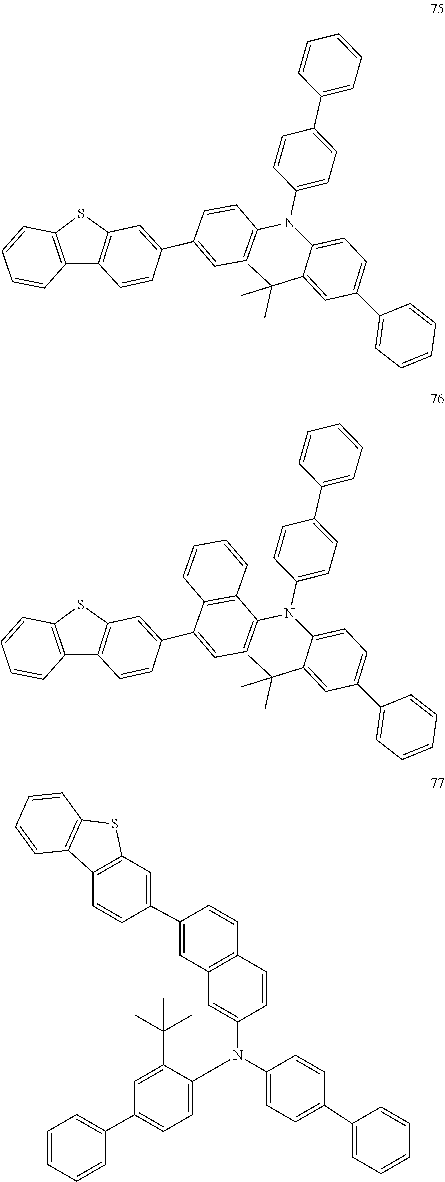 [SMILES: CC(C)(C)c1cc(-c2ccccc2)ccc1N(c1ccc(-c2ccccc2)cc1)c1ccc(-c2ccc3c(c2)sc2ccccc23)cc1 ; CC(C)(C)c1cc(-c2ccccc2)ccc1N(c1ccc(-c2ccccc2)cc1)c1ccc(-c2ccc3c(c2)sc2ccccc23)c2ccccc12 ; CC(C)(C)c1cc(-c2ccccc2)ccc1N(c1ccc(-c2ccccc2)cc1)c1ccc2ccc(-c3ccc4c(c3)sc3ccccc34)cc2c1]

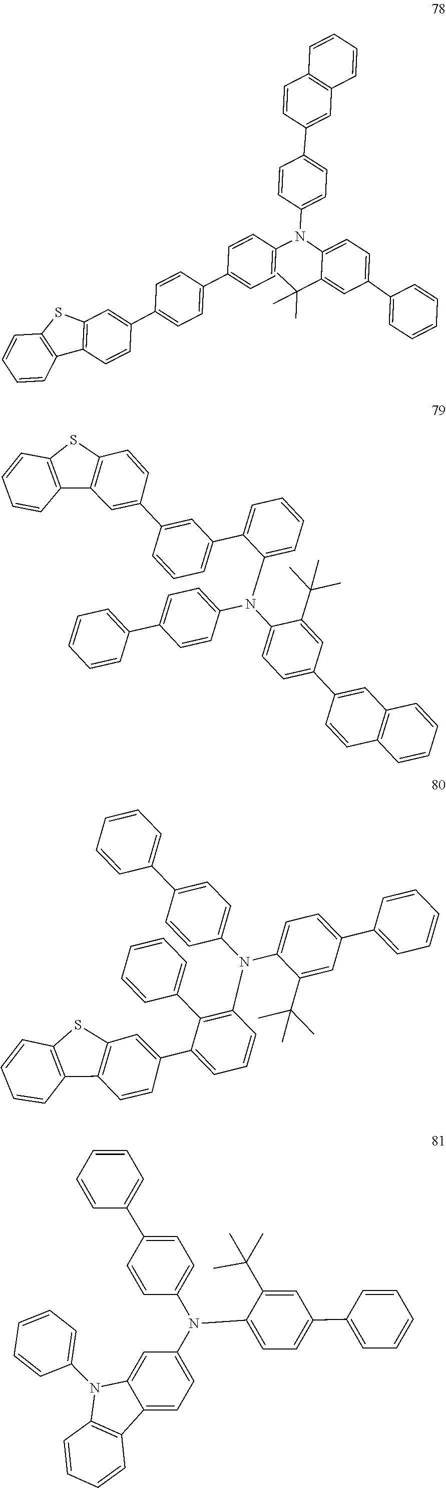 [SMILES: CC(C)(C)c1cc(-c2ccccc2)ccc1N(c1ccc(-c2ccc(-c3ccc4c(c3)sc3ccccc34)cc2)cc1)c1ccc(-c2ccc3ccccc3c2)cc1 ; CC(C)(C)c1cc(-c2ccc3ccccc3c2)ccc1N(c1ccc(-c2ccccc2)cc1)c1ccccc1-c1cccc(-c2ccc3sc4ccccc4c3c2)c1 ; CC(C)(C)c1cc(-c2ccccc2)ccc1N(c1ccc(-c2ccccc2)cc1)c1cccc(-c2ccc3c(c2)sc2ccccc23)c1-c1ccccc1 ; CC(C)(C)c1cc(-c2ccccc2)ccc1N(c1ccc(-c2ccccc2)cc1)c1ccc2c3ccccc3n(-c3ccccc3)c2c1]

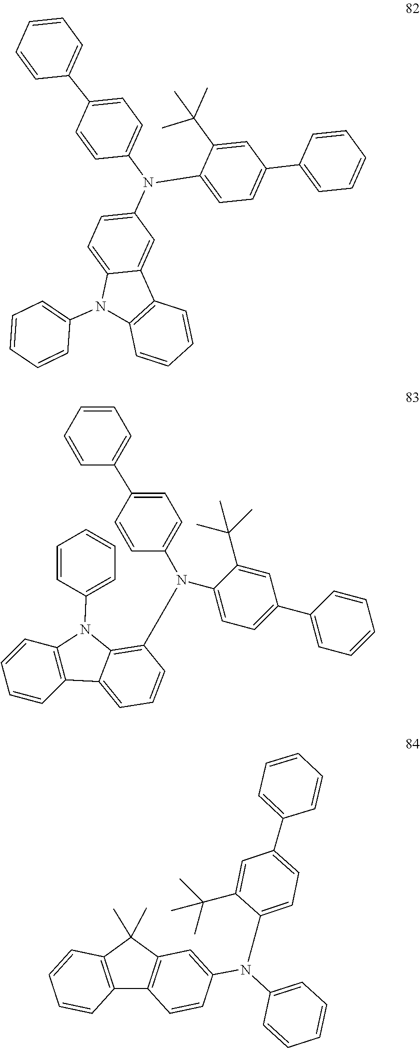 [SMILES: CC(C)(C)c1cc(-c2ccccc2)ccc1N(c1ccc(-c2ccccc2)cc1)c1ccc2c(c1)c1ccccc1n2-c1ccccc1 ; CC(C)(C)c1cc(-c2ccccc2)ccc1N(c1ccc(-c2ccccc2)cc1)c1cccc2c3ccccc3n(-c3ccccc3)c12 ; CC(C)(C)c1cc(-c2ccccc2)ccc1N(c1ccccc1)c1ccc2c(c1)C(C)(C)c1ccccc1-2]

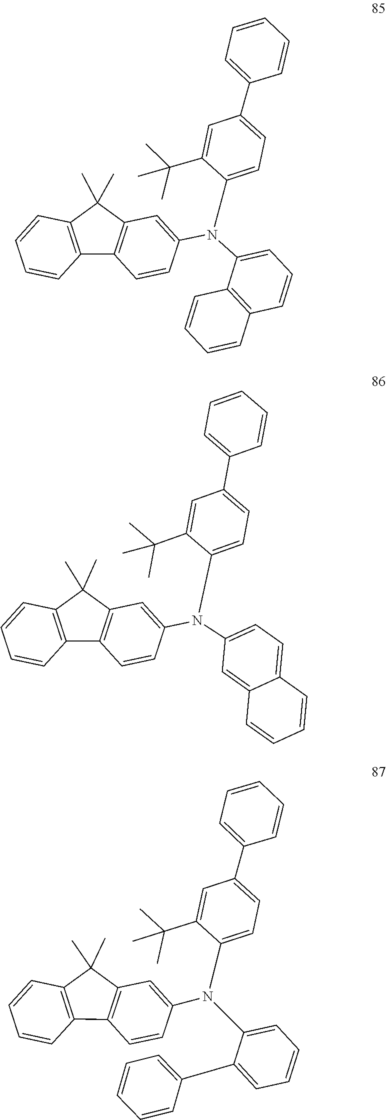

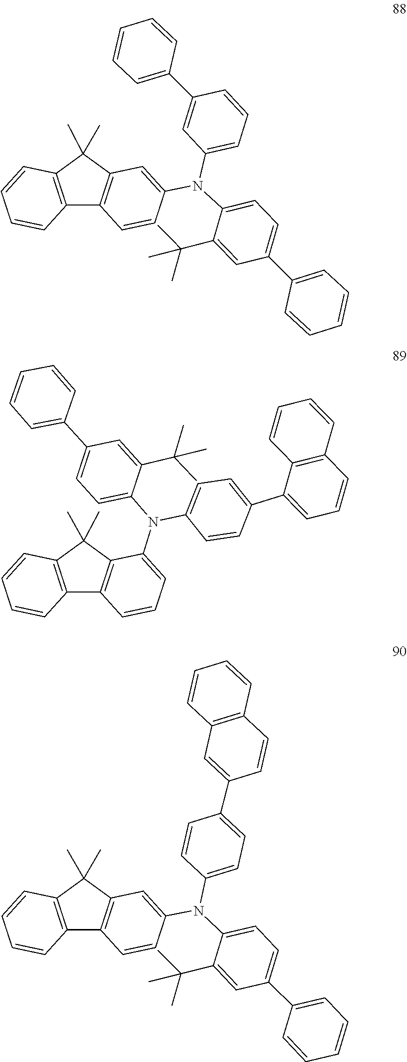 [SMILES: CC(C)(C)c1cc(-c2ccccc2)ccc1N(c1cccc(-c2ccccc2)c1)c1ccc2c(c1)C(C)(C)c1ccccc1-2 ; CC(C)(C)c1cc(-c2ccccc2)ccc1N(c1ccc(-c2cccc3ccccc23)cc1)c1cccc2c1C(C)(C)c1ccccc1-2 ; CC(C)(C)c1cc(-c2ccccc2)ccc1N(c1ccc(-c2ccc3ccccc3c2)cc1)c1ccc2c(c1)C(C)(C)c1ccccc1-2]

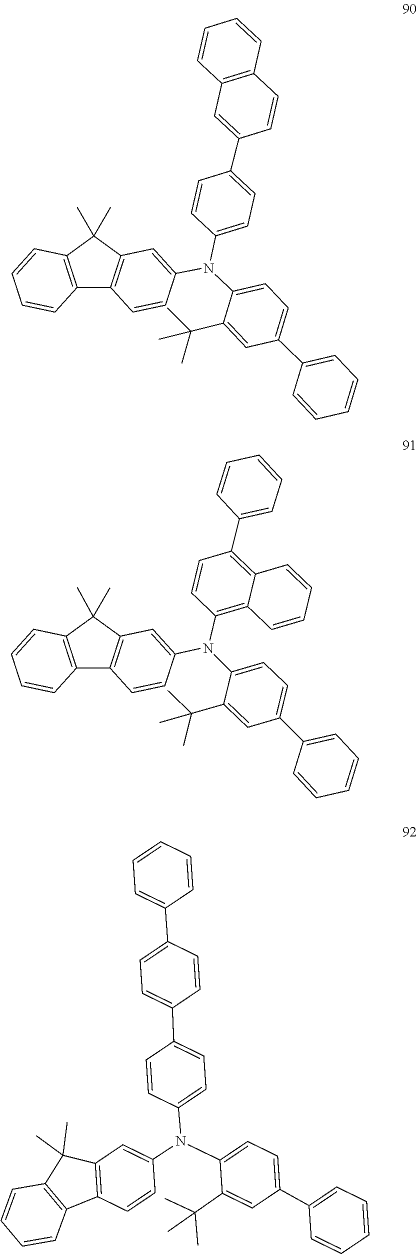 [SMILES: CC(C)(C)c1cc(-c2ccccc2)ccc1N(c1ccc(-c2ccc3ccccc3c2)cc1)c1ccc2c(c1)C(C)(C)c1ccccc1-2 ; CC(C)(C)c1cc(-c2ccccc2)ccc1N(c1ccc2c(c1)C(C)(C)c1ccccc1-2)c1ccc(-c2ccccc2)c2ccccc12 ; CC(C)(C)c1cc(-c2ccccc2)ccc1N(c1ccc(-c2ccc(-c3ccccc3)cc2)cc1)c1ccc2c(c1)C(C)(C)c1ccccc1-2]

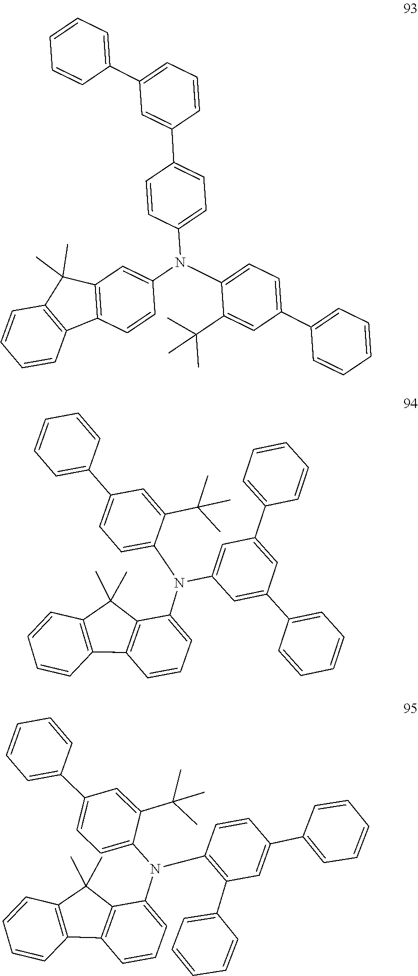

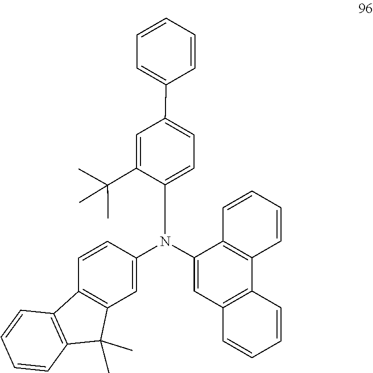 [SMILES: CC(C)(C)c1cc(-c2ccccc2)ccc1N(c1ccc2c(c1)C(C)(C)c1ccccc1-2)c1cc2ccccc2c2ccccc12]

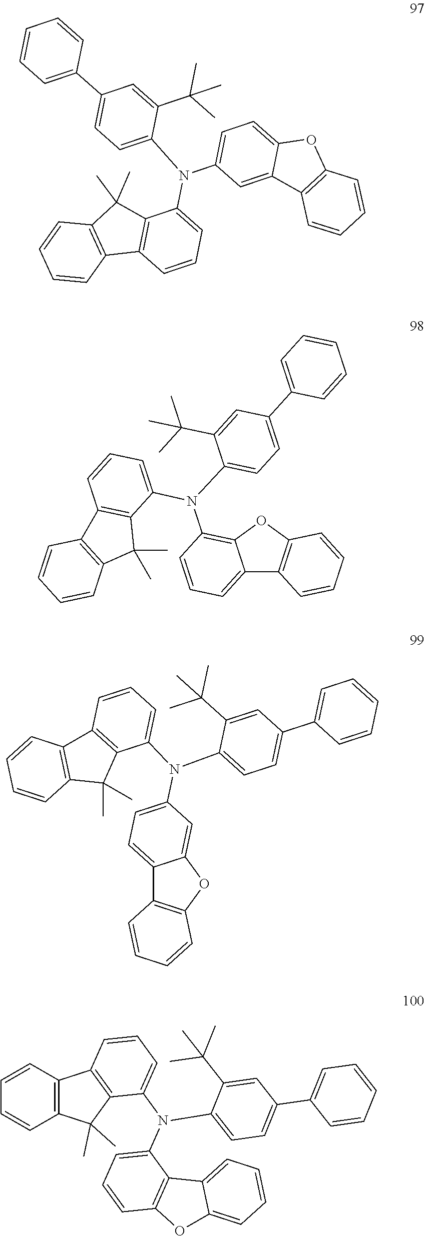 [SMILES: CC(C)(C)c1cc(-c2ccccc2)ccc1N(c1ccc2oc3ccccc3c2c1)c1cccc2c1C(C)(C)c1ccccc1-2 ; CC(C)(C)c1cc(-c2ccccc2)ccc1N(c1cccc2c1C(C)(C)c1ccccc1-2)c1cccc2c1oc1ccccc12 ; CC(C)(C)c1cc(-c2ccccc2)ccc1N(c1ccc2c(c1)oc1ccccc12)c1cccc2c1C(C)(C)c1ccccc1-2 ; CC(C)(C)c1cc(-c2ccccc2)ccc1N(c1cccc2c1C(C)(C)c1ccccc1-2)c1cccc2oc3ccccc3c12]

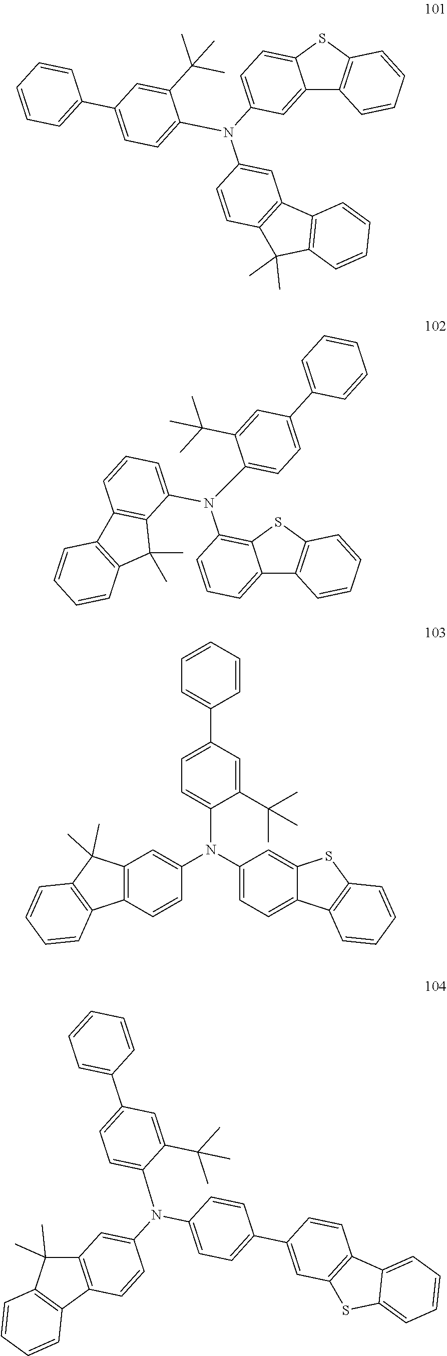 [SMILES: CC(C)(C)c1cc(-c2ccccc2)ccc1N(c1ccc2c(c1)-c1ccccc1C2(C)C)c1ccc2sc3ccccc3c2c1 ; CC(C)(C)c1cc(-c2ccccc2)ccc1N(c1cccc2c1C(C)(C)c1ccccc1-2)c1cccc2c1sc1ccccc12 ; CC(C)(C)c1cc(-c2ccccc2)ccc1N(c1ccc2c(c1)C(C)(C)c1ccccc1-2)c1ccc2c(c1)sc1ccccc12 ; CC(C)(C)c1cc(-c2ccccc2)ccc1N(c1ccc(-c2ccc3c(c2)sc2ccccc23)cc1)c1ccc2c(c1)C(C)(C)c1ccccc1-2]

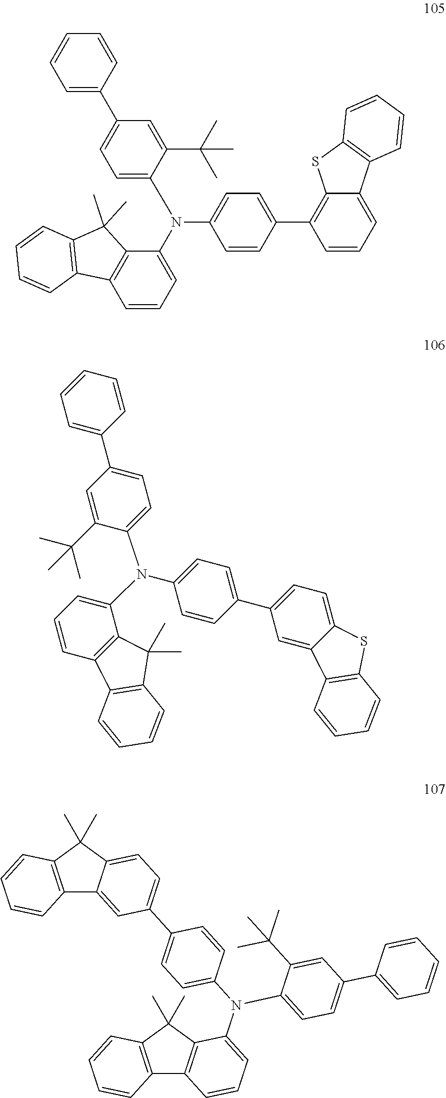 [SMILES: CC(C)(C)c1cc(-c2ccccc2)ccc1N(c1ccc(-c2cccc3c2sc2ccccc23)cc1)c1cccc2c1C(C)(C)c1ccccc1-2 ; CC(C)(C)c1cc(-c2ccccc2)ccc1N(c1ccc(-c2ccc3sc4ccccc4c3c2)cc1)c1cccc2c1C(C)(C)c1ccccc1-2 ; CC(C)(C)c1cc(-c2ccccc2)ccc1N(c1ccc(-c2ccc3c(c2)-c2ccccc2C3(C)C)cc1)c1cccc2c1C(C)(C)c1ccccc1-2]

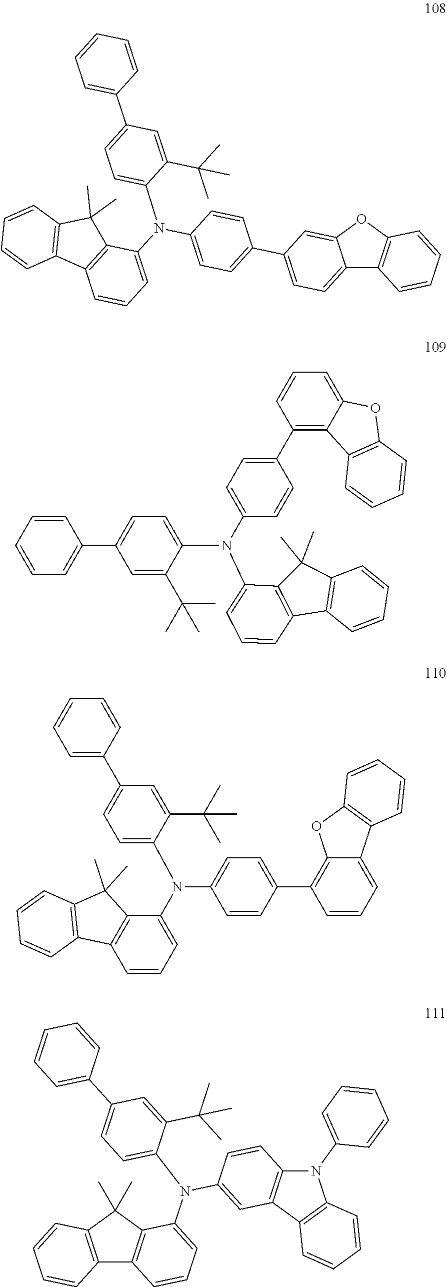 [SMILES: CC(C)(C)c1cc(-c2ccccc2)ccc1N(c1ccc(-c2ccc3c(c2)oc2ccccc23)cc1)c1cccc2c1C(C)(C)c1ccccc1-2 ; CC(C)(C)c1cc(-c2ccccc2)ccc1N(c1ccc(-c2cccc3oc4ccccc4c23)cc1)c1cccc2c1C(C)(C)c1ccccc1-2 ; CC(C)(C)c1cc(-c2ccccc2)ccc1N(c1ccc(-c2cccc3c2oc2ccccc23)cc1)c1cccc2c1C(C)(C)c1ccccc1-2 ; CC(C)(C)c1cc(-c2ccccc2)ccc1N(c1ccc2c(c1)c1ccccc1n2-c1ccccc1)c1cccc2c1C(C)(C)c1ccccc1-2]

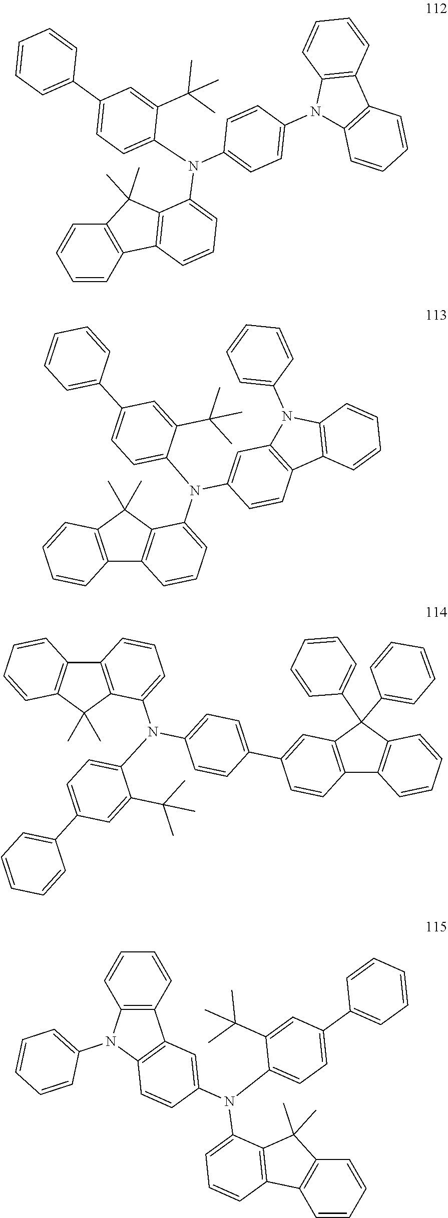 [SMILES: CC(C)(C)c1cc(-c2ccccc2)ccc1N(c1ccc(-n2c3ccccc3c3ccccc32)cc1)c1cccc2c1C(C)(C)c1ccccc1-2 ; CC(C)(C)c1cc(-c2ccccc2)ccc1N(c1ccc2c3ccccc3n(-c3ccccc3)c2c1)c1cccc2c1C(C)(C)c1ccccc1-2 ; CC(C)(C)c1cc(-c2ccccc2)ccc1N(c1ccc(-c2ccc3c(c2)C(c2ccccc2)(c2ccccc2)c2ccccc2-3)cc1)c1cccc2c1C(C)(C)c1ccccc1-2 ; CC(C)(C)c1cc(-c2ccccc2)ccc1N(c1ccc2c(c1)c1ccccc1n2-c1ccccc1)c1cccc2c1C(C)(C)c1ccccc1-2]

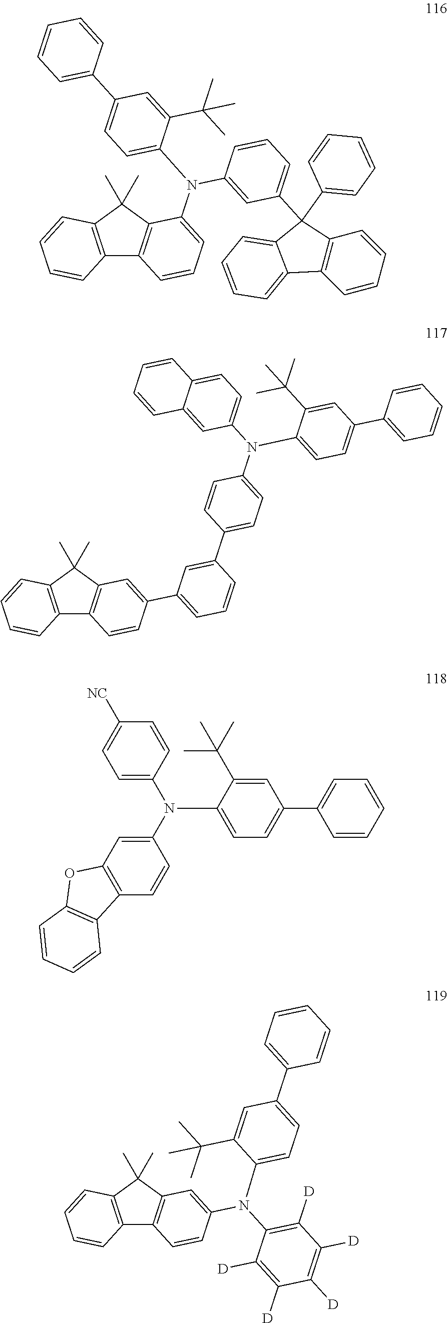 [SMILES: CC(C)(C)c1cc(-c2ccccc2)ccc1N(c1cccc(C2(c3ccccc3)c3ccccc3-c3ccccc32)c1)c1cccc2c1C(C)(C)c1ccccc1-2 ; CC(C)(C)c1cc(-c2ccccc2)ccc1N(c1ccc(-c2cccc(-c3ccc4c(c3)C(C)(C)c3ccccc3-4)c2)cc1)c1ccc2ccccc2c1 ; CC(C)(C)c1cc(-c2ccccc2)ccc1N(c1ccc(C#N)cc1)c1ccc2c(c1)oc1ccccc12 ; [2H]c1c([2H])c([2H])c(N(c2ccc3c(c2)C(C)(C)c2ccccc2-3)c2ccc(-c3ccccc3)cc2C(C)(C)C)c([2H])c1[2H]]

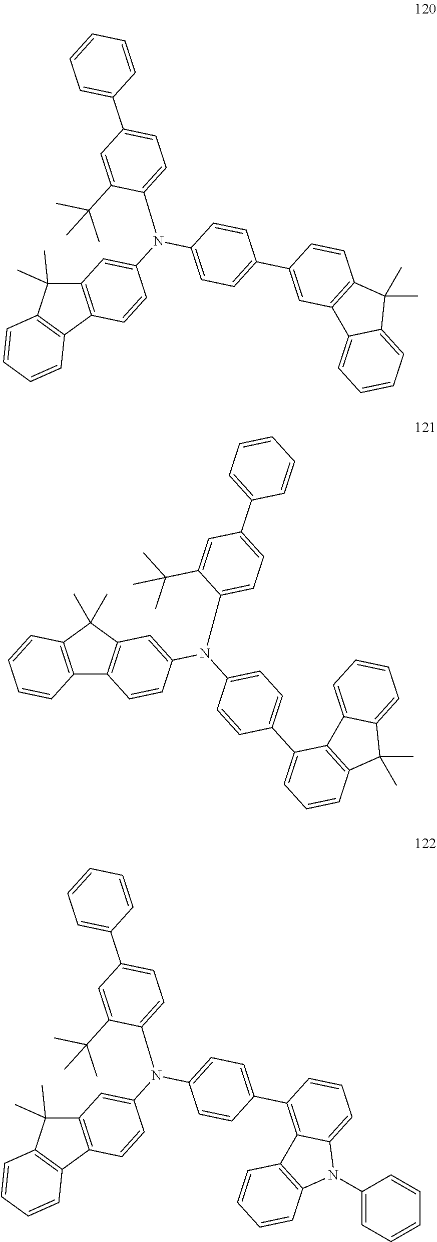 [SMILES: CC(C)(C)c1cc(-c2ccccc2)ccc1N(c1ccc(-c2ccc3c(c2)-c2ccccc2C3(C)C)cc1)c1ccc2c(c1)C(C)(C)c1ccccc1-2 ; CC(C)(C)c1cc(-c2ccccc2)ccc1N(c1ccc(-c2cccc3c2-c2ccccc2C3(C)C)cc1)c1ccc2c(c1)C(C)(C)c1ccccc1-2 ; CC(C)(C)c1cc(-c2ccccc2)ccc1N(c1ccc(-c2cccc3c2c2ccccc2n3-c2ccccc2)cc1)c1ccc2c(c1)C(C)(C)c1ccccc1-2]

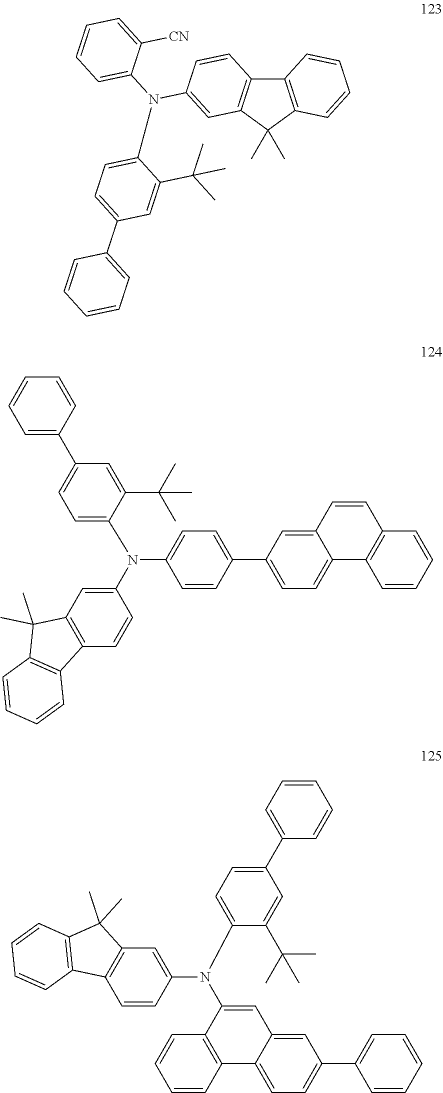 [SMILES: CC(C)(C)c1cc(-c2ccccc2)ccc1N(c1ccc2c(c1)C(C)(C)c1ccccc1-2)c1ccccc1C#N ; CC(C)(C)c1cc(-c2ccccc2)ccc1N(c1ccc(-c2ccc3c(ccc4ccccc43)c2)cc1)c1ccc2c(c1)C(C)(C)c1ccccc1-2 ; CC(C)(C)c1cc(-c2ccccc2)ccc1N(c1ccc2c(c1)C(C)(C)c1ccccc1-2)c1cc2cc(-c3ccccc3)ccc2c2ccccc12]

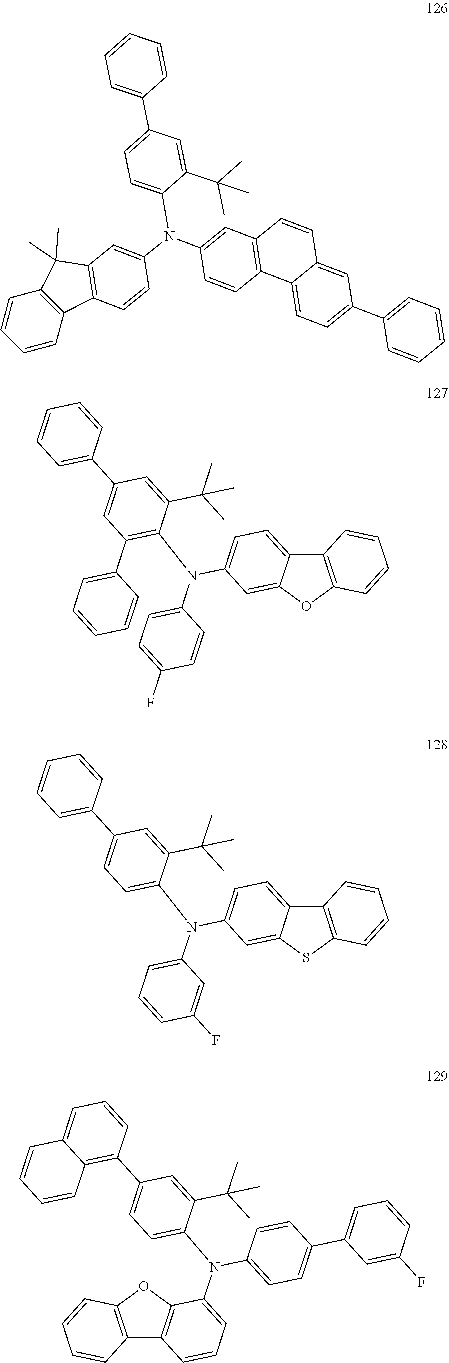 [SMILES: CC(C)(C)c1cc(-c2ccccc2)ccc1N(c1ccc2c(c1)C(C)(C)c1ccccc1-2)c1ccc2c(ccc3cc(-c4ccccc4)ccc32)c1 ; CC(C)(C)c1cc(-c2ccccc2)cc(-c2ccccc2)c1N(c1ccc(F)cc1)c1ccc2c(c1)oc1ccccc12 ; CC(C)(C)c1cc(-c2ccccc2)ccc1N(c1cccc(F)c1)c1ccc2c(c1)sc1ccccc12 ; CC(C)(C)c1cc(-c2cccc3ccccc23)ccc1N(c1ccc(-c2cccc(F)c2)cc1)c1cccc2c1oc1ccccc12]

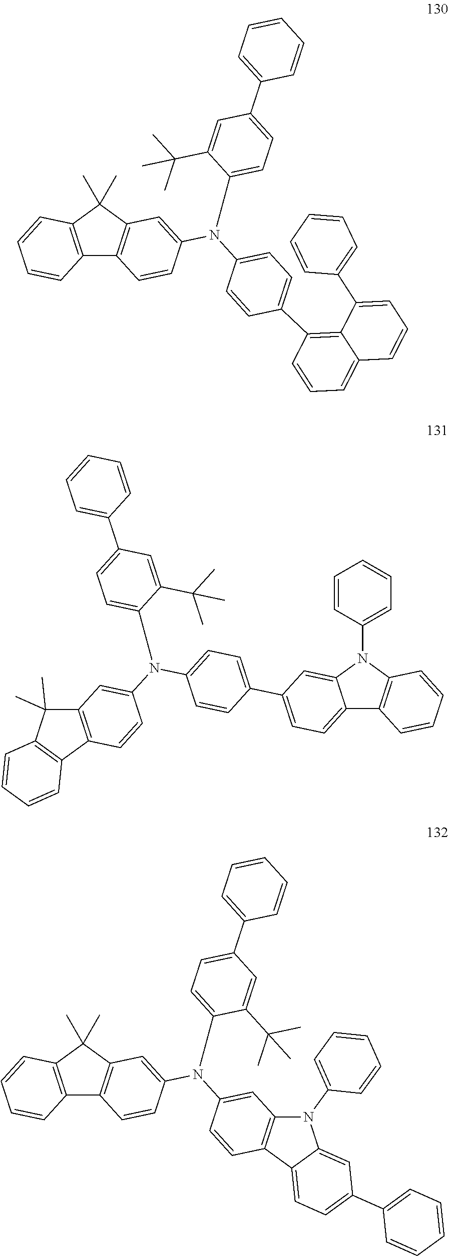 [SMILES: CC(C)(C)c1cc(-c2ccccc2)ccc1N(c1ccc(-c2cccc3cccc(-c4ccccc4)c23)cc1)c1ccc2c(c1)C(C)(C)c1ccccc1-2 ; CC(C)(C)c1cc(-c2ccccc2)ccc1N(c1ccc(-c2ccc3c4ccccc4n(-c4ccccc4)c3c2)cc1)c1ccc2c(c1)C(C)(C)c1ccccc1-2 ; CC(C)(C)c1cc(-c2ccccc2)ccc1N(c1ccc2c(c1)C(C)(C)c1ccccc1-2)c1ccc2c3ccc(-c4ccccc4)cc3n(-c3ccccc3)c2c1]

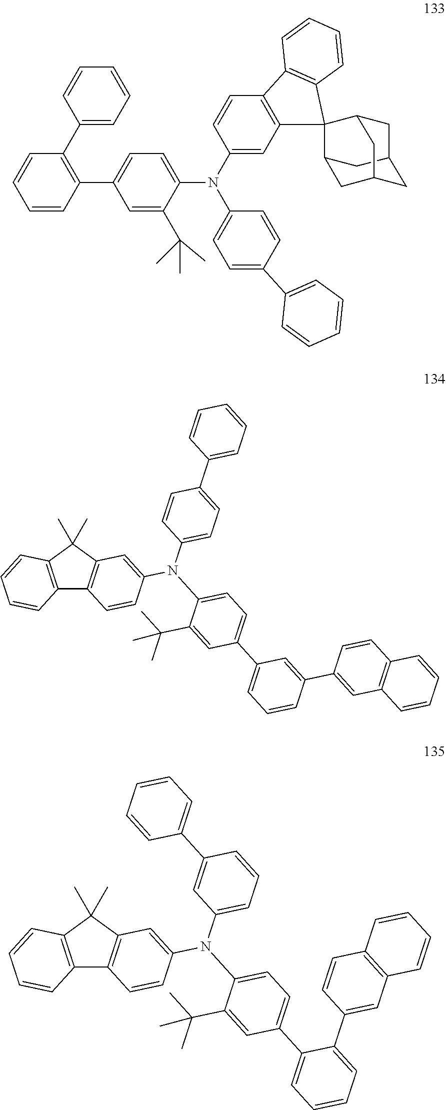 [SMILES: CC(C)(C)c1cc(-c2ccccc2-c2ccccc2)ccc1N(c1ccc(-c2ccccc2)cc1)c1ccc2c(c1)C1(c3ccccc3-2)C2CC3CC(C2)CC1C3 ; CC(C)(C)c1cc(-c2cccc(-c3ccc4ccccc4c3)c2)ccc1N(c1ccc(-c2ccccc2)cc1)c1ccc2c(c1)C(C)(C)c1ccccc1-2 ; CC(C)(C)c1cc(-c2ccccc2-c2ccc3ccccc3c2)ccc1N(c1cccc(-c2ccccc2)c1)c1ccc2c(c1)C(C)(C)c1ccccc1-2]

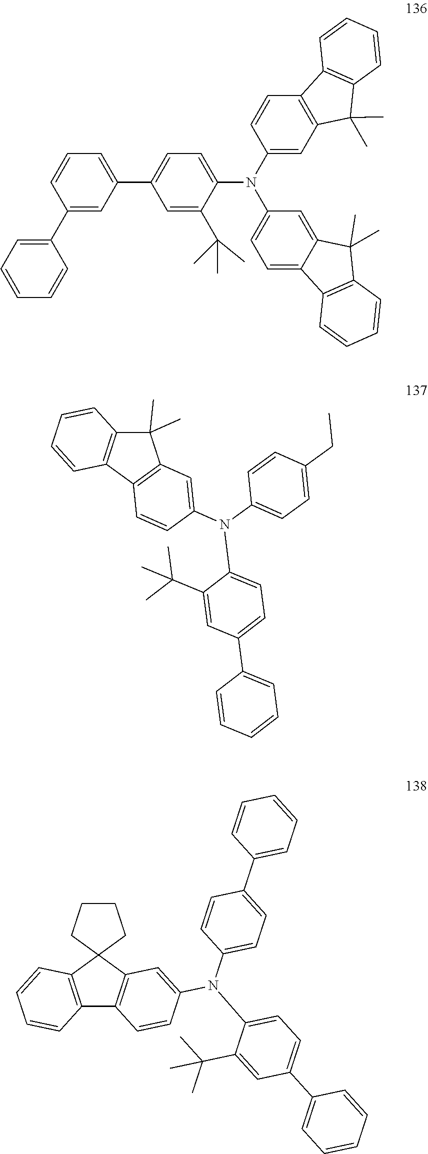 [SMILES: CC(C)(C)c1cc(-c2cccc(-c3ccccc3)c2)ccc1N(c1ccc2c(c1)C(C)(C)c1ccccc1-2)c1ccc2c(c1)C(C)(C)c1ccccc1-2 ; CCc1ccc(N(c2ccc3c(c2)C(C)(C)c2ccccc2-3)c2ccc(-c3ccccc3)cc2C(C)(C)C)cc1 ; CC(C)(C)c1cc(-c2ccccc2)ccc1N(c1ccc(-c2ccccc2)cc1)c1ccc2c(c1)C1(CCCC1)c1ccccc1-2]

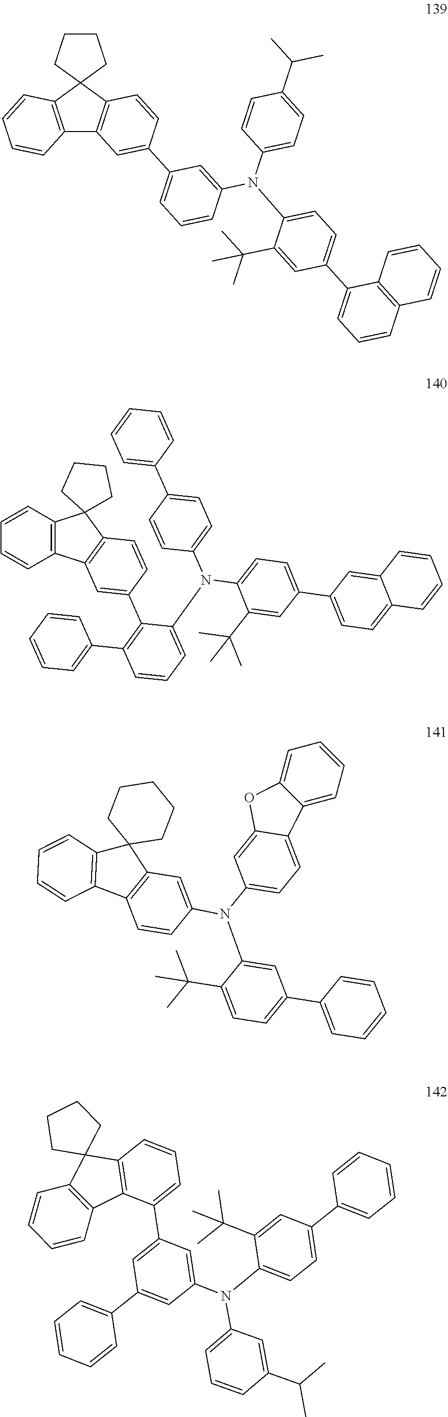 [SMILES: CC(C)c1ccc(N(c2cccc(-c3ccc4c(c3)-c3ccccc3C43CCCC3)c2)c2ccc(-c3cccc4ccccc34)cc2C(C)(C)C)cc1 ; CC(C)(C)c1cc(-c2ccc3ccccc3c2)ccc1N(c1ccc(-c2ccccc2)cc1)c1cccc(-c2ccccc2)c1-c1ccc2c(c1)-c1ccccc1C21CCCC1 ; CC(C)(C)c1ccc(-c2ccccc2)cc1N(c1ccc2c(c1)C1(CCCCC1)c1ccccc1-2)c1ccc2c(c1)oc1ccccc12 ; CC(C)c1cccc(N(c2cc(-c3ccccc3)cc(-c3cccc4c3-c3ccccc3C43CCCC3)c2)c2ccc(-c3ccccc3)cc2C(C)(C)C)c1]

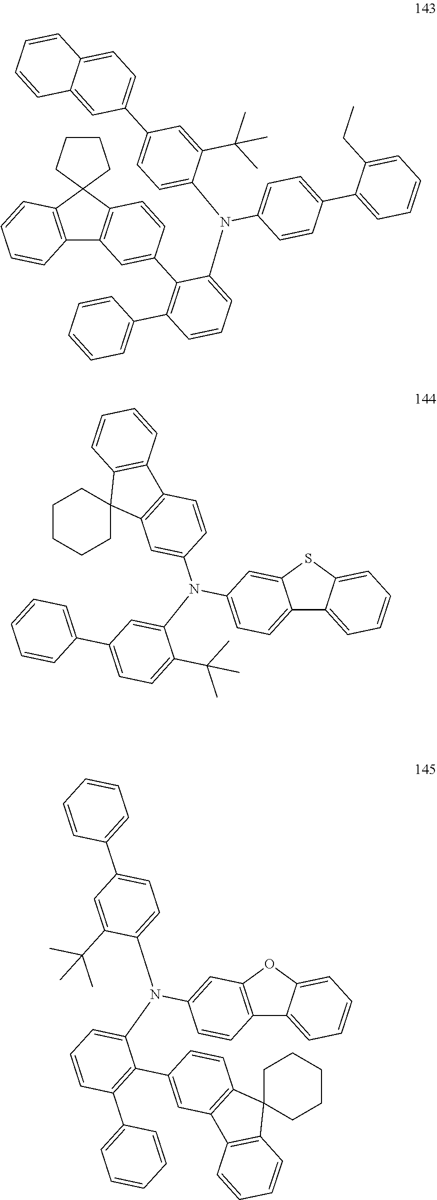 [SMILES: CCc1ccccc1-c1ccc(N(c2ccc(-c3ccc4ccccc4c3)cc2C(C)(C)C)c2cccc(-c3ccccc3)c2-c2ccc3c(c2)-c2ccccc2C32CCCC2)cc1 ; CC(C)(C)c1ccc(-c2ccccc2)cc1N(c1ccc2c(c1)C1(CCCCC1)c1ccccc1-2)c1ccc2c(c1)sc1ccccc12 ; CC(C)(C)c1cc(-c2ccccc2)ccc1N(c1ccc2c(c1)oc1ccccc12)c1cccc(-c2ccccc2)c1-c1ccc2c(c1)-c1ccccc1C21CCCCC1]

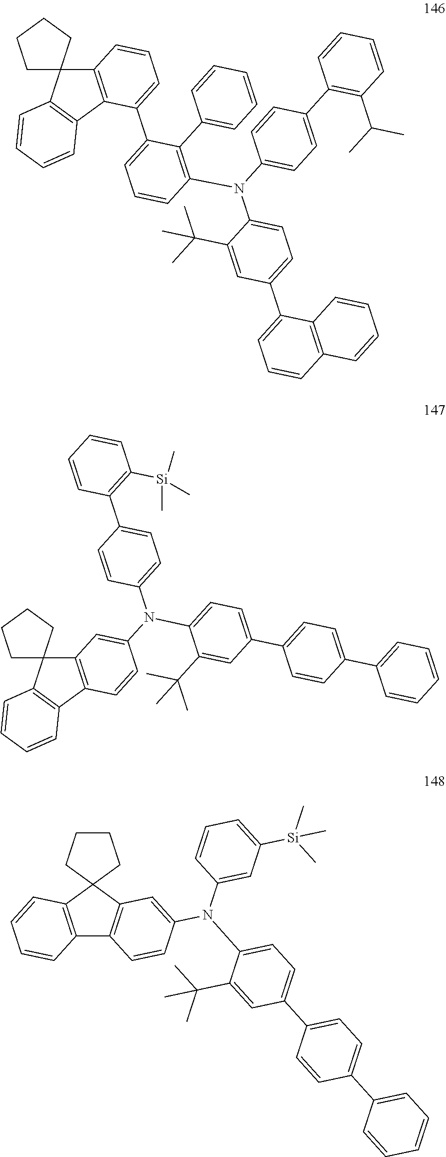 [SMILES: CC(C)c1ccccc1-c1ccc(N(c2ccc(-c3cccc4ccccc34)cc2C(C)(C)C)c2cccc(-c3cccc4c3-c3ccccc3C43CCCC3)c2-c2ccccc2)cc1 ; CC(C)(C)c1cc(-c2ccc(-c3ccccc3)cc2)ccc1N(c1ccc(-c2ccccc2[Si](C)(C)C)cc1)c1ccc2c(c1)C1(CCCC1)c1ccccc1-2 ; CC(C)(C)c1cc(-c2ccc(-c3ccccc3)cc2)ccc1N(c1cccc([Si](C)(C)C)c1)c1ccc2c(c1)C1(CCCC1)c1ccccc1-2]

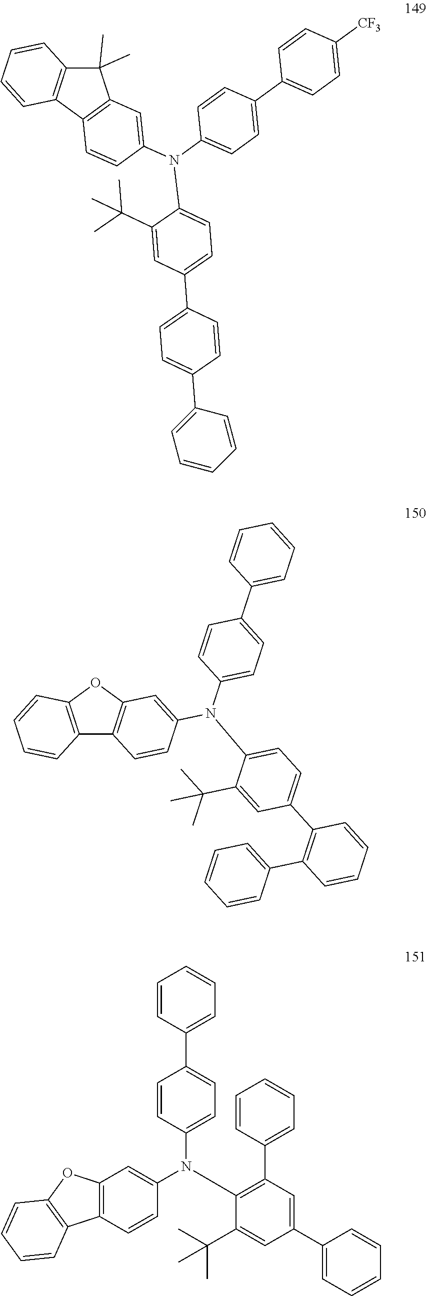 [SMILES: CC(C)(C)c1cc(-c2ccc(-c3ccccc3)cc2)ccc1N(c1ccc(-c2ccc(C(F)(F)F)cc2)cc1)c1ccc2c(c1)C(C)(C)c1ccccc1-2 ; CC(C)(C)c1cc(-c2ccccc2-c2ccccc2)ccc1N(c1ccc(-c2ccccc2)cc1)c1ccc2c(c1)oc1ccccc12 ; CC(C)(C)c1cc(-c2ccccc2)cc(-c2ccccc2)c1N(c1ccc(-c2ccccc2)cc1)c1ccc2c(c1)oc1ccccc12]

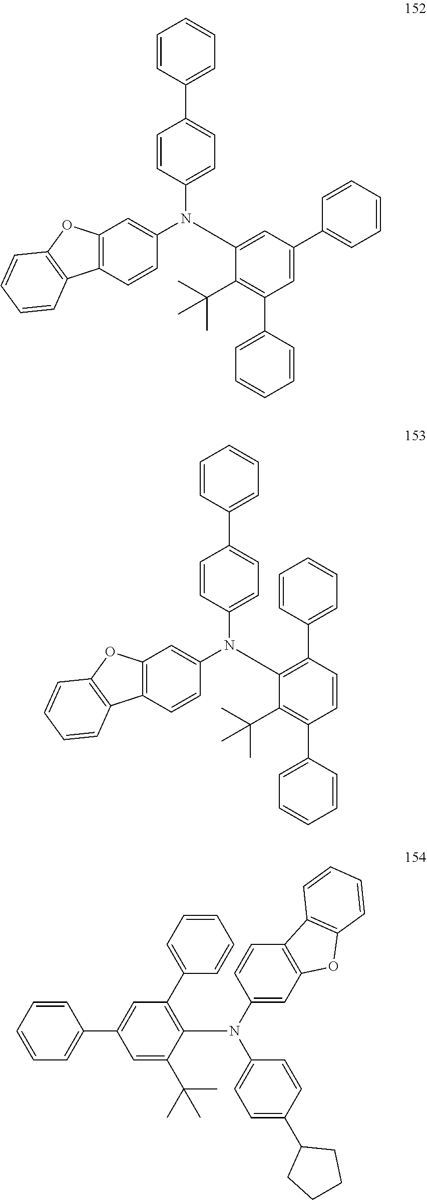 [SMILES: CC(C)(C)c1c(-c2ccccc2)cc(-c2ccccc2)cc1N(c1ccc(-c2ccccc2)cc1)c1ccc2c(c1)oc1ccccc12 ; CC(C)(C)c1c(-c2ccccc2)ccc(-c2ccccc2)c1N(c1ccc(-c2ccccc2)cc1)c1ccc2c(c1)oc1ccccc12 ; CC(C)(C)c1cc(-c2ccccc2)cc(-c2ccccc2)c1N(c1ccc(C2CCCC2)cc1)c1ccc2c(c1)oc1ccccc12]

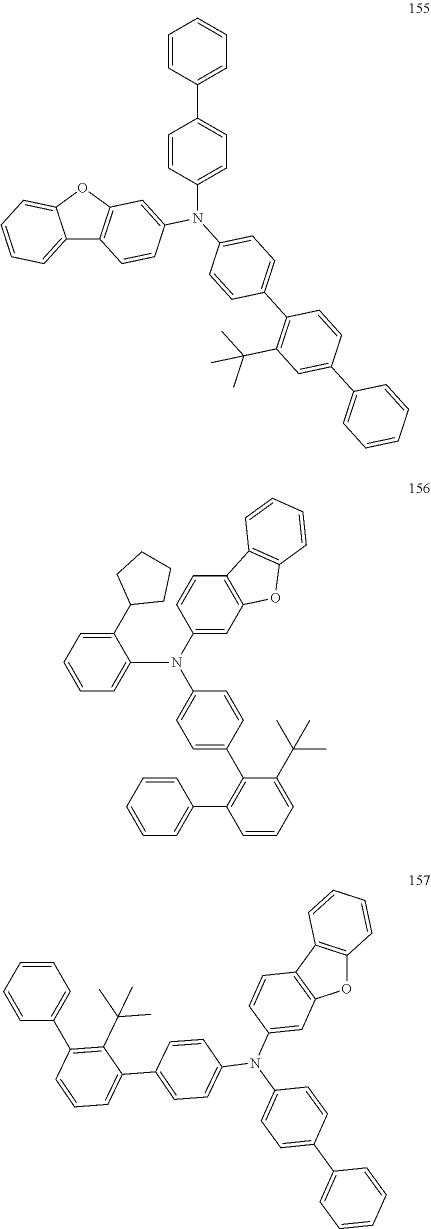 [SMILES: CC(C)(C)c1cc(-c2ccccc2)ccc1-c1ccc(N(c2ccc(-c3ccccc3)cc2)c2ccc3c(c2)oc2ccccc23)cc1 ; CC(C)(C)c1cccc(-c2ccccc2)c1-c1ccc(N(c2ccc3c(c2)oc2ccccc23)c2ccccc2C2CCCC2)cc1 ; CC(C)(C)c1c(-c2ccccc2)cccc1-c1ccc(N(c2ccc(-c3ccccc3)cc2)c2ccc3c(c2)oc2ccccc23)cc1]

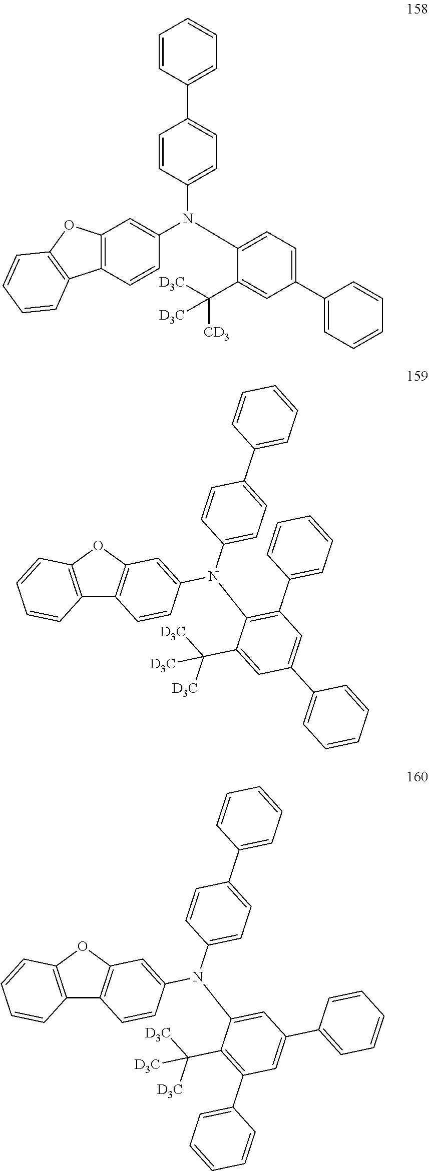 [SMILES: [2H]C([2H])([2H])C(c1cc(-c2ccccc2)ccc1N(c1ccc(-c2ccccc2)cc1)c1ccc2c(c1)oc1ccccc12)(C([2H])([2H])[2H])C([2H])([2H])[2H] ; [2H]C([2H])([2H])C(c1cc(-c2ccccc2)cc(-c2ccccc2)c1N(c1ccc(-c2ccccc2)cc1)c1ccc2c(c1)oc1ccccc12)(C([2H])([2H])[2H])C([2H])([2H])[2H] ; [2H]C([2H])([2H])C(c1c(-c2ccccc2)cc(-c2ccccc2)cc1N(c1ccc(-c2ccccc2)cc1)c1ccc2c(c1)oc1ccccc12)(C([2H])([2H])[2H])C([2H])([2H])[2H]]

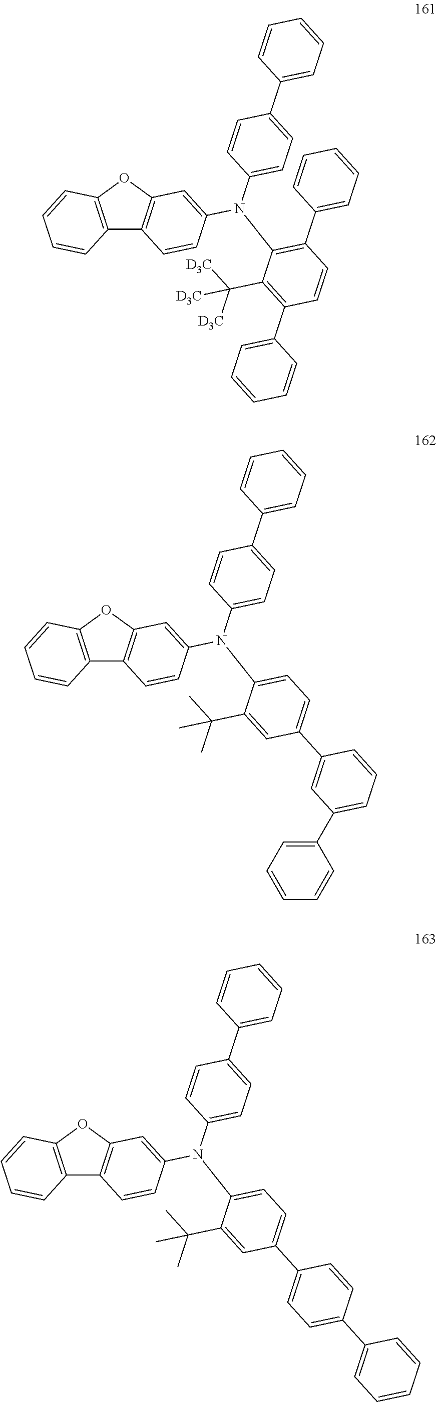 [SMILES: [2H]C([2H])([2H])C(c1c(-c2ccccc2)ccc(-c2ccccc2)c1N(c1ccc(-c2ccccc2)cc1)c1ccc2c(c1)oc1ccccc12)(C([2H])([2H])[2H])C([2H])([2H])[2H] ; CC(C)(C)c1cc(-c2cccc(-c3ccccc3)c2)ccc1N(c1ccc(-c2ccccc2)cc1)c1ccc2c(c1)oc1ccccc12 ; CC(C)(C)c1cc(-c2ccc(-c3ccccc3)cc2)ccc1N(c1ccc(-c2ccccc2)cc1)c1ccc2c(c1)oc1ccccc12]

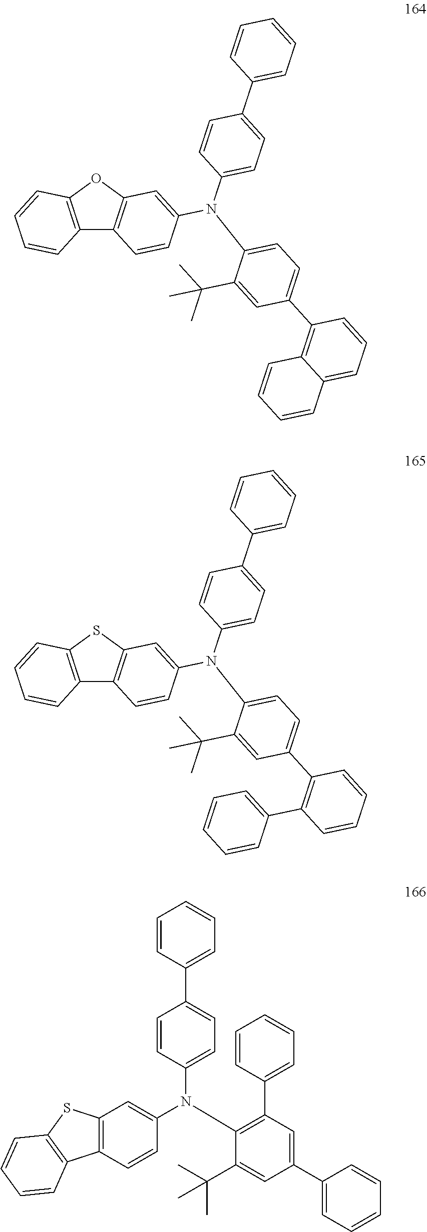 [SMILES: CC(C)(C)c1cc(-c2cccc3ccccc23)ccc1N(c1ccc(-c2ccccc2)cc1)c1ccc2c(c1)oc1ccccc12 ; CC(C)(C)c1cc(-c2ccccc2-c2ccccc2)ccc1N(c1ccc(-c2ccccc2)cc1)c1ccc2c(c1)sc1ccccc12 ; CC(C)(C)c1cc(-c2ccccc2)cc(-c2ccccc2)c1N(c1ccc(-c2ccccc2)cc1)c1ccc2c(c1)sc1ccccc12]

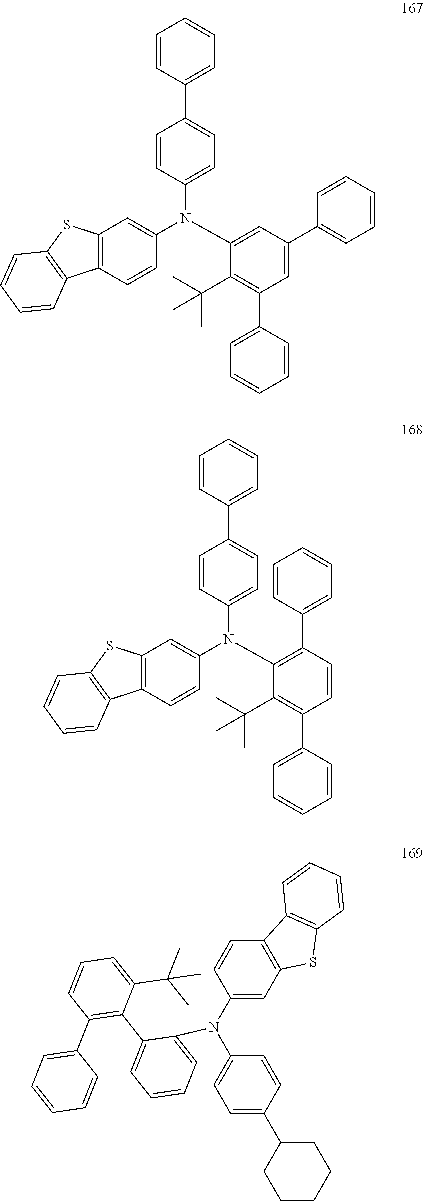 [SMILES: CC(C)(C)c1c(-c2ccccc2)cc(-c2ccccc2)cc1N(c1ccc(-c2ccccc2)cc1)c1ccc2c(c1)sc1ccccc12 ; CC(C)(C)c1c(-c2ccccc2)ccc(-c2ccccc2)c1N(c1ccc(-c2ccccc2)cc1)c1ccc2c(c1)sc1ccccc12 ; CC(C)(C)c1cccc(-c2ccccc2)c1-c1ccccc1N(c1ccc(C2CCCCC2)cc1)c1ccc2c(c1)sc1ccccc12]

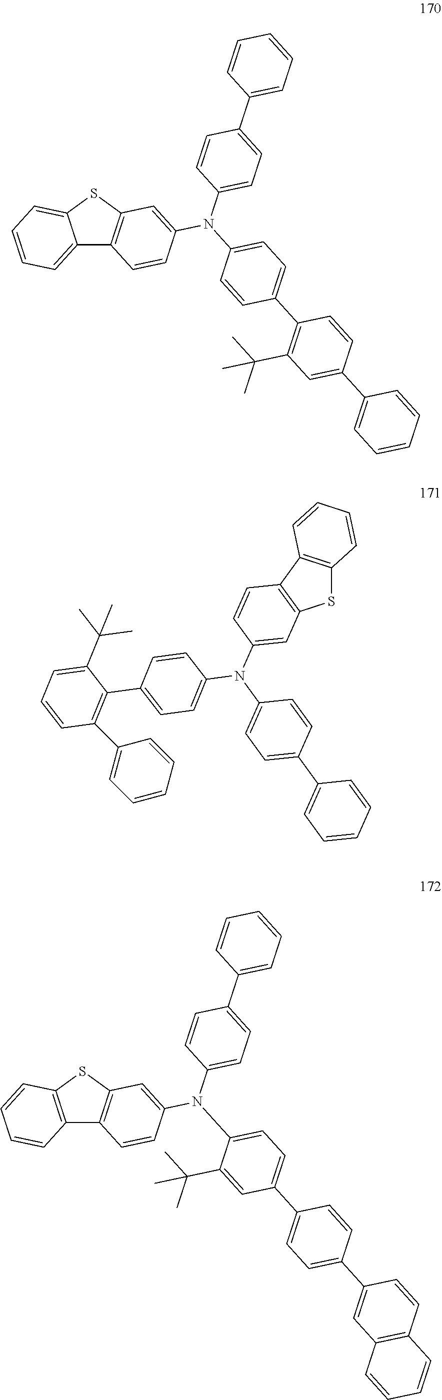 [SMILES: CC(C)(C)c1cc(-c2ccccc2)ccc1-c1ccc(N(c2ccc(-c3ccccc3)cc2)c2ccc3c(c2)sc2ccccc23)cc1 ; CC(C)(C)c1cccc(-c2ccccc2)c1-c1ccc(N(c2ccc(-c3ccccc3)cc2)c2ccc3c(c2)sc2ccccc23)cc1 ; CC(C)(C)c1cc(-c2ccc(-c3ccc4ccccc4c3)cc2)ccc1N(c1ccc(-c2ccccc2)cc1)c1ccc2c(c1)sc1ccccc12]

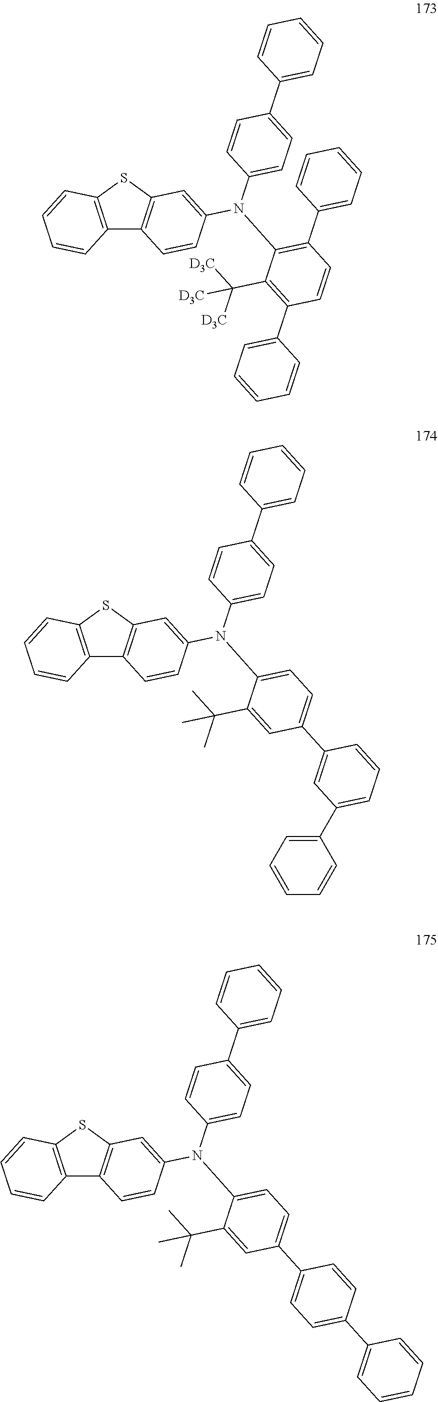 [SMILES: [2H]C([2H])([2H])C(c1c(-c2ccccc2)ccc(-c2ccccc2)c1N(c1ccc(-c2ccccc2)cc1)c1ccc2c(c1)sc1ccccc12)(C([2H])([2H])[2H])C([2H])([2H])[2H] ; CC(C)(C)c1cc(-c2cccc(-c3ccccc3)c2)ccc1N(c1ccc(-c2ccccc2)cc1)c1ccc2c(c1)sc1ccccc12 ; CC(C)(C)c1cc(-c2ccc(-c3ccccc3)cc2)ccc1N(c1ccc(-c2ccccc2)cc1)c1ccc2c(c1)sc1ccccc12]

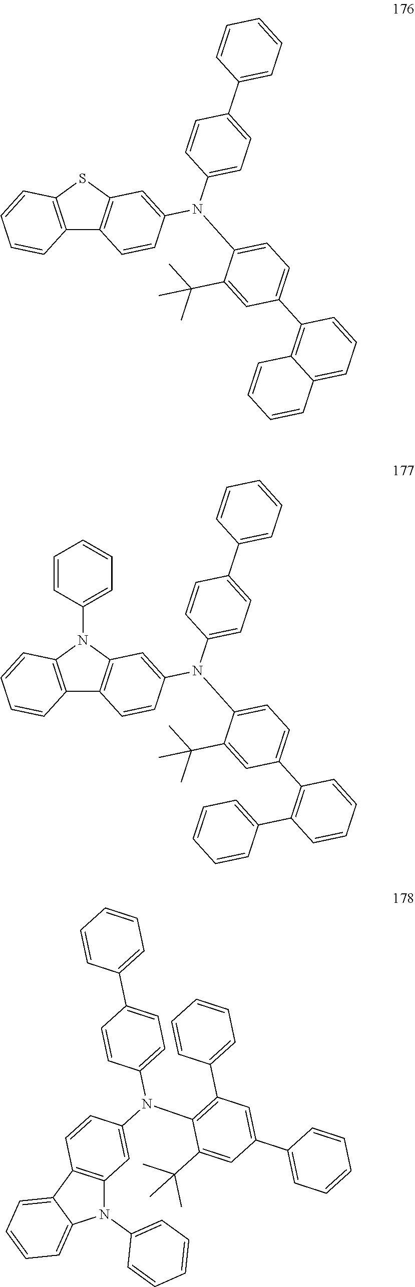 [SMILES: CC(C)(C)c1cc(-c2cccc3ccccc23)ccc1N(c1ccc(-c2ccccc2)cc1)c1ccc2c(c1)sc1ccccc12 ; CC(C)(C)c1cc(-c2ccccc2-c2ccccc2)ccc1N(c1ccc(-c2ccccc2)cc1)c1ccc2c3ccccc3n(-c3ccccc3)c2c1 ; CC(C)(C)c1cc(-c2ccccc2)cc(-c2ccccc2)c1N(c1ccc(-c2ccccc2)cc1)c1ccc2c3ccccc3n(-c3ccccc3)c2c1]

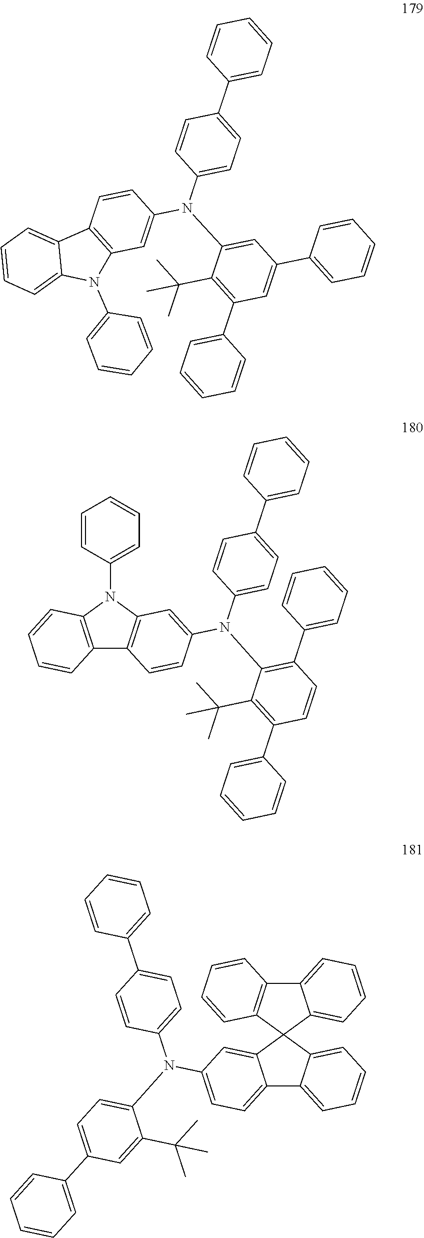 [SMILES: CC(C)(C)c1c(-c2ccccc2)cc(-c2ccccc2)cc1N(c1ccc(-c2ccccc2)cc1)c1ccc2c3ccccc3n(-c3ccccc3)c2c1 ; CC(C)(C)c1c(-c2ccccc2)ccc(-c2ccccc2)c1N(c1ccc(-c2ccccc2)cc1)c1ccc2c3ccccc3n(-c3ccccc3)c2c1 ; CC(C)(C)c1cc(-c2ccccc2)ccc1N(c1ccc(-c2ccccc2)cc1)c1ccc2c(c1)C1(c3ccccc3-c3ccccc31)c1ccccc1-2]

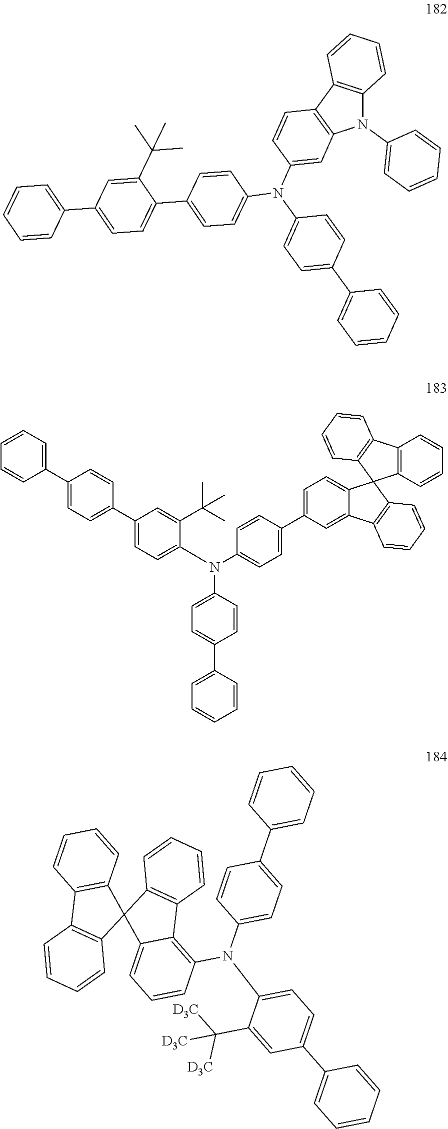 [SMILES: CC(C)(C)c1cc(-c2ccccc2)ccc1-c1ccc(N(c2ccc(-c3ccccc3)cc2)c2ccc3c4ccccc4n(-c4ccccc4)c3c2)cc1 ; CC(C)(C)c1cc(-c2ccc(-c3ccccc3)cc2)ccc1N(c1ccc(-c2ccccc2)cc1)c1ccc(-c2ccc3c(c2)-c2ccccc2C32c3ccccc3-c3ccccc32)cc1 ; [2H]C([2H])([2H])C(c1cc(-c2ccccc2)ccc1N(c1ccc(-c2ccccc2)cc1)c1cccc2c1-c1ccccc1C21c2ccccc2-c2ccccc21)(C([2H])([2H])[2H])C([2H])([2H])[2H]]

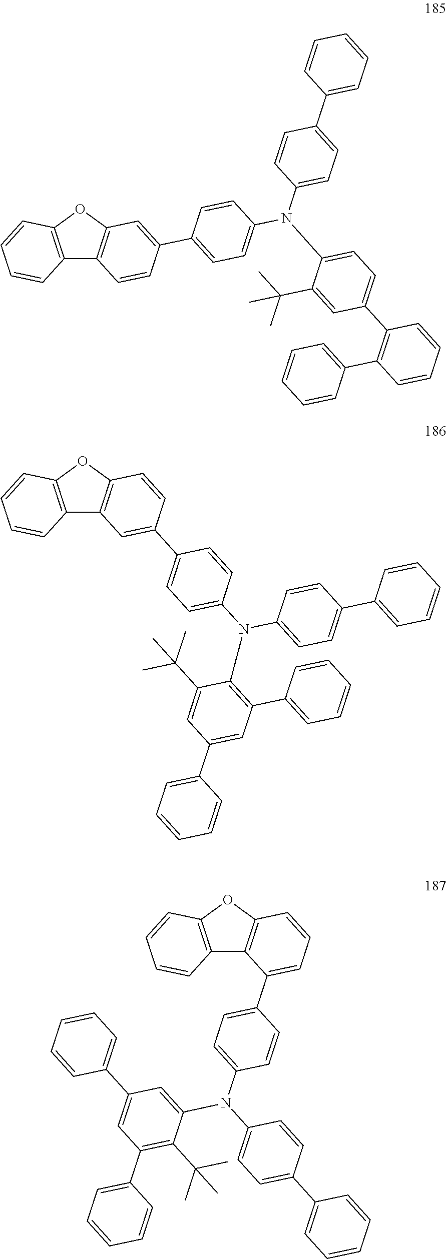 [SMILES: CC(C)(C)c1cc(-c2ccccc2-c2ccccc2)ccc1N(c1ccc(-c2ccccc2)cc1)c1ccc(-c2ccc3c(c2)oc2ccccc23)cc1 ; CC(C)(C)c1cc(-c2ccccc2)cc(-c2ccccc2)c1N(c1ccc(-c2ccccc2)cc1)c1ccc(-c2ccc3oc4ccccc4c3c2)cc1 ; CC(C)(C)c1c(-c2ccccc2)cc(-c2ccccc2)cc1N(c1ccc(-c2ccccc2)cc1)c1ccc(-c2cccc3oc4ccccc4c23)cc1]

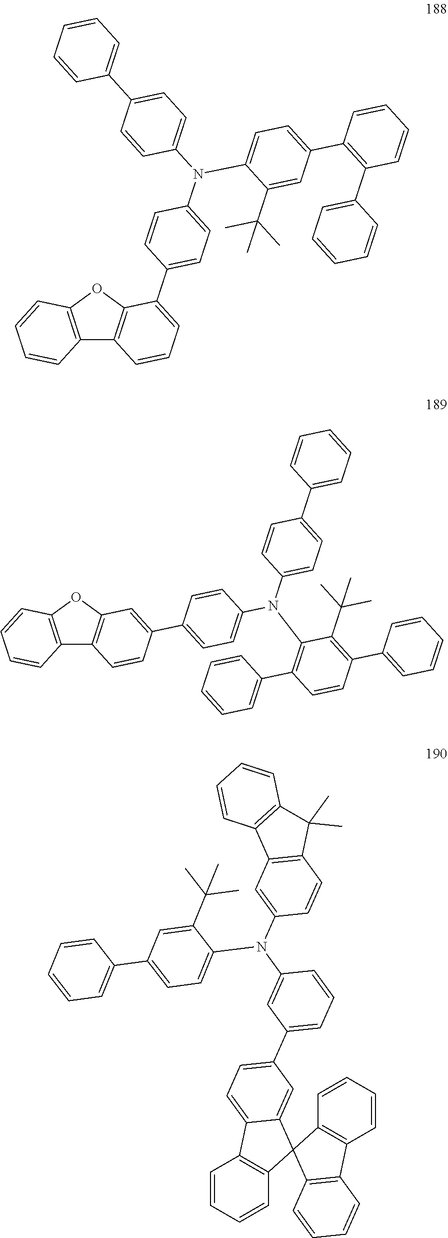 [SMILES: CC(C)(C)c1cc(-c2ccccc2-c2ccccc2)ccc1N(c1ccc(-c2ccccc2)cc1)c1ccc(-c2cccc3c2oc2ccccc23)cc1 ; CC(C)(C)c1c(-c2ccccc2)ccc(-c2ccccc2)c1N(c1ccc(-c2ccccc2)cc1)c1ccc(-c2ccc3c(c2)oc2ccccc23)cc1 ; CC(C)(C)c1cc(-c2ccccc2)ccc1N(c1cccc(-c2ccc3c(c2)C2(c4ccccc4-c4ccccc42)c2ccccc2-3)c1)c1ccc2c(c1)-c1ccccc1C2(C)C]

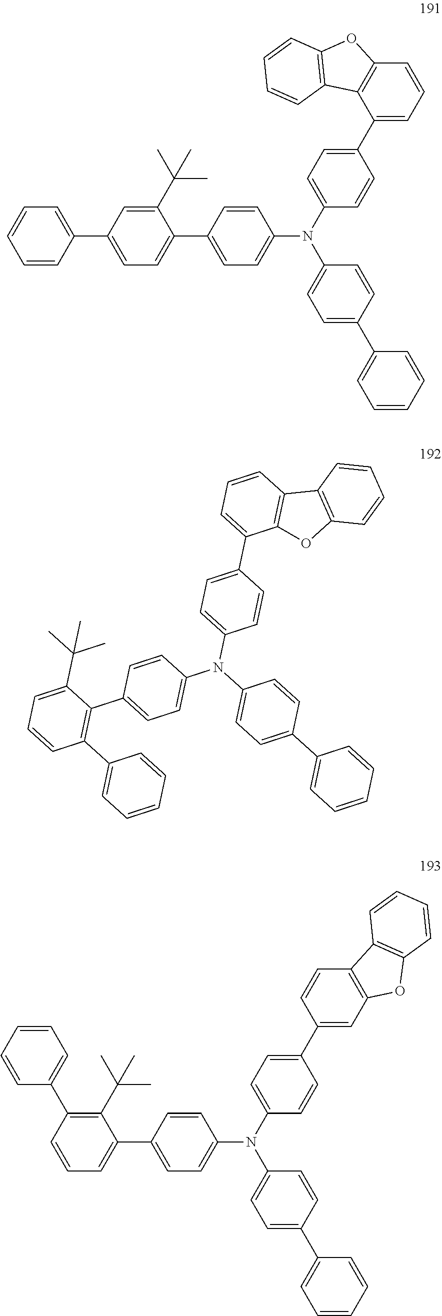 [SMILES: CC(C)(C)c1cc(-c2ccccc2)ccc1-c1ccc(N(c2ccc(-c3ccccc3)cc2)c2ccc(-c3cccc4oc5ccccc5c34)cc2)cc1 ; CC(C)(C)c1cccc(-c2ccccc2)c1-c1ccc(N(c2ccc(-c3ccccc3)cc2)c2ccc(-c3cccc4c3oc3ccccc34)cc2)cc1 ; CC(C)(C)c1c(-c2ccccc2)cccc1-c1ccc(N(c2ccc(-c3ccccc3)cc2)c2ccc(-c3ccc4c(c3)oc3ccccc34)cc2)cc1]

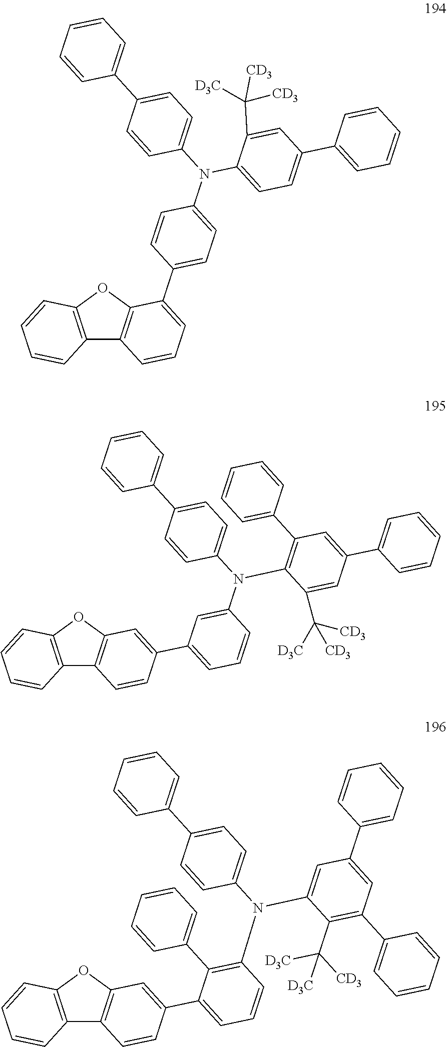 [SMILES: [2H]C([2H])([2H])C(c1cc(-c2ccccc2)ccc1N(c1ccc(-c2ccccc2)cc1)c1ccc(-c2cccc3c2oc2ccccc23)cc1)(C([2H])([2H])[2H])C([2H])([2H])[2H] ; [2H]C([2H])([2H])C(c1cc(-c2ccccc2)cc(-c2ccccc2)c1N(c1ccc(-c2ccccc2)cc1)c1cccc(-c2ccc3c(c2)oc2ccccc23)c1)(C([2H])([2H])[2H])C([2H])([2H])[2H] ; [2H]C([2H])([2H])C(c1c(-c2ccccc2)cc(-c2ccccc2)cc1N(c1ccc(-c2ccccc2)cc1)c1cccc(-c2ccc3c(c2)oc2ccccc23)c1-c1ccccc1)(C([2H])([2H])[2H])C([2H])([2H])[2H]]

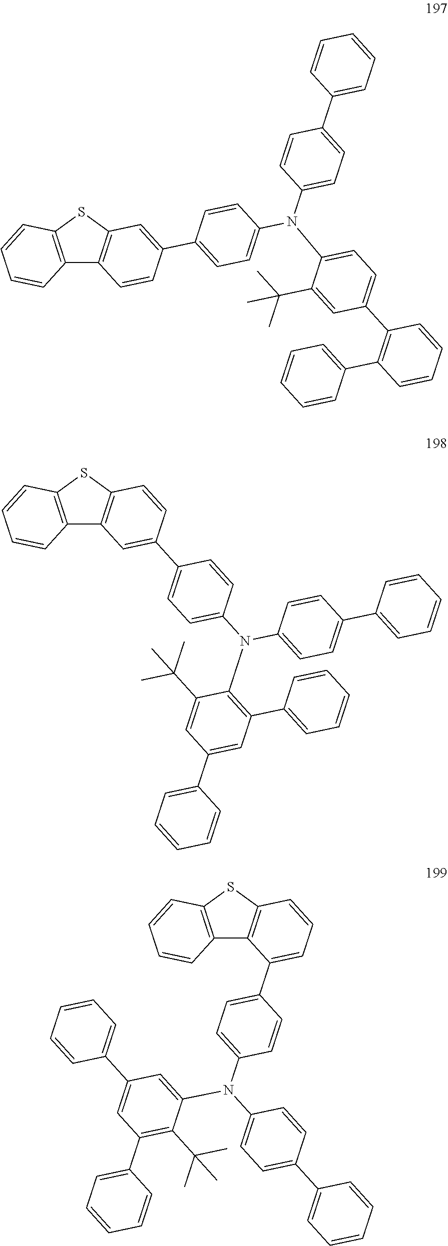 [SMILES: CC(C)(C)c1cc(-c2ccccc2-c2ccccc2)ccc1N(c1ccc(-c2ccccc2)cc1)c1ccc(-c2ccc3c(c2)sc2ccccc23)cc1 ; CC(C)(C)c1cc(-c2ccccc2)cc(-c2ccccc2)c1N(c1ccc(-c2ccccc2)cc1)c1ccc(-c2ccc3sc4ccccc4c3c2)cc1 ; CC(C)(C)c1c(-c2ccccc2)cc(-c2ccccc2)cc1N(c1ccc(-c2ccccc2)cc1)c1ccc(-c2cccc3sc4ccccc4c23)cc1]

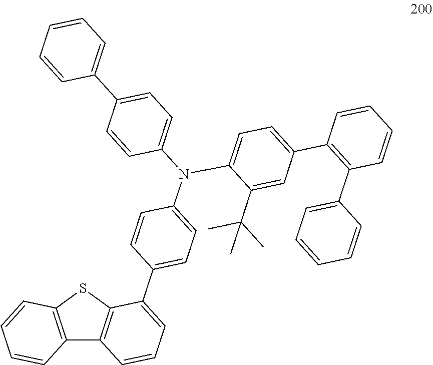 [SMILES: CC(C)(C)c1cc(-c2ccccc2-c2ccccc2)ccc1N(c1ccc(-c2ccccc2)cc1)c1ccc(-c2cccc3c2sc2ccccc23)cc1]

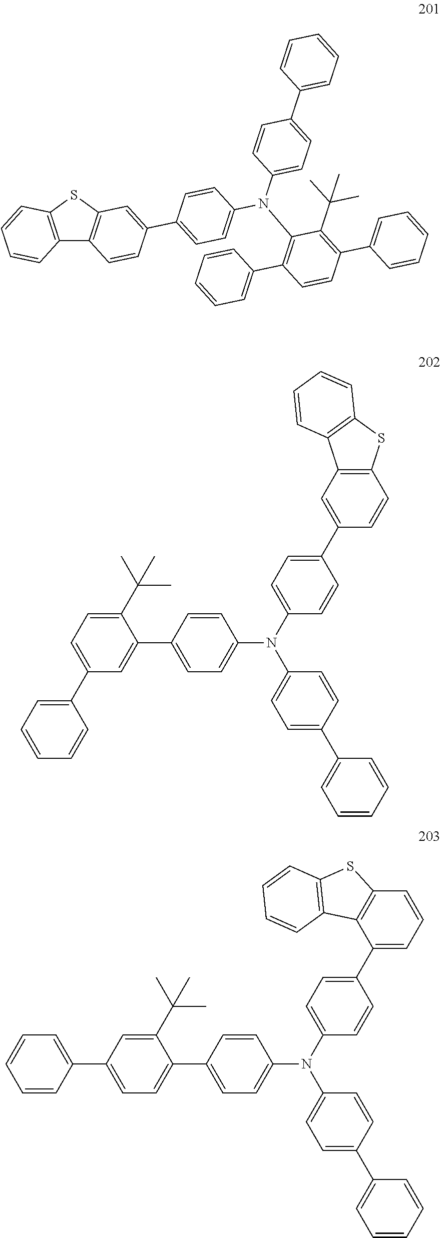 [SMILES: CC(C)(C)c1c(-c2ccccc2)ccc(-c2ccccc2)c1N(c1ccc(-c2ccccc2)cc1)c1ccc(-c2ccc3c(c2)sc2ccccc23)cc1 ; CC(C)(C)c1ccc(-c2ccccc2)cc1-c1ccc(N(c2ccc(-c3ccccc3)cc2)c2ccc(-c3ccc4sc5ccccc5c4c3)cc2)cc1 ; CC(C)(C)c1cc(-c2ccccc2)ccc1-c1ccc(N(c2ccc(-c3ccccc3)cc2)c2ccc(-c3cccc4sc5ccccc5c34)cc2)cc1]

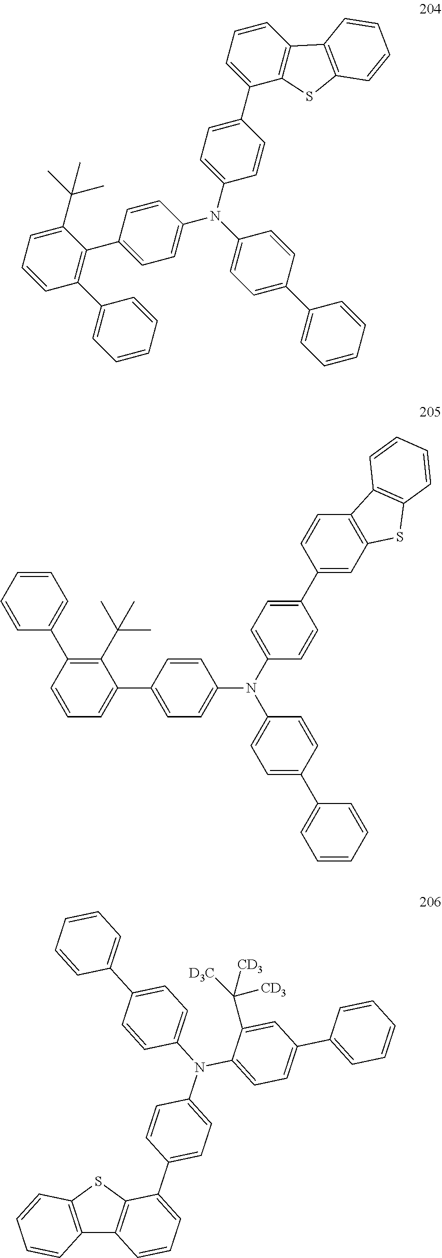 [SMILES: CC(C)(C)c1cccc(-c2ccccc2)c1-c1ccc(N(c2ccc(-c3ccccc3)cc2)c2ccc(-c3cccc4c3sc3ccccc34)cc2)cc1 ; CC(C)(C)c1c(-c2ccccc2)cccc1-c1ccc(N(c2ccc(-c3ccccc3)cc2)c2ccc(-c3ccc4c(c3)sc3ccccc34)cc2)cc1 ; [2H]C([2H])([2H])C(c1cc(-c2ccccc2)ccc1N(c1ccc(-c2ccccc2)cc1)c1ccc(-c2cccc3c2sc2ccccc23)cc1)(C([2H])([2H])[2H])C([2H])([2H])[2H]]

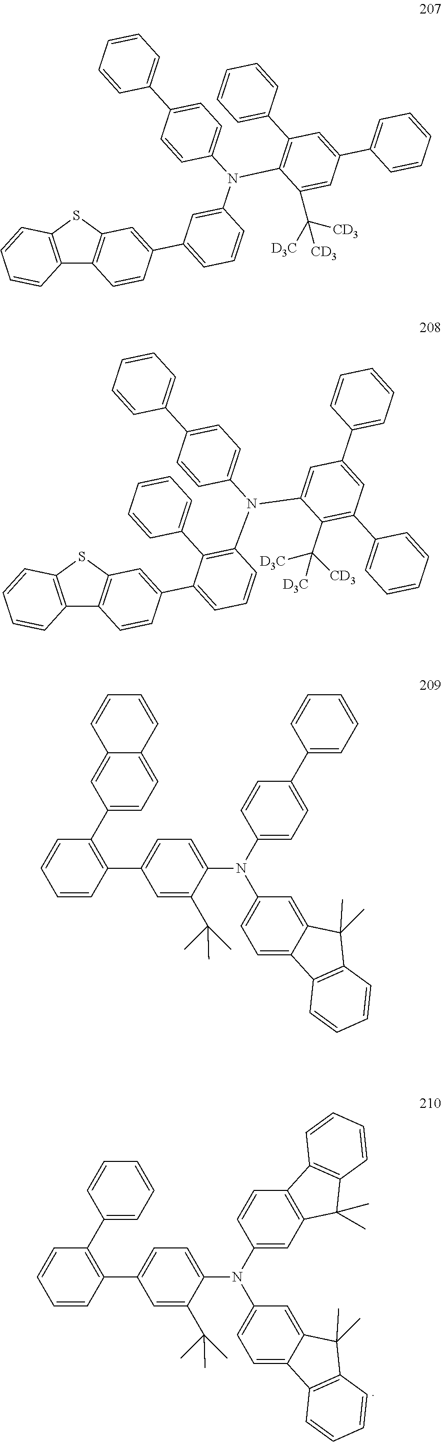 [SMILES: [2H]C([2H])([2H])C(c1cc(-c2ccccc2)cc(-c2ccccc2)c1N(c1ccc(-c2ccccc2)cc1)c1cccc(-c2ccc3c(c2)sc2ccccc23)c1)(C([2H])([2H])[2H])C([2H])([2H])[2H] ; [2H]C([2H])([2H])C(c1c(-c2ccccc2)cc(-c2ccccc2)cc1N(c1ccc(-c2ccccc2)cc1)c1cccc(-c2ccc3c(c2)sc2ccccc23)c1-c1ccccc1)(C([2H])([2H])[2H])C([2H])([2H])[2H] ; CC(C)(C)c1cc(-c2ccccc2-c2ccc3ccccc3c2)ccc1N(c1ccc(-c2ccccc2)cc1)c1ccc2c(c1)C(C)(C)c1ccccc1-2 ; CC(C)(C)c1cc(-c2ccccc2-c2ccccc2)ccc1N(c1ccc2c(c1)C(C)(C)c1ccccc1-2)c1ccc2c(c1)C(C)(C)c1ccccc1-2]

13. An electronic element, comprising an anode and a cathode disposed opposite to each other, and a functional layer disposed between the anode and the cathode; wherein the functional layer contains the organic compound according to claim 1.

14. The electronic element according to claim 13, wherein the functional layer comprises a hole transport layer, which contains the organic compound;
optionally, the electronic element is an organic electroluminescent device or a photoelectric conversion;
optionally, the electronic element is a red organic electroluminescent device.

15. An electronic apparatus, comprising the electronic element of claim 13.
